(19) United States
(12) Patent Application Publication
(10) Pub. No.: US 2026/0110037 A1
(43) Pub. Date: Apr. 23, 2026

(54) FRAGMENTOMICS IN CEREBROSPINAL FLUID

(71) Applicant: Centre for Novostics, Shatin (HK)

(72) Inventors: Yuk-Ming Dennis Lo, Hong Kong SAR (CN); Kwan Chee Chan, Hong Kong SAR (CN); Peiyong Jiang, Tai Po (CN); Yasine Malki, Hong Kong SAR (CN)

(21) Appl. No.: 19/319,134

(22) Filed: Sep. 4, 2025

Related U.S. Application Data
(60) Provisional application No. 63/690,764, filed on Sep. 4, 2024.

(51) Int. Cl. C12Q 1/6886

(52) U.S. Cl. CPC C12Q 1/6886; C12Q 2600/154

(57) ABSTRACT

Various embodiments are directed to the analysis of fragmentation patterns of cell-free DNA (cfDNA) circulating in cerebrospinal fluid (CSF) and the potential applications. CSF is an important liquid biopsy sample used to study the central nervous system and related disorders, such as infection and malignancies. The characterization of fragmentation patterns of cfDNA in CSF includes the size profile, end motif, cleavage profiles, and the determination of epigenetic features, including methylation. Various applications can use one or more properties of fragmentation pattern, for example, in the determination of the proportional contribution of a particular cell types in the CSF cfDNA pool. Another purpose is the diagnosis of pathology in the central nervous system, by the detection of clinically relevant DNA (e.g., tumor fraction, pathogen). DNA fragments in CSF can be analyzed in various ways, including using short-read sequencing, and/or long-read sequencer technologies.

### CROSS REFERENCE TO RELATED APPLICATIONS

This application claims the benefit of, and priority to, U.S. Provisional Application No. 63/690,764, filed on Sep. 4, 2024, which is hereby incorporated by reference in its entirety for all purposes.

### BACKGROUND

Plasma DNA is believed to consist of cell-free DNA shed from multiple tissues in the body, including but not limited to, hematopoietic tissues, brain, liver, lung, colon, pancreas and so on (Sun et al, Proc Natl Acad Sci USA. 2015; 112:E5503-12; Lehmann-Werman et al, Proc Natl Acad Sci USA. 2016; 113: E1826-34; Moss et al, Nat Commun. 2018; 9: 5068). Plasma DNA molecules (a type of cell-free DNA molecules) have been demonstrated to be generated through a non-random process, for example, its size profile showing 166-bp major peaks and 10-bp periodicities occurring in the smaller peaks (Lo et al, Sci Transl Med. 2010; 2:61ra91; Jiang et al, Proc Natl Acad Sci USA. 2015; 112:E1317-25).

Techniques have been used to determine various properties of the cell-free DNA and of the subject from which a sample has been obtained. It is desirable to identify additional techniques to increase accuracy and to determine new properties.

### BRIEF SUMMARY

Various embodiments are directed to the analysis of fragmentation patterns of cell-free DNA (cfDNA) circulating in cerebrospinal fluid (CSF) and the potential applications. CSF is an important liquid biopsy sample used to study the central nervous system and related disorders, such as infection and malignancies. The characterization of fragmentation patterns of cfDNA in CSF includes the size profile, end motif, cleavage profiles, and the determination of epigenetic features, including methylation. Various applications can use one or more properties of fragmentation pattern, for example, in the determination of the proportional contribution of a particular cell types in the CSF cfDNA pool. Another purpose is the diagnosis of pathology in the central nervous system, by the detection of clinically relevant DNA (e.g., tumor fraction, pathogen). DNA fragments in CSF can be analyzed in various ways, including using short-read sequencing, and/or long-read sequencer technologies.

A method may comprise receiving a biological sample of a subject. The biological sample can be cerebrospinal fluid, plasma, or serum. The method may further comprise measuring a sample size profile of cell-free DNA fragments in the biological sample. The method can further comprise comparing the sample size profile to one or more reference size profiles. The one or more reference size profiles may include a first reference size profile. The first reference size profile can be determined from cell-free DNA fragments in one or more first reference samples measured from one or more first reference subjects having a benign tumor or a glioma. The method may further comprise determining a classification of whether the subject has the benign tumor or the glioma based on a comparison.

Each of the following features can be separately incorporated as part of the method or can be incorporated together with one or more other following features. The biological sample may be plasma or serum. The first reference size profile can correspond to glioma. The first reference size profile may have a greater proportion of cell-free DNA fragments having a size of 110-120 bases than a second reference profile corresponding to the benign tumor. The first reference size profile may have a greater proportion of cell-free DNA fragments having a size of 120-130 bases than a second reference profile corresponding to the benign tumor. The first reference size profile may have a greater proportion of cell-free DNA fragments having a size of 130-140 bases than a second reference profile corresponding to the benign tumor. The first reference size profile may have a greater proportion of cell-free DNA fragments having a size of 140-150 bases than a second reference profile corresponding to the benign tumor. The first reference size profile may have a greater proportion of cell-free DNA fragments having a size of 150-160 bases than a second reference profile corresponding to the benign tumor. The first reference size profile may have a greater proportion of cell-free DNA fragments having a size of 160-170 bases than a second reference profile corresponding to the benign tumor. The first reference size profile may have a greater proportion of cell-free DNA fragments having a size of 170-180 bases than a second reference profile corresponding to the benign tumor. The first reference size profile may have a lower proportion of cell-free DNA fragments having a size of 180-190 bases than a second reference profile corresponding to the benign tumor. The first reference size profile may have a lower proportion of cell-free DNA fragments having a size of 190-200 bases than a second reference profile corresponding to the benign tumor. The first reference size profile may have a lower proportion of cell-free DNA fragments having a size of 200-300 bases than a second reference profile corresponding to the benign tumor. The first reference size profile may have a lower proportion of cell-free DNA fragments having a size of 300-400 bases than a second reference profile corresponding to the benign tumor. The biological sample may be cerebrospinal fluid. The first reference size profile can correspond to glioma. The first reference size profile may have a greater proportion of cell-free DNA fragments having a size of 160-170 bases than a second reference profile corresponding to the benign tumor. The first reference size profile may have a greater proportion of cell-free DNA fragments having a size of 170-180 bases than a second reference profile corresponding to the benign tumor. The first reference size profile may have a greater proportion of cell-free DNA fragments having a size of 180-190 bases than a second reference profile corresponding to the benign tumor. The first reference size profile may have a greater proportion of cell-free DNA fragments having a size of 190-200 bases than a second reference profile corresponding to the benign tumor. The first reference size profile may have a greater proportion of cell-free DNA fragments having a size of 200-300 bases than a second reference profile corresponding to the benign tumor. The first reference size profile may have a lower proportion of cell-free DNA fragments having a size of 50-60 bases than a second reference profile corresponding to the benign tumor. The first reference size profile may have a lower proportion of cell-free DNA fragments having a size of 60-70 bases than a second reference profile corresponding to the benign tumor. The first reference size profile may have a lower proportion of cell-free DNA fragments having a size of 70-80 bases than a second reference profile corresponding to the benign tumor. The first reference size profile may have a lower proportion of cell-free DNA fragments having a size of 80-90 bases than a second reference profile corresponding to the benign tumor. The first reference size profile may have a lower proportion of cell-free DNA fragments having a size of 90-100 bases than a second reference profile corresponding to the benign tumor. The first reference size profile may have a lower proportion of cell-free DNA fragments having a size of 100-110 bases than a second reference profile corresponding to the benign tumor. The first reference size profile may have a lower proportion of cell-free DNA fragments having a size of 110-120 bases than a second reference profile corresponding to the benign tumor. The first reference size profile may have a lower proportion of cell-free DNA fragments having a size of 120-130 bases than a second reference profile corresponding to the benign tumor. The first reference size profile may have a lower proportion of cell-free DNA fragments having a size of 130-140 bases than a second reference profile corresponding to the benign tumor. The first reference size profile may have a lower proportion of cell-free DNA fragments having a size of 140-150 bases than a second reference profile corresponding to the benign tumor. The first reference size profile may have a lower proportion of cell-free DNA fragments having a size of 300-400 bases than a second reference profile corresponding to the benign tumor. The first reference size profile may have a lower proportion of cell-free DNA fragments having a size of 400-600 bases than a second reference profile corresponding to the benign tumor.

A method may comprise receiving a sample of cerebrospinal fluid from a subject. The method can further comprise measuring a sample size profile of cell-free DNA fragments in the sample of cerebrospinal fluid. The method may further comprise comparing the sample size profile to one or more reference size profiles. The one or more reference size profiles can include a first reference size profile. The first reference size profile may be determined from cell-free DNA fragments in one or more first reference samples of cerebrospinal fluid measured from one or more first reference subjects having a brain tumor. The method can further comprise detecting whether the subject has the brain tumor based on the comparison.

Each of the following features can be separately incorporated as part of the method or can be incorporated together with one or more other following features. The one or more reference size profiles may further include a second reference size profile. The second reference size profile can be determined from cell-free DNA fragments in one or more second reference samples of cerebrospinal fluid measured from one or more second reference subjects having high intra-cranial pressure. The detecting may determine whether the subject has high intra-cranial pressure or the brain tumor. Measuring the sample size profile can comprise a physical separation technique. The physical separation technique may comprise filtration and/or electrophoresis. Measuring the sample size profile can comprise sequencing the cell-free DNA fragments to obtain sequence reads. Measuring the sample size profile may further comprise measuring sizes of the cell-free DNA fragments using the sequence reads. Measuring the sample size profile can further comprise generating the sample size profile using amounts of cell-free DNA fragments having a set of sizes. Any sequencing described herein may use a single-stranded library preparation or a double-stranded preparation. Measuring the sizes of the cell-free DNA fragments using the sequence reads can comprise aligning paired-end reads to a reference genome. Comparing the sample size profile to the one or more reference size profiles may comprise inputting the sample size profile into a machine learning model that is trained using a set of reference size profiles that include the one or more reference size profiles. The machine learning model can comprise a support vector machine. The sample size profile may comprise a size ratio of a first amount of the cell-free DNA fragments having a first size relative to a second amount of the cell-free DNA fragments having a second size. The second size can have a size range with a larger upper bound than the first size. Comparing the sample size profile to the one or more reference size profiles may comprise comparing the size ratio to a reference ratio of the one or more reference size profiles. The first size may be a first range having an upper bound that is between 120 bases and 180 bases. A size can be a size range.

A method may comprise receiving a sample of cerebrospinal fluid from a subject. The method can further comprise sequencing a set of cell-free DNA fragments to obtain sequence reads. The sequence reads may include ending sequences corresponding to ends of the set of cell-free DNA fragments. The method can further comprise generating a sample end-motif profile using, for each cell-free DNA fragment of the set of cell-free DNA fragments, an end motif for each of one or more ending sequences of the cell-free DNA fragment. The sample end-motif profile may represent one or more end motifs. The method may further comprise comparing the sample end-motif profile to one or more reference end-motif profiles. The one or more reference end-motif profiles can include a first reference end-motif profile. The first reference end-motif profile may be determined from cell-free DNA fragments in one or more first reference samples of cerebrospinal fluid measured from one or more first reference subjects having a brain tumor. The method can further comprise detecting a classification of a level of the brain tumor for the subject based on the comparison.

Each of the following features can be separately incorporated as part of the method or can be incorporated together with one or more other following features. The sample end-motif profile may represent at least four end motifs. Comparing the sample end-motif profile to the one or more reference end-motif profiles can comprise comparing a first aggregate for the sample end-motif profile to a second aggregate for the one or more reference end-motif profiles. Generating the sample end-motif profile may comprise generating the first aggregate using the ending sequences matching any one of the one or more end motifs. The method may further comprise receiving another sample of the subject, wherein the other sample can be plasma or serum. The method can further comprise sequencing a plurality of cell-free DNA fragments to obtain other sequence reads. The method may further comprise generating another end-motif profile using the other sequence reads. The other end-motif profile can represent the one or more end motifs in the other sample. Comparing the sample end-motif profile to one or more reference end-motif profiles may comprise generating a differential end-motif profile between the sample end-motif profile and the other end-motif profile. Comparing the sample end-motif profile to one or more reference end-motif profiles can further comprise comparing the differential end-motif profile to a reference differential end-motif profile generated using the first reference end-motif profile and another reference end-motif profile. The differential end-motif profile may comprise a change between the sample of cerebrospinal fluid and the other sample. The change can be compared to a reference change between the first reference end-motif profile and the other reference end-motif profile for the one or more first reference subjects having the brain tumor. The sample end-motif profile may represent a set of end motifs. The one or more reference end-motif profiles can comprise a set of reference F-profiles. The method may further comprise storing the set of reference F-profiles. Each reference F-profile of the set can identify, for each nucleotide of a set of nucleotides, a proportion of cell-free DNA molecules that end in the nucleotide. Each reference F-profile of the set may be associated with a type of fragmentation factors. Comparing the sample end-motif profile to the one or more reference end-motif profiles can comprise determining proportional contributions of the set of reference F-profiles whose proportional aggregation provides the sample end-motif profile. The proportional contributions may sum to one. Detecting the classification of the brain tumor for the subject can be based on a proportional contribution associated with a reference F-profile of the set of reference F-profiles. The subject may be determined to have the brain tumor based on the proportional contribution exceeding a threshold. The sample end-motif profile may represent a set of end motifs. Comparing the sample end-motif profile to the one or more reference end-motif profiles can comprise inputting the sample end-motif profile into a machine learning model. The machine learning model may be trained using a set of reference size profiles that include the one or more reference end-motif profiles. The machine learning model can comprise a support vector machine or clustering. Generating the sample end-motif profile may comprise generating, for each end motif of the set of end motifs, an aggregate of the ending sequences having the end motif. The one or more reference end-motif profiles may further include a second reference end-motif profile. The second reference end-motif profile can be determined from cell-free DNA fragments in one or more second reference samples of cerebrospinal fluid measured from one or more second reference subjects having high intra-cranial pressure. The one or more end motifs of the sample end-motif profile may include pre-end motif(s), EM5 end motif(s), EM3 end motif(s), post-end motif(s), or a combination thereof. The classification of the brain tumor may be whether the subject has the brain tumor. The classification of the brain tumor can be whether the brain tumor is benign or glioma. The one or more first reference samples may be measured from one or more first reference subjects having a benign tumor or a glioma. The one or more first reference samples may be measured from a first cohort of reference samples from subjects having a benign tumor and a second cohort of reference samples from subjects having glioma.

A method may comprise receiving a sample of cerebrospinal fluid from a subject. The method can further comprise performing an assay on a set of cell-free DNA fragments to obtain sequence reads. The sequence reads may include ending sequences corresponding to ends of the set of cell-free DNA fragments. For each of the set of cell-free DNA fragments, the method can comprise determining, using the sequence reads, a sequence motif for each of one or more ends of the cell-free DNA fragment. An end of a cell-free DNA fragment may have a first position at an outermost position, a second position that is next to the first position, and a third position that is next to the second position. The method can further comprise determining a first amount of a first set of one or more end sequence motifs of the set of cell-free DNA fragments. The first set of one or more end sequence motifs may have C at the first position and G at the second position. The first set of one or more end sequence motifs can have C at the second position and G at the third position. The method may further comprise determining a classification of whether the subject has a benign tumor or glioma based on a comparison of the first amount to a reference value. The reference value can be trained using a first cohort of reference samples from subjects having a benign tumor and a second cohort of reference samples from subjects having glioma.

Each of the following features can be separately incorporated as part of the method or can be incorporated together with one or more other following features. The first set of one or more end sequence motifs may have C at the first position and G at the second position. The first set of one or more end sequence motifs can include all end sequence motifs having C at the first position and G at the second position. The method may further comprise determining a respective amount of each 3-mer end sequence motif that has a C at the first position and that has G at the second position, thereby determining respective amounts. The method can further comprise generating a feature vector including the respective amounts, which include the first amount. The method may further comprise inputting the feature vector into a machine learning model as part of determining the classification. The machine learning model can be trained using the first cohort of reference samples from subjects having the benign tumor and the second cohort of reference samples from subjects having glioma. The first set of one or more end sequence motifs may have C at the second position and G at the third position. The first set of one or more end sequence motifs can include all end sequence motifs having C at the second position and G at the third position. The method may further comprise determining a respective amount of each 3-mer end sequence motif that has a C at the second position and G at the third position, thereby determining respective amounts. The method can further comprise generating a feature vector including the respective amounts, which include the first amount. The method may further comprise inputting the feature vector into a machine learning model as part of determining the classification. The machine learning model can be trained using the first cohort of reference samples from subjects having the benign tumor and the second cohort of reference samples from subjects having glioma. The method may further comprise determining a second amount of a second set of one or more end sequence motifs of the set of cell-free DNA fragments. The second set of one or more end sequence motifs can have C at the first position and G at the second position. The second set of one or more end sequence motifs may have C at the second position and G at the third position. The classification can be determined using the first amount and the second amount. The first set of one or more end sequence motifs may have C at the first position and G at the second position, and the second set of one or more end sequence motifs can have C at the second position and G at the third position. The classification can use a ratio of the first amount and the second amount, can use a difference of the first amount and the second amount, or can use a machine learning model that receives the first amount and the second amount as separate inputs. The set of cell-free DNA fragments may each be located within one or more regions that are each hypermethylated or hypomethylated for glioma. The one or more regions can each be hypermethylated. The one or more regions may each be hypomethylated. The method may further comprise determining another amount of another set of one or more end sequence motifs of another set of cell-free DNA fragments. The other set of cell-free DNA fragments may each be located within one or more regions that are each hypermethylated for glioma. Determining the classification of whether the subject has a benign tumor or glioma may further use the other amount. The first set of one or more end sequence motifs can include a plurality of end sequence motifs. The assay may comprise sequencing or digital PCR.

A method may comprise receiving a sample of cerebrospinal fluid from a subject. The method can further comprise performing a methylation-aware assay on a set of cell-free DNA fragments to obtain sequence reads. The methylation-aware assay may further obtain a methylation status of one or more sites for each of the set of cell-free DNA fragments. The method may thereby obtain methylation statuses of the set of cell-free DNA fragments at a set of sites. The set of cell-free DNA fragments can each be located within one or more regions that are each hypermethylated or hypomethylated for glioma. The method may further comprise determining a methylation level using the methylation statuses of the set of cell-free DNA fragments at the set of sites within the one or more regions. The method can further comprise determining a classification of whether the subject has a benign tumor or glioma based on a comparison of the methylation level to a reference value. The reference value may be trained using a first cohort of reference samples from subjects having a benign tumor and a second cohort of reference samples from subjects having glioma.

Each of the following features can be separately incorporated as part of the method or can be incorporated together with one or more other following features. The one or more regions may each be hypermethylated. The classification can be that the subject has glioma when the methylation level is greater than the reference value. The one or more regions may each be hypomethylated. The classification can be that the subject has glioma when the methylation level is less than the reference value. The method may further comprise determining another methylation level using the methylation statuses of another set of cell-free DNA fragments at another set of sites within one or more other regions that are each hypermethylated for glioma. Determining the classification of whether the subject has a benign tumor or glioma may further use the other methylation level. The methylation-aware assay can comprise methylation-aware sequencing. The methylation-aware sequencing may comprise bisulfite sequencing, sequencing after treatment using methylation-sensitive restriction enzymes, or single molecule techniques. The methylation level can be a methylation density at the set of sites within the one or more regions. The one or more regions may be a plurality of regions.

A method may comprise receiving a sample of cerebrospinal fluid from a subject. The method can further comprise performing an assay on a set of cell-free DNA fragments to obtain sequence reads. The method may further comprise aligning the sequence reads to a reference genome. The method can further comprise detecting one or more genomic regions that have a copy number aberration based on a copy number of sequence reads that align to each of the one or more genomic regions. The method may further comprise measuring a fraction of the set of cell-free DNA fragments from a brain tumor based on a separation of the copy number of each of the one or more genomic regions from a reference copy number for no aberration.

Each of the following features can be separately incorporated as part of the method or can be incorporated together with one or more other following features. Detecting the one or more genomic regions having the copy number aberration may comprise, for each of a plurality of genomic regions, determining a respective amount of DNA fragments within the genomic region from sequence tags having a genomic position within the genomic region. Detecting the one or more genomic regions having the copy number aberration can further comprise normalizing the respective amount to obtain a respective density. Detecting the one or more genomic regions having the copy number aberration may further comprise comparing the respective density to a reference density to identify whether the genomic region exhibits a 1-copy loss or a 1-copy gain. The method can further comprise calculating a first density from one or more respective densities identified as exhibiting a 1-copy loss or from one or more respective densities identified as exhibiting a 1-copy gain. Measuring the fraction of the set of cell-free DNA fragments from the brain tumor may comprise comparing the first density to another density to obtain a differential. The differential can be normalized with the reference density. Comparing the respective density to the reference density to identify whether the genomic region exhibits a 1-copy loss or a 1-copy gain may include computing a difference between the respective density and the reference density. Comparing the respective density to the reference density to identify whether the genomic region exhibits a 1-copy loss or a 1-copy gain can further include comparing the difference to a cutoff value. The differential may be normalized with the reference density by dividing the differential by the reference density. The other density can be the reference density. Measuring the fraction may further include multiplying the differential by two. The first density can be calculated using respective densities identified as exhibiting a 1-copy gain. The another density may be a second density calculated from respective densities identified as exhibiting a 1-copy loss. The differential can be normalized with the reference density by computing a first ratio of the first density and the reference density and by computing a second ratio of the second density and the reference density. The differential may be between the first ratio and the second ratio. Comparing the respective density to the reference density to identify whether the genomic region exhibits a 1-copy loss or a 1-copy gain can include fitting peaks to a distribution curve of a histogram of the respective densities. The first density may correspond to a first peak and the second density may correspond to a second peak. All genomic regions determined to exhibit a statistically significant gain in the respective density relative to the reference density can be identified as exhibiting a 1-copy gain. Normalizing the respective amount to obtain a respective density may include using a same total number of aligned reference tags to determine the respective density and the reference density. Normalizing the respective amount to obtain a respective density can include dividing the respective amount by a total number of aligned reference tags. The plurality of genomic regions may each have a same length. The genomic regions can be non-overlapping. The assay may comprise sequencing or digital PCR.

A method of analyzing a sample of cerebrospinal fluid from a subject may comprise, for each of M tissue types, obtaining N tissue-specific methylation levels at N genomic sites, with M being greater than two and N being greater than or equal to M. The tissue-specific methylation levels can form a matrix A of dimensions N by M. The method may further comprise receiving the sample of cerebrospinal fluid from the subject. The method can further comprise performing methylation-aware sequencing on a set of cell-free DNA fragments to obtain sequence reads. The method may further comprise locating, using the sequence reads, the set of cell-free DNA fragments in a reference genome. The method can further comprise measuring N mixture methylation levels at the N genomic sites using a first group of the set of cell-free DNA fragments that are each located at any one of the N genomic sites. The N mixture methylation levels may form a methylation vector b. The method can further comprise obtaining a composition vector x that provides the methylation vector b for the matrix A. For each of one or more components of the composition vector x, the method may comprise using the component to determine an amount of a corresponding tissue type of the M tissue types in the sample of cerebrospinal fluid.

Each of the following features can be separately incorporated as part of the method or can be incorporated together with one or more other following features. The method may further comprise identifying the N genomic sites. For one or more other samples, a first set of the N genomic sites may each have a coefficient of variation of methylation levels of at least 0.15 across the M tissue types. The first set of the N genomic sites can each have a difference between a maximum and a minimum methylation level for the M tissue types that exceeds 0.1. The first set may include at least 10 genomic sites. At least 10 of the N genomic sites can each have a coefficient of variation of methylation levels of at least 0.25 across the M tissue types. At least 10 of the N genomic sites may each have the difference between the maximum and the minimum methylation level for the M tissue types that exceeds 0.2. A first component of the one or more components can correspond to a brain tissue type. The method may further comprise comparing a first amount of the brain tissue type in the mixture to a threshold amount to determine a classification of whether the subject has a brain cancer. The threshold amount may be determined based on amounts of the brain tissue type in mixtures of a first set of organisms that are healthy for the brain tissue type or have high intracranial pressure and of a second set of organisms that have the brain cancer. A second set of the N genomic sites may each have a methylation level in one tissue type that is different from methylation levels in other tissue types by at least a threshold level. The second set of the N genomic sites can include at least 10 genomic sites. The threshold level may correspond to a difference of the methylation level in the one tissue type from a mean of the methylation levels in the other tissue types by at least a specified number of standard deviations. The N tissue-specific methylation levels at the N genomic sites can be obtained from a database. Locating the set of cell-free DNA fragments in the reference genome may comprise aligning the sequence reads to the reference genome. The N mixture methylation levels can be measured using sequence reads that each aligns to at least one of the N genomic sites of the reference genome. Solving for the composition vector x may include solving Ax=b. N can be greater than M. Solving Ax=b may involve a least squares optimization.

A method may comprise receiving a biological sample of cerebrospinal fluid from a subject having a brain tumor. The method can further comprise measuring a concentration of cell-free DNA in the biological sample. The method may further comprise comparing the concentration to a reference value. The reference value can be trained using a first cohort of reference samples from subjects having a benign tumor and a second cohort of reference samples from subjects having glioma. The method may further comprise determining a classification of whether the subject has a benign tumor or glioma based on the comparison.

Each of the following features can be separately incorporated as part of the method or can be incorporated together with one or more other following features. The concentration may be measured using quantitative PCR. The concentration can be measured using a spectrophotometer (UV-vis). The concentration may be measured using capillary electrophoresis. The concentration can be measured using digital PCR. The concentration may be measured using fluorometric techniques. The reference value can be between 16-19 ng/mL. The subject may have glioma, and the concentration can be greater than the reference value. The subject may have the benign tumor, and the concentration can be less than the reference value.

A method may comprise receiving a sample of cerebrospinal fluid from a subject. For each of a plurality of cell-free DNA fragments in the sample of cerebrospinal fluid, the method can comprise identifying a location of the cell-free DNA fragment in a reference genome of the subject, thereby obtaining identified locations. The method may further comprise identifying a plurality of chromosomal regions of the subject. For each of the plurality of chromosomal regions, the method can comprise identifying a respective group of cell-free DNA fragments as being from the chromosomal region based on the identified locations. The method may further comprise determining a respective amount of the respective group of cell-free DNA fragments, thereby determining respective amounts. The method can further comprise determining a variation in the respective amounts across the plurality of chromosomal regions. The method may further comprise determining a classification of whether the subject has a benign tumor or glioma based on a comparison of the variation to a reference value. The reference value can be trained using a first cohort of reference samples from subjects having a benign tumor and a second cohort of reference samples from subjects having glioma.

Each of the following features can be separately incorporated as part of the method or can be incorporated together with one or more other following features. Obtaining the identified locations may include sequencing the plurality of cell-free DNA fragments to obtain sequence reads. Obtaining the identified locations can further comprise aligning the sequence reads to the reference genome. The plurality of chromosomal regions may be non-overlapping. The plurality of chromosomal regions can cover at least 5%, 10%, 15%, 20%, 25%, 30%, 35%, 40%, 45%, 50%, 55%, 60%, 65%, 70%, 75%, 80%, 85%, 90%, or 95% of a genome of the subject. Each of the plurality of chromosomal regions may be a same size. The respective amounts can be normalized. The normalization may use a total number of the plurality of cell-free DNA fragments. The normalization can further include a difference from a mean and a scaling by a variation in the normalized amounts across the plurality of chromosomal regions. The variation may include an entropy term or a root-mean-square deviation. The entropy term can include a sum of a proportion of the plurality of cell-free DNA fragments mapped to each of the plurality of chromosomal regions.

A method may comprise receiving a biological sample of cerebrospinal fluid from a subject. For each cell-free DNA fragment of a set of cell-free DNA fragments, the method can comprise determining a location of the cell-free DNA fragment in a reference nuclear genome or a reference mitochondrial genome using one or more sequence reads for the cell-free DNA fragment. The method may thereby determine locations of the set of cell-free DNA fragments. The method can further comprise identifying whether the cell-free DNA fragment is a nuclear DNA fragment or a mitochondrial DNA fragment based on the location. The method may further comprise measuring a normalized amount of the set of cell-free DNA fragments that are identified as mitochondrial DNA fragments, the normalized amount being relative to a second amount of the set of cell-free DNA fragments including DNA fragments that are identified as nuclear DNA fragments. The method can further comprise determining a classification of whether the subject has a benign tumor or glioma based on a comparison of the normalized amount to a reference value. The reference value may be trained using a first cohort of reference samples from subjects having a benign tumor and a second cohort of reference samples from subjects having glioma.

Each of the following features can be separately incorporated as part of the method or can be incorporated together with one or more other following features. Determining the locations of the set of cell-free DNA fragments may be made only for the reference mitochondrial genome, thereby all of the set of cell-free DNA fragments whose location is determined can be mitochondrial DNA fragments. The method may further comprise measuring a first amount of the set of cell-free DNA fragments that are identified as mitochondrial DNA fragments. The method can further comprise measuring a total amount of DNA in the biological sample, the total amount of DNA being of nuclear DNA fragments and mitochondrial DNA fragments. The total amount may correspond to the second amount. Measuring the normalized amount can use a ratio of the first amount and the total amount. The normalized amount of the set of cell-free DNA fragments that are identified as mitochondrial DNA fragments may correspond to a concentration of mitochondrial DNA in the biological sample. The method can further comprise determining a first amount of the set of cell-free DNA fragments that are mitochondrial DNA fragments. The method may further comprise determining the second amount of the set of cell-free DNA fragments by counting the nuclear DNA fragments. The method can further comprise computing a ratio of the first amount and the second amount, and the normalized amount of the set of cell-free DNA fragments that are identified as mitochondrial DNA fragments may be determined using the ratio. Determining the locations of the cell-free DNA fragments may include sequencing a set of cell-free DNA fragments to obtain sequence reads. Determining the locations of the cell-free DNA fragments can further comprise performing a mapping of the sequence reads to the reference nuclear genome and to the reference mitochondrial genome to determine which sequence reads are located on the reference nuclear genome and which sequence reads are located on the reference mitochondrial genome. The sequencing may be a random sequencing of the set of cell-free DNA fragments from the biological sample. Determining the locations of the cell-free DNA fragments can use digital PCR. The classification can be that the subject has glioma when the normalized amount is less than the reference value.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows an illustration of the brain and the circulation of cerebrospinal fluid around various anatomical features.

FIG. 2 shows an illustration of anatomical barriers of the brain and blood circulation.

FIGS. 3A-3B show illustrations of two techniques for cerebrospinal fluid extraction.

FIG. 4 is a table summarizing patient information and corresponding cfDNA concentration from plasma and cerebrospinal fluid.

FIG. 5 is a table summarizing the paired CSF and plasma collection information from patients having benign brain tumors.

FIG. 6 is a table summarizing the paired CSF and plasma collection information from patients having glioma brain tumors.

FIG. 7 shows a diagram of a workflow performed to analyze cfDNA in cerebrospinal fluid and plasma.

FIG. 8 shows the total cfDNA concentrations in paired plasma and CSF samples from cases of benign tumors of the central nervous system.

FIGS. 9A and 9B show the total cfDNA concentrations in plasma and CSF samples from cases of benign tumors and high-grade glioma tumors.

FIG. 10 is a flowchart illustrating a method for detecting a brain tumor in a subject based on cfDNA concentration.

FIGS. 11A-11B show plots of size distribution of paired plasma and CSF DNA from two individuals with high intra-cranial pressure, plotted on a linear scale (0-600 bp).

FIG. 12 depicts plasma and CSF cfDNA size profiles from each of eight patients having benign CNS tumors.

FIGS. 13A and 13B depict the mean of eight cfDNA size profiles between plasma and CSF from patients having benign tumors of the CNS.

FIGS. 14A and 14B show plots of size distribution of DNA fragments from CSF and plasma from individuals with high intracranial pressure and brain tumors.

FIG. 15 is a flowchart illustrating a method for detecting a brain tumor in a subject based on cfDNA size.

FIGS. 16A and 16B depict cfDNA size profiles of cfDNA plasma and CSF samples prepared using dsDNA sequencing libraries from cases of benign brain tumor and high-grade glioma.

FIGS. 17A and 17B depict the difference in the frequency of plasma and CSF cfDNA molecules in different size ranges of cfDNA from benign brain tumors and high-grade glioma patients using dsDNA libraries.

FIG. 18A is a box plot depicting cancer probabilistic scores predicted by SVM models using the difference in plasma cfDNA frequency between patients with benign brain tumors and gliomas in 10 bp bin sizes as inputs into the model.

FIG. 18B depicts an ROC curve analysis using the 10 bp bin sizes of plasma cfDNA to differentiate between subjects with benign brain tumors or high-grade gliomas.

FIG. 18C is a box plot depicting cancer probabilistic scores predicted by SVM models using the difference in CSF cfDNA frequency between patients with benign brain tumors and gliomas in 10 bp bin sizes as inputs into the model.

FIG. 18D depicts an ROC curve analysis using the 10 bp bin sizes of plasma or CSF cfDNA to differentiate between subjects with benign brain tumors or high-grade gliomas.

FIGS. 19A and 19B depict cfDNA size profiles of cfDNA plasma and CSF samples prepared using ssDNA sequencing libraries from cases of benign tumor and high-grade glioma.

FIGS. 20A and 20B depict the difference in the frequency of plasma and CSF cfDNA molecules in different size ranges of cfDNA from benign brain tumors and high-grade glioma patients using ssDNA libraries.

FIG. 21 is a flowchart illustrating a method for differentiating brain tumor stages in a subject based on cfDNA size.

FIG. 22A shows an illustration of the determination of pre-end motifs (PREM) and post-end motifs (POEM), as well as 5′ end motifs and 3′ end motifs.

FIG. 22B depicts an illustration of comparisons of end information obtained from existing dsDNA sequencing and ssDNA sequencing analysis, according to embodiments of the present disclosure.

FIGS. 23A and 23B show bar plots of 1-mer 5′ end motif frequencies of paired plasma and cerebrospinal fluid DNA from two individual with high intra-cranial pressure.

FIGS. 24A and 24B show plots of the correlation between 4-mer 5′ end motif rankings of plasma and CSF cfDNA from two individuals with high intra-cranial pressure.

FIGS. 25A and 25B show plots of motif frequency of 4-mer 5′ end motifs between paired plasma and CSF cfDNA from two individuals with high intracranial pressure.

FIG. 26 shows a plot of the motif frequency of 4-mer 5′ end motifs between paired plasma and CSF cfDNA from eight individuals with benign brain tumors.

FIG. 27A is a table listing the top 6 representative motifs of DNASE1L3 for two patients with high intracranial pressure.

FIG. 27B is a table listing the top 6 representative motifs of DNASE1 for two patients with high intracranial pressure.

FIG. 28 is a table listing the top 25 end motifs with highest fold change difference from individuals with high intracranial pressure.

FIG. 29 is a table listing the top 25 end motifs with the highest fold change difference from individuals with a brain tumor.

FIG. 30 is a table listing the top 25 end motifs with the lowest fold change difference from individuals with high intracranial pressure.

FIG. 31 is a table listing the top 25 end motifs with the lowest fold change difference from individuals with a brain tumor.

FIG. 32 is a table listing the top 25 end motifs with the highest fold change difference between high intracranial pressure and brain tumor from CSF derived cfDNA.

FIG. 33 is an illustration of an example end motif profile for 4-mer end motifs.

FIG. 34 is an illustration of a schematic diagram of comparing an end-motif profile of a human subject to reference F-profiles determined based on murine samples, according to some embodiments of the present disclosure.

FIG. 35 is a bar chart representing deconvolution analysis of F-profiles in paired plasma and CSF cfDNA samples from patients having high intracranial pressure.

FIG. 36 is a bar chart representing deconvolution analysis of F-profiles in paired plasma and CSF cfDNA samples from cases of benign tumors.

FIG. 37 is a box plot of the percentages of F-profiles from paired plasma and CSF cfDNA samples from cases of benign brain tumors.

FIG. 38 is a bar chart showing deconvolution analysis of F-profiles for paired plasma and CSF cfDNA from individuals with high intra-cranial pressure (non-tumor) and from individuals with brain tumor (n=2 per group).

FIG. 39A is a bar chart representing deconvolution F-profiles of 5′ end motifs in paired plasma and CSF cfDNA samples from cases of benign tumors in patients with benign and glioma tumors.

FIG. 39B is a bar chart representing deconvolution F-profiles of 5′ end motifs in paired plasma and CSF cfDNA samples from cases of benign tumors in patients with benign and glioma tumors.

FIG. 40 shows box plots of F-profile contributions in plasma cfDNA between benign and high-grade glioma tumors.

FIG. 41 shows box plots of F-profile contributions in CSF cfDNA between benign and high-grade glioma tumors.

FIG. 42A is a hierarchical clustering heatmap analysis of PREM from plasma cfDNA of patients having benign brain tumors or high-grade gliomas.

FIG. 42B is a hierarchical clustering heatmap analysis of PREM from CSF cfDNA of patients having benign brain tumors or high-grade gliomas.

FIG. 43 is a volcano plot analysis showing differential PREM frequency in CSF cfDNA between patients with benign brain tumors and high-grade gliomas.

FIG. 44A is a box plot depicting cancer probabilistic scores predicted by SVM models using PREM from plasma cfDNA as inputs into the model.

FIG. 44B depicts an ROC curve analysis using the PREM from plasma cfDNA to differentiate between subjects with benign brain tumors or high-grade gliomas.

FIG. 44C is a box plot depicting cancer probabilistic scores predicted by SVM models using PREM from CSF cfDNA as inputs into the model.

FIG. 44D depicts an ROC curve analysis using the PREM from CSF cfDNA to differentiate between subjects with benign brain tumors or high-grade gliomas.

FIG. 45A is a hierarchical clustering heatmap analysis of EM5 from plasma cfDNA of patients having benign brain tumors or high-grade gliomas.

FIG. 45B is a hierarchical clustering heatmap analysis of EM5 from plasma cfDNA of patients having benign brain tumors or high-grade gliomas.

FIG. 46 is a volcano plot analysis showing differential EM5 frequency in CSF cfDNA between patients with benign brain tumors and high-grade gliomas.

FIG. 47A is a box plot depicting cancer probabilistic scores predicted by SVM models using EM5 from plasma cfDNA as inputs into the model.

FIG. 47B depicts an ROC curve analysis using the EM5 from plasma cfDNA to differentiate between subjects with benign brain tumors or high-grade gliomas.

FIG. 47C is a box plot depicting cancer probabilistic scores predicted by SVM models using EM5 from CSF cfDNA as inputs into the model.

FIG. 47D depicts an ROC curve analysis using the EM5 from CSF cfDNA to differentiate between subjects with benign brain tumors or high-grade gliomas.

FIG. 48A is a hierarchical clustering heatmap analysis of EM3 from plasma cfDNA of patients having benign brain tumors or high-grade gliomas.

FIG. 48B is a hierarchical clustering heatmap analysis of EM3 from plasma cfDNA of patients having benign brain tumors or high-grade gliomas.

FIG. 49 is a volcano plot analysis showing differential EM3 frequency in CSF cfDNA between patients with benign brain tumors and high-grade gliomas.

FIG. 50A is a box plot depicting cancer probabilistic scores predicted by SVM models using EM3 from plasma cfDNA as inputs into the model.

FIG. 50B depicts an ROC curve analysis using the EM3 from plasma cfDNA to differentiate between subjects with benign brain tumors or high-grade gliomas.

FIG. 50C is a box plot depicting cancer probabilistic scores predicted by SVM models using EM3 from CSF cfDNA as inputs into the model.

FIG. 50D depicts an ROC curve analysis using the EM3 from CSF cfDNA to differentiate between subjects with benign brain tumors or high-grade gliomas.

FIG. 51A is a hierarchical clustering heatmap analysis of POEM from plasma cfDNA of patients having benign brain tumors or high-grade gliomas.

FIG. 51B is a hierarchical clustering heatmap analysis of POEM from plasma cfDNA of patients having benign brain tumors or high-grade gliomas.

FIG. 52 is a volcano plot analysis showing differential POEM frequency in CSF cfDNA between patients with benign brain tumors and high-grade gliomas.

FIG. 53A is a box plot depicting cancer probabilistic scores predicted by SVM models using POEM from plasma cfDNA as inputs into the model.

FIG. 53B depicts an ROC curve analysis using the POEM from plasma cfDNA to differentiate between subjects with benign brain tumors or high-grade gliomas.

FIG. 53C is a box plot depicting cancer probabilistic scores predicted by SVM models using POEM from CSF cfDNA as inputs into the model.

FIG. 53D depicts an ROC curve analysis using the POEM from CSF cfDNA to differentiate between subjects with benign brain tumors or high-grade gliomas.

FIG. 54A is a box plot depicting cancer probabilistic scores predicted by SVM models using PREM, EM5, EM3, and POEM from CSF cfDNA as inputs into the model.

FIG. 54B depicts an ROC curve analysis using the PREM, EM5, EM3, and POEM from CSF cfDNA to differentiate between subjects with benign brain tumors or high-grade gliomas.

FIG. 55 is a flowchart illustrating a method for detecting a brain tumor in a subject based on end motifs, according to some embodiments of the present disclosure.

FIG. 56 illustrates cutting positions relative to CpG sites (also referred to as CG sites) according to embodiments of the present disclosure.

FIGS. 57A-57B show CGN/NCG motif ratio analysis across the whole genome, Alu regions, and CpG islands in cfDNA from CSF and plasma.

FIG. 58 is a box plot of methylation levels from bisulfite sequencing of paired plasma and CSF cfDNA from patients having benign brain tumors.

FIG. 59A shows the cleavage profile of methylated and unmethylated CpG sites of CSF cfDNA in a 11-nt cleavage measurement window.

FIG. 59B shows the cleavage profile of methylated and unmethylated CpG sites of plasma cfDNA in a 11-nt cleavage measurement window.

FIG. 60A is a box plot of CGN/NCG motif ratios of methylated and unmethylated CpG sites in CSF cfDNA from patients having benign brain tumors.

FIG. 60B is a box plot of CGN/NCG motif ratios of methylated and unmethylated CpG sites in plasma cfDNA from patients having benign brain tumors.

FIG. 61A is a box plot of CGN/NCG motif ratios across the whole genome (overall methylation level), Alu regions, and CpG islands (CGI) in CSF cfDNA from patients with benign brain tumors.

FIG. 61B is a box plot of CGN/NCG motif ratios across the whole genome (overall methylation level), Alu regions, and CpG islands (CGI) in plasma cfDNA from patients with benign brain tumors.

FIG. 62A is a box plot of the 5′ CGN/NCG end motif ratios for glioma specific hypermethylated CpG sites in plasma cfDNA in patients with benign brain tumors or patients with gliomas.

FIG. 62B is a box plot of the 5′ CGN/NCG end motif ratios for glioma specific hypomethylated CpG sites in plasma cfDNA in patients with benign brain tumors or patients with gliomas.

FIG. 63A is a box plot of the 5′ CGN/NCG end motif ratios for glioma specific hypermethylated CpG sites in CSF cfDNA in patients with benign brain tumors or patients with gliomas.

FIG. 63B is a box plot of the 5′ CGN/NCG end motif ratios for glioma specific hypomethylated CpG sites in CSF cfDNA in patients with benign brain tumors or patients with gliomas.

FIG. 64A is a box plot of the frequency of 5′ CGN motifs at glioma specific hypermethylated CGN CpG sites in plasma cfDNA in patients with benign brain tumors or patients with gliomas.

FIG. 64B is a box plot of the frequency of 5′ CGN motifs at glioma specific hypomethylated CGN CpG sites in plasma cfDNA in patients with benign brain tumors or patients with gliomas.

FIG. 65A is a box plot of the frequency of 5′ CGN motifs at glioma specific hypermethylated CGN CpG sites in CSF cfDNA in patients with benign brain tumors or patients with gliomas.

FIG. 65B is a box plot of the frequency of 5′ CGN motifs at glioma specific hypomethylated CGN CpG sites in CSF cfDNA in patients with benign brain tumors or patients with gliomas.

FIG. 66A is a box plot of the frequency of 5′ NCG motifs at glioma specific hypermethylated NCG CpG sites in plasma cfDNA in patients with benign brain tumors or patients with gliomas.

FIG. 66B is a box plot of the frequency of 5′ NCG motifs at glioma specific hypomethylated NCG CpG sites in plasma cfDNA in patients with benign brain tumors or patients with gliomas.

FIG. 67A is a box plot of the frequency of 5′ NCG motifs at glioma specific hypermethylated NCG CpG sites in CSF cfDNA in patients with benign brain tumors or patients with gliomas.

FIG. 67B is a box plot of the frequency of 5′ NCG motifs at glioma specific hypomethylated NCG CpG sites in CSF cfDNA in patients with benign brain tumors or patients with gliomas.

FIG. 68A is a box plot of methylation density for glioma specific hypermethylated CpG sites in CSF cDNA in patients with benign brain tumors or patients with gliomas.

FIG. 68B is a box plot of methylation density for glioma specific hypomethylated CpG sites in CSF cDNA in patients with benign brain tumors or patients with gliomas.

FIG. 69 is a flowchart illustrating a method for differentiating between brain tumor types in a subject based on CGN and NCG motifs at CpG sites.

FIG. 70 is a flowchart illustrating a method for differentiating between brain tumor types in a subject based on methylated levels of CpG sites.

FIG. 71 shows results of tissue-of-origin deconvolution performed using methylation status (bisulfite sequencing of cfDNA samples) according to embodiments of the present disclosure.

FIG. 72 is a bar chart representing methylation-based cell-type deconvolution analysis of cfDNA from paired plasma and CSF cfDNA samples from cases of benign tumors.

FIG. 73A-73D are box plots showing the percent contribution of specific cell types to the tissue-of-origin analysis from methylation-based cell-type deconvolution analysis of cfDNA from paired plasma and CSF cfDNA.

FIG. 74A is a bar chart representing methylation-based cell-type deconvolution analysis of plasma cfDNA samples from patients having benign brain tumors and patients having high-grade gliomas.

FIG. 74B is a bar chart representing methylation-based cell-type deconvolution analysis of CSF cfDNA samples from patients having benign brain tumors and patients having high-grade gliomas.

FIG. 75A-75I are box plots showing the percent contribution of specific cell types to the tissue-of-origin analysis from methylation-based cell-type deconvolution analysis of cfDNA from CSF cfDNA.

FIG. 76 is a flowchart illustrating a method for analyzing a sample of cerebrospinal fluid form a subject, according to some embodiments of the present disclosure.

FIG. 77 is a visual representation of copy number alterations in plasma cfDNA from patients having benign brain tumors.

FIG. 78 is a visual representation of copy number alterations in plasma cfDNA from patients having high-grade glioma.

FIG. 79 shows tumor fraction of paired plasma and CSF determined by copy number aberration from patients having high intracranial pressure or benign brain tumor.

FIG. 80 is a flowchart illustrating a method for measuring a fraction of cell-free DNA fragments from a brain tumor, according to some embodiments of the present disclosure.

FIG. 81 is a visual representation of copy number alterations in CSF cfDNA from patients having benign brain tumors.

FIG. 82 is a visual representation of copy number alterations in CSF cfDNA from patients having high-grade glioma.

FIGS. 83A and 83B depict the genomic coverage of cfDNA from plasma for patients with benign or high-grade glioma.

FIGS. 84A and 84B depict the genomic coverage of cfDNA from CSF for patients with benign or high-grade glioma.

FIGS. 85A and 85B are box plots showing the coefficient of variance (CV) of cfDNA fragmentation density coverage in patients with benign and high-grade glioma, from paired plasma and CSF cfDNA.

FIGS. 85C and 85D are box plots showing the entropy score of cfDNA fragmentation density coverage in patients with benign and high-grade glioma, from paired plasma and CSF cfDNA.

FIG. 86 is a flowchart illustrating a method for measuring copy number aberrations from a brain tumor.

FIG. 87 is a box plot depicting the percentage of mtDNA fragments in paired plasma and CSF samples from cases of benign tumors.

FIGS. 88A and 88B are box plots depicting the mtDNA proportion between dsDNA and ssDNA sequencing libraries from paired plasma or CSF cfDNA from patients with benign brain tumors.

FIGS. 89A and 89B are box plots depicting the mtDNA proportion between dsDNA and ssDNA sequencing libraries from paired plasma or CSF cfDNA from patients with high-grade gliomas.

FIGS. 90A and 90B are box plots depicting the percentage of mtDNA fragments in patients with benign and glioma tumors from dsDNA libraries of paired plasma and CSF samples.

FIGS. 91A and 91B are box plots depicting the percentage of mtDNA fragments in patients with benign and glioma tumors from ssDNA libraries of paired plasma and CSF samples.

FIG. 92 is a flowchart illustrating a method for measuring the proportion of mtDNA from a brain tumor to differentiate between brain tumor types.

FIG. 93 illustrates a system according to an embodiment of the present invention.

FIG. 94 shows a block diagram of an example computer system usable with system and methods, according to certain embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest (e.g., DNA and/or RNA). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, peritoneal fluid, discharge fluid from the nipple, aspiration fluid from different parts of the body (e.g., thyroid, breast), intraocular fluids (e.g., the aqueous humor), amniotic fluid, etc. Stool samples can also be used. In various embodiments, the majority of DNA in a biological sample (e.g., that has been enriched for cell-free DNA, such as a plasma sample obtained via a centrifugation protocol) can be cell-free, e.g., greater than 50%, 60%, 70%, 80%, 90%, 95%, or 99% of the DNA can be cell-free. A centrifugation protocol for enriching cell-free DNA from a biological sample can include, for example, centrifuging the biological sample at 1,600 g×10 minutes, obtaining the fluid part of the centrifuged sample, and re-centrifuging at for example, 16,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

The term “fractional fetal DNA concentration” is used interchangeably with the terms “fetal DNA proportion” and “fetal DNA fraction,” and refers to the proportion of fetal DNA molecules that are present in a biological sample (e.g., maternal plasma or serum sample) that is derived from the fetus (Lo et al, Am J Hum Genet. 1998; 62:768-775; Lun et al, Clin Chem. 2008; 54:1664-1672). Similarly, tumor fraction or tumor DNA fraction can refer to the fractional concentration of tumor DNA in a biological sample, or tissue fraction can refer to the fractional concentration of DNA from one or more particular tissue(s), e.g., from a transplant organ.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

The term “assay” generally refers to a technique for determining a property of a nucleic acid or a sample of nucleic acids (e.g., a statistically significant number of nucleic acids), as well as a property of the subject from which the sample was obtained. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity (e.g., based on selection of one or more cutoff values), and their relative usefulness as a diagnostic tool can be measured using Receiver Operating Characteristic (ROC) Area-Under-the-Curve (AUC) statistics.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location. Various alignment tools can be used, such as BLAST, BLASTZ, FASTA, G-PAS, SSEARCH, BOWTIE, AMAP, or SOAP.

A “site” (also called a “genomic site”) corresponds to a single site, which may be a single base position or a group of correlated base positions, e.g., a CpG site, TSS site, DNase hypersensitivity site, or larger group of correlated base positions. A “locus” may correspond to a region that includes multiple sites. A locus can include just one site, which would make the locus equivalent to a site in that context. A region can be defined around a site, e.g., a symmetric or asymmetric region around a site. As examples, a region can include at least +/−50 bases before and after a site (e.g., 101 bases), +/−60 bases, +/−70 bases, +/−80 bases, +/−90 bases, +/−100 bases, +/−150 bases, +/−200 bases, +/−300 bases, +/−400 bases, +/−500 bases, +/−600 bases, +/−700 bases, +/−800 bases, +/−900 bases, and +/−1,000 bases. As other examples a region can be at least 100 bases, 140 bases, 147 bases, or 167 bases long. One or more regions can be analyzed, e.g., to provide a level of a pathology (e.g., cancer) or a fraction of a particular tissue. Various number of regions, sites, or loci can be analyzed, e.g., 50, 100, 200, 500, 1,000, 5,000, 10,000, 50,000, 100,000, 500,000, one million, or more. Various techniques can determine where a DNA molecule is located at one or more genomic positions in a reference genome, e.g., alignment of a sequence read to the reference genome or using position-specific probes. The position determination can be to some or all of the reference genome, e.g., if only part of the genome is being analyzed. As examples, the amount of the genome analyzed can be greater than 0.01%, 0.1%, 1%, 5%, 10%, or 50%. A “cutting site” can refer to a location that DNA was cut by a nuclease, thereby resulting in a DNA fragment.

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned. Various numbers of motifs can be used, e.g., at least 1, 2, 3, 4, 5, 6, 7, 8, 9, 10, 16, 20, 30, 40, 50 60, 64, 70, 80, 90, 100, 150, 200, 250, or 256 end motifs. Further details about end motifs can be found in U.S. Patent Publications 2020/0199656, 2022/0010353, 2023/0313314, and 2024/0043935.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. As another example, a DNA fragment having an A at the 5′ end of one strand and an T at the 3′ end of the same strand can be defined as having a sequence motif pair of A< >T, which would correspond to an A< >A fragment defined using the 5′ ends of the two strands. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

An “end motif type” can indicate which end (3′ or 5′ end) of a DNA fragment or strand that the end motif corresponds, as well as whether the end motif occurs on (3′-EM or 5′EM), before (pre-end motif), or after (post-end) the DNA fragment, as well as the specific positions. Additionally, an end motif type can include which strand (Watson or Crick) is used. For example, a pre-end motif can be composed of positions −1, −3, −4, −6), represented as PREM(W, −1:−3:−4:−6). Thus, there can be a gap between the nucleotides when the positions are non-continuous. As examples, the pre-end motif can include before the 5′ end at least 2, 3, 4, 5, 6, 7, 8, 9, 10, 11, 12, 13, 14, 15, 16, 17, 18, 19, or 20 nucleotides, which may or may not be consecutive with each other. A distance of the pre-end motif to the 5′ end can be at least, e.g.: 0, 1, 2, 3, 4, 5, 6, 7, 8, 9, 10, 11, 12, 13, 14, 15, 16, 17, 18, 19, or 20 nucleotides. In some embodiments, a maximum distance of the pre-end motif to the 5′ end can be equal to or less than 50, 49, 48, 47, 46, 45, 44, 43, 42, 41, or 40 nucleotides. As other examples, the post-end motif can include after the 3′ end at least 2, 3, 4, 5, 6, 7, 8, 9, 10, 11, 12, 13, 14, 15, 16, 17, 18, 19, or 20 nucleotides, which may or may not be consecutive with each other. A distance of the post-end motif to the 3′ end can be at least, e.g.: 0, 1, 2, 3, 4, 5, 6, 7, 8, 9, 10, 11, 12, 13, 14, 15, 16, 17, 18, 19, or 20 nucleotides. In some embodiments, a maximum distance of the post-end motif to the 3′ end can be equal to or less than 50, 49, 48, 47, 46, 45, 44, 43, 42, 41, or 40 nucleotides.

A “end-motif profile” may refer to the relationship of ending sequences (e.g., 1-30 bases) of cell-free DNA fragments (also just referred to as DNA fragments) in a sample. Various relationships can be provided, e.g., an amount of cell-free DNA fragments with a particular ending sequence (end motif), a relative frequency of cell-free DNA fragments with a particular ending sequence compared to one or more other ending sequences. In some instances, the end-motif profiles are determined using other types of parameters, such as size. For example, the end-motif profile can be provided in various ways that illustrate an amount of cell-free DNA fragments having one or more particular ending sequences for a given size (single length or size range). A “reference end-motif profile” or an “F-profile” refers to an end-motif profile that can be generated by applying a factorization algorithm (e.g., non-negative matrix factorization) to relative frequencies of DNA molecules of a given biological sample across a plurality of end motifs (e.g., 256 end motifs). Further details about end motif profiles can be found in U.S. Patent Publication 2024/0182982.

“Single-molecule sequencing” refers to sequencing of a single template DNA molecule to obtain a sequence read without the need to interpret base sequence information from clonal copies of a template DNA molecule. The single-molecule sequencing may sequence the entire molecule or only part of the DNA molecule. A majority of the DNA molecule may be sequenced, e.g., greater than 50%, 55%, 60%, 65%, 70%, 75%, 80%, 85%, 90%, 95%, or 99%. A sequence read (or reads from both ends) can be aligned to a reference genome. When both ends are aligned (e.g., as part of a read of the entire fragment or for paired-ends), greater accuracy can be achieved in the alignment and a length of the fragment can be obtained. Embodiments of the present disclosure can use single-molecule sequencing.

The term “alleles” refers to alternative DNA sequences at the same physical genomic locus, which may or may not result in different phenotypic traits. In any particular diploid organism, with two copies of each chromosome (except the sex chromosomes in a male human subject), the genotype for each gene comprises the pair of alleles present at that locus, which are the same in homozygotes and different in heterozygotes. A population or species of organisms typically include multiple alleles at each locus among various individuals. A genomic locus where more than one allele is found in the population is termed a polymorphic site. Allelic variation at a locus is measurable as the number of alleles (i.e., the degree of polymorphism) present, or the proportion of heterozygotes (i.e., the heterozygosity rate) in the population. As used herein, the term “polymorphism” refers to any inter-individual variation in the human genome, regardless of its frequency. Examples of such variations include, but are not limited to, single nucleotide polymorphism, simple tandem repeat polymorphisms, insertion-deletion polymorphisms, sequence variants/mutations (which may be disease causing) and copy number variations (also referred to as a copy number aberration). The term “haplotype” can refer to a combination of alleles or epigenetic markers (e.g., methylation) at multiple loci that are transmitted together on the same chromosome or chromosomal region. A haplotype may refer to as few as one pair of loci or to a chromosomal region, or to an entire chromosome or chromosome arm.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range. Other parameters can include an average, median, mode, or mean. Further details about size profiles can be found in U.S. Patent Publications 2011/0276277, 2013/0040824, 2016/0201142, and 2016/0217251.

“DNA methylation” in mammalian genomes typically refers to the addition of a methyl group to the 5′ carbon of cytosine residues (i.e., 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported.

The “methylation index” for each genomic site (e.g., a CpG site) can refer to the proportion of DNA fragments (e.g., as determined from sequence reads or probes) showing methylation at the site over the total number of reads covering that site. A “methylation status” can refer to whether a particular site is methylated at a particular site of a DNA fragment or whether a particular site in a genome has a particular differential methylation status, e.g., hypermethylation or hypomethylation. A “read” can include information (e.g., methylation status at a site) obtained from a DNA fragment. A read can be obtained using reagents (e.g., primers or probes) that preferentially hybridize to DNA fragments of a particular methylation status. Typically, such reagents are applied after treatment with a process that differentially modifies or differentially recognizes DNA molecules depending on their methylation status, e.g., bisulfite conversion, or methylation-sensitive restriction enzyme, or methylation binding proteins, or anti-methylcytosine antibodies, or single molecule sequencing techniques that recognize methylcytosines and hydroxymethylcytosines.

The “methylation density” of a region or a set of sites can refer to the number of reads at site(s) within the region (also referred to as a bin) or the set of sites showing methylation divided by the total number of reads covering the site(s) in the region or the set of sites. A region can include one or more sites of interest, including at least 1, 2, 3, 4, 5, 10, 20, 50, 100, 200, 500, and 1,000 sites. The site(s) may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer to the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci USA 2013; 110: 18910-18915) and by the Pacific Biosciences single molecule real time analysis (Tse et al. Proc Natl Acad Sci USA 2021; 118: e2019768118).

A “methylation level” is an example of a relative abundance, e.g., between methylated DNA molecules (e.g., at one or more particular sites) and other DNA molecules (e.g., all other DNA molecules or just unmethylated DNA molecules at the one or more particular sites). The amount of other DNA molecules can act as a normalization factor. As another example, an intensity of methylated DNA molecules (e.g., fluorescent or electrical intensity) relative to intensity of all or unmethylated DNA molecules at one or more sites can be determined. The relative abundance can also include an intensity per volume. A methylation level can be determined using a methylation-aware assay such as methylation-aware sequencing or PCR. Example methylation-aware sequencing can include bisulfite sequencing, sequencing after treatment using methylation-sensitive restriction enzymes, or single molecule techniques, e.g., using nanopores or single molecule real-time sequencing from Pacific Biosciences as described in U.S. Patent Publication 2021/0047679.

A differentially methylated region (DMR) is a genomic region (e.g., set of sites) with different DNA methylation level across two or more biological samples. The different DNA methylation level may be defined by the certain difference in methylation index or density, such as but not limited to 1%, 5%, 10%, 20%, 30%, 40%, 50%, 60%, 70%, 80%, 90%, 95%, 99%, etc. A differentially methylated site (DMS) may be defined in a similar manner.

The term “hypomethylation” can refer to a site (hypomethylated site) or set of sites (e.g., a region) that has below a specified threshold for a methylation level, e.g., at or below 50%, 45%, 40%, 35%, 30%, 25%, or 20% for the methylation level. A site in a genome may be considered unmethylated if the methylation level is below a threshold. The term “hypermethylation” can refer to a site (hypermethylated site) or set of sites (e.g., a region) that has above a specified value for a methylation level, e.g., at or above 95%, 90%, 80%, 75%, 70%, 65%, or 60% for the methylation level. A site in a genome may be considered methylated if the methylation level is greater than a threshold. Hypomethylation or hypermethylation can occur for a particular tissue or across a set of tissues.

A “relative frequency” (also referred to just as “frequency”) may refer to a relative value of one amount determined from nucleic acid fragments having a particular characteristic (e.g., an end motif or a size, such as a specified length) to one or more other amounts determined from nucleic acid fragments having a different characteristic. Examples include a ranking or a proportion (e.g., a percentage, fraction (ratio), or concentration). For example, a relative frequency of a particular end motif (e.g., A, CG, TAG, etc.) or end motif pair (e.g., A< >A) can provide a proportion of cell-free DNA fragments that have that end motif or that particular pair end motif pair. Such a proportion can be out of all the end motifs for a set of DNA molecules. As another example, the proportion can be a ratio of an amount for a particular end motif (or pair) relative to an amount of one or more other end motifs. As other examples, the relative frequency can be a ranking of amounts, e.g., raw counts of end motifs. The ranking can be of proportions (ratios) for each end motifs, as another example. Similar relative frequencies can be determined for size. As another example, a relative frequency can correspond to a proportion of cfDNA fragments that end at a site or one of a group of sites.

An “aggregate value” may refer to a collective property, e.g., of relative frequencies of a set of end motifs. Examples include a mean, a median, a sum of relative frequencies, a variation among the relative frequencies (e.g., entropy, standard deviation (SD), the coefficient of variation (CV), interquartile range (IQR) or a certain percentile cutoff (e.g., 95^{th} or 99^{th} percentile) among different relative frequencies), or a difference (e.g., a distance) from a reference pattern of relative frequencies, as may be implemented in clustering. As another example, an aggregate value can comprise an array/vector of relative frequencies, which can be compared to a reference vector (e.g., representing a multidimensional data point).

A “calibration sample” can correspond to a biological sample whose fractional concentration of clinically-relevant DNA (e.g., tissue-specific DNA fraction) is known or determined via a calibration method, e.g., using an allele specific to the tissue, such as in transplantation whereby an allele present in the donor's genome but absent in the recipient's genome can be used as a marker for the transplanted organ. As another example, a calibration sample can correspond to a sample from which end motifs can be determined. A calibration sample can be used for both purposes.

A “calibration data point” includes a “calibration value” (e.g., an amount of fragments with a particular end motif or with a particular size) and a measured or known value that is desired to be determined for other test samples (e.g., a fractional concentration of the clinically-relevant DNA such as DNA of particular tissue type). The calibration value can be determined from various types of data measured from DNA molecules of the sample, (e.g., an amount of fragments with an end motif or with a particular size, such as relative frequencies (e.g., an aggregate value) as determined for a calibration sample). The calibration value corresponds to a parameter that correlates to the desired property, e.g., classification of a genetic disorder, nuclease activity, or efficacy of anticoagulant dosage. For example, a calibration value can be determined from measured values as determined for a calibration sample, for which the desired property is known. The measured or known value (e.g., a fractional concentration) can be determined in various ways, e.g., using a tissue-specific allele, a tissue-specific methylation value or pattern, and a size distribution of a sample with a known fractional concentration. The calibration data points may be defined in a variety of ways, e.g., as discrete points or as a calibration function (also called a calibration curve or calibration surface). The calibration function could be derived from additional transformation of the calibration data points.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The term “parameter” as used herein can refer to a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A normalized amount, e.g., a relative frequency, is an example of a parameter.

A “separation value” corresponds to a difference or a ratio involving two values, e.g., two fractional contributions or two methylation levels. A separation value is an example of a parameter. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (ln) of the two values. A separation value can include a difference and a ratio. A separation value can be compared to a threshold to determine whether the separation between the two values is statistically significant.

A “separation value” and an “aggregate value” (e.g., of relative frequencies) are two examples of a parameter (also called a metric) that provides a measure of a sample that varies between different classifications (states), and thus can be used to determine different classifications. An aggregate value can be a separation value, e.g., when a difference is taken between a set of relative frequencies of a sample and a reference set of relative frequencies, as may be done in clustering.

The term “relative abundance” may generally refer to a ratio of a first amount of nucleic acid fragments having a particular characteristic (e.g., a specified length, ending at one or more specified coordinates/ending positions, or aligning (mapping) to a particular region of the genome) to a second amount nucleic acid fragments having a particular characteristic (e.g., a specified length, ending at one or more specified coordinates/ending positions, or aligning (mapping) to a particular region of the genome). In one example, relative abundance may refer to a ratio of the number of DNA fragments ending at a first set of genomic positions (e.g., open chromatin regions) to the number (e.g., a mean or a median) of DNA fragments ending at a second set of genomic positions, which may be all genomic positions. Such a relative abundance may be referred to as an end density. In some aspects, “relative abundance” is a type of separation value that relates an amount (one value) of cell-free DNA molecules ending within one window of genomic position to an amount (other value) of cell-free DNA molecules ending within another window of genomic positions. The two windows may overlap but would be of different sizes. In other implementations, the two windows would not overlap. Further, the windows may be of a width of one nucleotide, and therefore be equivalent to one genomic position. An end density is a type of relative abundance. In some instances, an observed-to-expected (O/E) ratio is another type of relative abundance.

The term “sequence imbalance” or “aberration” or “copy number aberration (CNA)” as used herein means any significant deviation as defined by at least one cutoff value in a quantity of the clinically relevant chromosomal region from a reference quantity. A sequence imbalance can include chromosome dosage imbalance, allelic imbalance, mutation dosage imbalance, copy number imbalance, haplotype dosage imbalance, and other similar imbalances. As an example, an allelic imbalance can occur when a tumor has one allele of a gene deleted or one allele of a gene amplified or differential amplification of the two alleles in its genome, thereby creating an imbalance at a particular locus in the sample. As another example, a patient could have an inherited mutation in a tumor suppressor gene. The patient could then go on to develop a tumor in which the non-mutated allele of the tumor suppressor gene is deleted. Thus, within the tumor, there is mutation dosage imbalance. When the tumor releases its DNA into the plasma of the patient, the tumor DNA will be mixed in with the constitutional DNA (from normal cells) of the patient in the plasma. An aberration can include a deletion or amplification of a chromosomal region.

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The terms “cancer” or “tumor” may be used interchangeably and generally refer to an abnormal mass of tissue wherein the growth of the mass surpasses and is not coordinated with the growth of normal tissue. A cancer or tumor may be defined as “benign” or “malignant” depending on the following characteristics: degree of cellular differentiation including morphology and functionality, rate of growth, local invasion, and metastasis. A “benign” tumor is generally well differentiated, has characteristically slower growth than a malignant tumor, and remains localized to the site of origin. In addition, a benign tumor does not have the capacity to infiltrate, invade, or metastasize to distant sites. A “malignant” tumor is generally poorly differentiated (anaplasia), has characteristically rapid growth accompanied by progressive infiltration, invasion, and destruction of the surrounding tissue. Furthermore, a malignant tumor has the capacity to metastasize to distant sites. “Stage” can be used to describe how advanced a tumor is. Early stage cancer or malignancy is associated with less tumor burden in the body, generally with less symptoms, with better prognosis, and with better treatment outcome than a late stage malignancy. Late or advanced stage cancer or malignancy is often associated with distant metastases and/or lymphatic spread. As examples, a benign tumor may correspond to stages 1 and 2, whereas glioma can correspond to any stage and may include high grade tumors at stages 3 and 4.

A “level of pathology” (also referred to as a condition) can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g., cirrhosis), fatty infiltration (e.g., fatty liver diseases), degenerative processes (e.g., Alzheimer's disease) and ischemic tissue damage (e.g., myocardial infarction or stroke). A healthy state of a subject can be considered a classification of no pathology.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer. A level for various types of cancer can be determined, e.g., carcinoma or sarcoma, melanoma, lymphoma, and leukemia, as well as in various tissue of origin, including by way of example: breast, lung, liver, colon, pancreas, stomach, bone, blood, head and neck (e.g., head and neck squamous cell carcinoma), throat, bladder, kidney, prostate, uterine, rectal, bile duct, brain, eye, esophageal, ovarian, oral cavity, Nasopharyngeal, thyroid, urethral, testicular, vaginal, and pituitary.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, one million, ten million, 100 million, or one billion parameters. An ML model can be generated using sample data (e.g., training samples) to make predictions on test data. Various number of training samples can be used, e.g., at least 10, 100, 1,000, 5,000, 10,000, 50,000, 100,000, or 200,000 training samples. One example is reinforcement learning such as Q-Learning, Deep Q-Networks (DQN), Double DQN, Dueling DQN, Policy Gradient Methods, Actor-Critic, Advantage Actor-Critic (A2C), Proximal Policy Optimization (PPO), Trust Region Policy Optimization (TRPO), and Soft Actor-Critic (SAC). Another example is an unsupervised learning model such as hidden Markov model (HMM), clustering (e.g., hierarchical clustering, k-means, mixture models, model-based clustering, density-based spatial clustering of applications with noise (DBSCAN), and OPTICS algorithm), approaches for learning latent variable models such as Expectation-maximization algorithm (EM), method of moments, and blind signal separation techniques (e.g., principal component analysis, independent component analysis, non-negative matrix factorization, singular value decomposition), and anomaly detection (e.g., local outlier factor and isolation forest). Another example type of model is supervised learning that can be used with embodiments of the present disclosure. Example supervised learning models may include different approaches and algorithms including analytical learning, statistical models, artificial neural network (e.g. including convolutional and/or transformer layers) that may have 1-10 layers as examples, recurrent neural network (e.g., long short term memory, LSTM), boosting (meta-algorithm), bootstrap aggregating (bagging) such as random forests, support vector machine (SVM), support vector (SVR), Bayesian statistics, case-based reasoning, decision tree learning (e.g., CART (classification and regression trees), gradient boosted trees, or random forest), inductive logic programming, linear regression, logistic regression, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct (PAC) learning, ripple down rules, a knowledge acquisition methodology, symbolic machine learning algorithms, subsymbolic machine learning algorithms, minimum complexity machines (MCM), ordinal classification, data pre-processing, handling imbalanced datasets, statistical relational learning, or Proaftn (a multicriteria classification algorithm), or an ensemble of any of these types. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

A “report” can include any data described herein, including one or more classifications or output values by a machine learning model. A report can be provided to a user in various ways, e.g., via display on a monitor, email, or text message.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

In this disclosure, we develop methods to evaluate the fragmentomics and epigenetic profiles of cfDNA from CSF, as a diagnostic tool to studying malignancies and other pathologies in the CNS. Fragmentomic related alterations in cfDNA, such as the size distribution, end motif patterns, and cleavage profiles around CpG sites, and the potential to deduce the epigenetic profile, such as methylation, would provide a powerful diagnostic tool in examining CNS tumors from the CSF. In some instances, plasma may be used as well. The applications may also be expanded to staging and localization of tumor growth and can be utilized in monitoring cancer progression, treatment response, cancer recurrence, and minimal residual disease of the CNS. Overall, fragmentomic and epigenetic profiling can provide further biological insights and clinical utility to CSF samples collected from patients from various neurological conditions.

A size-based analysis can detect a present of a brain tumor, e.g., differentiating between intra-cranial pressure and a brain tumor. Additionally or alternatively, s size-based analysis can determine a stage of a brain cancer, e.g., differentiating between a benign tumor (e.g., stages 0-2) and a glioma (e.g., at stages 3-4). A sample size profile can be formulated in various ways and proportions of cfDNA fragments at one or more sizes can be used to make such differentiations.

End motifs can be used in various ways to determine a classification of a level of a brain tumor for the subject, e.g., detecting a presence/absence or a stage of a brain tumor (e.g., benign or glioma). The end motifs can be of various types (e.g. pre-end motifs (PREM), 5′ end motifs (EM5), 3′ end motifs (EM3), or post-end motifs (POEM)). An end-motif profile can comprise a single end motif or a plurality, e.g., for which reference F-profiles can be used. When a plurality of end motifs are used, a machine learning model can be used.

A particular set of end motifs can be CGN and/or NCG end motifs, which can be used in a ratio of their sums or as separate values. Amounts of such CGN and NCG end motifs may be determined in particular regions that are hypermethylated or hypomethylated in glioma. Such amount(s) can be used in conjunction with a model (e.g., a reference value or a machine learning model), where the model is trained using a first cohort of reference samples from subjects having a benign tumor and a second cohort of reference samples from subjects having glioma. Various techniques described here can used various amounts in a similar manner in conjunction with such a model.

Methylation levels at CpG sites in particular regions that are hypermethylated or hypomethylated in glioma may be used to determine a stage of a brain tumor, e.g., benign or glioma. The methylation levels can be determined in various ways with various methylation-aware assays. As another example, methylation levels at sites can be used in a deconvolution process to determine tissue proportions in the cfDNA of CSF.

Genomic coverage (e.g., fragmentation density) can be used in various ways, e.g., measure a tumor fraction in the cfDNA or to differentiate a stage of a brain tumor. For the tumor fraction, an extent of copy number aberration can be used. For the stage, a variability in genomic coverage across regions can differentiate, where more variability occurs for higher stages.

A concentration of cfDNA in CSF can also differentiate a stage of a brain tumor, e.g., a benign tumor or glioma. A higher concentration in CSF provides surprisingly accurate results to differentiate the stage.

Besides nuclear DNA, mitochondrial DNA (mtDNA) can also be used. A normalized amount of mtDNA can be used to determine a classification of whether the subject has a benign tumor or glioma. Surprisingly, a lower amount (e.g., less than a threshold/reference) of mtDNA indicates Glioma.

### I. CEREBROSPINAL FLUID EXTRACTION AND PREPARATION

Malignancies and various medical conditions within the central nervous system (CNS) are common worldwide and are associated with high morbidity and mortality. Over the past decade, the detection of circulating tumor DNA (ctDNA) from liquid biopsy has emerged as a common, non-invasive approach to cancer diagnosis. As a result of anatomical barriers, such as the blood brain barrier (BBB), which is believed to restrict the passage of cfDNA molecules from the CNS to the peripheral blood circulation, the detection of ctDNA from CNS malignancies remain to be challenging (Berzero et al. 2023). Existing studies on detecting various tumor types from plasma cfDNA have demonstrated that brain-related malignancies showed the lowest fraction of individuals with detectable ctDNA (Bettegowda et al. 2014).

A more promising approach has been to study cell-free DNA (cfDNA) derived from the cerebrospinal fluid (CSF), a colorless bodily fluid that circulates between the ventricles of the brain and spinal cord. The primary function of CSF is to provide protection to the brain and can act as a shock absorber for the brain. CSF can also provide nutrients and perform waste removal.

Several groups have reported that cfDNA derived from CSF is more informative than plasma in the diagnosis of malignancies restricted to the CNS, such as in the diagnosis of medulloblastoma (Escudero et al. 2020), meningeal carcinomatosis (Zhao et al. 2019), spinal cord tumors (Chai et al. 2024) and glioma (Mouliere et al. 2018). Most studies assessing ctDNA from the CSF adopt the use of targeted sequencing methods to detect for mutational patterns and copy number aberrations. These methods of molecular profiling have shown strong diagnostic abilities in cancer sub-typing and studying a wide spectrum of primary and metastasis tumors in the brain (Bale et al. 2021). The CSF allows for greater sensitivity for CNS related pathologies as it is in closer and more direct contact with cells of the CNS. Shedding of tumors in the CNS is more easily accessible in the CSF reservoir compared to blood circulation. CSF is also less likely to be contaminated by peripheral blood cells or other cellular components, with an overall low cell count of white blood cells, which provides a clearer picture of CNS pathology over other cell types.

In addition or alternatively to studying patterns of genomic alterations in cfDNA to diagnose cancer, the study of the fragmentomic and epigenetic profiles provides an alternative approach, allowing for a multi-modal analysis of cfDNA features for molecular diagnosis.

Fragmentomics characteristics, including size distribution, end motifs, and jagged end patterns, offer insights into the structural components of DNA, as well as the biological processes involved in cell death and nuclease-mediated DNA fragmentation. For example, the characteristic 166-bp modal peak of cfDNA, and series of peaks with 10-bp periodicity patterns reflect the nucleosome structural of DNA (Lo et al. 2010). Various DNA nucleases contribute to the fragmentation process of DNA, as determined through the study of nuclease deficient mouse, include the major serum nuclease, deoxyribonuclease 1 like 3 (DNASE1L3); DNA fragmentation factor subunit beta (DFFB) fragments DNA during the apoptotic process, and deoxyribonuclease 1 (DNASE1) also contributes to downstream cutting of DNA (Serpas et al. 2019; Han et al. 2020).

The study of fragmentomic features also allow for deduced epigenetic status, such as DNA methylation (Zhou et al. 2022) and histone modifications. The use of fragmentomics-based methylation analysis allows for tissue-of-origin analysis and can provide diagnostic abilities of using plasma cfDNA. We performed an analysis of cfDNA derived from CSF in the diagnosis and assessment of cancer in the central nervous system.

In various embodiments, CSF can be extracted from patients along with paired plasma samples and cfDNA can be extracted from the CSF and plasma samples to measure fragmentomic features.

### A. Location of CSF within the Brain

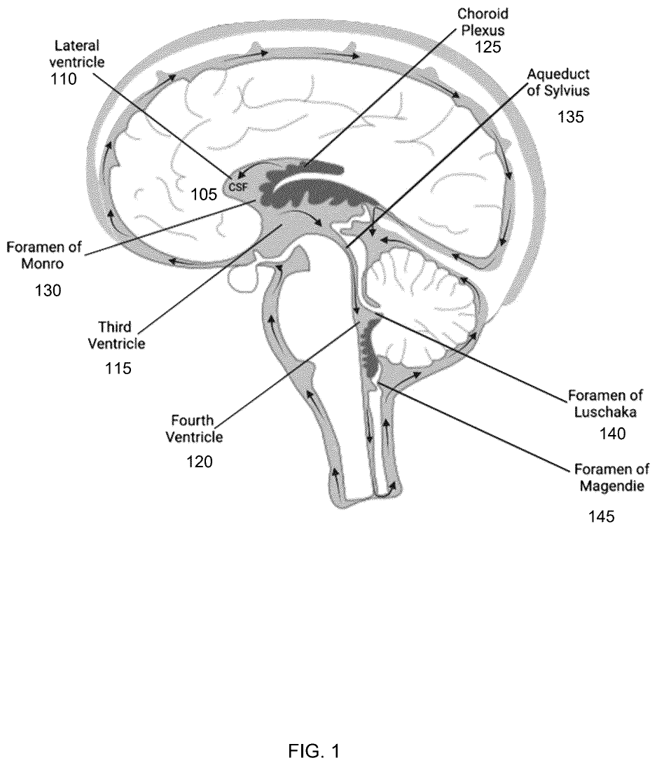

FIG. 1 shows an illustration of the brain and the circulation of CSF around various anatomical features. CSF 105 is a colorless bodily fluid that circulated between the ventricles (e.g. lateral ventricle 110, third ventricle 115, fourth ventricle 120) of the brain and the spinal cord. CSF 105 circulates within the empty chambers of the brain providing protection, nutrients, and waste removal. CSF 105 is secreted primarily from a class of specialized cells called choroid plexuses 125. A foramen in the brain refers to a channel or opening that allows for the passage of structures (e.g., blood vessels, nerves, etc) or CSF 105 from one brain region to another brain region. For example, foramen involved in CSF 105 circulation include the Foramen of Monro 130, which connects the lateral ventricles 110 to the third ventricles 115. The Aqueduct of Sylvius 135 connects the third ventricle 115 and the fourth ventricle 120. The Foramen of Luschka 140 and the Foramen of Magendie 145 direct CSF 105 flow the from fourth ventricle 120 to the subarachnoid space (not shown). The mean volume of CSF 105 is around 125 ml and about 500 mL of CSF 105 is produced every day, resulting in constant production and turnover of CSF 105. CSF 105 is drained in a layer of the brain called the arachnoid granulations (not shown). CSF 105 has decreased protein levels compared to the levels in plasma and may have less than 1% of the levels of proteins circulating in plasma. The pH of CSF 105 is similar to that of plasma at around 7.30-7.36.

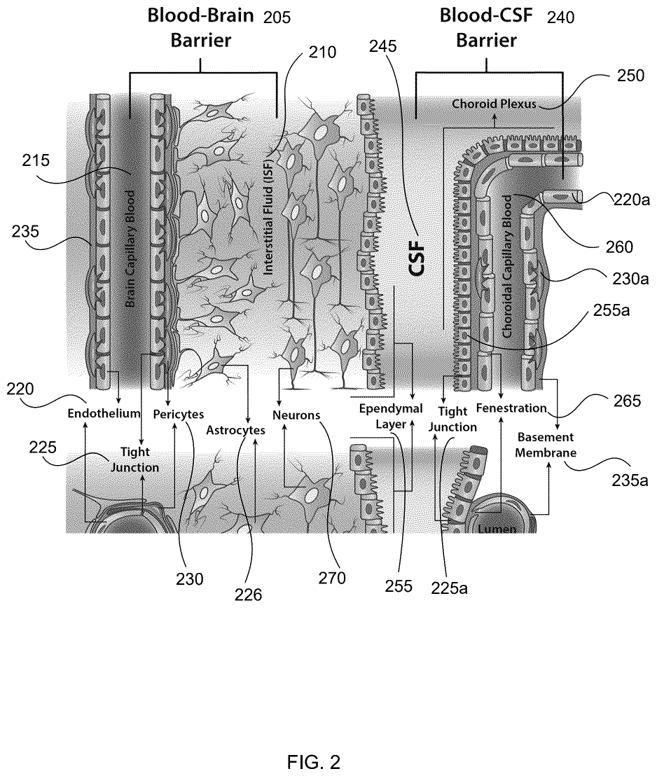

FIG. 2 shows an illustration of the anatomical barriers of the brain and blood circulation. A layer called the blood-brain barrier 205 is a selective semi-permeable barrier that separates cells and interstitial fluid 210 within the brain from blood circulation (e.g., blood in a brain capillary 215). The endothelium (or endothelial cells) 220 of a brain capillary 215 forms the basis of the blood-brain barrier 205. Briefly, endothelial cells 220 line the luminal surface of brain capillaries 215 and form a continuous monolayer of cells. The endothelial cells 220 can directly interact with one another using a variety of proteins to create a tight junction 225, which can create a physical barrier between the interstitial fluid 210 and the blood in a brain capillary 215. Through direct contact or secretion of soluble factors, astrocytes 226 and pericytes 230 embedded in the basement membrane 235 can provide structural support to and regulate endothelial cells 220 associated with tight junctions 225 of the blood-brain barrier 205. Usually, there is believed to be a low amount of brain-derived DNA circulating in blood due to this barrier of protection that allows for very specialized and tight regulation of molecules within the blood and the brain.

There is another layer called the blood-CSF barrier 240. The blood-CSF barrier 240 is a protective, semipermeable barrier between blood and CSF 245 and is primarily located in the choroid plexus 250 (also feature 125 with respect to FIG. 1). The choroid plexus 250 is lined with a specialized layer of epithelial cells referred to as the ependymal layer 255. Cells of the ependymal layer 255 produce CSF 245 and assist in circulating CSF 245 through ventricles of the brain. A subset of ependymal cells 255a can be joined together with tight junctions 225b and can surround a choroidal capillary 260 to form the basis of the blood-CSF barrier 240. Pericytes 230a embedded within a basement membrane 235a can provide support to the subset of ependymal cells 255a with tight junctions 225b and endothelial cells 220a of the choroidal capillary 260. This is in contrast to the BBB 205 where tight junctions 225a between endothelial cells 220 of a brain capillary 215 form the basis of the barrier. Small openings (fenestrations 265) between the endothelial cells 220a of the choroidal capillary 260 can allow for the passage of certain components of circulating blood and plasma (e.g., plasma proteins, ions, etc.) into the area surrounding the choroid plexus 250. Cells of the ependymal layer 255 (including the subset of ependymal cells 255a) can use these components in the production of CSF 245. It is also well-established that CSF 245 has better circulation and contact with many cells in the brain such as neurons 270 and glial cells. CSF 245 may play a better role than plasma in diagnosis of malignancies in the central nervous system because of this increased contact with neurons 270 and glial cells.

### B. Extraction of CSF

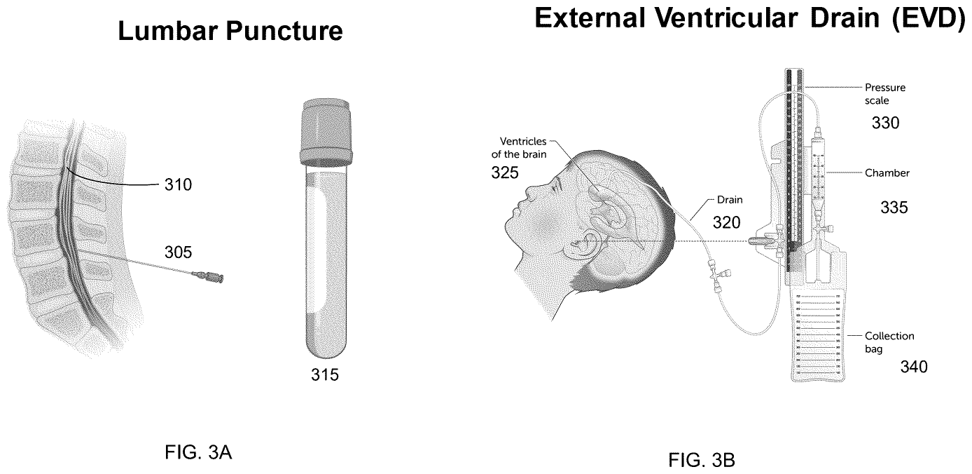

FIGS. 3A-3B show illustrations of two example techniques for CSF extraction. FIG. 3A shows an illustration of lumbar puncture, where a needle 305 is inserted in a particular part of the spinal cord 310 to extract CSF into a collection tube 315.

FIG. 3B shows an illustration of external ventricular drain (EVD), where CSF is drained into a collection. A drain 320 is inserted in the ventricles of the brain 325 to remove excess CSF. A pressure scale 330 marks a level of drainage as CSF drained from the ventricles of the brain 325 moves through a drip chamber 335 and into a collection bag 340.

CSF can be extracted for diagnostic purposes including identifying neurological disorders and testing for the presence of infections such as bacterial infection, inflammation and malignancies. Diagnostic purposes can be identified by looking at the protein levels, inflammatory markers, or white blood cells in CSF. Certain patients may have a buildup of CSF in the brain that can cause increased swelling and intracranial pressure. Extracting and draining excess CSF may help release intracranial pressure.

### C. Samples

Paired CSF and plasma samples were collected from patients having a variety of neurological conditions, including patients having high intracranial pressure, various benign brain tumors, and high-grade gliomas. Analyses of paired CSF and plasma samples from the same patients and time-point were performed to characterize and identify cfDNA features of each sample type between patient cohorts (e.g., comparing patients with a benign brain tumor to patients with a high-grade glioma). Patients with secondary complications, including hemorrhage (as blood and CSF would be mixed), cases without paired plasma, and cases with primary tumor elsewhere (e.g., outside of the brain) were excluded from the analyses. The sections below describe the characteristics of the patient cohorts having the various neurological conditions.

### 1. ICP and Brain Tumor

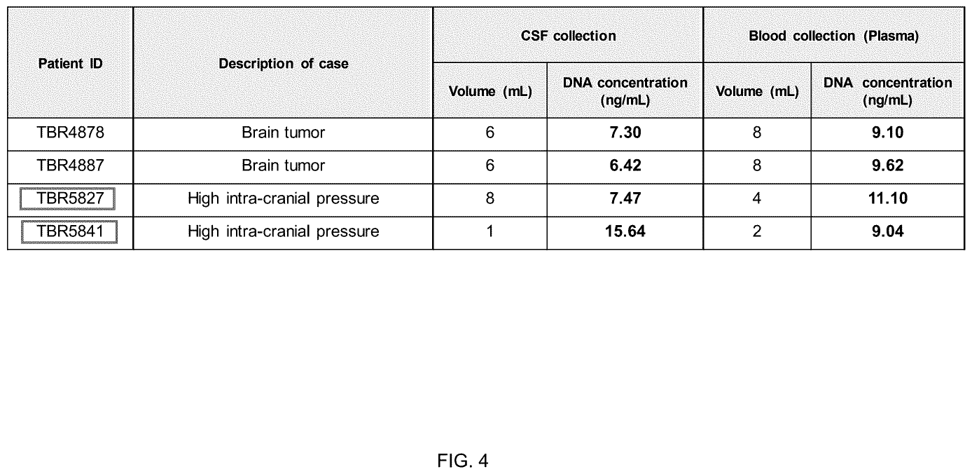

FIG. 4 is a table summarizing patient information and corresponding cfDNA concentration from plasma and CSF. We collected paired plasma and CSF samples from two individuals with high intra-cranial pressure (ICP), where the draining of CSF was performed by external ventricular drainage (EVD) as described in FIG. 3B. High intracranial pressure can occur in cases with high production of CSF but low absorption, causing an increase in pressure. We have also collected paired plasma and CSF from two individuals with benign brain tumors. Both biological fluids were collected in EDTA tubes.

We performed pair-end sequencing (100 bp×2) using Illumina-sequencing platforms, with 100 million pair-end sequencing reads per sample. From one individual with high ICP (TBR5841), bisulfite sequencing was performed to compare the tissue-of-origin of the cfDNA pool from plasma and CSF. FIG. 4 shows slightly decreased cfDNA concentration in CSF compared to cfDNA concentration in the paired plasma collection for each patient, with exception of the last case (TBR5841).

In some example data, we focused on comparing the cases of high intracranial pressure to patients with benign brain tumors because patients with late-stage brain tumors could add aconfounding variable from the circulating tumor DNA. Because the patients are not all healthy individuals, there may be some limitations due to abnormalities in the samples. CSF collected from healthy individuals can be rare because methods of collection can be invasive. CSF may also be collected due to concerns a patient may have meningitis or an infection of the brain, and when CSF is collected, it may look like pus. The samples collected from non-cancer patients were collected to release intracranial pressure through external ventricular drainage as described in FIG. 3B.

### 2. Benign

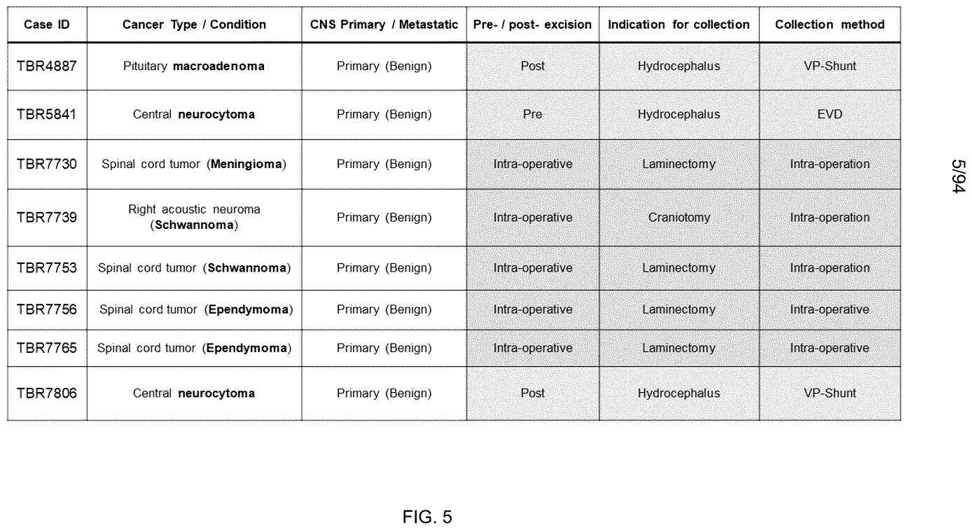

FIG. 5 is a table summarizing the paired CSF and plasma collection information from patients having benign brain tumors. CSF and plasma from a total of 8 patients having a variety of low-grade (e.g., early stage) benign primary brain tumors were collected. The benign cancer types included pituitary macroadenoma; central neurocytoma; spinal cord tumors, including meningioma, schwannoma and ependyomas; and right acoustic neuroma (schwannoma). The indications for collection included hydrocephalus, laminectomy, and craniotomy. CSF was collected using a ventriculoperitoneal shunt (VP), EVD as described in FIG. 3B, or intraoperatively.

### 3. Glioma

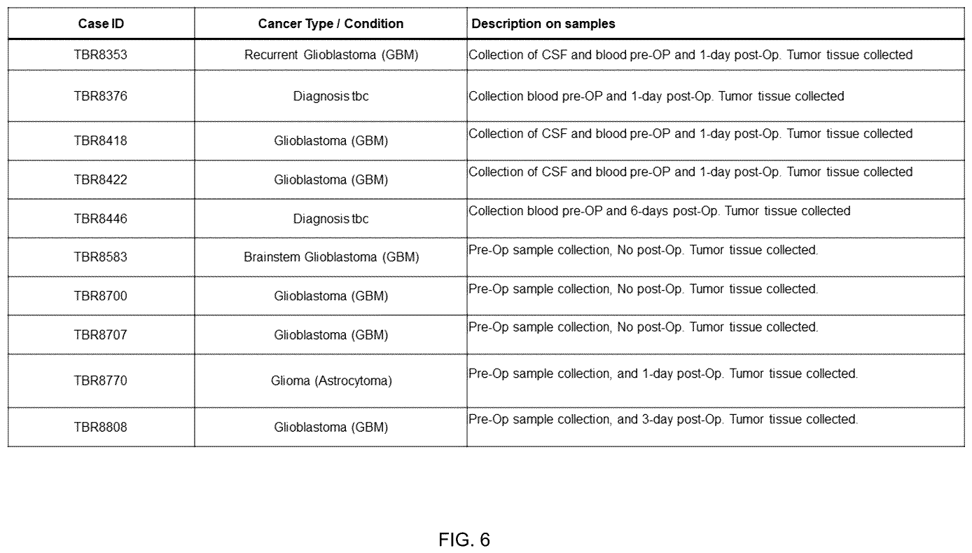

FIG. 6 is a table summarizing the paired CSF and plasma collection information from patients having glioma brain tumors. CSF and plasma from a total of 10 patients having a variety of high-grade (e.g., late stage) glioma primary brain tumors were collected. Seven of the 10 gliomas were diagnosed as glioblastoma. One glioma was diagnosed as an astrocytoma and the remaining two samples had an undetermined glioma type. CSF and blood were collected pre-operatively for each patient. Seven patients had CSF and blood collected between 1-6 days post-operatively. Tumor tissue was also collected from each patient.

### D. Sample Preparation and Analysis

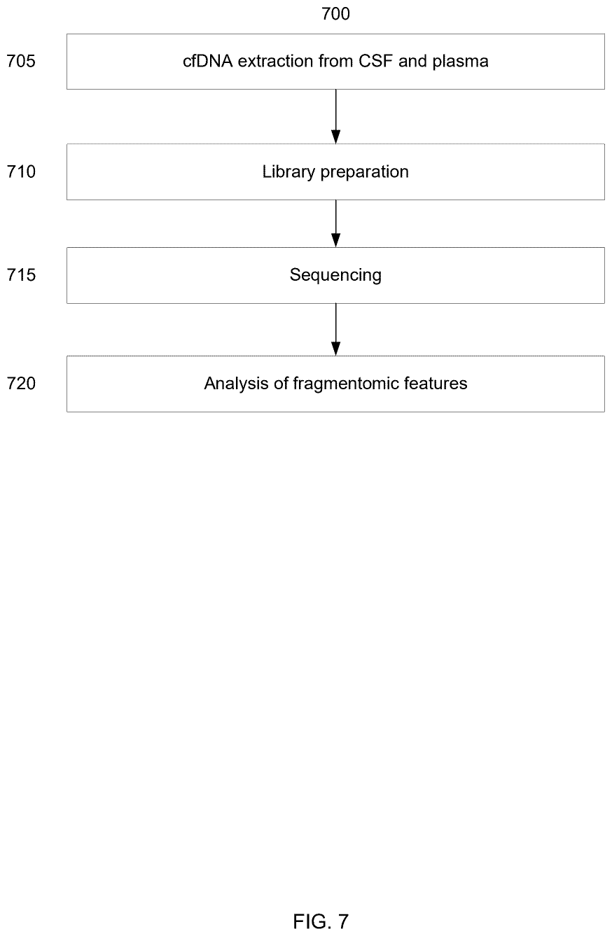

FIG. 7 shows a diagram of an example workflow 700 performed to analyze cfDNA in CSF and plasma.

At step 705, cfDNA is extracted from paired CSF and plasma samples. Methods for cDNA extraction can include ethanol precipitation, anion exchange resin (coupled with ethanol precipitation), silica gel membrane binding, and magnetic silica particle binding technologies. Examples of commercially available cfDNA extraction kits using these methods can include QIAamp circulating nucleic acid kit (Qiagen), NucleoSpin Plasma XS (Machery-Nagel), QIAmp MinElute ccfDNA Mini Kit (Qiagen), cfPure cell-free DNA extraction kit (BioChain), MagMAX cell-free DNA isolation kit (ThermoFisher scientific), EZ2 Connect kit (Qiagen), and MagNA Pure 24 Total NA Isolation kit (Roche). cfDNA extraction methods are not so limited to these examples.

In some examples below, the QIAamp Circulating Nucleic Acid Kit and the EZ2 Connect Kit (used for ssDNA sequencing library preparation methods) were used for cfDNA extraction.

At step 710, a sequencing library is prepared. Example sequencing library preparations can include double stranded DNA (dsDNA) or single stranded DNA (ssDNA) methods. The sequencing library preparations can further include methods compatible with whole genome sequencing (WGS), bisulfite sequencing, and/or 5-hydroxymethylcytosine (5hmC) sequencing (e.g., APOBEC-Coupled Epigenetic sequencing also referred to as ACE-seq). The library preparation methods need not be so limited to these examples.

In some examples below, the New England Biolabs dsDNA Library Preparation Kit (New England Biolabs), EpiTect Bisulfite Kit (Qiagen), and SRSLY NGS DNA Library Preparation Kits (for ssDNA libraries) with bisulfite sequencing version performed as a modification of the protocol (Claret Biosciences) were used to prepare sequencing libraries.

At step 715, sequencing is performed to identify fragmentomic features. Various types of sequencing on various sequencing platforms can be used, as described herein, e.g., Illumina platforms, Pacific Biosciences, or Oxford Nanopore. Sequence reads can be obtained, which can be aligned to a reference genome, e.g., a human reference genome.

At step 720, fragmentomic features are analyzed. Example fragmentomic features for analysis can include size profile (Section III), end motifs (Section IV), cleavage profiles (Sections IV and V), determination of epigenetic features, including methylation (Section V), copy number aberrations (Section VII), and mitochondrial DNA (Section VIII).

Table 1 shows the dsDNA library preparation methods used to prepare sequencing libraries from paired CSF and plasma of the cohort of the patients having benign tumors (e.g., as described in FIG. 7). cfDNA from paired CSF and plasma samples of eight patients was used to prepare dsDNA sequencing libraries compatible with whole genome sequencing (WGS) and bisulfite sequencing. One dsDNA library was prepared from cfDNA of a single patient using an ACE-seq compatible library preparation method. The fold coverage refers to the sequencing depth (e.g., the average number of sequencing reads for each base in a region of DNA). For example, a fold coverage of 5 can mean that, on average, each base in a target region of DNA has been sequenced 5 times. The dsDNA sequencing libraries prepared from CSF and plasma from patients having benign tumors had fold coverage (e.g., sequencing depth) of 5 or 10.

TABLE 1
dsDNA sequencing library preparation methods from paired CSF and
plasma from the cohort of the patients having benign tumors

| Library preparation | Number of cases | Fold |
|---|---|---|
| method | (paired plasma + CSF) | Coverage |
| dsDNA library (WGS) | 8 | 5 |
| dsDNA bisulfite sequencing | 8 | 5 |

Table 2 shows the DNA library preparation methods used to prepare sequencing libraries from paired CSF and plasma from cohorts of patients having either benign tumors or high-grade glioma tumors (e.g., as described in FIGS. 5 and 6, respectively). cfDNA from paired CSF and plasma samples of eight patients in the benign cohort was used to prepare dsDNA sequencing libraries compatible with WGS and bisulfite sequencing. cfDNA from paired CSF and plasma samples of six patients in the benign cohort was used to prepare ssDNA sequencing libraries compatible with WGS and bisulfite sequencing. The following case IDs from the benign cohort (as depicted in FIG. 5) were selected for ssDNA library preparation: TBR7730, TBR7739, TBR7753, TBR7756, TBR7765, and TBR7806. cfDNA from paired CSF and plasma samples of six patients in the glioma cohort was used to prepare ssDNA and dsDNA sequencing libraries compatible with WGS. ssDNA sequencing libraries from six patients in the high-grade glioma cohorts were also prepared using methods compatible with bisulfite sequencing. The following case IDs from the high-grade glioma cohort (as depicted in FIG. 6) were selected for ssDNA library preparation: TBR8353, TBR8418, TBR8422, TBR8700, TBR8707, and TBR8808. The ssDNA and dsDNA sequencing libraries prepared from CSF and plasma from cohorts of patients having either benign tumors or glioma tumors had fold coverage (e.g., sequencing depth) of 5.

TABLE 2
Sequencing library preparation methods from paired
CSF and plasma from cohorts of patients having
either benign tumors or glioma tumors

| | | Number of cases | |
|---|---|---|---|
| | Tumor | (paired plasma + | Fold |
| Library preparation method | cohort | CSF) | Coverage |
| dsDNA library (WGS) | Benign | 8 | 5 |
| dsDNA bisulfite sequencing | Benign | 8 | 5 |
| ssDNA library (WGS) | Benign | 6 | 5 |
| ssDNA (bisulfite sequencing) | Benign | 6 | 5 |
| dsDNA library (WGS) | Benign | 6 | 5 |
| ssDNA library (WGS) | Glioma | 6 | 5 |
| ssDNA (bisulfite sequencing) | Glioma | 6 | 5 |

### II. CFDNA CONCENTRATION

Various properties in CSF differ to that of plasma, including cell types of origin, composition of circulating proteins, including nucleases, analyte concentrations, and mechanisms of DNA clearance. Additionally, the total concentrations of cfDNA are generally elevated with more advanced stages of cancer, as demonstrated in plasma cfDNA (van der Pol and Mouliere 2019; Mattox et al. 2023). But the accuracy is quite low as the elevation is not very significant, making the total concentration generally not viewed as a viable marker.

### A. Measurements

A variety of techniques can be used to quantify nucleic acids in a sample. The cfDNA concentration of each extracted sample in the examples provided below was measured using a Qubit fluorometer instrument (Invitrogen). A fluorescent dye can correlate a mass using samples of a known mass of DNA to an intensity of the signal. Such a calibration using samples manufactured to have a particular concentration can provide a measured of the cfDNA concentration. For example, a function or a table can convert a measured intensity to a specific cfDNA concentration by finding an entry having a matching intensity or inputting the measuring intensity into a calibration function determined from the calibration data points for the known samples manufactured to have a particular cfDNA concentration. Various techniques can be used to measure the concentration besides the use of a fluorescent dye. For example, cfDNA concentration can also be quantified using: 1) Quantitative PCR methods, 2) Spectrophotometer (UV-vis), 3) Capillary electrophoretic methods, 4) digital PCR methods (e.g., droplet digital PCR), and fluorometric techniques.

### B. Differences Between CSF and Plasma

Below we characterize total cfDNA concentrations between paired plasma and CSF samples of patients having benign tumors of the CNS.

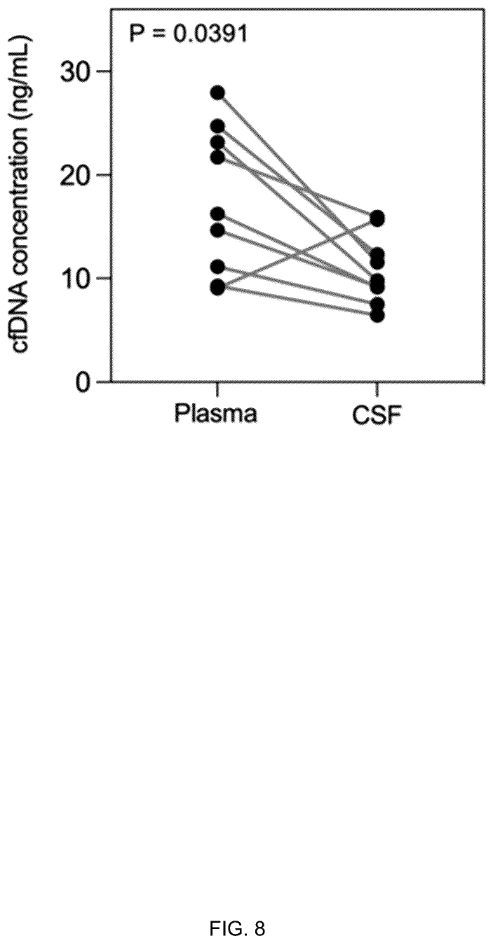

FIG. 8 represents the total concentrations of cfDNA in paired plasma and CSF samples from cases of benign tumors of the central nervous system (n=8 cases). The mean and median plasma cfDNA concentrations were 17.52 ng/mL and 16.22 ng/mL, respectively. The interquartile range (IQR) of plasma cfDNA concentrations was 11.1-23.13 ng/mL. The mean and median CSF cfDNA concentrations was 10.81 ng/mL and 9.75 ng/mL, respectively. The IQR of CSF cfDNA was 9.13-12.28 ng/mL. The mean cfDNA concentrations were determined to be statistically different (p=0.0391) as assessed by Wilcoxon matched-pairs signed rank test. These results suggest that the total cell-free DNA concentrations in CSF is lower compared to paired plasma samples.

### C. Differentiation Between Benign and Glioma

We also characterize the total cfDNA concentrations of paired plasma and CSF samples between patients having benign tumors of the CNS and those having gliomas.

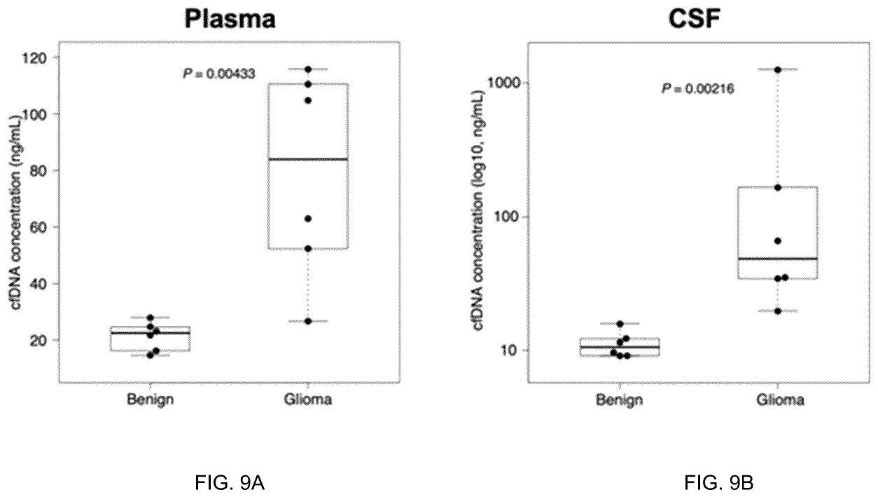

FIG. 9A represents the total cfDNA concentrations in plasma samples from cases of benign tumors and high-grade glioma tumors (n=6 cases per tumor type). Total cfDNA concentrations were measured using fluorometric methods (Qubit) and normalized by sample volume (expressed as ng/mL). The mean cfDNA concentration between tumor types was determined to be statistically different (p=0.00433) as assessed by non-parametric unpaired t-test. These data support that patients having high-grade glioma tumors have elevated plasma cfDNA concentrations as compared to patients having benign CNS tumors.

FIG. 9B represents the total cfDNA concentrations in CSF samples from cases of benign tumors and high-grade glioma tumors (n=6 cases per tumor type). Total cfDNA concentrations were measured using fluorometric methods (Qubit) and normalized by sample volume (expressed as ng/mL). The mean cfDNA concentration between tumor types was determined to be statistically different (p=0.00216) as assessed by non-parametric unpaired t-test. These data support that patients having high-grade glioma tumors have elevated CSF cfDNA concentrations as compared to patients having benign CNS tumors.

Table 3 shows the paired plasma and CSF cfDNA concentrations from patients having benign brain tumors or high-grade gliomas. The values correspond to the data presented in FIGS. 9A and 9B.

TABLE 3
Paired plasma and CSF cfDNA concentrations from patients
having benign brain tumors or high-grade gliomas.

| Plasma cfDNA concentration (ng/mL) | | | | CSF cfDNA concentration (ng/mL) | | | |
|---|---|---|---|---|---|---|---|
| Benign | | Glioma | | Benign | | Glioma | |
| TBR7753 | 23.13 | TBR8353 | 26.66 | TBR7753 | 9.75 | TBR8353 | 19.79 |
| TBR7730 | 14.61 | TBR8418 | 110.64 | TBR7730 | 9.19 | TBR8418 | 166.90 |
| TBR7739 | 24.71 | TBR8422 | 115.92 | TBR7739 | 12.28 | TBR8422 | 66.55 |
| TBR7756 | 27.94 | TBR8700 | 63.00 | TBR7756 | 11.53 | TBR8700 | 1264.00 |
| TBR7765 | 21.70 | TBR8770 | 104.88 | TBR7765 | 15.89 | TBR8770 | 34.48 |
| TBR7806 | 16.22 | TBR8808 | 52.32 | TBR7806 | 9.13 | TBR8808 | 35.7 |

These data combined data suggest that total cell-free DNA concentrations in CSF is lower compared to paired plasma samples. Furthermore, patients having high-grade gliomas have elevated plasma and CSF cfDNA concentration compared to patients having benign CNS tumors.

### D. Method

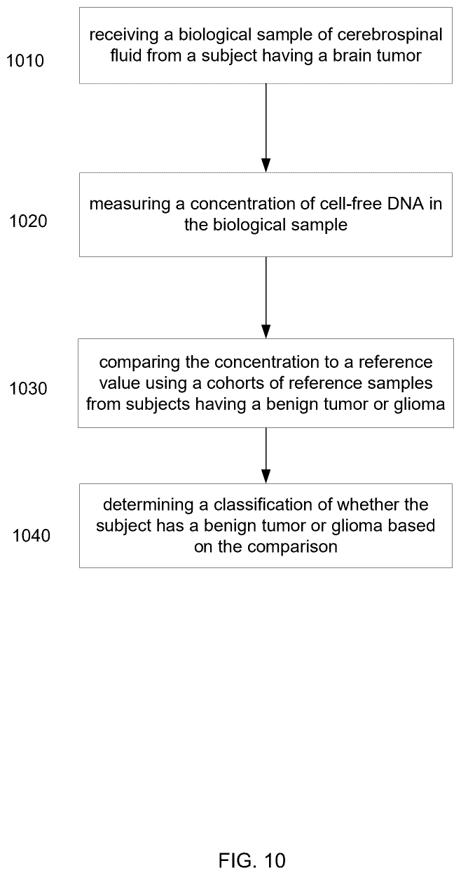

FIG. 10 is a flowchart illustrating a method 1000 for detecting a brain tumor in a subject based on cfDNA concentration, according to some embodiments of the present disclosure. Portions or all steps of method 1000 can be performed by a computer system, including one or more processors. The method 1000 can use a trained ML model that was trained by the computer system or another computer system. The computer system can comprise various devices, e.g., one device that performed the training and another that uses the trained model.

At block 1010, method 1000 can include receiving a biological sample of cerebrospinal fluid from a subject having a brain tumor. The sample can be received from another party that obtained the sample from the subject.

At block 1020, method 1000 can include measuring a concentration of cell-free DNA in the biological sample. The concentration can be measured using various techniques, such as quantitative PCR, a spectrophotometer (UV-vis), capillary electrophoresis, digital PCR, or fluorometric techniques.

At block 1030, method 1000 can include comparing the concentration to a reference value. The reference value can be trained using a first cohort of reference samples from subjects having a benign tumor and a second cohort of reference samples from subjects having glioma. As an example, the reference value (also referred to as a cutoff value or threshold value) can be between 16-19 ng/mL. The specific reference value can be selected based on a desired sensitivity and specificity as can any reference value described herein.

At block 1040, method 1000 can include determining a classification of whether the subject has a benign tumor or glioma based on the comparison. For a subject having a benign brain tumor, the concentration can be less than the reference value, e.g., as shown in FIG. 9B. For a subject having a glioma, the concentration can be greater than the reference value. Responsive to determining the classification, treatment can be provided to the subject, e.g., as described herein. Treatment can be provided responsive to any of the techniques described herein.

### III. SIZE

A fragment size can relate to a number of base pairs (also referred to as bases for length of a single strand) that make up a cell-free DNA (cfDNA) fragment. cfDNA fragments can be relatively short. For example, cfDNA fragments may be around 160-180 base pairs long. The size distribution of cfDNA fragments can provide valuable insights into their cellular origins and the physiological or pathological processes occurring within a subject. Techniques such as next-generation sequencing (e.g., of entire fragment or alignment of ends to a reference genome), physical separation techniques (e.g., filtering and/or electrophoresis), or other bioanalytical platforms can be used to determine the fragment sizes. A variety of analysis methods can be used to analyze the differences in sizes of cfDNA, including machine learning approaches, to perform classifications.

In some embodiments, sizes of cfDNA fragments in CSF can be analyzed. The sizes can be measured by performing paired-end sequencing of the DNA fragments to get paired-end reads, which were then aligned to the reference genome. The coordinates of the aligned paired-end reads provide a length (an example of size) of the DNA fragment.

### A. Differences Between Plasma and CSF

We first characterized the cfDNA fragment sizes in plasma and CSF from patients having a variety of neurological conditions, including patients having high intracranial pressure and various benign brain tumors.

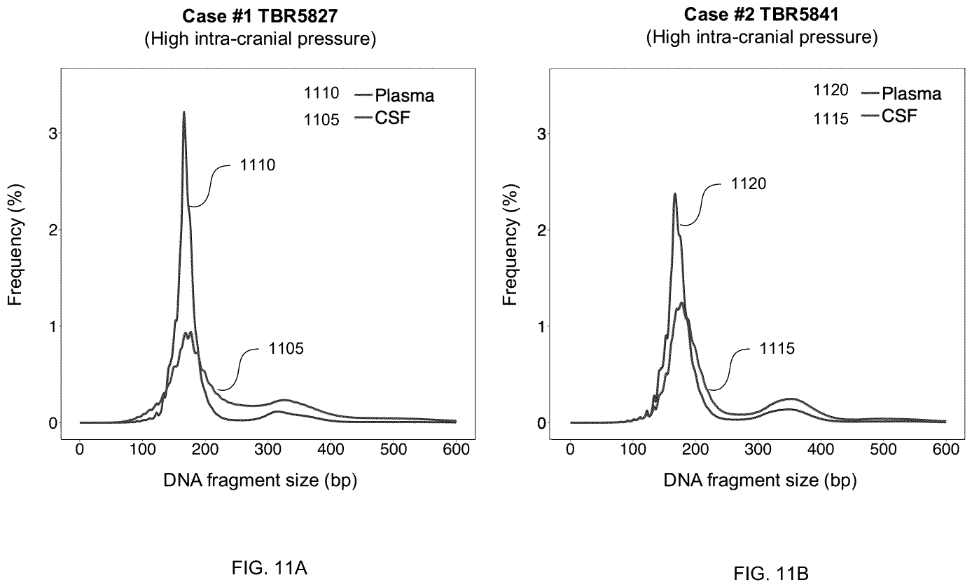

FIGS. 11A-11B show plots of cfDNA size distribution of paired plasma and CSF from two individuals with high intra-cranial pressure, plotted on a linear scale (0-600 bp). FIG. 11A shows results from frequency analysis on CSF 1105 and plasma 1110 samples collected from patient TBR5827 described in FIG. 4. FIG. 11B shows results from frequency analysis on CSF 1115 and plasma 1120 samples collected from patient TBR58431 described in FIG. 4.

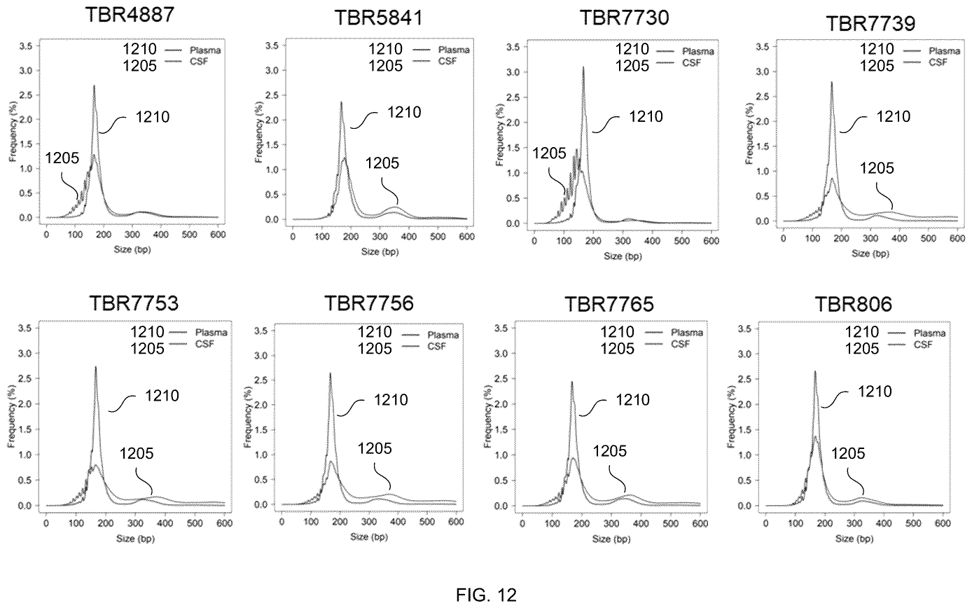

FIG. 12 depicts plasma and CSF cfDNA size profiles from each of eight patients having benign CNS tumors. Samples were collected from patients as described in FIG. 5. Each plot in the figure represents an analysis of a specific patient's cfDNA frequency from CSF 1205 and plasma 1210. The feature numbers for CSF 1205 and plasma 1210 are maintained in each panel for consistency and clarity. The size distributions are plotted on a linear scale from 0-600 bp.

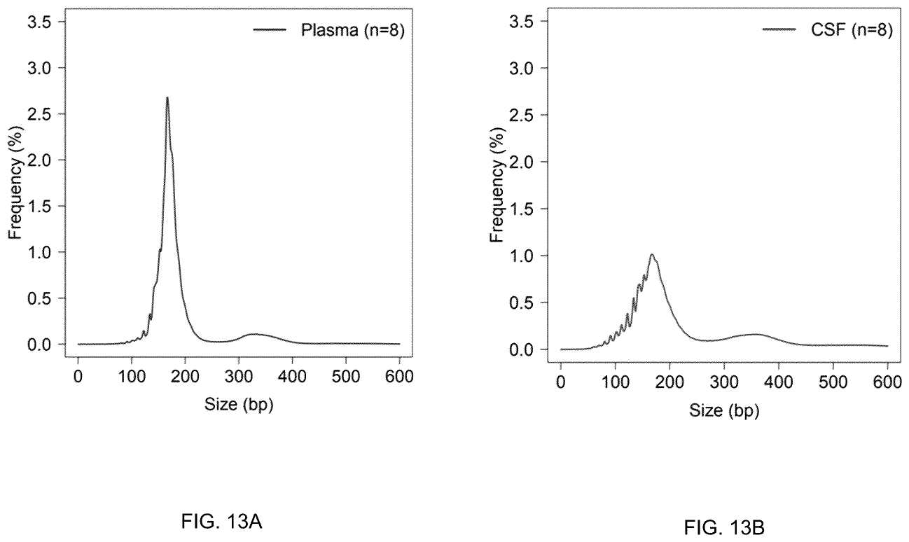

FIGS. 13A and 13B depict the mean of eight cfDNA size profiles between plasma and CSF from patients having benign tumors of the CNS (e.g., size profiles from FIG. 12). Samples were collected from patients as described in FIG. 5. The mean frequencies of each bp are plotted.

The size distributions are plotted on a linear scale from 0-600 bp.

In each example of FIGS. 11A, 11B, 12, 13A, and 13B, the size distribution plots depict a peak frequency of −166 bp, which is reminiscent of the mononucleosome sized units of circulating DNA (Lo et al. 2010; Serpas et al. 2019). The ˜166 bp peaks are present in cfDNA from plasma and CSF. A di-nucleosome peak at around ˜350 bp is also present in each sample, which provides support for plasma and CSF cfDNA having a similar cleavage pattern. The frequency of the 166-bp peak in cfDNA from CSF appears to have a decreased frequency compared to plasma cfDNA, while the proportion of DNA molecules above 200 bp appears to be increased in CSF.

In the examples of patients having benign CNS tumors (e.g., as depicted in FIGS. 12, 13A, and 13B) CSF cfDNA exhibited 10-bp periodicies and higher frequencies of DNA within the 80-140 bp range. This could reflect differences in the clearance mechanism and nuclease fragmentation profile. One reason may be linked to differences in the uptake system of cfDNA and retention time, resulting in more intra-nucleosomal fragmentation.

Overall, these findings provide support for CSF cfDNA having a longer size profile compared to paired plasma cfDNA. The longer size profiles may reflect existing pathology in the patients collected in this study. Individuals were diagnosed with increased intra-cranial pressure, which may lead to increased cell death or physiological trauma that result in longer DNA size profiles. The size profile may serve as a clinical tool, such as in the diagnosis of brain tumors, or a possible marker of neurodegeneration or inflammation which may produce characteristic size patterns.

B. Differentiation of ICP v. Brain Cancer

We next compared the plasma and CSF cfDNA fragment sizes in patients having high intracranial pressure to patients having benign brain tumors.

### 1. Results

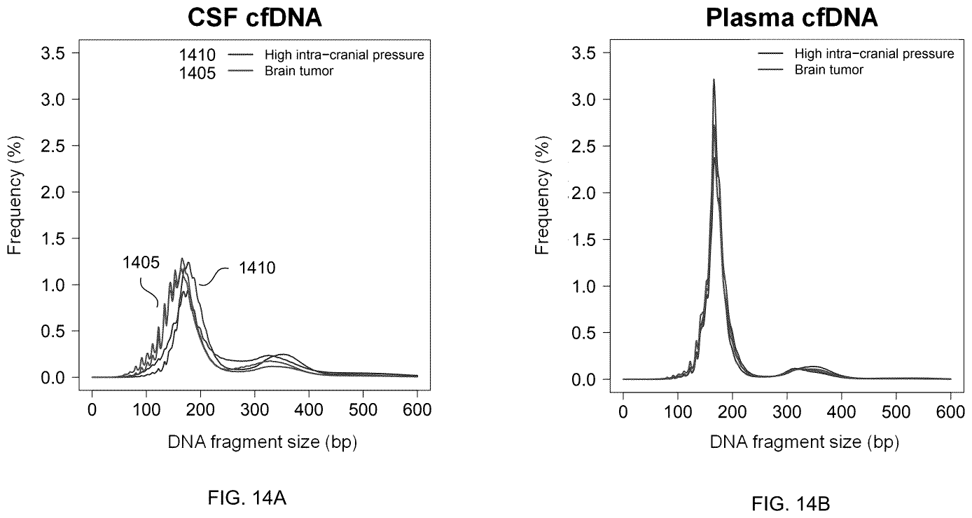

FIGS. 14A-14B show plots of size distribution of DNA fragments from CSF and plasma from individuals with high intracranial pressure and benign brain tumors. In FIG. 14A, shortening of the size profile was observed in the CSF cfDNA from individuals with benign brain tumors 1405 compared to individuals with high intra-cranial pressure (non-tumor) 1410. The CSF cfDNA from patients having benign brain tumors 1405 exhibited 10-bp periodicies and higher frequencies of DNA within the 80-140 bp range whereas patients having high-intracranial pressure 1410 did not. In FIG. 14B, alterations in size distribution were not observed in plasma cfDNA. The plasma cfDNA size profiles are overlapping and are not labeled in the figure. The size distributions are plotted on a linear scale from 0-600 bp.

A peak frequency of ˜166 bp and a di-nucleosome peak at around ˜350 bp were also present in each sample. These results are consistent with those presented in FIGS. 11A, 11B, 12, 13A, and 13B. One possible explanation for observing differences in the cfDNA size distribution of CSF and not plasma is that CSF is in more direct contact with the cell types in the brain and tumor than plasma. As such, fragmentomic features of cfDNA from CSF could be utilized to study pathology specifically restricted within the CNS.

### 2. Method

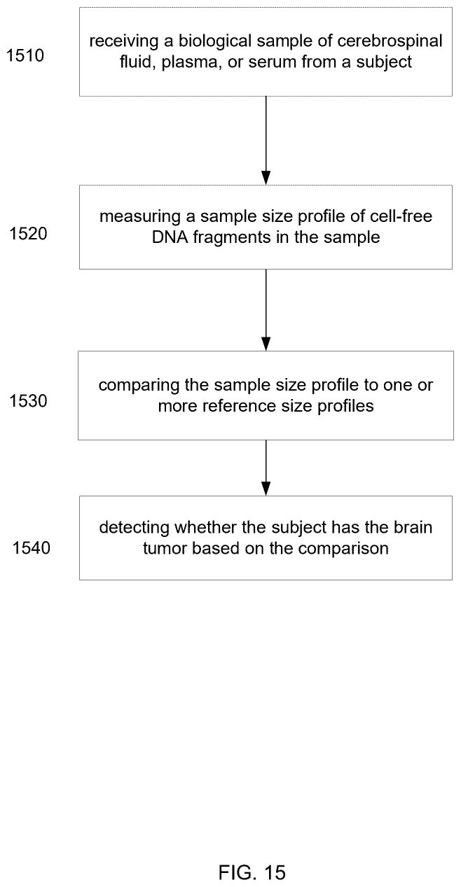

FIG. 15 is a flowchart illustrating a method 1500 for detecting a brain tumor in a subject based on cfDNA size, according to some embodiments of the present disclosure. Portions or all steps of method 1500 can be performed by a computer system, including one or more processors. Method 1500 can use a trained ML model that was trained by the computer system or another computer system. The machine learning model can be comprised of a support vector machine. The computer system can comprise various devices, e.g., one device that performed the training and another that uses the trained model.

In some embodiments, block 1510 and block 1520 are optional and the method 1500 can include receiving a size profile.

At block 1510, method 1500 can include receiving a sample of cerebrospinal fluid from a subject. The sample can be received from another party that obtained the sample from the subject, as can other samples for other methods described herein.

At block 1520, method 1500 can include measuring a sample size profile of cell-free DNA fragments in the biological sample. Measuring a sample size profile can include a physical separation technique. As examples, the physical separation techniques can include filtration and/or electrophoresis. Measuring a sample size profile can also include sequencing the cell-free DNA fragments to obtain sequence reads, measuring sizes of the cell-free DNA fragments using the sequence reads, and generating the sample size profile using amounts of cell-free DNA fragments having a set of sizes. The sequencing can use a single-stranded library preparation technique, as can any of the sequencing described herein. Measuring the sizes of the cell-free DNA fragments can include aligning paired-end reads to a reference genome.

A size can be a size range such as a 5 or 10 bases in width, and many size ranges can be measured, e.g., spanning 100, 200, 300, 400, 500, or 600 bases.

The sample size profile can include a size ratio of a first amount of the cell-free DNA fragments having a first size relative to a second amount of the cell-free DNA fragments having a second size. For example, the first size can be a first range having an upper bound between 120 bases and 180 bases. The second size can be of a size range with a larger upper bound than the first size, e.g., all of the cfDNA fragments

As another example, the sample size profile can be a frequency (percentage) of cfDNA fragments at many sizes (e.g., size ranges spanning 0-600 bases). Various sizes can be used and do not have to be contiguous, e.g., 0-200 bp, 300-400, and 500-600 bases could be used. Sizes greater than 600 bases can also be analyzed.

At block 1530, method 1500 can include comparing the sample size profile to one or more reference size profiles. The one or more reference size profiles can include a first reference size profile determined from cell-free DNA fragments in one or more first reference samples of cerebrospinal fluid measured from one or more first reference subjects having a brain tumor. Comparing the sample size profile to one or more reference size profiles can include inputting the sample size into a machine learning model that is trained using a set of references profiles that include the one or more reference size profiles.

Comparing the sample size profile to the one or more reference size profiles can include comparing the size ratio to a reference ratio of the one or more reference size profiles.

The one or more reference size profiles can further include a second reference size profile determined from cell-free DNA fragments in one or more second reference samples of cerebrospinal fluid measured from one or more second reference subjects having high intra-cranial pressure.

At block 1540, method 1500 can include detecting whether a subject has a brain tumor based on the comparison. The method can also include detecting whether a subject has high intra-cranial pressure or a brain tumor. Block 1540 can be performed in a similar manner as block 1040. Responsive to determining the classification, treatment can be provided to the subject, e.g., as described herein. Treatment can be provided responsive to any of the techniques described herein.

### C. Differentiation Between Benign and Glioma

We next compared the plasma and CSF cfDNA fragment sizes in patients having benign brain tumors to patients having gliomas. Comparisons were performed using samples prepared from ssDNA and dsDNA sequencing libraries. Samples were collected from patients having benign brain tumors and gliomas as described in FIGS. 5 and 6, respectively.

### 1. Results

We first present data using dsDNA sequencing libraries prepared from plasma and CSF cfDNA fragment sizes in patients having benign brain tumors to patients having gliomas. In some embodiments we utilized the cfDNA fragment sizes according to the embodiments in this disclosure as features and trained SVM models for differentiating patients with diseases from subjects without said diseases. The cfDNA fragment sizes for each sample can binned and the bins (regions) can be input into a machine learning model. The machine learning model can then process the feature vectors.

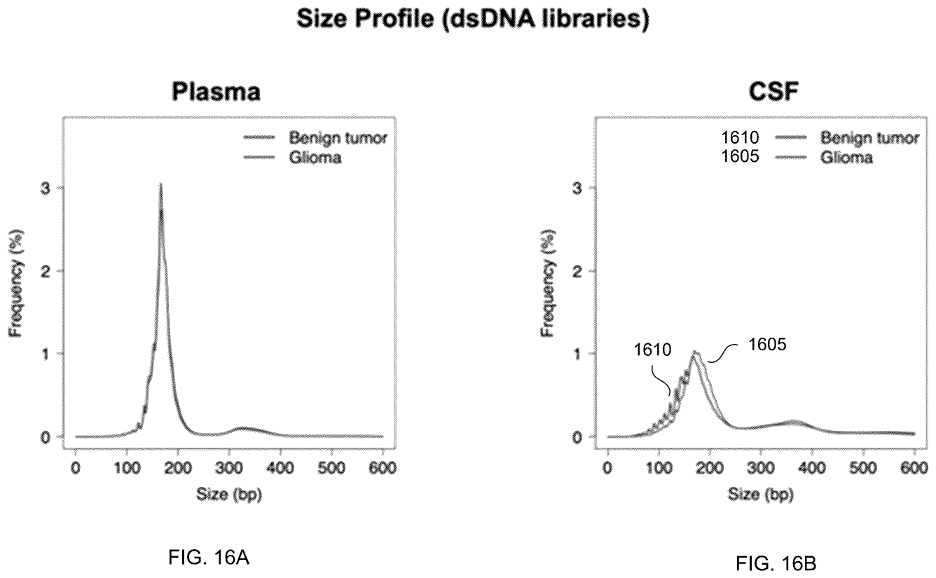

FIG. 16A depicts cfDNA size profiles of cfDNA plasma samples from cases of benign tumor and high-grade glioma. Sequencing libraries were prepared from cfDNA using double-stranded DNA libraries. The size distributions are plotted on a linear scale from 0-600 bp.

Interestingly, plasma cfDNA features showed no major differences in size profiles between benign and glioma tumors. The plasma cfDNA size profiles are overlapping and are not labeled in the figure.

FIG. 16B depicts cfDNA size profiles of cfDNA CSF samples from cases of benign tumor and high-grade glioma. Sequencing libraries were prepared from cfDNA using double-stranded DNA libraries. The size distributions are plotted on a linear scale from 0-600 bp. The size of cfDNA in glioma cases 1505 were shown to have longer size profile (sizes around 150-210 bp) compared to benign tumors 1610. The frequency of 10-bp periodicities is also reduced in cfDNA from glioma patients 1605. This differs from existing work in plasma where higher tumor fraction is characterized by the shortening of cfDNA (Jiang et al. 2015; Lo et al. 2021).

A difference in the frequency of cfDNA molecules can also be calculated. For example, cfDNA can be placed into bins based on fragment size ranges and a difference in the cfDNA frequency between patient cohorts can be calculated. In examples below, cfDNA sizes were binned into 10 bp bins starting from 50 bp up to 200 bp. Additional bins of 200-300 bp, 300-400 bp, and 400-600 bp were added to the analysis. Percent differences in the frequency of cfDNA were calculated for each bin using the median frequencies of cfDNA fragments of the bin. Positive values for the differences indicate a higher proportion of benign cfDNA in a bin. Negative values for the differences indicate a higher proportion of glioma cfDNA in a bin.

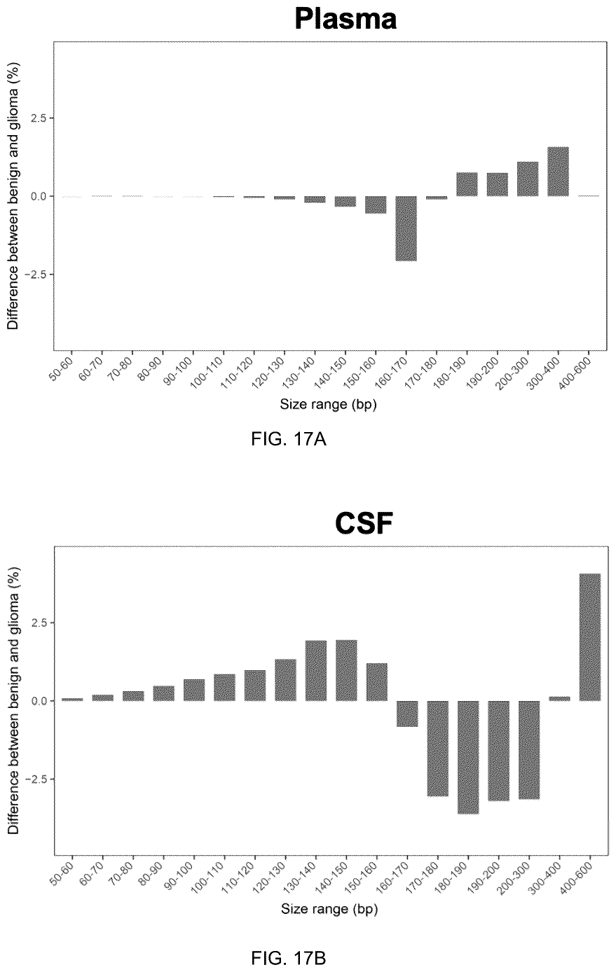

FIG. 17A depicts the difference in the frequency of plasma cfDNA molecules in different size ranges between cfDNA from benign brain tumors and high-grade glioma patients (n=6 per group), using double-stranded DNA libraries. It can be appreciated that patients having high-grade gliomas have higher proportions of cfDNA (e.g., negative differences) in bins between 110-120 bases, 120-130 bases, 130-140 bases, 140-150 bases, 150-160 bases, 160-170 bases, and 170-180 bases compared to patients with benign brain tumors. Additionally, patients having benign brain tumors have higher proportions of cfDNA (e.g., positive differences) in bins between 180-190 bases, 190-200 bases, 200-300 bases, and 300-400 bases. Little, if any, changes in the proportions of cfDNA were observed in the other bins.

FIG. 17B depicts the difference in the frequency of CSF cfDNA molecules in different size ranges between cfDNA from benign brain tumors and high-grade glioma patients (n=6 per group), using double-stranded DNA libraries. It can be appreciated that patients having benign brain tumors have higher proportions of cfDNA (e.g., positive differences) in bins between 50-60 bases, 60-70 bases, 70-80 bases, 80-90 bases, 90-100 bases, 100-110 bases, 110-120 bases, 120-130 bases, 130-140 bases, 130-140 bases, 140-150 bases, 300-400 bases, or 400-600 bases compared to patients with high-grade gliomas. Additionally, patients having high-grade gliomas have higher proportions of cfDNA (e.g., negative differences) in bins between 160-170 bases, 170-180 bases, 180-190 bases, 190-200 bases, and 200-300 bases.

We have also performed a size-based SVM model using 10 bp bin sizes (difference between patients with benign and glioma tumors) using cfDNA from plasma and CSF.

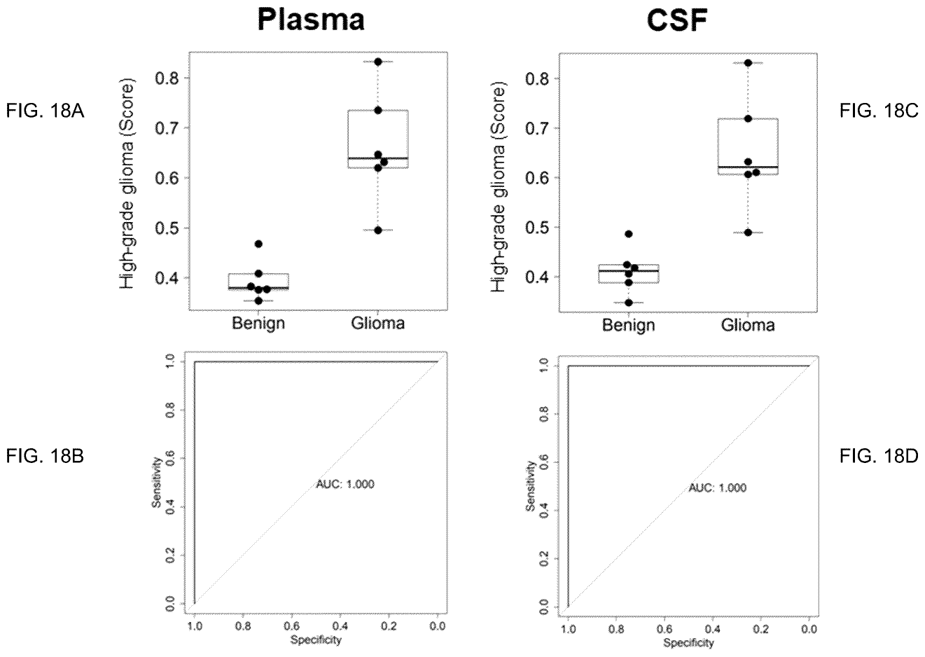

FIG. 18A is a box plot depicting cancer probabilistic scores predicted by SVM models using the difference in plasma cfDNA frequency between patients with benign brain tumors and gliomas in 10 bp bin sizes as inputs into the model. Compared with subjects having benign brain tumors, cancer probabilistic scores predicted by the models were observed to be elevated in patients with high-grade gliomas using the difference in 10 bp bin sizes plasma cfDNA.

FIG. 18B depicts an Receiver Operating Characteristic (ROC) curve analysis using the 10 bp bin sizes of plasma cfDNA to differentiate between subjects with benign brain tumors or high-grade gliomas. We show that the Areas Under the Curve (AUC) for differentiating patients having benign brain tumors from patients with high-grade gliomas was 1.000 using 10 bp bin sizes of plasma cfDNA.

FIG. 18C is a box plot depicting cancer probabilistic scores predicted by SVM models using the difference in CSF cfDNA frequency between patients with benign brain tumors and gliomas in 10 bp bin sizes as inputs into the model. Compared with subjects having benign brain tumors, cancer probabilistic scores predicted by the models were observed to be elevated in patients with high-grade gliomas using the difference in 10 bp bin sizes CSF cfDNA.

FIG. 18D depicts an ROC curve analysis using the 10 bp bin sizes of plasma or CSF cfDNA to differentiate between subjects with benign brain tumors or high-grade gliomas. We show that the AUC for differentiating patients having benign brain tumors from patients with high-grade gliomas was 1.000 using 10 bp bin sizes of CSF cfDNA.

The combined data from dsDNA sequencing libraries prepared from plasma and CSF cfDNA fragment sizes suggest that plasma and CSF have differing size profiles between patients having benign brain tumors and gliomas. For example, in plasma, patients having gliomas have an increased frequency of shorter cfDNA sizes (e.g., for fragments below ˜180 bp) and a reduced frequency of longer cfDNA sizes (e.g., for fragments ˜180-400 bp) compared to patients having benign brain tumors. In contrast to plasma, size profile analysis in CSF indicates that patients having gliomas have a reduced frequency of shorter cfDNA sizes (e.g., for fragments below ˜160 bp) and an increased frequency of longer cfDNA sizes (e.g., for fragments ˜160-300 bp) compared to patients having benign brain tumors, and then a reduced frequency for cfDNA fragments between 400-600 bp. The frequency of 10-bp periodicities are also reduced in cfDNA from glioma patients.

We next present analyses using ssDNA sequencing libraries prepared from plasma and CSF cfDNA fragment sizes in patients having benign brain tumors to patients having gliomas.

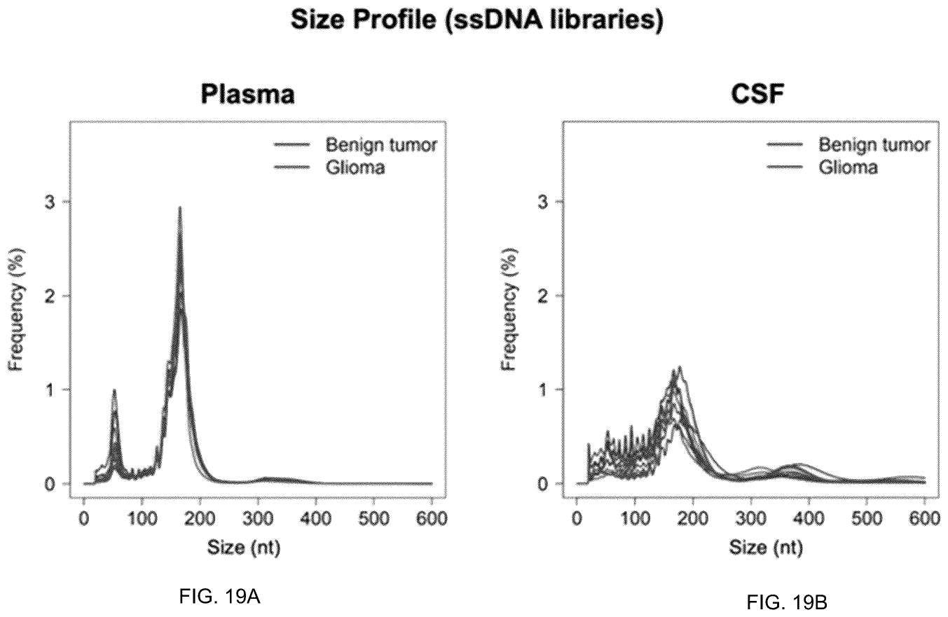

FIG. 19A depicts cfDNA size profiles of cfDNA plasma samples from cases of benign tumor and high-grade glioma. Sequencing libraries were prepared from cfDNA using double-stranded DNA libraries. The size distributions are plotted on a linear scale from 0-600 bp. A peak frequency of ˜166 bp and a di-nucleosome peak at around ˜350 bp were also present in each sample. This is consistent with cfDNA fragment size analysis using dsDNA sequencing libraries. For each patient cohort, an additional peak in the frequency of cfDNA is observed at ˜60 bp fragment sizes. The plasma cfDNA size profiles are overlapping and are not labeled in the figure.

FIG. 19B depicts cfDNA size profiles of cfDNA CSF samples from cases of benign tumor and high-grade glioma. Sequencing libraries were prepared from cfDNA using double-stranded DNA libraries. The size distributions are plotted on a linear scale from 0-600 bp. The additional peak in the frequency of cfDNA observed at ˜60 bp in plasma was not observed in CSF. However, the presence of 10-bp periodicities is also reduced in cfDNA from glioma patients, which is consistent with the data obtained from samples prepared using dsDNA sequencing libraries (e.g., FIG. 16B). The plasma cfDNA size profiles are overlapping and are not labeled in the figure.

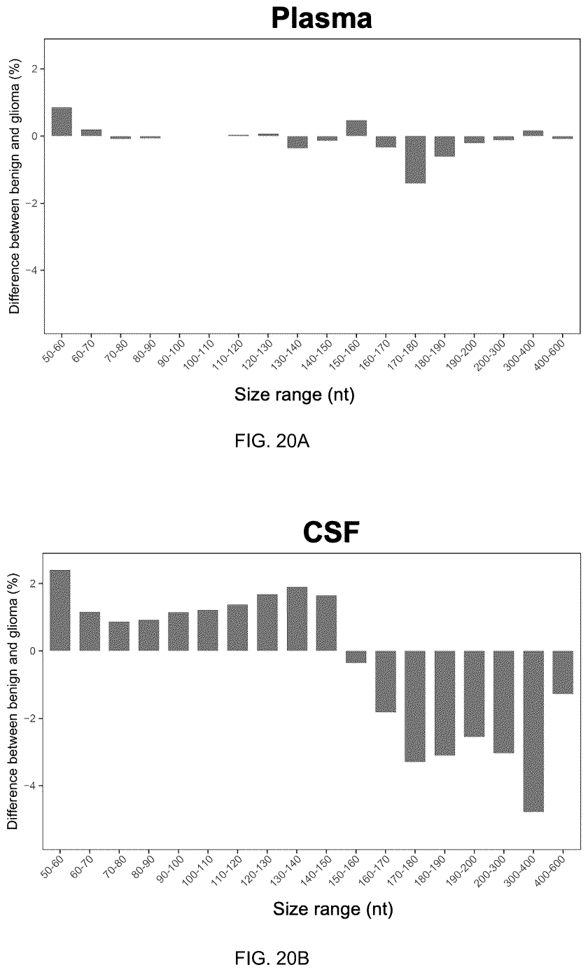

FIG. 20A depicts the difference in the frequency of plasma cfDNA molecules in different size ranges between cfDNA from benign brain tumors and high-grade glioma patients (n=6 per group), using ssDNA libraries. Median frequencies of DNA fragments were used in calculating the difference between patient cohorts. The pattern of cfDNA fragment sizes in plasma samples prepared using ssDNA sequencing libraries is not as clear as that observed in plasma samples prepared using dsDNA sequencing libraries (e.g., FIG. 17A). For example, small, if any, differences (e.g., less than ˜1%) in cfDNA fragment sizes between patient cohorts were observed across the range of sizes.

FIG. 20B depicts the difference in the frequency of CSF cfDNA molecules in different size ranges between cfDNA from benign brain tumors and high-grade glioma patients (n=6 per group), using ssDNA libraries. Median frequencies of DNA fragments were used in calculating the difference between patient cohorts. In contrast to plasma, CSF exhibited a pattern of differences in cfDNA fragment sizes. It can be appreciated that patients having benign brain tumors have higher proportions of cfDNA (e.g., positive differences) in bins between approximately 50-150 bp compared to patients with high-grade gliomas. Additionally, patients having high-grade gliomas have higher proportions of cfDNA (e.g., negative differences) in bins between 150-600 bp.

The combined data from ssDNA sequencing libraries prepared from plasma and CSF cfDNA fragment sizes suggest that CSF, but not plasma, has a differing size profile between patients having benign brain tumors and gliomas. For example, in plasma, a significant difference in the frequency (e.g., a shift to an increased or decreased frequency) of cfDNA fragment sizes was not observed between patients having benign brain tumors and patients having high-grade gliomas, except for an increase between 170-180 bases. In contrast to plasma, the size profile analysis in CSF suggests that patients having gliomas have a reduced frequency of shorter cfDNA sizes (e.g., for fragments below ˜150 bp) and an increased frequency of longer cfDNA sizes (e.g., for fragments ˜150-400 bp) compared to patients having benign brain tumors. This finding is also consistent with the data obtained for dsDNA sequencing libraries (e.g., as shown in FIG. 15B). The frequency of 10-bp periodicities are also reduced in cfDNA from glioma patients.

Overall, the use of various DNA size ranges and cfDNA frequencies can be used in the tumor staging using cfDNA from CSF.

### 2. Method

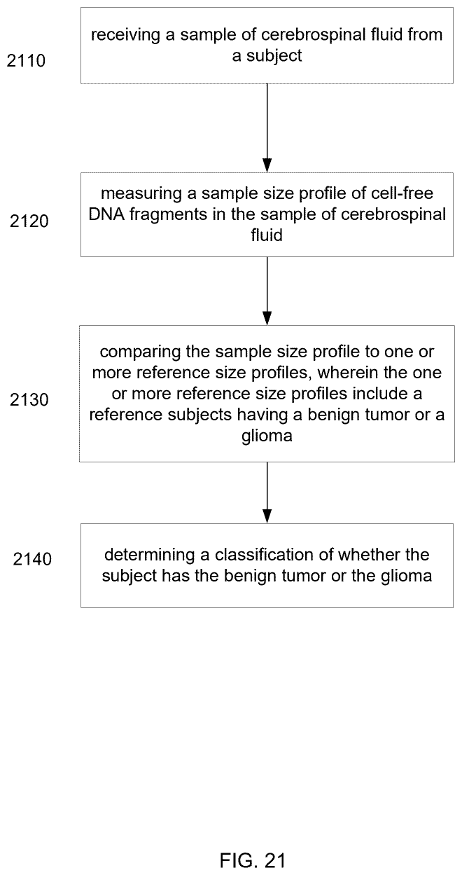

FIG. 21 is a flowchart illustrating a method 2100 for determining a classification of whether the subject has a benign tumor or the glioma based on cfDNA size, according to some embodiments of the present disclosure. Portions or all steps of method 2100 can be performed by a computer system, including one or more processors. The method 2100 can use a trained ML model that was trained by the computer system or another computer system. The computer system can comprise various devices, e.g., one device that performed the training and another that uses the trained model. Certain blocks of method 2100 can be performed in a similar manner as corresponding blocks of method 1500.

At block 2110, method 2100 can include receiving a biological sample of a subject, the biological sample being cerebrospinal fluid, plasma, or serum. Block 2110 can be performed in a similar manner as block 1510.

At block 2120, method 2100 can include measuring a sample size profile of cell-free DNA fragments in the biological sample. Measuring a sample size profile can include a physical separation technique. The physical separation techniques can include filtration and/or electrophoresis. Measuring a sample size profile can also include sequencing the cell-free DNA fragments to obtain sequence reads, measuring sizes of the cell-free DNA fragments using the sequence reads, and generating the sample size profile using amounts of cell-free DNA fragments having a set of sizes. The sequencing can use a single-stranded library preparation method. Measuring the sizes of the cell-free DNA fragments can include aligning paired-end reads to a reference genome. A size can be a size range. Block 2120 can be performed in a similar manner as block 1520.

At block 2130, method 2100 can include comparing the sample size profile to one or more reference size profiles. The one or more reference size profiles can include a first reference size profile determined from cell-free DNA fragments in one or more first reference samples measured from one or more first reference subjects having a benign tumor or a glioma. Block 2130 can be performed in a similar manner as block 1530

For samples of plasma or serum, the first reference size profile can correspond to a glioma and can have a greater proportion of cell-free DNA fragments having a size of 110-120 bases, 120-130 bases, 130-140 bases, 140-150 bases, 150-160 bases, 160-170 bases, or 170-180 bases than a second reference profile corresponding to a benign tumor. The first reference size profile may also correspond to glioma and can have a lower proportion of cell-free DNA fragments having a size of 180-190 bases, 190-200 bases, 200-300 bases, or 300-400 bases than a second reference profile corresponding to a benign tumor.

For samples of cerebrospinal fluid, the first reference size profile can correspond to glioma and can have a greater proportion of cell-free DNA fragments having a size of 160-170 bases, 170-180 bases, 180-190 bases, 190-200 bases, or 200-300 bases than a second reference profile corresponding to a benign tumor. The first reference size profile may also corresponds to glioma and can have a lower proportion of cell-free DNA fragments having a size of 50-60 bases, 60-70 bases, 70-80 bases, 80-90 bases, 90-100 bases, 100-110 bases, 110-120 bases, 120-130 bases, 130-140 bases, 130-140 bases, 140-150 bases, 300-400 bases, or 400-600 bases than a second reference profile corresponding to the benign tumor.

At block 2140, method 2100 can include determining a classification of whether the subject has a benign tumor or a glioma based on a comparison. Block 2140 can be performed in a similar manner as block 1540

### IV. END MOTIFS

Additionally or alternatively, end motifs can be identified and analyzed within CSF samples. Certain end motifs can be analyzed, as described below. An end motif relates to the ending sequence of a cell-free DNA fragment, e.g., the sequence for the K bases at either end of the fragment. The ending sequence can be a k-mer having various numbers of bases, e.g., 1, 2, 3, 4, 5, 6, 7, etc. The end motif (or “sequence motif”) relates to the sequence itself as opposed to a particular position in a reference genome. Thus, a same end motif may occur at numerous positions throughout a reference genome. The end motif may be determined using a reference genome, e.g., to identify bases just before a start position or just after an end position. Such bases will still correspond to ends of cell-free DNA fragments, e.g., as they are identified based on the ending sequences of the fragments.

### A. End Motif Types and ssDNA Sequencing

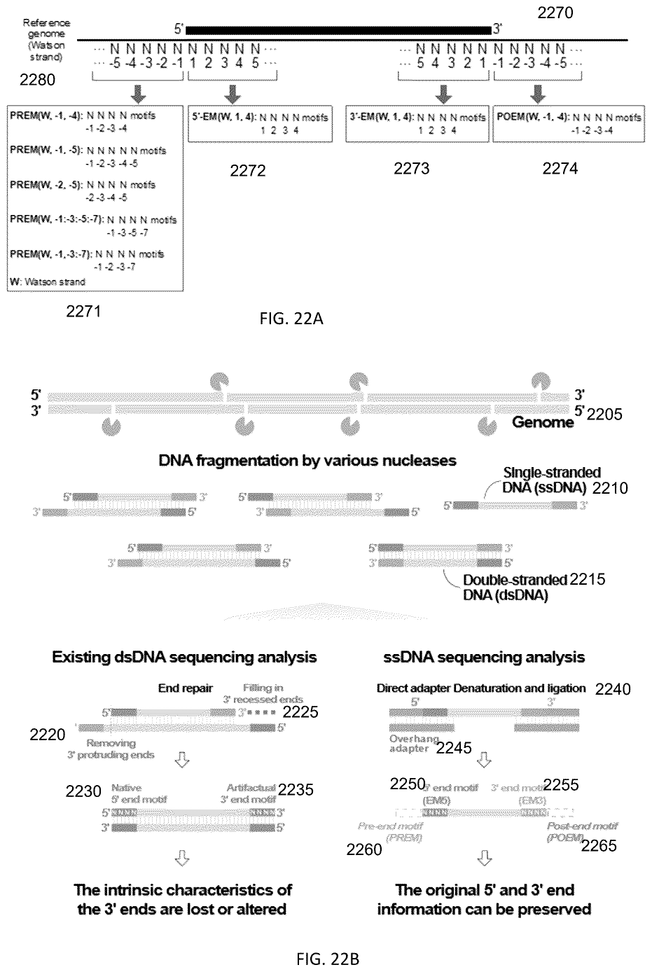

FIG. 22A shows an illustration of the determination of pre-end motifs (PREM) and post-end motifs (POEM), as well as 5′ end motifs and 3′ end motifs. The sequenced paired-end reads (e.g., sequenced separately or taken from a single read) were aligned to a human reference genome with a direction from 5′ to 3′. In one example for any embodiment described herein, the human reference genome, e.g., GRCh37 (hg19), can be considered the Watson strand, whose reverse-complement counterpart (i.e., the Crick strand) can be in silico determined.

Based on the alignment result of DNA fragment 2270, the nucleotides (nt) at the ends of DNA fragment and in a reference sequence 2280 proximal to the 5′ end and 3′ end of a sequenced fragment can be identified. Such nucleotides at various positions can be used to generate various end motifs. The proximality (position) of a particular nucleotide in the end motif can be defined as the distance between nucleotide and the 5′ outmost coordinate for PREM 2271 and the 3′ outmost coordinate for POEM 174.

The different positions are labeled with minus positions and negative positions relative to an end. The −1 position for a PREM corresponds to the position in the reference sequence just before the genomic coordinate of the 5′ end. Similarly, the −5 position for PREM is five nucleotides in the reference sequence before the genomic coordinate of the 5′ end. The +1 position for the 5′ end 2272 (motif denoted as 5′EM or EM5) corresponds to the last nucleotide in the fragment at the 5′ end, with other positions increasing to the right toward the other end of the fragment. The +1 position at the 3′ end 2273 (motif denoted as 3′EM or EM3) corresponds to the last nucleotide in the fragment at the 3′ end. The −1 position corresponds to the next nucleotide after the 3′ end in the reference sequence 2280 (e.g., a reference genome).

The number of nucleotides can be, but not limited to, at least 2 nt, 3 nt, 4 nt, 5 nt, 6 nt, 7 nt, 8 nt, 9 nt, 10 nt, 15 nt, 20 nt, etc. Nucleotides at various positions (possibly non-contiguous) can be used in the end motif. For PREM and POEM, the position farthest from the outermost coordinate of a particular end can be within a threshold, which may be but not limited to 50 nt, 45 nt, 40 nt, 35 nt, 30 nt, 25 nt, 20 nt, 15 nt, 10 nt, 5 nt, 4 nt, 3 nt, 2 nt, etc. PREM and POEM can be examined individually or in combination according to the embodiments present in the disclosure. For example, one or more nucleotides of one end motif type can be combined with one or more nucleotides of one or more other end motif types, thereby providing combined end motif types. In some embodiments, the number of nucleotides involving combinations of PREM, POEM, 5′EM (also referred to as EM5), and/or 3′EM (also referred to as EM3) can be, but not limited to, at least 2 nt, 3 nt, 4 nt, 5 nt, 6 nt, 7 nt, 8 nt, 9 nt, 10 nt, 15 nt, 20 nt, etc.

Accordingly, the genomic positions preceding the 5′ end of the aligned fragment are denoted by negative numbers. For example, −1, −2, −3, −4, and −5 indicate the 1st position, 2nd position, 3rd position, 4th position, and 5th position preceding the 5′ end, respectively. The genomic positions following the 3′ end of the aligned fragment are denoted by negative numbers. For example, −1, −2, −3, −4, and −5 indicate the 1st position, 2nd position, 3rd position, 4th position, and 5th position following the 3′ end, respectively. In other words, the absolute value of a negative number herein represents its distance from the 5′ end or 3′ end of a fragment. PREM is defined as two or more nucleotides from these coordinates with negative numbers.

For example, the combination of 4 nucleotides from positions of −1, −2, −3, and −4 preceding the 5′ end of the fragment forms 4-mer PREM, with a total of 256 types (4⁴), referred to as PREM(W, −1, −4) (“W” herein refers to Watson strand). The combination of 5 nucleotides from positions of −1, −2, −3, −4, and −5 forms 5-mer PREM, with a total of 1,024 types (4′), referred to as PREM(W, −1, −5). The combination of 4 nucleotides from positions of −1, −2, −3, and −4 following the 3′ end of the fragment can form 4-mer POEM, with a total of 256 types (4⁴), referred to as POEM(W, −1, −4). The combination of 4 nucleotides from positions of 1, 2, 3, and 4 from the 5′ end of the fragment can form 4-mer 5′ end motifs, with a total of 256 types (4⁴), referred to as 5′-EM(W, 1, 4) in this disclosure. The combination of 4 nucleotides from positions of 1, 2, 3, and 4 from 3′ end of the fragment can form 4-mer 3′ end motifs, with a total of 256 types (4⁴), referred to as 3′-EM(W, 1, 4).

In some embodiments, a motif defined in this disclosure comprises a series of nucleotides that are not necessary to be consecutive in terms of genomic positions. For example, the nucleotides at positions of −1, −3, −5, and −7 preceding the 5′ end of a sequenced fragment aligned to the Watson strand can form PREM, which can be denoted as PREM(W, −1:−3:−5:−7). If a motif consists of both consecutive and non-consecutive nucleotides (e.g., positions −1, −2, −3, and −7), such a motif can be denoted as PREM(W, −1, −3:−7), where two numbers separated by a comma (‘,’) suggest consecutive positions ranging from −1 to −3, and two numbers separated by a colon (‘:’) suggest non-consecutive positions. Thus, there can be a gap between the nucleotides when the positions are non-continuous.

A DNA fragment can have a jagged end that protrudes relative to the other strand on either end (i.e., ends are not blunt-ended). A DNA fragment can have jagged ends on both ends having 5′ ends that overhang the corresponding 3′ ends and vice versa. Sequencing can be performed of both strands so that the actual outermost coordinates of both stands can be determined. Based on the alignment result of each strand of DNA fragment, PREMS and POEMS can be defined. The positions and properties for jagged ended fragment can be defined in the same as for blunt-ended fragments.

For example, the number of nucleotides can be, but not limited to, 1 nt, 2 nt, 3 nt, 4 nt, 5 nt, 6 nt, 7 nt, 8 nt, 9 nt, 10 nt, 15 nt, 20 nt, etc. The proximality can be defined as the distance between the 3′ outermost coordinate of PREM and the 5′ outermost coordinate of the 5′ end motif within, but not limited to, 20 nt, 15 nt, 10 nt, 5 nt, 4 nt, 3 nt, 2 nt, 1 nt, 0 nt, etc. Post-end motifs (POEM) refer to the number of nucleotides (nt) in a reference sequence proximal to the 3′ end of a sequenced fragment. The number of nucleotides can be, but not limited to, 1 nt, 2 nt, 3 nt, 4 nt, 5 nt, 6 nt, 7 nt, 8 nt, 9 nt, 10 nt, 15 nt, 20 nt, etc. The proximality can be defined as the distance between the 5′ outermost coordinate of POEM and the 3′ outermost coordinate of the 3′ end motif within, but not limited to, 20 nt, 15 nt, 10 nt, 5 nt, 4 nt, 3 nt, 2 nt, 1 nt, 0 nt, etc. PREM and POEM can be examined individually or in combination according to the embodiments present in the disclosure.

The proximality of the closest nucleotide in the end motif to the outermost coordinate can include more than one positions. For example, a closest position of a PREM to the 5′ outermost coordinate can include a position at −2 or greater. And a closest position of a POEM to the 3′ outermost coordinate can include a position at −2 or greater.

To effectively utilize this information for diagnostic purposes, an analytical framework on the basis of various large language models was developed. This analytical framework makes use of features derived from various sequencing technologies, which capture comprehensive structural and sequence information from each cfDNA molecule. The sequencing technologies include but not limited to short-read sequencing (Illumina) and long-read sequencing (Pacific Biosciences (PacBio) or Oxford Nanopore Technologie). These features may include positional information of each base, terminal base compositions, fragment lengths, jagged ends, as well as upstream and downstream sequence information of ends. To model these complex features, different deep learning approaches capable of capturing both local and global signal patterns within and between cfDNA molecules were developed. For illustration purposes, the framework includes three implementations on the basis of language models: (1) Encoder-decoder based language model; (2) Masked language modeling; and (3) Bunch-molecule embedding (BME) based language model.

The experimental assays can be used to determine ending sequences of one or more ends of a cfDNA molecule (fragment). The experimental assays can include sequencing. The cfDNA fragments may be single- or double-stranded. For the one strand fragment of a single-stranded molecule, one or more sequence reads (e.g., paired-end reads or a single long fragment read for the entire strand fragment) can include ending sequences of one or both ends, e.g., EM5 or EM3. Such end motifs may be used or additionally or alternatively one or more other motifs, PREM or POEM, can be used for each strand fragment. Each strand fragment of a double-stranded molecule can be sequenced, with corresponding end motifs used. Given that up to four end motifs can be obtained for each strand fragment, up to eight end motifs can be obtained for a double-stranded molecule.

FIG. 22B shows a comparison of end information obtained from existing dsDNA sequencing and ssDNA sequencing analysis. As illustrated in FIG. 22B, circulating DNA in plasma consists of a mixture of single-stranded DNA (ssDNA) 2210 and double-stranded DNA (dsDNA) 2215 fragments.

The existing dsDNA library preparation involves an end-repair process that removes the 3′ protruding single-stranded ends 2220 and elongates the 3′ recessed ends 2225 using the opposite 5′ protruding single strand as a DNA template. The resultant molecule can include a native 5′ end motif 2230, but an artifactual 3′ end motif 2235 due to changes to the native 3′ ends. As a result, the intrinsic characteristics of the 3′ ends are lost or altered, and their potential diagnostic value has been unexplored. This method of library preparation is widely used in next generation sequencing, which may indicate that currently widely practiced library preparation cannot capture such 3′ end information.

Instead, the single-strand library preparation can involve direct denaturation and ligation 2240. This can include separating a double-stranded DNA molecule 2205 and directly ligating an overhang adapter 2245 to the single-stranded molecule 2210 to preserve the 3′ end. The overhang adapter 2245 can also be added to the strand fragments resulting from the denaturation of double-stranded DNA molecules.

In one embodiment, we adapted ssDNA library preparation followed by paired-end sequencing on the Illumina platform, referred to as 2-end sequencing. 2-end sequencing involved DNA denaturation followed by direct adapter ligation, but omitting the DNA end-repair process. This method thus preserves the original end information of both ssDNA and dsDNA fragments in sequencing data. From 2-end sequencing results, the 5′ end motifs 2250 and 3′ end motifs 2255 that are directly measured from individual strands are referred to as EM5 and EM3, respectively. As the footprint of DNA nucleases acting on the cfDNA fragmentation may involve several nucleotides surrounding the cleavage sites, the end motifs located upstream of 5′ end (PREM, pre-end motif 2260) and downstream of 3′ end (POEM, post-end motif 2265) may be analyzed, which may be inferred from the reference genome. As DNA nucleases may engage several nucleotides surrounding the cleavage site, including the PREM and POEM when analyzing molecule information may increase cancer diagnosis and disease diagnosis accuracies.

In one example, plasma DNA was extracted from 2 mL of plasma using the EZ1&2 ccfDNA Kit (QIAGEN) that was compatible with the automation equipment EZ2 Connect (QIAGEN). DNA library preparation was constructed using the SRSLY PicoPlus DNA NGS Library Preparation Base Kit with the UMI-UDI Primer Set (Claret Bioscience) according to the manufacturer's instructions. In brief, plasma DNA containing both dsDNA and ssDNA was denatured into ssDNA molecules and subsequently ligated with SRSLY splint adapters. Each SRSLY splint adapter contains a 7-nt random single-stranded overhang, allowing the complementary pairing between SRSLY splint adapters and ending sequences of ssDNA molecules. The adapter-ligated molecules were subsequently amplified through PCR, during which unique molecular identifiers (UMIs) and sample-specific indexes were incorporated. As there was no end-repairing step, the native ends of cfDNA fragments could be retained. The libraries were sequenced on the NovaSeq 6000 system (Illumina) in a 100-bp×2 paired-end mode.

### B. Difference Between CSF and Plasma

DNA nucleases produce preferential end motif cutting, with DNASE1L3 preferentially creating 5′ C-end fragments when cutting DNA molecules, DNASE1 producing 5′T-end fragments and DFFB producing 5′A-end and G-end fragments (Han et al. 2020). The terminal base of DNA fragments could be used to define nuclease specific cutting signatures. DNASE1L3 can be actively secreted by the choroid plexus and may be the major nuclease in CSF. The presence of other nucleases (DNASE1) and other biological factors related to DNA fragmentation may vary in CSF as compared to plasma.

### 1. 1-Mer End Motifs

To assess the profile of end motifs between cfDNA from CSF and plasma, we first evaluated the 5′ 1-mer end motif ranking in patients having high intracranial pressure.

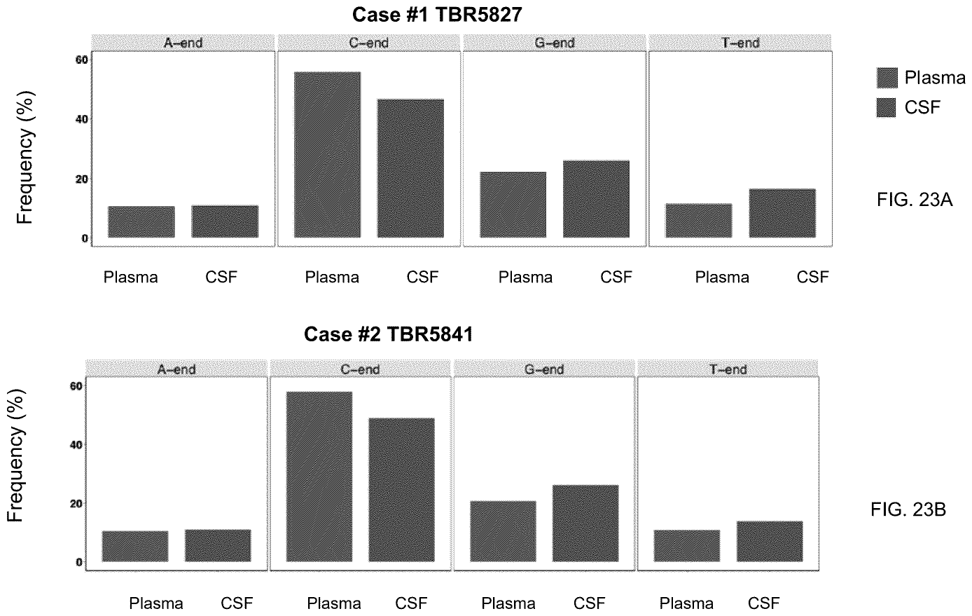

FIGS. 23A-23B show bar plots of 1-mer 5′ end motif frequencies of paired plasma and CSF DNA from two individual with high intra-cranial pressure. We have analyzed the frequences of the terminal base (1-mer end motif) from paired plasma and CSF cfDNA (FIGS. 23A-23B). Some difference in the frequency of 1-mer end motifs is observed, such as the decrease in C-end motifs, increase in G and T-end motifs. This may reflect potential differences in nuclease composition in the CSF, or a possible effect of high intra-cranial pressure that affects the fragmentomic process. In both FIG. 23A and FIG. 23B the C-end was the most predominant end motif in CSF and plasma. However, there was a slight decrease in the C-end in the C-end frequency in CSF compared to plasma, and a slight increase in G and T-end frequency in CSF compared to plasma.

### 2. 4-Mer End Motifs

To further assess the profile of end motifs between cfDNA from CSF and plasma, we evaluated the 5′ 4-mer end motif ranking and motif frequencies in patients having high intracranial pressure and patients having benign brain tumors.

### a) ICP

To first assess the profile of end motifs between cfDNA from CSF and plasma, we evaluated the 5′ 4-mer end motif ranking in patients with high intracranial pressure.

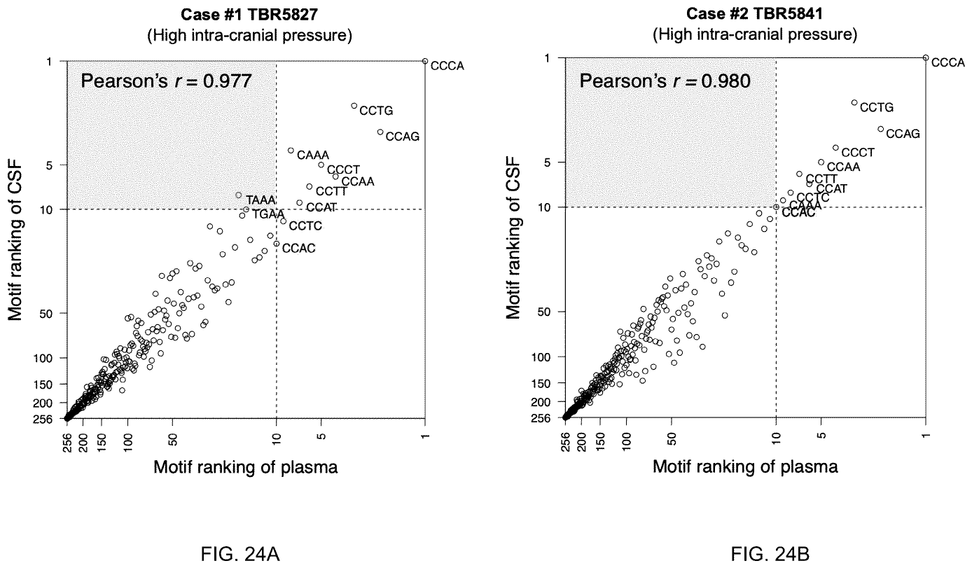

FIGS. 24A-24B show plots of the correlation between 4-mer 5′ end motif rankings of plasma and CSF cfDNA from two individuals with high intra-cranial pressure. The motif ranking (e.g., from 1 to 256) of 4-mer end motifs is strongly positively correlated between cfDNA from CSF and plasma (Pearson's r >0.97). Overall, this demonstrates that the end motif patterns are highly similar between CSF and plasma. CC ends are known to be a result of DNASE1L3 activity (Serpas et al. 2019). There is a possibility of DNASE1L3 being present within the CSF circulation which performs fragmentation of cfDNA. Secretion or release of DNASE1L3 into the CSF may be contributed by white blood cells or cells of the choroid plexus, which is known to have increase DNASE1L3 expression (from Protein Atlas). As DNASE1L3 may be a major contribution factor of DNA fragmentation in the CSF, it could also allow for numbers applications, such as the FRAGMA analysis, and possible end motif frequencies to study the effects of cancer (Jiang et al. 2020), which could be used for clinical utility and diagnosis.

We see that the rank is highly consistent between CSF and plasma. CCCA, which is known to be the preferred end motif of DNASE1L3, is the most preferred end motif of cfDNA from CSF. We see a very strong Pearson correlation of about 0.977 and 0.98 for the two cases shown in FIG. 24A and FIG. 24B, respectively.

To further assess the profile of end motifs between cfDNA from CSF and plasma, we evaluated the 5′ 4-mer end motif frequencies in patients with high intracranial pressure.

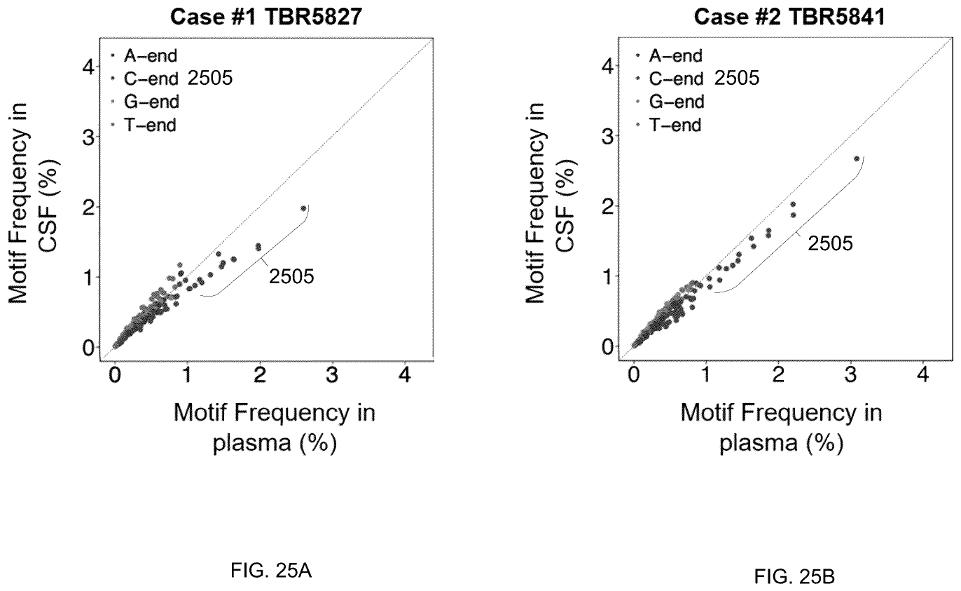

FIGS. 25A-25B show plots of motif frequency of 4-mer 5′ end motifs between paired plasma and CSF cfDNA from two individuals with high intracranial pressure. C-end end motifs 2505 (labeled as such in FIGS. 25A and 25B) generally showed increased frequencies in plasma over paired CSF cfDNA. T and G-end motifs (not labeled in the figures) generally showed increased frequency in CSF (e.g., data points falling above the dotted line in each figure) over paired plasma cfDNA.

Overall, the 4-mer 5′ end motif rankings are consistent between CSF and that of plasma.

### b) Benign

We next assessed the 5′ 4-mer end motif frequencies between cfDNA from CSF and plasma in patients with benign brain tumors.

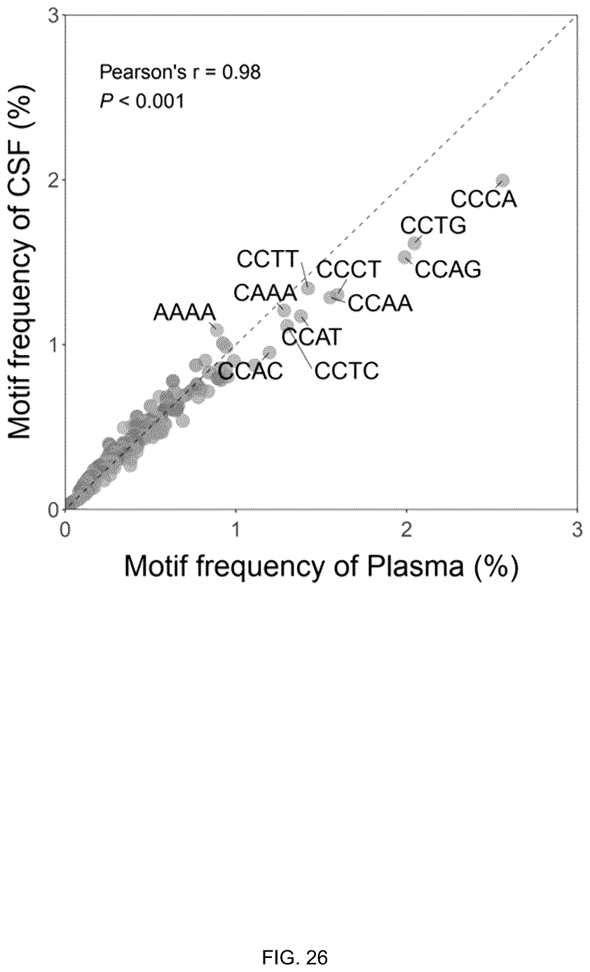

FIG. 26 shows a plot of the motif frequency of 4-mer 5′ end motifs between paired plasma and CSF cfDNA from eight individuals with benign brain tumors. The motif frequency of 5′ 4-mer end motifs is strongly positively correlated between cfDNA from CSF and plasma (Pearson's r>0.98, p<0.001). Overall, this demonstrates that the end motif patterns are highly correlated between CSF and plasma. Additionally, the motif frequency of top end motifs, e.g., CCCA, CCTG, are decreased in CSF compared to plasma. The top ten end motifs each had CC ends, consistent with a result of DNASE1L3 cleavage.

These combined results assessing the 5′ 4-mer end motifs between cfDNA from CSF and plasma and patients with high intracranial pressure and benign brain tumors support the finding that DNASE1L3 is a major nuclease in CSF. Furthermore, the nuclease composition between plasma and CSF differs, which may be due in part to varying expression or activity of other nucleases (e.g., DNASE1) and other biological factors of DNA fragmentation.

### C. Top End Motifs

End motifs can be ranked (e.g., by frequency). Thus, the top ranked end motifs can be determined. We investigated a set of representative top end motifs for DNASE1L3 and DNASE1 in the plasma and CSF of patients having high intracranial pressure and benign brain tumors.

### 1. ICP v Brain Tumor

We first evaluated the top DNASE1L3 and DNASE1 end motifs between cfDNA from CSF and plasma of patients with high intracranial pressure.

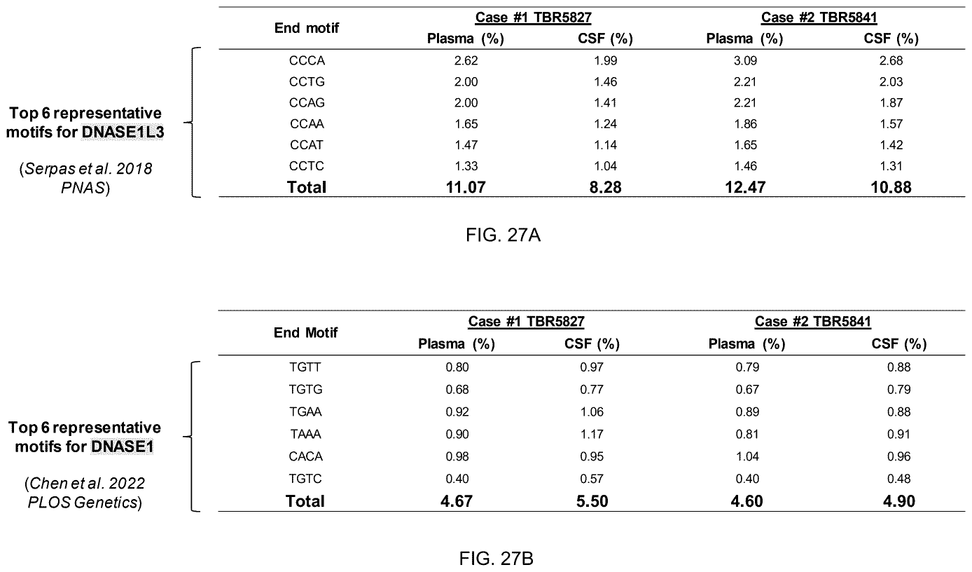

FIG. 27A is a table listing the frequency of the top 6 representative motifs of DNASE1L3 for the two patients with high intracranial pressure. CC-end motifs are present in each of the top 6 DNASE1L3 end motifs. For both patients, the frequency of each of the top 6 end motifs is reduced in CSF as compared to plasma. The top 6 end motifs include: CCCA, CCTG, CCAG, CCAA, CCAT, and CCTC. The sum of frequencies for the top 6 end motifs of each patient are higher in plasma than in CSF. For example, for patient TBR5827, the sum of the top 6 end motifs frequencies was 11.07% in plasma, compared to 8.28% in CSF. For patient TBR5841, the sum of the top 6 end motifs frequencies was 12.47% in plasma, compared to 10.88% in CSF. The data suggest that the contribution of DNASE1L3 is decreased in CSF compared to plasma. These findings are also supported in FIGS. 23A and 23B where the overall frequency of C-end motifs is reduced in the CSF of the same patients compared to plasma.

FIG. 27B is a table listing the top 6 representative motifs of DNASE1 for both cases with high intracranial pressure. The top 6 end motifs include: TGTT, TGTG, TGAA, TAAA, CACA, and TGTC. We see with DNASE1 an increase of those end motif frequencies in the CSF compared to that of plasma. The sum of frequencies for the top 6 end motifs of each patient are higher in CSF than in plasma. For example, for patient TBR5827, the sum of the top 6 end motifs frequencies was 4.67% in plasma, compared to 5.50% in CSF. For patient TBR5841, the sum of the top 6 end motifs frequencies was 4.60% in plasma, compared to 4.90% in CSF. The data suggest that the contribution of DNASE1 is decreased in plasma compared to CSF. T-end fragments are present in 5 of the top 6 DNASE1 motifs. These findings are also supported in FIGS. 23A and 23B where the overall frequency of T-end motifs is increased in the CSF of the same patients compared to plasma.

We next assessed the top 25 4-mer end motifs with the highest and lowest fold change differences from individuals with high intracranial pressure and benign brain tumors.

A difference between top end motifs can also be calculated using a fold change between the frequency of cfDNA end motifs in plasma to that of CSF. In some examples, we calculated a fold change as ratio of the frequency of cfDNA 4-mer end motifs in plasma and the frequency to cfDNA end motifs in CSF. 4-mer 5′end motifs were ranked by highest or lowest fold change between paired plasma and CSF cfDNA. The highest fold change was determined by using the highest ratio of plasma frequency to CSF frequency. The lowest fold change was determined by using the lowest ratio of plasma frequency to CSF frequency. A fold change of a motif was calculated for the paired samples of a patient. The data are reported as the mean fold change from the two patients. A motif having a mean fold change higher than 1.0 indicates that the motif is present at a higher frequency in plasma as compared to CSF. Likewise, a motif having a mean fold change lower than 1.0 indicates that the motif is present at a higher frequency in CSF as compared to plasma.

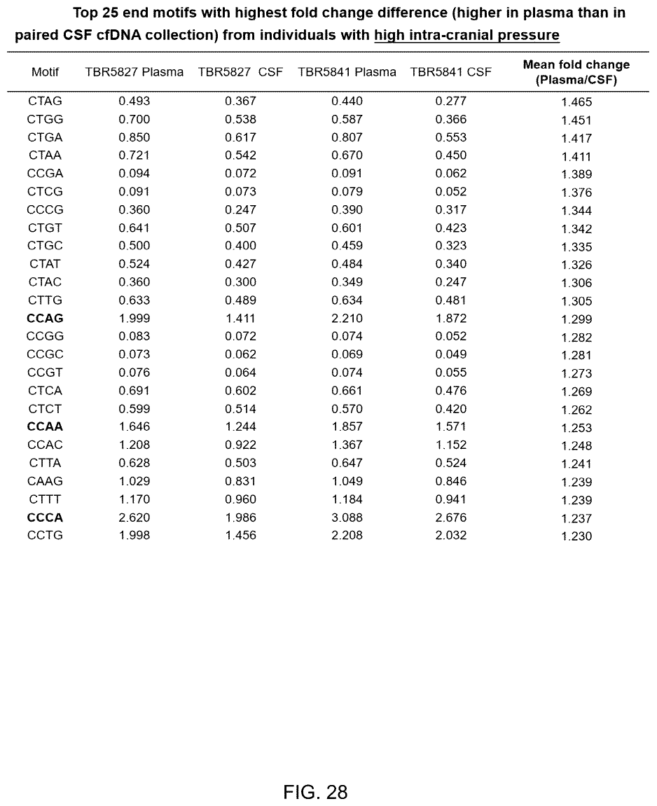

FIG. 28 is a table listing the top 25 end motifs with highest fold change difference between paired plasma and CSF cfDNA from individuals with high intracranial pressure. C-end motifs are present in each of the top 25 motifs. Additionally, 24 of the top 25 4-mer end motifs had a CC-end or a CT-end. The mean fold changes of the top 25 end motifs having the highest fold change range from 1.465 to 1.230. Representative DNASE1L3 motifs, including CCCA, CCTG, and CCAG are present in the top 25 end motifs with the highest fold change difference between paired plasma and CSF cfDNA from individuals with high intracranial pressure.

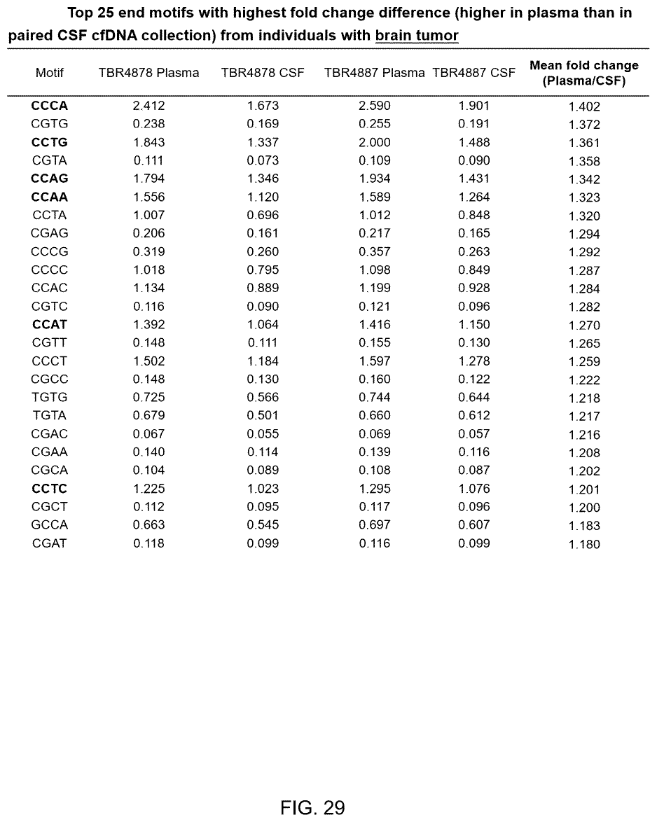

FIG. 29 is a table listing the top 25 end motifs with highest fold change difference between paired plasma and CSF cfDNA from individuals with a benign brain tumor. C-end motifs are present in 22 of the top 25 motifs. Additionally, the top 25 end motifs had a higher frequency in plasma compared to CSF (e.g., mean fold change higher than 1.0). For example, the mean fold changes of the top 25 end motifs having the highest fold change range from 1.402 to 1.180. The end motif with the highest fold change in individuals with a brain tumor was CCCA (e.g., are presentative DNASE1L3 motif). Other CC-end motifs, including CCTG, CCAG, CCAA, CCAT, and CCTC which can also be representative DNASE1L3 motifs, are also present in the top 25 end motifs with the highest fold change difference between paired plasma and CSF cfDNA from individuals with high intracranial pressure.

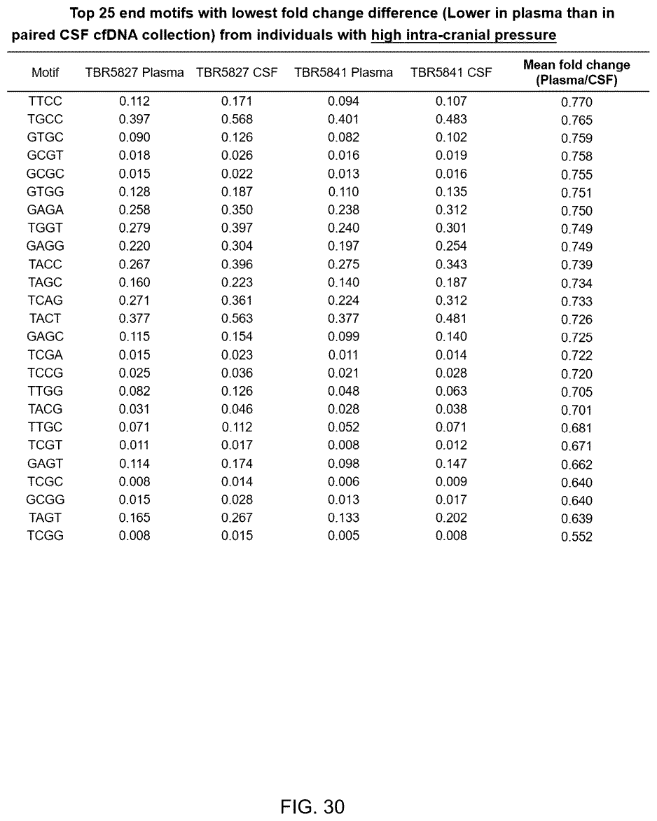

FIG. 30 is a table listing the top 25 end motifs with the lowest fold change difference between paired plasma and CSF cfDNA from individuals with high intracranial pressure. End motifs listed had a higher frequency in CSF compared to plasma. Thus, the mean fold changes of the top 25 end motifs having the lowest fold change are lower than 1.0. The mean fold changes range from 0.552 to 0.777. T-end or G-end motifs were present in each of the top 25 end motifs with the lowest fold change between paired plasma and CSF cfDNA.

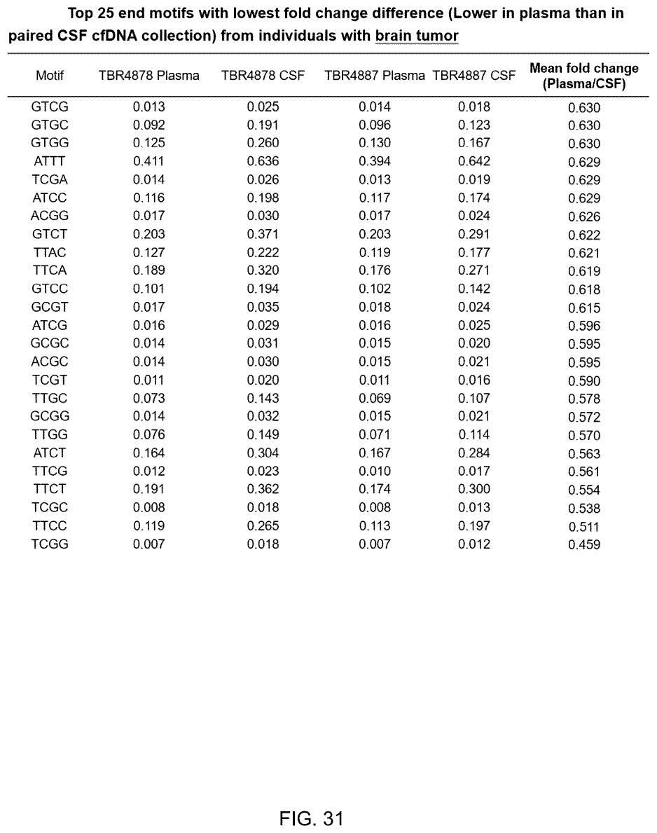

FIG. 31 is a table listing the top 25 end motifs with the lowest fold change difference from individuals with a brain tumor. End motifs listed had a higher frequency in CSF compared to plasma. Thus, the mean fold changes of the top 25 end motifs having the lowest fold change are lower than 1.0. The mean fold changes range from 0.459 to 0.630. T-end or G-end motifs were present in 19 of the top 25 end motifs with the lowest fold change between paired plasma and CSF cfDNA.

We next assessed the top 25 4-mer end motifs with the highest and lowest fold change differences between high intracranial pressure and benign brain tumors from CSF cfDNA. The frequency of each motif was used to calculate a mean motif frequency for each CSN condition (e.g., for high intracranial pressure and benign brain tumors). The mean motif frequency was then used to calculate a mean fold change between CNS conditions for each motif. A motif having a mean fold change higher than 1.0 indicates that the motif is present at a higher frequency in patients with high intracranial pressure as compared to benign brain tumors. Likewise, a motif having a mean fold change lower than 1.0 indicates that the motif is present at a higher frequency in benign brain tumors as compared to high intracranial pressures.

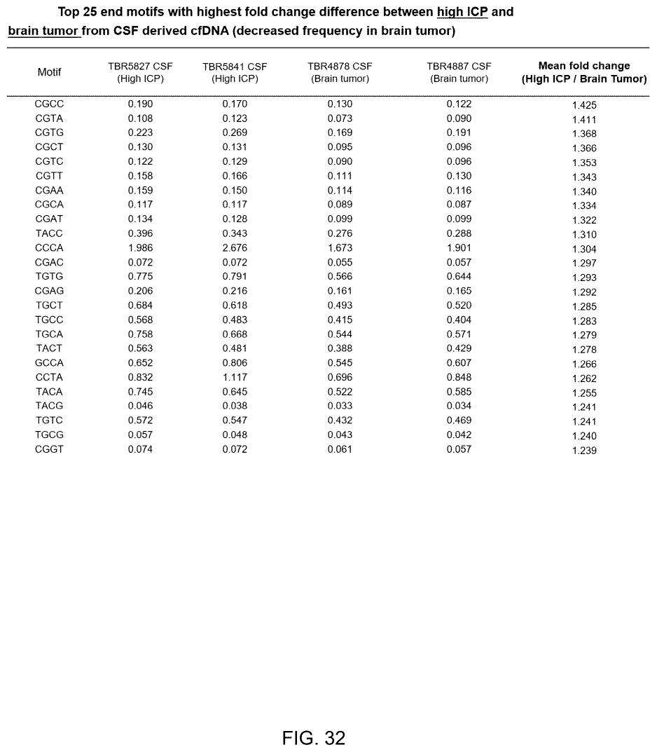

FIG. 32 is a table listing the top 25 end motifs with the highest fold change difference between high intracranial pressure and brain tumor from CSF derived cfDNA. 4-mer 5′ end motifs list had a higher frequency in CSF from subjects with high intracranial pressure compared to CSF from subjects with benign brain tumors. CG-end motifs were present in 12 of the top 25 end motifs with the highest fold change between individuals with high intracranial pressure and brain tumors.

Some embodiments can use any number of the end motifs listed in this table of FIG. 32, or any of the other tables. For example, individual or aggregated amount of at least 1, 2, 3, 4, 5, 6, 7, 8, 9, 10, 20, 30, 40, 50 60, 70, 80, 90, 100, 150, 200, 250, or 256 end motifs can be used. When using the individual amounts, a resulting feature vector can be input into a machine learning model trained using training samples having a known classification of a level of brain cancer. When an aggregated amount is used (e.g., a sum of the individual amounts), the aggregated amount (or a normalized version) can be compared to a reference value (e.g., a cutoff or threshold), where different classifications (e.g., presence or absence) are above and below the reference value. Such a comparison to a reference value can occur implicitly when using a machine learning model.

### D. F-Profile Analysis

Through the non-negative matrix factorization (NMF) algorithm of plasma DNA of mice, our group previously demonstrated that we could use 256 5′ 4-mer end motifs to identify distinct types of cfDNA cleavage patterns, referred to as “founder” end-motif profiles (F-profiles) (Zhou et al. Proc Natl Acad Sci USA. 2023; 120:e2220982). In one example, F-profiles were associated with different DNA nucleases based on whether such patterns were disrupted in nuclease-knockout mouse models. Such an example is of an organism that has a deficiency in a nuclease. Accordingly, the set of reference F-profiles can include one or more reference F-profiles determined from an organism that has a deficiency in a nuclease. However, the reference end profiles can be determined by any means, including directly from human samples.

Instead of specific end motifs, a profile of relative of amounts of different end motifs can be used. Such F-profiles are described in more detail below. The percentage contribution of each F-profile in a cfDNA sample can be deduced using a deconvolution procedure, e.g., using non-negative least squares in a deconvolution analysis of the previous established data matrix (Zhou et al. 2023).

### 1. Example End-Motif Profile (F-Profile)

The end motifs can be used in a variety of ways, e.g., as described above. For example, the amount of one or more end motifs can be determined in a variety of ways. And the classification can be determined in a variety of ways using the amount(s). In some embodiments, the amounts can form an end-motif profile, which can be deconvolved (e.g., as a linear combination) into a set of reference end-motif profiles. The coefficients of the linear combination can be used as features (factors) to perform the classification. Deconvolution (also referred to as decomposition) can be performed in various ways, e.g., non-negative matrix factorization (NMF) or principal component analysis, e.g., constrained to have non-negative coefficients.

Such reference end-motif profiles can relate to particular DNA nucleases. Cell-free DNA (cfDNA) fragmentation is nonrandom, at least partially mediated by various DNA nucleases, forming characteristic cfDNA end motifs. A reference end-motif profile may relate to a particular nuclease, which might be underrepresented or overrepresented in a particular pathology.

After sequencing and obtaining end motifs of any type, the amount of cfDNA fragments having respective end motifs can be determined. For example, a frequency of cfDNA fragments in the sample can be determined for each end motif, e.g., each 2-mer, 3-mer, or 4-mer.

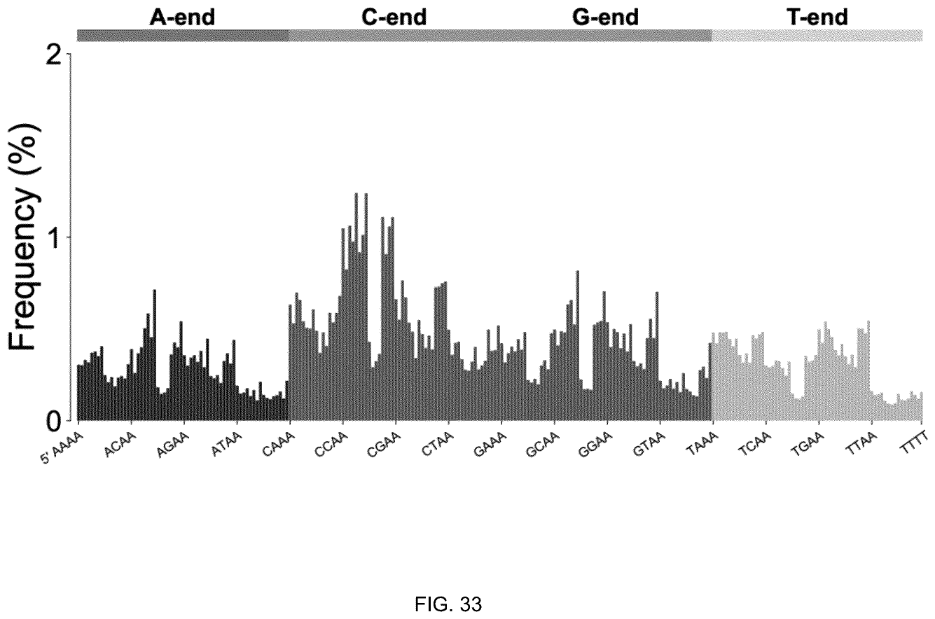

FIG. 33 shows an example end motif profile for 4-mer end motifs. The horizontal axis corresponds to each of the 256 different end motifs for 4-mers. The end-motifs are organized by the first nucleotide in the 4-mer, with A-end grouped on the left, then C-end motifs next, G-end motifs next, and then T-end motifs. The vertical axis is the frequency of each end motif.

Techniques described below can represent this sample end motif profile as a linear combination of reference end-motif profiles, where the coefficient (contribution) for each reference end-motif profile provides how much a particular reference profile is represented in the sample profile. Such concepts and use of them are provided below.

An F-profile (also referred to as an “end-motif profile”) can correspond to a set of relative frequencies for a set of end motifs, where the sum of the relative frequencies is 100% or 1, depending on the how the relative frequency is defined. For example, if the F-profile was for the 256 4-mer end motifs, the F-profile would have 256 frequency values, which are normalized so that they sum to 100% or 1. Each reference F-profile and sample end-motif profile can have a separate proportion for each K-mer end motif of a set of K-mer end motifs. Accordingly, each reference F-profile of the set of reference F-profiles can specify the proportion of cell-free DNA molecules that end in each K-mer end motif of a set of K-mer end motifs, wherein K is one or two or more.

An F-profile can be chosen arbitrarily or chosen based on a biological process, e.g., correspond to a particular nuclease or other fragmentation process. For example, each reference F-profile can be associated with a type of fragmentation factors. The type of fragmentation factor can identify a particular enzyme (e.g., DNASE1L3, DNASE1), protein (e.g., DFFB), or other biological components or processes that cause fragmentation in cell-free DNA molecules. In some instances, the set of reference F-profiles include 1, 2, 3, 4, 5, 6, 7, 8, 9, 10, 15, 20, 25, 30, 40, 45, 50, or more than 50 F-profiles. For example, the set of reference F-profiles can include six F-profiles I-VI.

In some embodiments, a normalized end motif frequency can be calculated as a ratio of observed and expected frequencies (e.g., for a particular region) and then divided by the sum of all 256 normalized motif frequencies. The total normalized end motif frequency can be equal to 100%. The end motif frequency mentioned in this NMF-based nuclease usage analysis was termed the normalized end motif frequency.

### 2. Deconvolution Using Reference End-Motif Profiles (F-Profiles)

A number of nuclease activities or other fragmentation processes can be assessed simultaneously using deduced relative contributions concerning the different types of cell-free DNA cleavage. For example, relative frequencies of DNA molecules corresponding to 256 end motifs can be determined for a subject with a known disease diagnosis (e.g., HCC). The relative frequencies of DNA molecules can be factorized to a set of “F-profiles” that identify the relationship of ending sequences (e.g., 1-30 bases) of cell-free DNA fragments (also just referred to as DNA fragments) in the sample. The set of F-profiles can then be used in deconvolution of relative frequencies of DNA molecules obtained from another subject to predict cfDNA concentration or fraction of clinically-relevant DNA molecules.

In some instances, the set of reference F-profiles are determined using one or more reference samples. The reference samples can be obtained from non-human subjects (e.g., murine samples) whose classification of genetic disorders are known (e.g., WT, DNASE1L3−/−, DNASE1−/−). To determine a reference F-profile of the set of reference profiles, a factorization algorithm (e.g., NMF, PCA) is used to decompose the relative frequencies of the cell-free DNA molecules of the reference samples into several F-profiles. For example, reference cell-free DNA samples with different genotypes of DNA nuclease knockouts were selected. After obtaining the end-motif frequencies of the reference samples, a data matrix (M) is constructed in a way that each row indicates a cell-free DNA sample (e.g., a total of 93 murine cell-free DNA samples) and each column represents a type of end motif (e.g., a total of 256 4-mer end motifs), thus having the dimension of 93×256. The data matrix can then be subjected to NMF analysis for obtaining two matrices W and F.

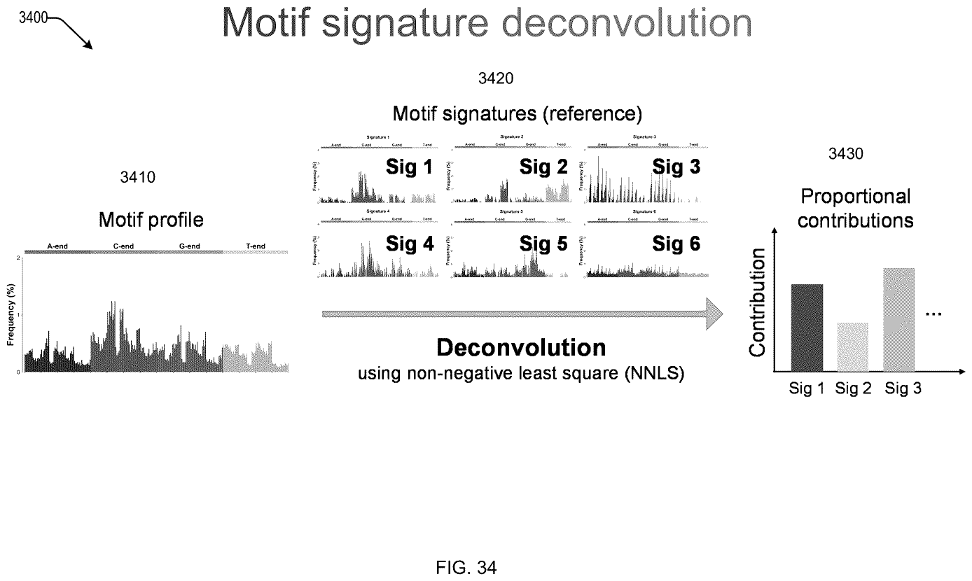

FIG. 34 shows a schematic diagram 3300 of comparing an end-motif profile of a human subject to reference F-profiles determined based on murine samples, according to some embodiments. To make the motif patterns directly comparable between human and mice, the frequencies of 4-mer end motifs related to the human and murine cell-free DNA can be normalized by the genomic contexts of the human and mouse genomes, respectively. For example, an expected 4-mer end-motif frequency can be used for the normalization step, in which the expected end-motif frequency was determined by simulating 4-mer end motifs from a reference genome using a 4-bp sliding window across each chromosome. The normalized end motif frequency was calculated as a ratio of observed and expected frequencies and then divided by the sum of all 256 normalized motif frequencies. The total normalized end motif frequency can be equal to 100%. The end motif frequency mentioned in this NMF-based nuclease usage analysis was termed the normalized end motif frequency.

Once the normalization is complete, proportional contributions of the F-profiles can be determined for the normalized end frequencies of the human sample. The proportional contributions can be determined by applying deconvolution to the normalized end frequencies. For example, a data matrix M generated from W by F can be used, in which: (i) M can represent the normalized end frequencies across 256 end motifs for each biological sample, where each row corresponds to a different biological sample and the columns correspond to the number of end motifs; (ii) F can represent end frequencies of the reference F-profiles obtained from murine samples, where each row corresponds to a different reference end profile and the columns correspond to the number of end motifs; and (iii) W can represent relative weights corresponding to the proportional contributions of each F-profile, where each row corresponds to a different biological sample and the columns correspond to the different reference end profiles. Accordingly, F corresponds to the set of reference end profiles.

The F end frequencies can be determined based on the proportions of the cell-free DNA molecules of the set of reference F-profiles. The proportional contributions can be determined by solving for the W relative weights based on using non-negative least square (NNLS) on values from the data matrix M and the reference F-profiles. The proportional contributions determined using deconvolution can be used to identify an extent of each of the reference end profiles in certain human biological samples, e.g., nuclease activity levels (such as relative decrease of F-profile I contribution) in certain human biological samples.

Accordingly, after obtaining the end-motif frequencies, a data matrix (M) can be constructed in a way that each row indicates a cfDNA sample (a total of p cfDNA samples), and each column represents a type of k-mer end motif (a total of q end motifs), thus having the dimension of p×q. The data matrix was subjected to NMF analysis to obtain two matrices, W and F. The mathematical relationship among M, W, and F were shown below:

$$M = WF .$$

M is the result of the product of W and F, where W is the relative weight for each factor in a p×n matrix, where n corresponds to the number of factors (also referred to as reference end profiles and F-profiles). F represents factors in a n×q matrix. W and F can be determined by minimizing the objective function below:

$$‖ M - WF ‖ , subject to W \geq 0 and F \geq 0.$$

The number of F-profiles is set at a desired value, e.g., 2, 3, 4, 5, 7, 8, 9, 10, 15, 20, or 30 or at least any of these numbers.

Singular value decomposition (SVD) can be used to initialize the procedure of NMF. Such factorization analysis can be implemented in the Python language by using the function of sklearn.decomposition.NMF (v1.1.1). In one embodiment, the optimal number of factors (n) can be determined based on the maximization of performance for a target disease classification (e.g. maximizing AUC value) by using one or more factor levels.

Contributions 3430 of individual F-profiles in a cfDNA sample could be determined by deconvolutional analysis applied to a sample motif profile 3410, e.g., obtained by sequencing DNA molecules from a new biological sample. Each F-profile can be viewed as a different dimension that can separate subjects with different classifications of the pathology. The established factors (a total of n factors) can be deduced via NMF, as mentioned above to obtain the F-profiles 3420 (also referred to as reference end-motif provides). The percentage contribution of each factor in a cfDNA sample could be determined using non-negative least squares (NNLS) based deconvolution analysis. We let a matrix of F represent the deduced factors. The end-motif frequencies of cfDNA molecules can be represented by a vector of X. The percentage contribution of an established factor is denoted as P which can be determined by NNLS:

$$X = {\sum }_{i} ( {P}_{i} \times {F}_{i} ) .$$

where i represented an integer index of a particular factor, ranging from 1 to n. Furthermore, all the factor levels would be required to be non-negative with a sum of 100%:

$${P}_{i} \geq 0 , ∀ i ;$$

$${\sum }_{i} {P}_{i} = 100 \% .$$

NNLS can be implemented based on the Python function of scipy.optimize.nnls (v1.8.1).

However, in conventional sequencing library preparation, the end-repair step involved using a DNA polymerase to polish the ends, making them suitable for ligation with sequencing adaptors (Zhou et al., Proc Natl Acad Sci USA, 2023; 120:e2220982). This DNA polymerase had both 3′->5′ exonuclease activity and 5′->3′ polymerization. Plasma DNA fragments could have 3′ protruding single-strand ends, 5′ protruding single-strand ends, or blunt ends. During end repair, the 3′ protruding single-strand ends were removed, and the 3′ recessed ends were elongated using the opposite 5′ protruding single strand as a template. Consequently, the original 3′ ends were modified, while the original 5′ ends were preserved. Hence, EM3 has not been analyzed in the conventional studied. In addition, the concepts regarding PREM and POEM have been established in this disclosure. Applying NMF to EM5, EM3, PREM, and POEM profiles, either individually or in combination, would significantly enhance the informativeness of end profile analysis.

### 3. Differences Between CSF and Plasma

Deconvolution analysis can be performed on 4-mer end motif patterns of cfDNA molecules, which can include six “founder” end motif cleavage signature profiles, also referred to as reference end-motif profiles or F-profiles. We have performed the deconvolution analysis on paired CSF and plasma cfDNA to evaluate the contribution of the six cleavage patterns. The analyses were performed on patients having high intracranial pressure and those with benign brain tumors.

### a) ICP

We first performed the deconvolution analysis on paired CSF and plasma cfDNA from the two patients having high intracranial pressure.

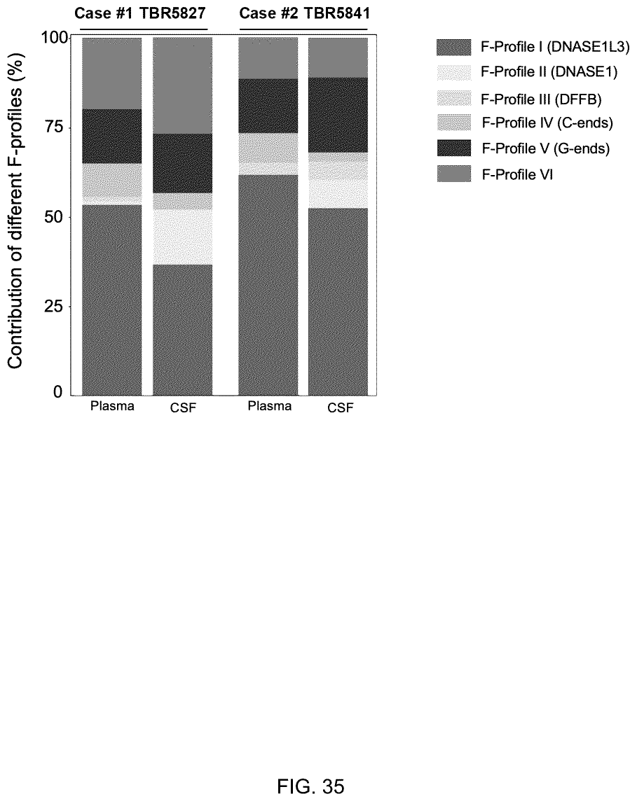

FIG. 35 is a bar chart representing deconvolution analysis of reference end-motif profiles (F-profiles) in paired plasma and CSF cfDNA samples from patients having high intracranial pressure. It is observed that cfDNA from CSF comprises the presence of all six F-profiles, with DNASE1L3 being the largest contributing profile, suggesting similarities in the fragmentation process between plasma and CSF. The relative contribution of DNASE1L3 is decreased in CSF cfDNA compared to paired plasma sample, while relative contribution of DNASE1 is increased compared to paired plasma collection. This provides hints that the nuclease composition may differ in the CSF compared to the plasma. This may also be a result from processes of high intra-cranial pressure.

The paired comparison shows a decreased contribution of Profile I, which is known to be DNASE1L3, in CSF compared to that of plasma. We also see an increased contribution of Profile II, which is DNASE1, contribution in CSF compared to that of plasma for both patients. For the other profiles, an overall increase or decrease may not be identifiable. We see a decrease in the Profile IV, but we don't know exactly the biological meaning of Profile IV. Profile V and VI don't appear to have a larger trend for the two cases in terms of plasma and CSF difference. Overall, the end motif ranking is very highly correlated to that of plasma, but we see an overall decreased contribution of DNASE1L3 and an increased contribution of DNASE1 indicating that there may be some difference in nuclease levels and contributions in the CSF compared to that of plasma.

### b) Benign

We next performed the deconvolution analysis on paired CSF and plasma cfDNA from the eight patients having benign brain tumors.

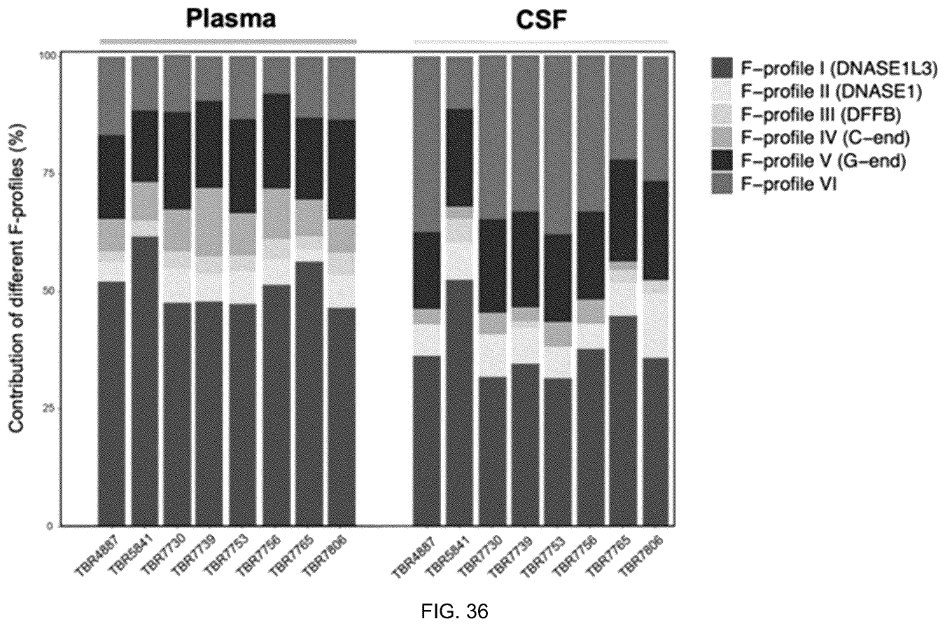

FIG. 36 is a bar chart representing deconvolution analysis of F-profiles in paired plasma and CSF cfDNA samples from cases of benign tumors. Differences are observed in the contribution of various profiles between CSF and plasma. Such as the decreased contributions of Profile I and Profile IV, and increased contributions of Profiles II and Profile VI observed in CSF compared to plasma.

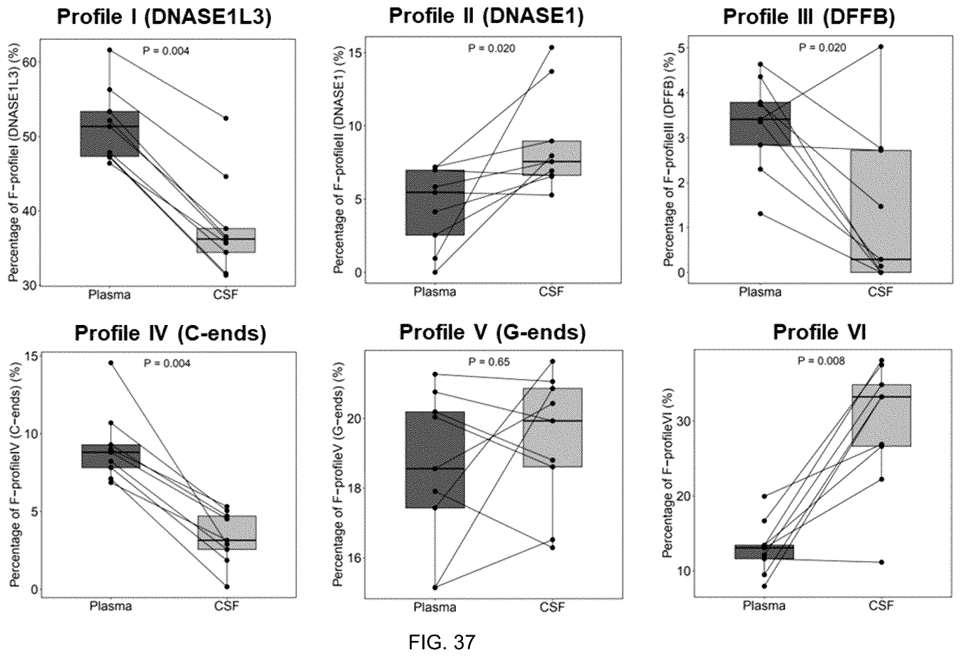

FIG. 37 is a box plot of the percentages of F-profiles from paired plasma and CSF cfDNA samples from cases of benign brain tumors. The box plots summarize the data from the F-profiles shown in FIG. 36. It can be appreciated that the contribution of F-profiles from DNASE1L3 (Profile I), DFFB (Profile III), C-ends (Profile IV) were significantly reduced (p=0.004, 0.020, 0.004, respectively) in CSF compared to plasma of patients with benign brain tumors. The contribution of DNASE1 (Profile II), and Profile VI were significantly increased (p=0.020, 0.008, respectively) in CSF compared to plasma of patients with benign brain tumors. The contribution of G-ends (Profile V) was not different between plasma and CSF. Statistical significance was assessed by Wilcoxon matched-pairs signed rank test.

The differences observed in size profiles and end motif deconvolutions highlight the difference in the fragmentomic landscape between plasma and CSF. The unique properties of cfDNA in CSF indicate that the fragmentation patterns, as a result of differences in molecular composition, nuclease expression and activity, and other biological factors, may differ significantly from those observed in plasma cfDNA, suggesting that previously characterized features in plasma cfDNA may not be applicable. Therefore, identification of novel biomarkers and characteristics specific to CSF cfDNA is required for applicable diagnostic potential.

### 4. Differentiation of ICP Vs Benign Brain Tumor

To evaluate possible alterations in the 4-mer end motif profiles of cfDNA from CSF from individual with brain tumor, the F-profile analysis was performed.

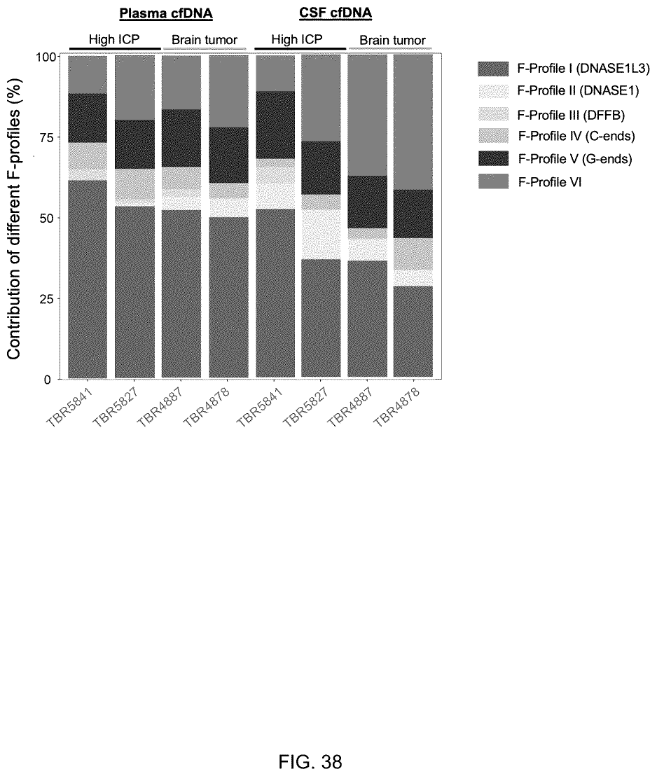

FIG. 38 is a bar chart showing deconvolution analysis of F-profiles for paired plasma and CSF cfDNA from individuals with high intra-cranial pressure (non-tumor) and from individuals with benign brain tumor (n=2 per group). Paired comparisons of cfDNA from plasma and CSF generally showed decreased contributions of DNASE1L3. However, individuals with brain tumor showed an increased contribution of Profile VI in cfDNA from CSF compared to high ICP (non-tumor) subjects. Profile VI was previously defined as a cleavage pattern with no specific end preference, potentially by mechanisms of physical DNA damage (e.g., oxidative stress). No observable differences in the contribution of F-profiles in plasma cfDNA. Overall, this supports that fragmentomic features of CSF may enhance detection of tumors within the central nervous system, with no significant observable changes in plasma cfDNA.

To detect brain cancer (or other level of cancer), an individual contribution of an F-profile can be compared to a reference value (e.g., a threshold). In other implementations, each of the contributions can be used in a feature vector that is input into a machine learning model trained using training samples having a known classification of a level of brain cancer.

### 5. Differentiation Between Benign and Glioma

We performed F-profile analyses on 4-mer end motif profiles from paired plasma and CSF from patients having benign brain tumors and high-grade gliomas. We evaluated differences in the F-profiles between tumor types in both plasma and CSF.

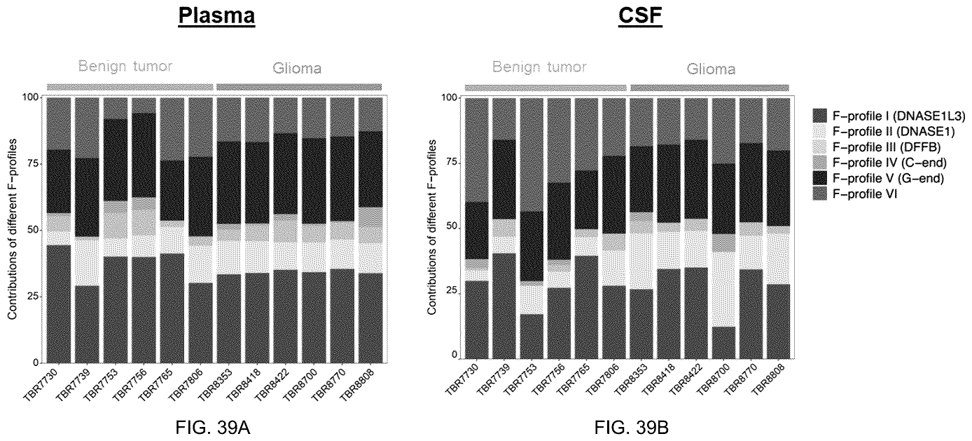

FIG. 39A is a bar chart representing deconvolution F-profiles of 5′ end motifs in plasma cfDNA samples from six patients with benign brain tumors or glioma tumors. No distinguishable patterns of changes in plasma F-profiles were observed between benign brain tumors and gliomas.

FIG. 39B is a bar chart representing deconvolution F-profiles of 5′ end motifs in CSF cfDNA samples from six patients with benign brain tumors or glioma tumors. Differences can be observed in the contribution of various profiles between benign brain tumors and glioma. For example, the increased contributions of Profile II (DNASE1) and decreased contribution of Profile VI (C-ends) observed in glioma tumors compared to benign brain tumors.

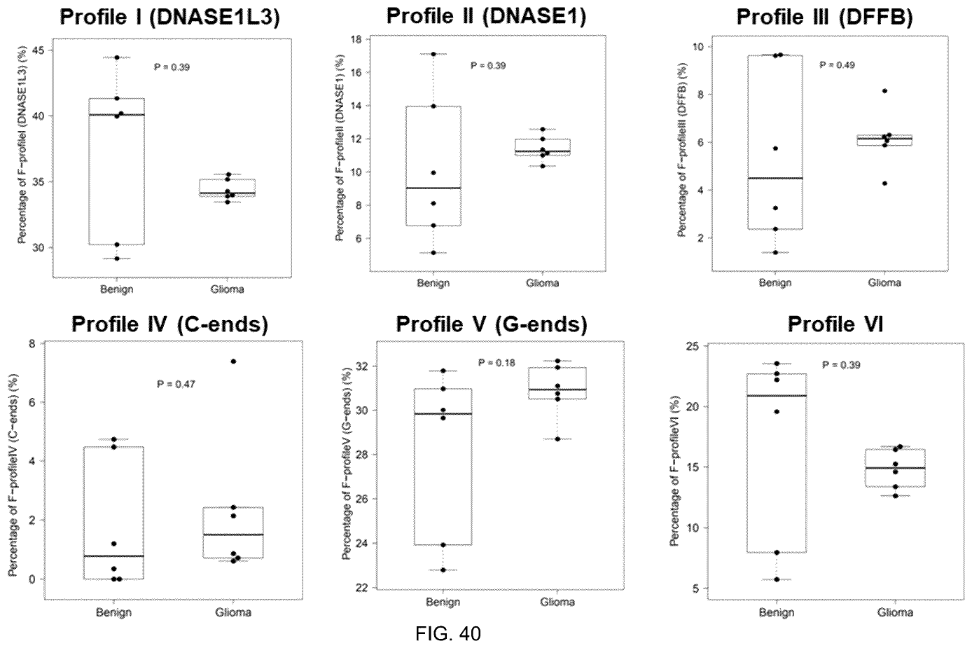

FIG. 40 shows box plots of F-profile contributions in plasma cfDNA between benign and high-grade glioma tumors. The box plots summarize the data from the F-profiles shown in FIG. 39A. The data support the observation from FIG. 39A that there are no distinguishable patterns of changes in the plasma cfDNA F-profiles between benign brain tumors and gliomas.

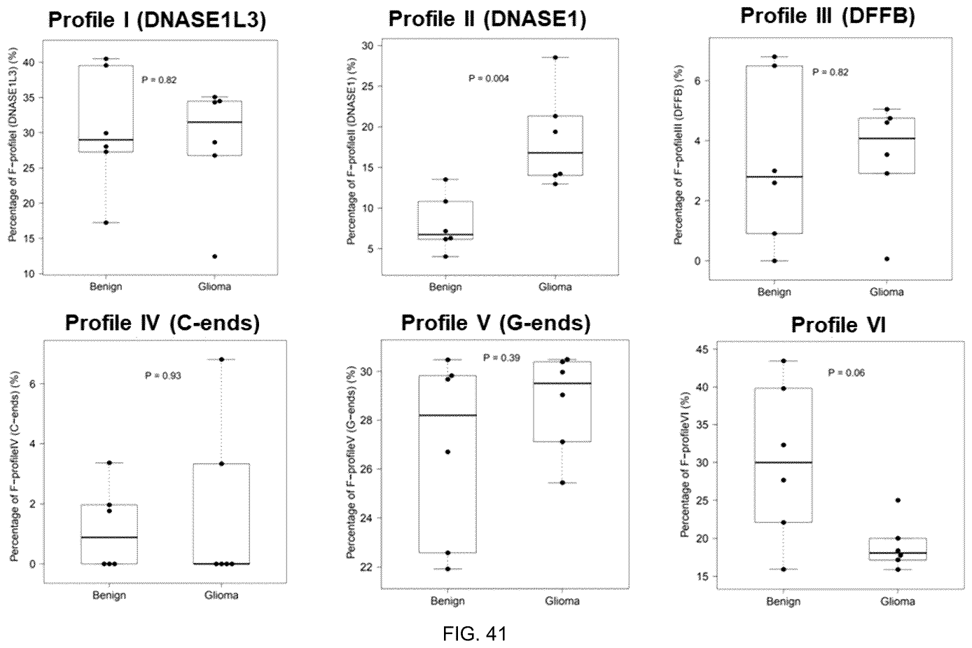

FIG. 41 shows box plots of F-profile contributions in CSF cfDNA between benign and high-grade glioma tumors. The box plots summarize the data from the F-profiles shown in FIG. 39B. Significant differences can be observed in the contribution of various profiles between benign brain tumors and glioma. For example, the increased contributions of Profile II (DNASE1, p=0.004) and decreased contribution of Profile VI (C-ends, p=0.06) observed in glioma tumors compared to benign brain tumors.

In summary, there was no observable difference in the plasma F-profiles between patients with benign brain tumors and high-grade gliomas. However, the CSF F-profiles in patients with high-grade glioma can be characterized by increased profile II contribution (DNASE1) and decreased profile VI contribution compared to patients with benign brain tumors. Glioblastomas (GBM) can be characterized by increased DNASE1 expression, but non-significant changes in DNASE1L3 (Bai et. al. 2023—Pan-cancer analysis of the deoxyribonuclease gene family). An increased DNASE1 expression in patient with gliomas in may explain difference in F-profile contribution. Therefore, F-profiles II and VI could serve as a useful diagnostic marker for GBM.

### E. Use of Other End Motif Types

Analyses of other end motif types can also be used to differentiate between patients having a variety of neurological conditions. For example, pre-end motifs (PREM), post-end motifs (POEM), as well as 5′ end motifs (EM5) and 3′ end motifs (EM3) can be used. A variety of analysis methods can be used, including machine learning approaches.

In some embodiments we utilized the PREM, EM5, EM3, and POEM according to the embodiments in this disclosure as features and trained SVM models for differentiating patients with diseases from subjects without said diseases. The PREM, EM5, EM3, and POEM for each sample can be input into a machine learning model. The machine learning model can then process the feature vectors.

Relationships between the tumor types can be visualized using a variety of methods, including hierarchical clustering heatmaps. A hierarchical clustering heatmap is a method to organize data into a multilevel hierarchy, creating a tree-like structure called a dendrogram that shows relationships between data. The resulting dendrogram can allow for visualization of relationships between data. For example, a heatmap can include a matrix where each colored cell corresponds to a specific data value (e.g., a Z-score) for a particular sample and end motif. A color key can be provided to indicate the values represented by the cell colors. The rows of the matrix can represent end motifs. For example, a hierarchical clustering heatmap using 4-mer end motifs would have 256 rows of data. The columns of the heatmap can represent the experimental samples. Rows and columns of the hierarchical clustering heatmap can each have a dendrogram which show relationships between the samples and end motifs. Branchpoints in a dendrogram can indicate that data points connected below it have been joined into a single, larger cluster. A branchpoint lower on the dendrogram can indicates a higher degree of similarity between merged clusters. A branchpoint higher on the dendrogram can indicate a lower degree of similarity between merged clusters.

Volcano plots can be another way of visualizing significant differences between patients having benign brain tumors and patients with high-grade gliomas. For example, a scatter plot can be created to plot the magnitude of a difference between end motifs against the p-value of the difference.

We performed analyses on 4-mer end motif profiles from paired plasma and CSF from six patients having benign brain tumors and six patients having high-grade gliomas. We evaluated differences in the PREM, EM5, EM3, and POEM between tumor types in both plasma and CSF. The data were obtained from samples prepared using single stranded sequencing libraries.

### 1. PREM

We first tested the ability of PREM to differentiate between patients having benign brain tumors and patients with high-grade gliomas in both plasma and CSF cfDNA. The data were visualized using hierarchical clustering heatmaps with each column corresponding to a sample and each row corresponding to a PREM. PREMs for each sample were also used as features as input into the model. Volcano plot analyses were also performed to show differential PREM frequency in CSF cfDNA between patients with benign brain tumors and high-grade gliomas.

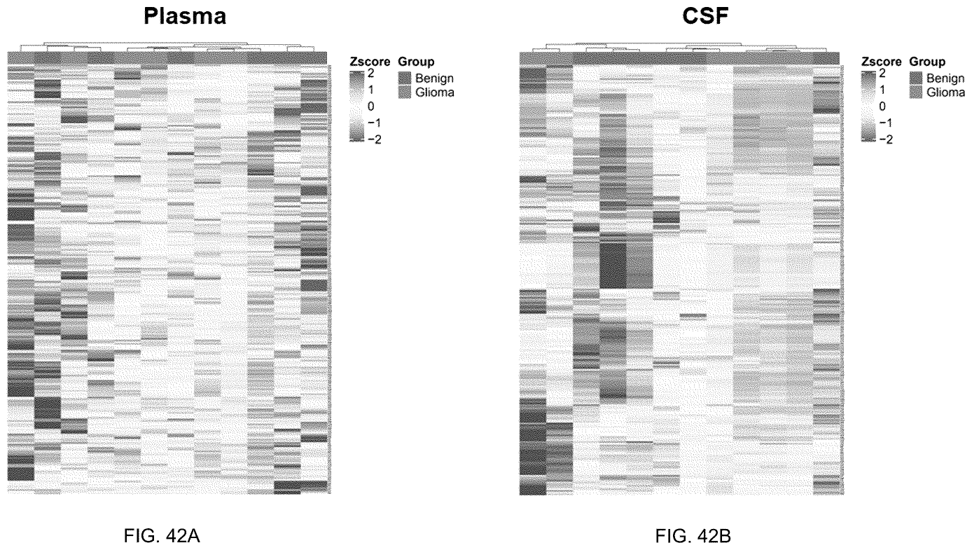

FIG. 42A is a hierarchical clustering heatmap analysis of PREM from plasma cfDNA of patients having benign brain tumors or high-grade gliomas. Visualization of the patterns of Z-score coloring in the heatmap generally indicates a low degree of clustering of each sample type. For example, the six samples of the benign brain tumor group were clustered into a total of 4 clusters. Additionally, three of the samples were in a cluster as a single sample. Similarly, the six samples of the high-grade glioma group were clustered into a total of 4 clusters, with two of the samples being in a cluster as a single sample.

FIG. 42B is a hierarchical clustering heatmap analysis of PREM from CSF cfDNA of patients having benign brain tumors or high-grade gliomas. Visualization of the patterns of Z-score coloring in the heatmap generally indicates a higher degree of clustering of each sample type, compared to plasma. For example, the six samples of the benign brain tumor group were clustered into a total of 2 clusters. Similarly, the six samples of the high-grade glioma group were also clustered into a total of 2 clusters.

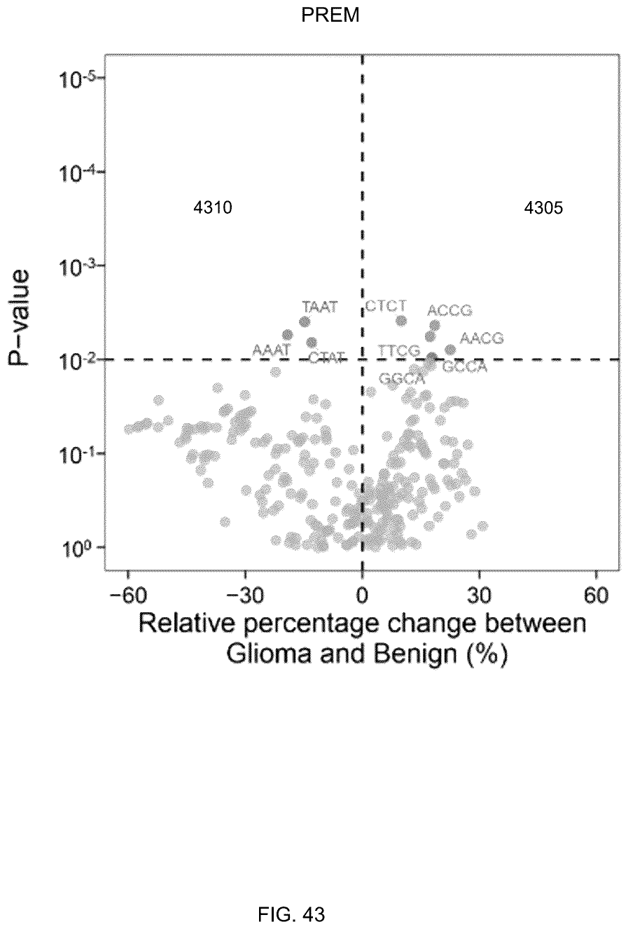

FIG. 43 is a volcano plot analysis showing differential PREM frequency in CSF cfDNA between patients with benign brain tumors and high-grade gliomas. The volcano plots are shown as a scatter plot where the horizontal axis represents the relative percent change of PREM frequency between patients with glioma and patients with benign brain tumors. The vertical axis represents the p-value of the difference between the relative percent change of PREM between patients with glioma and patients with benign brain tumors. The vertical hashed line represents zero percent change. Data points for PREM frequencies falling to the right of the vertical hashed line have a positive percent difference between glioma and benign, meaning the PREM motif frequencies are higher in the glioma group compared to the benign group. Likewise, data points for PREM frequencies falling to the left of the vertical hashed line have a negative percent difference between glioma and benign, meaning the PREM motif frequencies are higher in the benign group compared to the glioma group.

The horizontal line represents a p-value established to determine statistical significance. For example, the hashed line is located at 10⁻² (e.g., p=0.01). Data points for PREM frequencies falling above the hashed line have p-values less than 0.01 and are statistically different between the glioma group and the benign group. Data points for PREM frequencies falling below the hashed line have p-values greater than 0.01 and are not statistically different between the glioma group and the benign group. As such, the hashed lines can separate the volcano plot into quadrants to visualize PREM frequencies that are significantly different between the glioma group and the benign group. It can be appreciated that there are more PREMs having increased frequency in the glioma group 4305 compared to PREMs having increased frequency in the benign group 4310.

Table 4 shows the median PREM frequencies in cfDNA from CSF in patients with benign and glioma tumors. PREM motifs are ranked by p-value of difference in motif frequency. Data in the table are representative of the data presented in FIG. 43.

TABLE 4
Median PREM frequencies in cfDNA from CSF
in patients with benign and glioma tumors.

| | | | | Fold | |
|---|---|---|---|---|---|
| | | Motif | Motif | change | |
| | | frequency | frequency | (Glioma/ | |
| Rank | Motif | (Benign) | (Glioma) | Benign) | P-value |
| 1 | CTCT | 0.679782648 | 0.747673427 | 1.099871302 | 0.003874823 |
| 2 | TAAT | 0.354986091 | 0.302459323 | 0.852031476 | 0.003964392 |
| 3 | ACCG | 0.041743052 | 0.04948714 | 1.185518 | 0.004345512 |
| 4 | AAAT | 0.528951472 | 0.427582899 | 0.808359408 | 0.005446625 |
| 5 | TTCG | 0.050173912 | 0.058879336 | 1.173505003 | 0.005666163 |
| 6 | CTAT | 0.338712863 | 0.294741968 | 0.870182389 | 0.006571331 |
| 7 | AACG | 0.056158915 | 0.068803932 | 1.225164912 | 0.007885804 |
| 8 | GCCA | 0.582145063 | 0.686255851 | 1.17883994 | 0.009495905 |
| 9 | GGCA | 0.484183207 | 0.569869931 | 1.176971698 | 0.009707117 |
| 10 | TGCA | 0.592743859 | 0.692879324 | 1.168935475 | 0.010923596 |
| 11 | GTCA | 0.541858532 | 0.637282621 | 1.17610517 | 0.011636185 |
| 12 | AGCC | 0.388271488 | 0.439735293 | 1.132545927 | 0.012704102 |
| 13 | TTGC | 0.343863324 | 0.396826394 | 1.154023606 | 0.013458084 |
| 14 | ATAC | 0.269506334 | 0.20954465 | 0.777512895 | 0.013514445 |
| 15 | ACCC | 0.262817768 | 0.283417026 | 1.078378483 | 0.018751183 |
| 16 | CGAT | 0.021798335 | 0.013706233 | 0.628774296 | 0.01994839 |
| 17 | GTGA | 0.407453121 | 0.416392088 | 1.021938639 | 0.022003182 |
| 18 | GTCG | 0.029748569 | 0.033427848 | 1.123679202 | 0.022421721 |
| 19 | AGAC | 0.158873005 | 0.111064069 | 0.699074516 | 0.023803865 |
| 20 | TGCT | 0.679446724 | 0.791506686 | 1.164928255 | 0.023949601 |

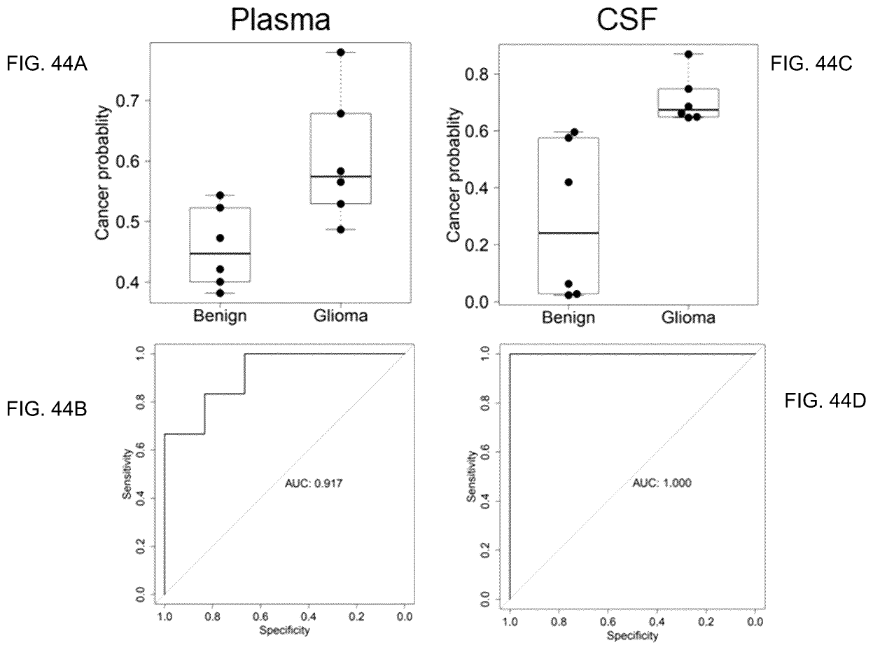

FIG. 44A is a box plot depicting cancer probabilistic scores predicted by SVM models using PREM from plasma cfDNA as inputs into the model. Compared with subjects having benign brain tumors, cancer probabilistic scores predicted by the models were observed to be elevated in patients with high-grade gliomas using PREM motifs from plasma cfDNA.

FIG. 44B depicts an Receiver Operating Characteristic (ROC) curve analysis using the PREM from plasma cfDNA to differentiate between subjects with benign brain tumors or high-grade gliomas. We show that the Areas Under the Curve (AUC) for differentiating patients having benign brain tumors from patients with high-grade gliomas was 0.917 using PREM from plasma cfDNA.

FIG. 44C is a box plot depicting cancer probabilistic scores predicted by SVM models using PREM from CSF cfDNA as inputs into the model. Compared with subjects having benign brain tumors, cancer probabilistic scores predicted by the models were observed to be elevated in patients with high-grade gliomas using PREM from CSF cfDNA.

FIG. 44D depicts an ROC curve analysis using the PREM from CSF cfDNA to differentiate between subjects with benign brain tumors or high-grade gliomas. We show that the AUC for differentiating patients having benign brain tumors from patients with high-grade gliomas was 1.000 using PREM from CSF cfDNA.

Overall, these results demonstrate better clustering and sensitivity in differentiating high-grade gliomas from benign brain tumors in CSF cfDNA as compared to plasma cfDNA when using PREM to differentiate between tumor types.

### 2. EM5

We next tested the ability of EM5 to differentiate between patients having benign brain tumors and patients with high-grade gliomas in both plasma and CSF cfDNA. The data were visualized using hierarchical clustering heatmaps with each column corresponding to a sample and each row corresponding to an EM5. EM5s for each sample were used as features as input into the model. Volcano plot analyses were also performed to show differential EM5 frequency in CSF cfDNA between patients with benign brain tumors and high-grade gliomas.

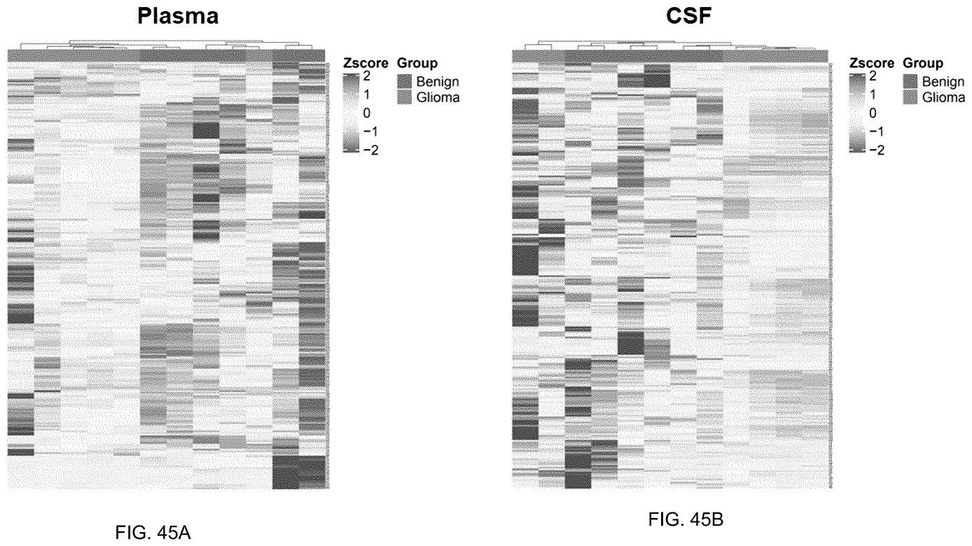

FIG. 45A is a hierarchical clustering heatmap analysis of EM5 from plasma cfDNA of patients having benign brain tumors or high-grade gliomas. Visualization of the patterns of Z-score coloring in the heatmap generally indicates a moderate degree of clustering of each sample type. For example, the six samples of the benign brain tumor group were clustered into a total of 2 clusters. Similarly, the six samples of the high-grade glioma group were clustered into a total of 4 clusters. However, one of the glioma samples was in a cluster as a single sample.

FIG. 45B is a hierarchical clustering heatmap analysis of EM5 from plasma cfDNA of patients having benign brain tumors or high-grade gliomas. Visualization of the patterns of Z-score coloring in the heatmap generally indicates a higher degree of clustering of each sample type, compared to plasma. For example, the six samples of the benign brain tumor group were clustered into a single cluster containing all six samples. The six samples of the high-grade glioma group were clustered into a total of 2 clusters.

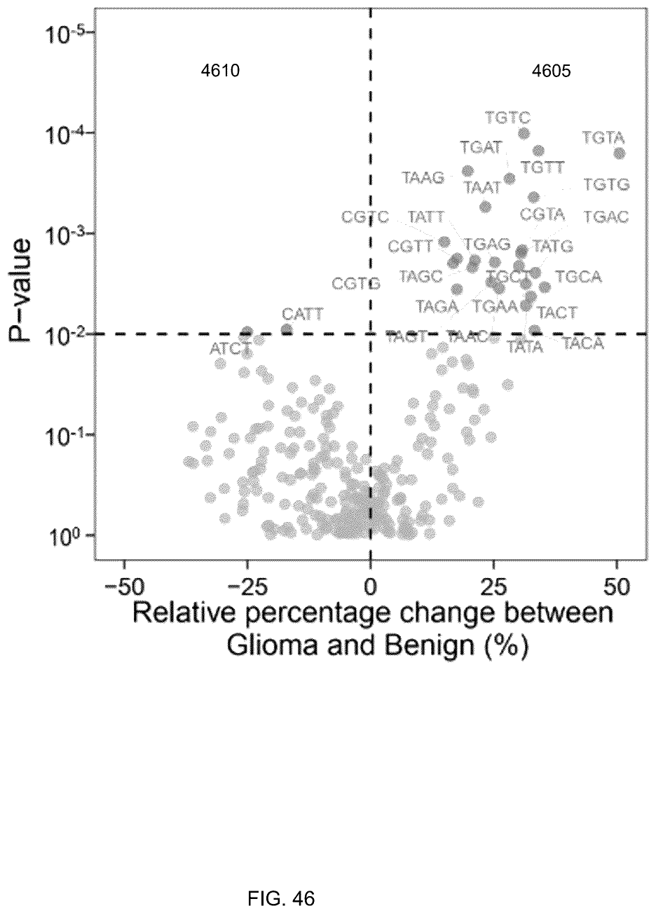

FIG. 46 is a volcano plot analysis showing differential EM5 frequency in CSF cfDNA between patients with benign brain tumors and high-grade gliomas. The volcano plots are shown as a scatter plot where the horizontal axis represents the relative percent change of EM5 frequency between patients with glioma and patients with benign brain tumors. The vertical axis represents the p-value of the difference between the relative percent change of EM5 between patients with glioma and patients with benign brain tumors. The vertical hashed line represents zero percent change. Data points for EM5 frequencies falling to the right of the vertical hashed line have a positive percent difference between glioma and benign, meaning the EM5 motif frequencies are higher in the glioma group compared to the benign group. Likewise, data points for EM5 frequencies falling to the left of the vertical hashed line have a negative percent difference between glioma and benign, meaning the EM5 motif frequencies are higher in the benign group compared to the glioma group. The horizontal line represents a p-value established to determine statistical significance. For example, the hashed line is located at 10² (e.g., p=0.01). Data points for EM5 frequencies falling above the hashed line have p-values less than 0.01 and are statistically different between the glioma group and the benign group. Data points for EM5 frequencies falling below the hashed line have p-values greater than 0.01 and are not statistically different between the glioma group and the benign group. As such, the hashed lines can separate the volcano plot into quadrants to visualize EM5 frequencies that are significantly different between the glioma group and the benign group. It can be appreciated that there are more EM5 having increased frequency in the glioma group 4605 compared to EM5 having increased frequency in the benign group 4610.

Table 5 shows the median EM5 frequencies in cfDNA from CSF in patients with benign and glioma tumors. EM5 motifs are ranked by p-value of difference in motif frequency. Data in the table are representative of the data presented in FIG. 46.

TABLE 5
Median EM5 frequencies in cfDNA from CSF in
patients with benign and glioma tumors.

| | | | | Fold | |
|---|---|---|---|---|---|
| | | Motif | Motif | change | |
| | | frequency | frequency | (Glioma/ | |
| Rank | Motif | (Benign) | (Glioma) | Benign) | P-value |
| 1 | TGTC | 0.443957155 | 0.582032123 | 1.311009671 | 0.00010124 |
| 2 | TGTT | 0.643058688 | 0.861963802 | 1.340412342 | 0.00014974 |
| 3 | TGTA | 0.493478292 | 0.742651544 | 1.50493255 | 0.00015967 |
| 4 | TAAG | 0.433270157 | 0.51853542 | 1.196794684 | 0.00023789 |
| 5 | TGAT | 0.546062939 | 0.699953899 | 1.28181909 | 0.00028386 |
| 6 | TGTG | 0.71712028 | 0.954254691 | 1.330675924 | 0.00043464 |
| 7 | TAAT | 0.587060704 | 0.72369706 | 1.232746554 | 0.00054361 |
| 8 | CGTC | 0.094447653 | 0.108570522 | 1.149531178 | 0.00120765 |
| 9 | CGTA | 0.106803992 | 0.139609607 | 1.307157199 | 0.00145625 |
| 10 | TGAC | 0.37780708 | 0.493027418 | 1.304971358 | 0.00157734 |
| 11 | CGTT | 0.142993287 | 0.168063848 | 1.175326844 | 0.00177036 |
| 12 | TATT | 0.75714873 | 0.917080519 | 1.211229026 | 0.00185131 |
| 13 | TGAG | 0.615150075 | 0.770053617 | 1.251814229 | 0.00192915 |
| 14 | CGTG | 0.198685206 | 0.23196192 | 1.167484607 | 0.0019709 |
| 15 | TGCT | 0.528642406 | 0.687651273 | 1.300787198 | 0.00210009 |
| 16 | TAGC | 0.213646732 | 0.257726008 | 1.20631851 | 0.00216696 |
| 17 | TATG | 0.533708676 | 0.712061363 | 1.334176105 | 0.00245338 |
| 18 | TAGT | 0.215332387 | 0.268024606 | 1.244701782 | 0.00304204 |
| 19 | TGAA | 0.674048398 | 0.886305157 | 1.314898395 | 0.00316749 |
| 20 | TGCA | 0.653769373 | 0.884730101 | 1.353275539 | 0.00341845 |

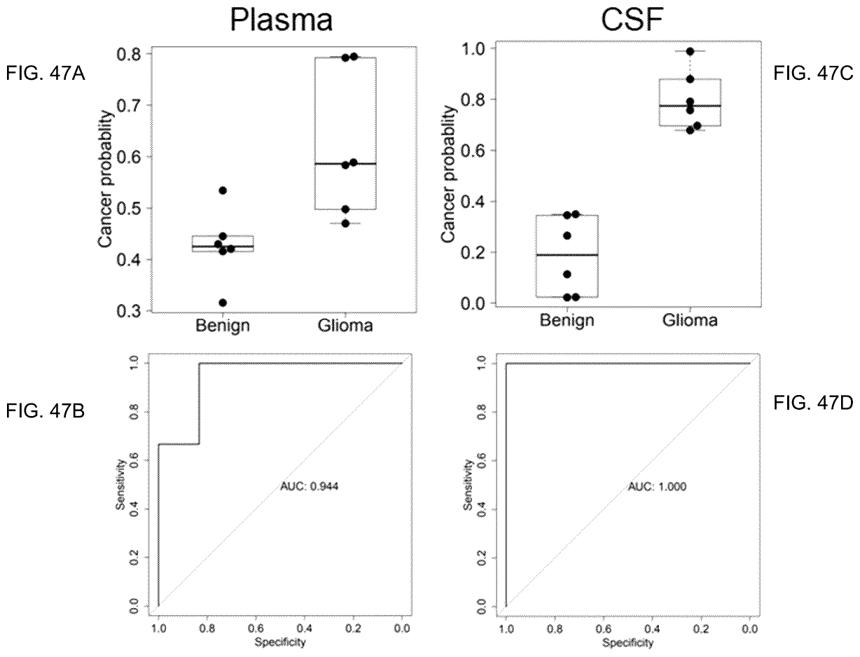

FIG. 47A is a box plot depicting cancer probabilistic scores predicted by SVM models using EM5 from plasma cfDNA as inputs into the model. Compared with subjects having benign brain tumors, cancer probabilistic scores predicted by the models were observed to be elevated in patients with high-grade gliomas using EM5 from plasma cfDNA.

FIG. 47B depicts an ROC curve analysis using the EM5 from plasma cfDNA to differentiate between subjects with benign brain tumors or high-grade gliomas. We show that the AUC for differentiating patients having benign brain tumors from patients with high-grade gliomas was 0.944 using EM5 from plasma cfDNA.

FIG. 47C is a box plot depicting cancer probabilistic scores predicted by SVM models using EM5 from CSF cfDNA as inputs into the model. Compared with subjects having benign brain tumors, cancer probabilistic scores predicted by the models were observed to be elevated in patients with high-grade gliomas using EM5 from CSF cfDNA.

FIG. 47D depicts an ROC curve analysis using the EM5 from CSF cfDNA to differentiate between subjects with benign brain tumors or high-grade gliomas. We show that the AUC for differentiating patients having benign brain tumors from patients with high-grade gliomas was 1.000 using EM5 from CSF cfDNA.

Overall, these results demonstrate better clustering and sensitivity in differentiating high-grade gliomas from benign brain tumors in CSF cfDNA as compared to plasma cfDNA when using EM5 to differentiate between tumor types.

### 3. EM3

We next tested the ability of EM3 to differentiate between patients having benign brain tumors and patients with high-grade gliomas in both plasma and CSF cfDNA. The data were visualized using hierarchical clustering heatmaps with each column corresponding to a sample and each row corresponding to a EM3. EM3s for each sample were used as features as input into the model. Volcano plot analyses were also performed to show differential EM3 frequency in CSF cfDNA between patients with benign brain tumors and high-grade gliomas.

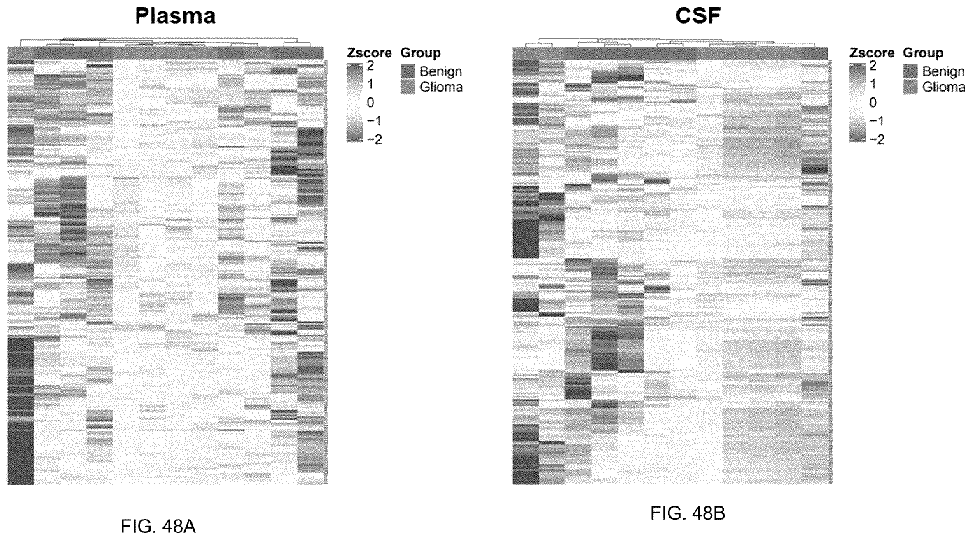

FIG. 48A is a hierarchical clustering heatmap analysis of EM3 from plasma cfDNA of patients having benign brain tumors or high-grade gliomas. Visualization of the patterns of Z-score coloring in the heatmap generally indicates a low degree of clustering of each sample type. For example, the six samples of the benign brain tumor group were clustered into a total of three clusters, with one of the samples being in a cluster as a single sample. Similarly, the six samples of the high-grade glioma group were clustered into a total of three clusters, with two of the samples being in a cluster as a single sample.

FIG. 48B is a hierarchical clustering heatmap analysis of EM3 from plasma cfDNA of patients having benign brain tumors or high-grade gliomas. Visualization of the patterns of Z-score coloring in the heatmap generally indicates a higher degree of clustering of each sample type, compared to plasma. For example, the six samples of the benign brain tumor group were clustered into two clusters, with one samples being in a cluster as a single sample. The six samples of the high-grade glioma group were clustered into a total of two clusters.

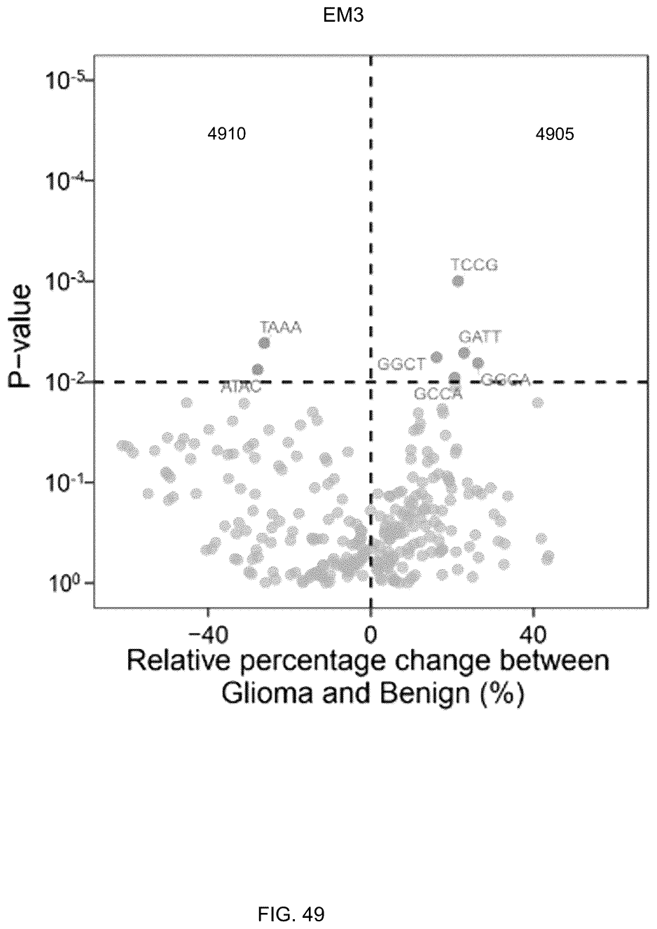

FIG. 49 is a volcano plot analysis showing differential EM3 frequency in CSF cfDNA between patients with benign brain tumors and high-grade gliomas. The volcano plots are shown as a scatter plot where the horizontal axis represents the relative percent change of EM3 frequency between patients with glioma and patients with benign brain tumors. The vertical axis represents the p-value of the difference between the relative percent change of EM3 between patients with glioma and patients with benign brain tumors. The vertical hashed line represents zero percent change. Data points for EM3 frequencies falling to the right of the vertical hashed line have a positive percent difference between glioma and benign, meaning the EM3 motif frequencies are higher in the glioma group compared to the benign group. Likewise, data points for EM3 frequencies falling to the left of the vertical hashed line have a negative percent difference between glioma and benign, meaning the EM3 motif frequencies are higher in the benign group compared to the glioma group. The horizontal line represents a p-value established to determine statistical significance. For example, the hashed line is located at 10⁻² (e.g., p=0.01). Data points for EM3 frequencies falling above the hashed line have p-values less than 0.01 and are statistically different between the glioma group and the benign group. Data points for EM3 frequencies falling below the hashed line have p-values greater than 0.01 and are not statistically different between the glioma group and the benign group. As such, the hashed lines can separate the volcano plot into quadrants to visualize EM3 frequencies that are significantly different between the glioma group and the benign group. It can be appreciated that there are more EM3 having increased frequency in the glioma group 4905 compared to EM3 having increased frequency in the benign group 4910.

Table 6 shows the median EM3 frequencies in cfDNA from CSF in patients with benign and glioma tumors. EM3 motifs are ranked by p-value of difference in motif frequency. Data in the table are representative of the data presented in FIG. 49.

TABLE 6
Median EM3 frequencies in cfDNA from CSF in
patients with benign and glioma tumors.

| | | | | Fold | |
|---|---|---|---|---|---|
| | | Motif | Motif | change | |
| | | frequency | frequency | (Glioma/ | |
| Rank | Motif | (Benign) | (Glioma) | Benign) | P-value |
| 1 | TCCG | 0.064140069 | 0.077888357 | 1.214347882 | 0.00099442 |
| 2 | TAAA | 0.528182697 | 0.38985717 | 0.738110453 | 0.00410304 |
| 3 | GATT | 0.411861511 | 0.50623073 | 1.229128521 | 0.0051424 |
| 4 | GGCT | 0.551730713 | 0.640951026 | 1.16170989 | 0.00567909 |
| 5 | GGCA | 0.473774168 | 0.598643403 | 1.263562776 | 0.0065002 |
| 6 | ATAC | 0.305517512 | 0.220523063 | 0.721801709 | 0.00753433 |
| 7 | GCCA | 0.630371159 | 0.760432476 | 1.206324979 | 0.00903496 |
| 8 | TGCA | 0.559731731 | 0.676479007 | 1.208577198 | 0.01144773 |
| 9 | AGCA | 0.67716718 | 0.954872811 | 1.41009907 | 0.01601757 |
| 10 | CAAA | 0.447845464 | 0.244955773 | 0.546964951 | 0.0160308 |
| 11 | CCAA | 0.315529768 | 0.217080194 | 0.687986416 | 0.01632061 |
| 12 | TTCG | 0.056720299 | 0.066673994 | 1.175487349 | 0.01853971 |
| 13 | TGAA | 0.430881343 | 0.369192773 | 0.856831652 | 0.01975923 |
| 14 | AGTC | 0.393606058 | 0.439459246 | 1.116495127 | 0.02026345 |
| 15 | TGCT | 0.715486158 | 0.842908797 | 1.178092388 | 0.02038632 |
| 16 | GTAA | 0.40890462 | 0.354988935 | 0.86814606 | 0.02397826 |
| 17 | CTAA | 0.372556373 | 0.245889636 | 0.660006522 | 0.02424747 |
| 18 | ATCG | 0.048718788 | 0.054640851 | 1.121556027 | 0.02598766 |
| 19 | CTTA | 0.39920682 | 0.330055793 | 0.826778944 | 0.02659808 |
| 20 | TCGT | 0.045303107 | 0.050616583 | 1.11728722 | 0.02957491 |

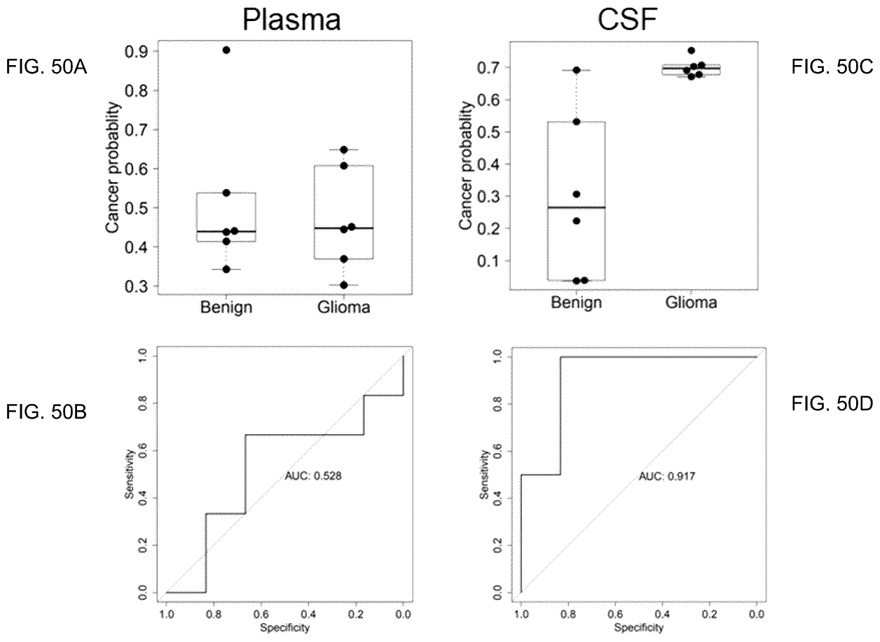

FIG. 50A is a box plot depicting cancer probabilistic scores predicted by SVM models using EM3 from plasma cfDNA as inputs into the model. Interestingly, cancer probabilistic scores predicted by the models were observed to be similar between patients with high-grade gliomas using EM3 from plasma cfDNA.

FIG. 50B depicts an ROC curve analysis using the EM3 from plasma cfDNA to differentiate between subjects with benign brain tumors or high-grade gliomas. We show that the AUC for differentiating patients having benign brain tumors from patients with high-grade gliomas was 0.528 using EM3 from plasma cfDNA.

FIG. 50C is a box plot depicting cancer probabilistic scores predicted by SVM models using EM3 from CSF cfDNA as inputs into the model. Compared with subjects having benign brain tumors, cancer probabilistic scores predicted by the models were observed to be elevated in patients with high-grade gliomas using EM3 from CSF cfDNA.

FIG. 50D depicts an ROC curve analysis using the EM3 from CSF cfDNA to differentiate between subjects with benign brain tumors or high-grade gliomas. We show that the AUC for differentiating patients having benign brain tumors from patients with high-grade gliomas was 0.917 using EM3 from CSF cfDNA.

Overall, these results demonstrate better clustering and sensitivity in differentiating high-grade gliomas from benign brain tumors in CSF cfDNA as compared to plasma cfDNA when using EM3 to differentiate between tumor types.

### 4. POEM

We next tested the ability of POEM to differentiate between patients having benign brain tumors and patients with high-grade gliomas in both plasma and CSF cfDNA. The data were visualized using hierarchical clustering heatmaps with each column corresponding to a sample and each row corresponding to a POEM. POEMs for each sample were used as features as input into the model. Volcano plot analyses were also performed to show differential POEM frequency in CSF cfDNA between patients with benign brain tumors and high-grade gliomas.

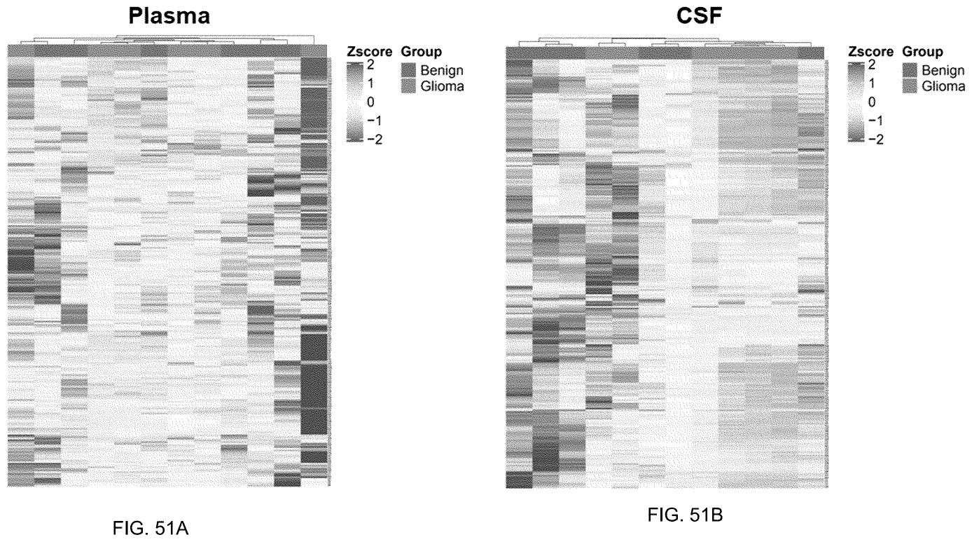

FIG. 51A is a hierarchical clustering heatmap analysis of POEM from plasma cfDNA of patients having benign brain tumors or high-grade gliomas. Visualization of the patterns of Z-score coloring in the heatmap generally indicates a low degree of clustering of each sample type. For example, the six samples of the benign brain tumor group were clustered into a total of three clusters, with one of the samples being in a cluster as a single sample. The six samples of the high-grade glioma group were clustered into a total of three clusters, with two of the samples being in a cluster as a single sample.

FIG. 51B is a hierarchical clustering heatmap analysis of POEM from plasma cfDNA of patients having benign brain tumors or high-grade gliomas. Visualization of the patterns of Z-score coloring in the heatmap generally indicates a higher degree of clustering of each sample type, compared to plasma. For example, the six samples of the benign brain tumor group were clustered into three clusters, with one samples being in a cluster as a single sample. The six samples of the high-grade glioma group were clustered into a total of two clusters.

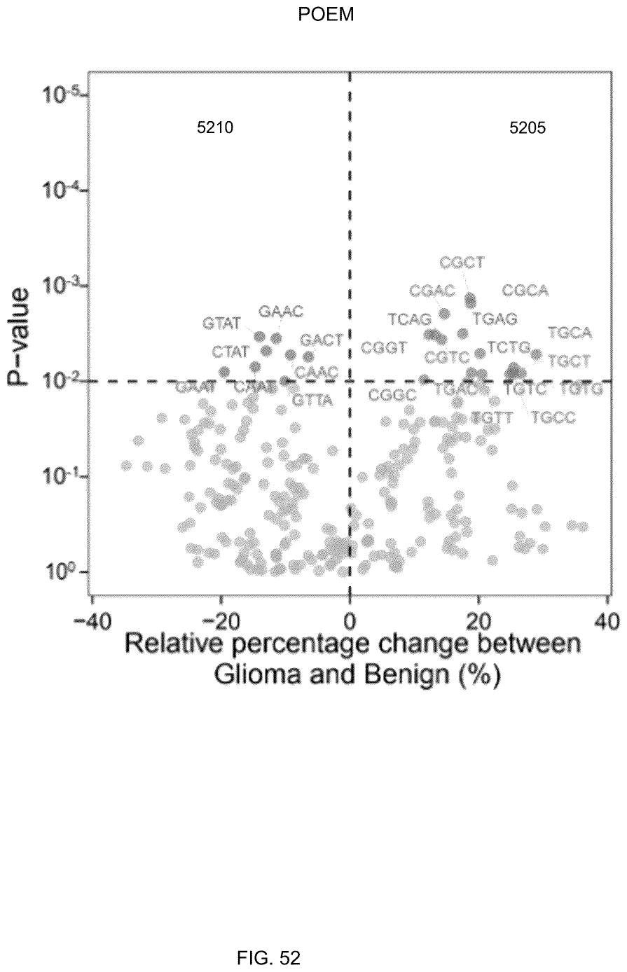

FIG. 52 is a volcano plot analysis showing differential POEM frequency in CSF cfDNA between patients with benign brain tumors and high-grade gliomas. The volcano plots are shown as a scatter plot where the horizontal axis represents the relative percent change of POEM frequency between patients with glioma and patients with benign brain tumors. The vertical axis represents the p-value of the difference between the relative percent change of POEM between patients with glioma and patients with benign brain tumors. The vertical hashed line represents zero percent change. Data points for POEM frequencies falling to the right of the vertical hashed line have a positive percent difference between glioma and benign, meaning the POEM motif frequencies are higher in the glioma group compared to the benign group. Likewise, data points for POEM frequencies falling to the left of the vertical hashed line have a negative percent difference between glioma and benign, meaning the POEM motif frequencies are higher in the benign group compared to the glioma group. The horizontal line represents a p-value established to determine statistical significance. For example, the hashed line is located at 10⁻² (e.g., p=0.01). Data points for POEM frequencies falling above the hashed line have p-values less than 0.01 and are statistically different between the glioma group and the benign group. Data points for POEM frequencies falling below the hashed line have p-values greater than 0.01 and are not statistically different between the glioma group and the benign group. As such, the hashed lines can separate the volcano plot into quadrants to visualize POEM frequencies that are significantly different between the glioma group and the benign group. It can be appreciated that there are more POEM having increased frequency in the glioma group 5205 compared to POEM having increased frequency in the benign group 5210.

Table 7 shows the median POEM frequencies in cfDNA from CSF in patients with benign and glioma tumors. POEM motifs are ranked by p-value of difference in motif frequency. Data in the table are representative of the data presented in FIG. 52.

TABLE 7
Median POEM frequencies in cfDNA from CSF
in patients with benign and glioma tumors.

| | | | | Fold | |
|---|---|---|---|---|---|
| | | Motif | Motif | change | |
| | | frequency | frequency | (Glioma/ | |
| Rank | Motif | (Benign) | (Glioma) | Benign) | P-value |
| 1 | CGCT | 0.071170039 | 0.084414576 | 1.18609709 | 0.00133781 |
| 2 | CGCA | 0.065926829 | 0.078282559 | 1.18741582 | 0.0015236 |
| 3 | CGAC | 0.040742095 | 0.046708409 | 1.146441021 | 0.00195352 |
| 4 | TGAG | 0.642933916 | 0.755392778 | 1.174915119 | 0.00314275 |
| 5 | CGGT | 0.048613838 | 0.054574086 | 1.122603942 | 0.00323245 |
| 6 | TCAG | 0.41466678 | 0.469398014 | 1.131988471 | 0.00324395 |
| 7 | GTAT | 0.246551973 | 0.211879105 | 0.859368928 | 0.00338248 |
| 8 | GAAC | 0.327729326 | 0.290093568 | 0.885162068 | 0.00353349 |
| 9 | CGTC | 0.080046278 | 0.091441227 | 1.142354513 | 0.00363123 |
| 10 | CTAT | 0.341432128 | 0.296986799 | 0.869826751 | 0.00478628 |
| 11 | TCTG | 0.518672715 | 0.623346849 | 1.201811528 | 0.00507517 |
| 12 | TGCA | 0.554209239 | 0.714389022 | 1.289024022 | 0.00520786 |
| 13 | CAAC | 0.414076877 | 0.375772923 | 0.90749555 | 0.00525671 |
| 14 | GACT | 0.313838866 | 0.293485119 | 0.93514587 | 0.0055196 |
| 15 | CAAT | 0.664819328 | 0.566871015 | 0.852669276 | 0.00704262 |
| 16 | TGCT | 0.532522584 | 0.667671204 | 1.25378946 | 0.00715948 |
| 17 | GAAT | 0.585790035 | 0.471459144 | 0.80482616 | 0.00794102 |
| 18 | TGAC | 0.337489198 | 0.401059385 | 1.188362137 | 0.00812903 |
| 19 | TGTG | 0.722777128 | 0.914631078 | 1.265439985 | 0.00823792 |
| 20 | TGCC | 0.418234317 | 0.524344811 | 1.253710633 | 0.00828967 |

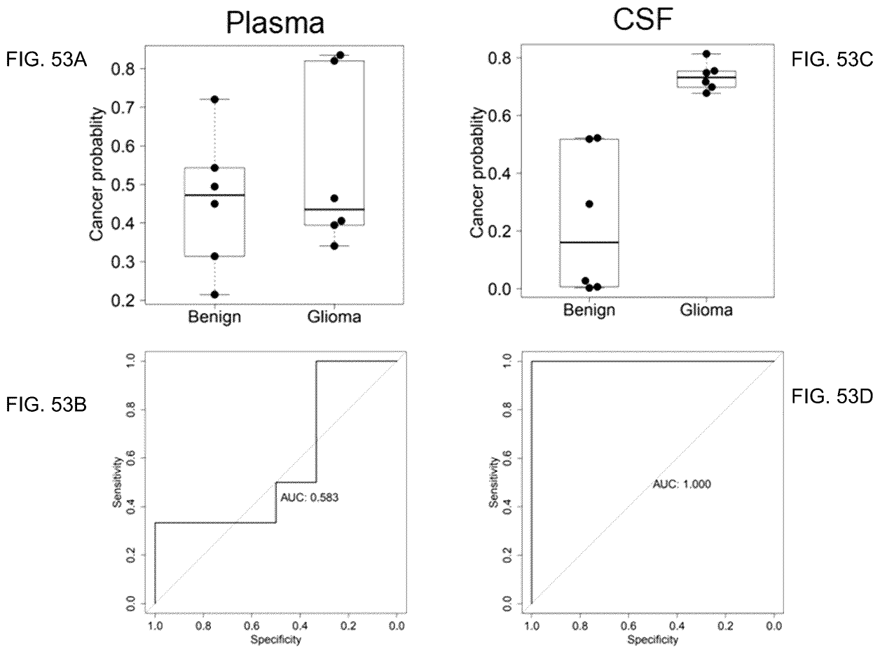

FIG. 53A is a box plot depicting cancer probabilistic scores predicted by SVM models using POEM from plasma cfDNA as inputs into the model. Interestingly, cancer probabilistic scores predicted by the models were observed to be similar between patients with high-grade gliomas using POEM from plasma cfDNA.

FIG. 53B depicts an ROC curve analysis using the POEM from plasma cfDNA to differentiate between subjects with benign brain tumors or high-grade gliomas. We show that the AUC for differentiating patients having benign brain tumors from patients with high-grade gliomas was 0.583 using POEM from plasma cfDNA.

FIG. 53C is a box plot depicting cancer probabilistic scores predicted by SVM models using POEM from CSF cfDNA as inputs into the model. Compared with subjects having benign brain tumors, cancer probabilistic scores predicted by the models were observed to be elevated in patients with high-grade gliomas using POEM from CSF cfDNA.

FIG. 53D depicts an ROC curve analysis using the POEM from CSF cfDNA to differentiate between subjects with benign brain tumors or high-grade gliomas. We show that the AUC for differentiating patients having benign brain tumors from patients with high-grade gliomas was 1.000 using POEM from CSF cfDNA.

Overall, these results demonstrate better clustering and sensitivity in differentiating high-grade gliomas from benign brain tumors in CSF cfDNA as compared to plasma cfDNA when using POEM to differentiate between tumor types.

### 5. Combined Analysis

In some embodiments, any two or more features, such as PREM, EM5, EM3, and POEM, can be combined and used by various machine learning approaches such as SVM, As such, we performed a combined analysis of various types of features to determine if an increased performance is achieved in differentiating between patients having benign brain tumors and patients with high-grade gliomas in both plasma and CSF cfDNA. PREM, EM5, EM3, and POEM for each sample were used as features as input into the model.

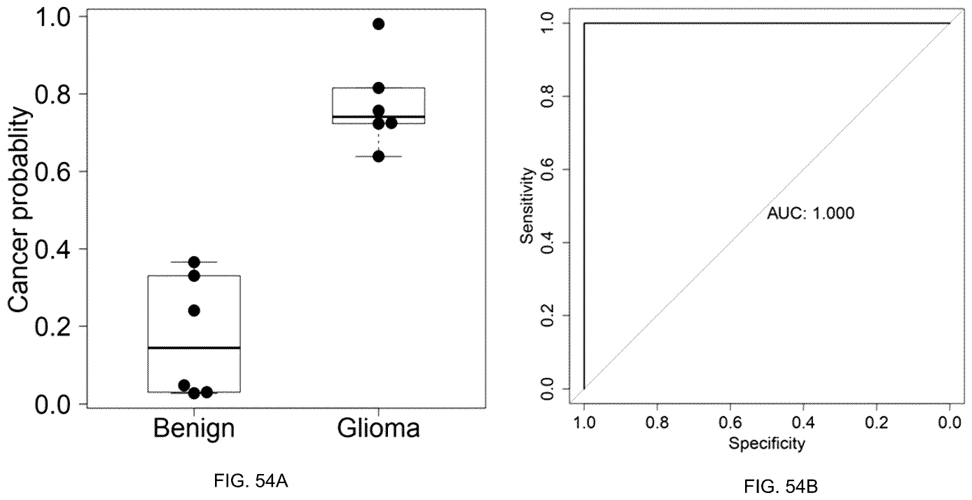

FIG. 54A is a box plot depicting cancer probabilistic scores predicted by SVM models using PREM, EM5, EM3, and POEM from CSF cfDNA as inputs into the model. Compared with subjects having benign brain tumors, cancer probabilistic scores predicted by the models were observed to be elevated in patients with high-grade gliomas using POEM from CSF cfDNA.

FIG. 54B depicts an ROC curve analysis using the PREM, EM5, EM3, and POEM from CSF cfDNA to differentiate between subjects with benign brain tumors or high-grade gliomas. We show that the AUC for differentiating patients having benign brain tumors from patients with high-grade gliomas was 1.000 using POEM from CSF cfDNA.

Leveraging a machine learning model to utilize the end motif types, such as PREM, EM5, EM3, and POEM, could significantly enhance diagnostic performance in distinguishing patients with neurological conditions.

### F. Method

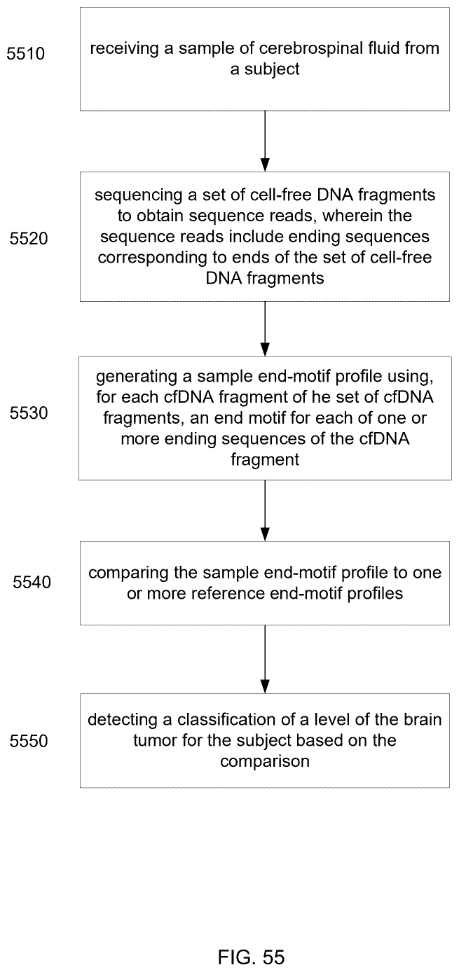

FIG. 55 is a flowchart illustrating a method 5500 for detecting a brain tumor in a subject based on end motifs, according to some embodiments of the present disclosure. Portions or all steps of method 5500 can be performed by a computer system, including one or more processors. The method 5500 can use a trained ML model that was trained by the computer system or another computer system. The ML models can include a support vector machine or clustering. The computer system can comprise various devices, e.g., one device that performed the training and another that uses the trained model.

At block 5510, method 5500 can include receiving a sample of cerebrospinal fluid from a subject, e.g., obtained via techniques described herein.

At block 5520, method 5500 can include sequencing a set of cell-free DNA fragments to obtain sequence reads. The sequence reads can include ending sequences corresponding to ends of the set of cell-free DNA fragments. Various sequencing techniques can be used, e.g., as described herein.

At block 5530, method 5500 can include generating a sample end-motif profile using, for each cell-free DNA fragment of the set of cell-free DNA fragments, an end motif for each of one or more ending sequences of the cell-free DNA fragment. The sample end-motif profile can represent one or more end motifs. The one or more end motifs of the sample end-motif profile can include pre-end motif(s), EM5 end motif(s), EM3 end motif(s), post-end motif(s), or a combination thereof. As examples, the sample end-motif profile may represent at least 2, 3, 4, 5, 6, 7, 8, 9, 10, 20, 30, 40, 50 60, 70, 80, 90, 100, 150, 200, 250, or 256 end motifs.

Generating the sample end-motif profile can include generating the first aggregate using the ending sequences matching any one of the one or more end motifs. Generating the sample end-motif profile can include generating, for each end motif of the set of end motifs, an aggregate of the ending sequences having the end motif.

The sample end-motif profile may represent a set of end motifs, and one or more reference end-motif profiles can include a set of reference F-profiles. The method 5500 can include storing the set of reference F-profile. Each reference F-profile of the set may identify, for each nucleotide of a set of nucleotides, a proportion of cell-free DNA molecules that end in the nucleotide, and may be associated with a type of fragmentation factors.

At block 5540, method 5500 can include comparing the sample end-motif profile to one or more reference end-motif profiles. The one or more reference end-motif profiles can include a first reference end-motif profile determined from cell-free DNA fragments in one or more first reference samples of cerebrospinal fluid measured from one or more first reference subjects having a brain tumor. The one or more reference end-motif profiles can further include a second reference end-motif profile determined from cell-free DNA fragments in one or more second reference samples of cerebrospinal fluid measured from one or more second reference subjects having high intra-cranial pressure. The one or more first reference samples can be measured from one or more first reference subjects having a benign tumor or a glioma. The one or more first reference samples can be measured from a first cohort of reference samples from subjects having a benign tumor and a second cohort of reference samples from subjects having glioma.

Comparing the sample end-motif profile to the one or more reference end-motif profiles can include comparing a first aggregate for the sample end-motif profile to a second aggregate for the one or more reference end-motif profiles.

Comparing the sample end-motif profile to the one or more reference end-motif profiles can include determining proportional contributions of the set of reference F-profiles whose proportional aggregation provide the sample end-motif profile. The proportional contributions may sum to one.

Comparing the sample end-motif profile to the one or more reference profiles can include inputting the sample end-motif profile into a machine learning model that is trained using a set of reference end-motif profiles that include the one or more reference end-motif profiles.

At block 5550, method 5500 can include detecting a classification of a level of the brain tumor for the subject based on the comparison. Classifying whether the subject has the brain tumor can be based on a proportional contribution associated with a reference F-profile of the set of reference F-profiles. The subject may be determined to have the brain tumor based on the proportional contribution exceeding a threshold. The classification of the brain tumor can include whether the subject has the brain tumor. The classification of the brain tumor can include whether the brain tumor is benign or glioma.

In some embodiments, a plasma sample can be used, e.g., as described in section IV.B. For example, a method can comprise: receiving a plasma sample of the subject; sequencing a plurality of cell-free DNA fragments to obtain plasma sequence reads; and generating a plasma end-motif profile using the plasma sequence reads. The plasma end-motif profile can represent the one or more end motifs in the plasma sample.

Comparing the sample end-motif profile to one or more reference end-motif profiles can comprise generating a differential end-motif profile between the sample end-motif profile and the plasma end-motif profile (e.g., the mean fold change from the tables in FIGS. 28-31) and comparing the differential end-motif profile to a reference differential end-motif profile generated using the first reference end-motif profile and a plasma reference end-motif profile for the one or more first reference subjects having the brain tumor (e.g., via reference values determined for end motifs in FIG. 32). A reference value can be selected to differentiate between brain tumor samples and non-brain cancer samples (e.g., with high ICP). For example, a fold change less than 0.15 for CGCC can differentiate between high ICP and a brain tumor.

Accordingly, in some implementations, the differential end-motif profile can comprise a change between the sample of cerebrospinal fluid and the plasma sample. The change can be compared to a reference change between the first reference end-motif profile and the plasma reference end-motif profile for the one or more first reference subjects having the brain tumor (e.g., 0.13 or 0.122 for the two samples in FIG. 32).

### V. METHYLATION AND CG-RELATED MOTIFS

The fragmentomic patterns of cfDNA can be linked to the methylation index of a CpG site. Preferential cutting of cfDNA at methylated CpG sites is demonstrated by a higher cleavage ratio at that particular site, also denoted by an increased motif ratio (CGN/NCG) or to individual values. Briefly, the CGN/NCG motif ratio can quantify the relative abundance of 5′ end motifs in cfDNA, particularly at CpG sites. The CGN/NCG ratio can be calculated as the number of cfDNA fragments with 5′ CGN motifs (e.g., CGA, CGT, CGG, and CGC) divided by the number of 5′ NCG motifs (e.g., ACG, TCG, GCG, and CCG).

### A. Cutting Positions and Cleavage Profile

The addition of a methyl group to a cytosine nucleotide can affect chromatin accessibility to that region of DNA. A change in the accessibility of regions of DNA can hence impact positions where nucleases can cleave DNA.

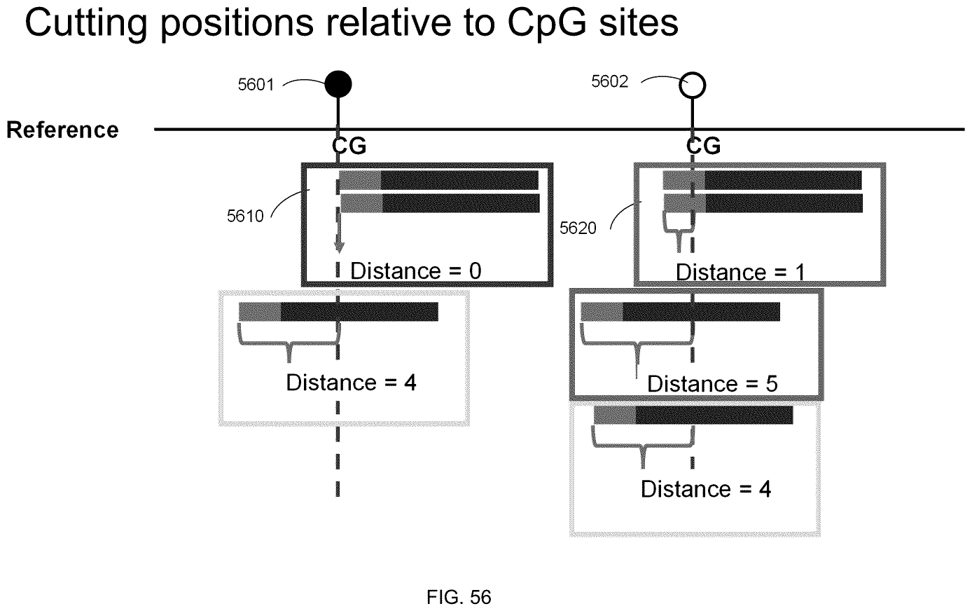

FIG. 56 illustrates cutting positions relative to CpG sites (also referred to as CG sites) according to embodiments of the present disclosure. The horizontal line refers to a reference sequence, e.g., part of a reference genome, which contains two CpG sites. After sequencing, the resulting reads can be mapped to this region. The distance between the fragment ends and the position relative to the CpG sites can be calculated. For example, the fragments 5610 ended exactly at the CG position, and thus have a distance of 0 (also referred to as position 0).

The fragments 5620 end one position to the left of CpG site 5602. Since the fragment was cut one base before the CpG site, the distance is considered one. In this manner, we can calculate the distance between the cutting ends and the CpG sites and group the fragments with the same distance together. This distance is associated with the methylation level.

DNA fragments can be grouped depending on distance from the 5′ ends to the CpG site. If the first two nucleotides at the 5′ end of a fragment are CG, the aforementioned distance is 0. If there is one nucleotide at the 5′ end immediately preceding the CG, the aforementioned distance is 1, which corresponds to fragments having an NCG motif, where N is any one of the 4 bases. The sequenced CpG sites could be grouped into different categories according to their distances relative to 5′ ends.

The CGN/NCG motif ratio can provide a method to reflect differences between methylated and unmethylated CpG sites. For example, there is an increased probability of nuclease cutting at a methylated C-site compared to that of an unmethylated C-site. The increased probability results in higher CGN/NCG motif ratios at methylated sites compared to unmethylated sites.

### B. Differences Between Plasma and CSF

Fragmentomic-based methylation analysis (FRAGMA) allows for the methylation status to be deduced from end motif patterns of cfDNA (Zhou et al. 2022). We analyzed whether this methylation aware cleavage patterns are also observed in CSF cfDNA by evaluating the CGN/NCG motif ratio of different parts of the genome for patients having high intracranial pressure.

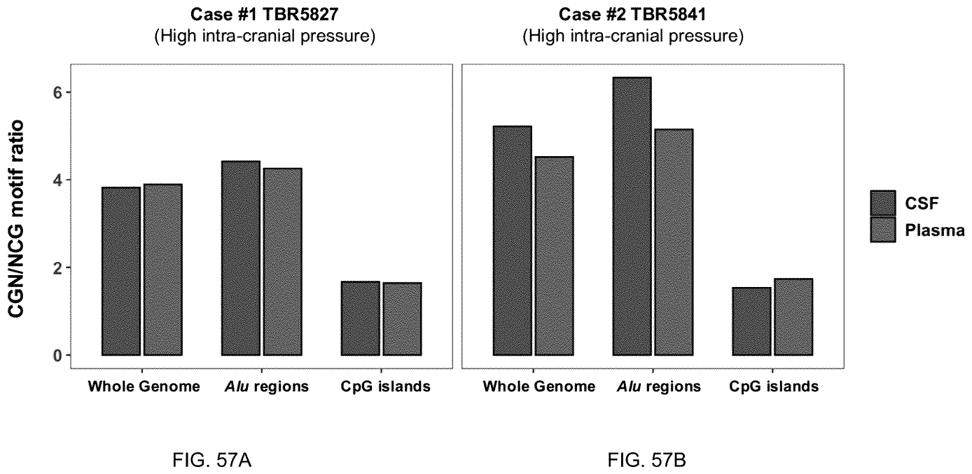

FIGS. 57A-57B show CGN/NCG motif ratio analysis across the whole genome, Alu regions, and CpG islands in cfDNA from CSF and plasma. Methylation aware cleavage pattern are generally observed in CSF cfDNA-CGN/NCG motif in whole genome, Alu regions and CpG islands, correspond to the methylation density in those regions, which reflects a similar pattern to plasma cfDNA. This allows for the potential utility of FRAGMA to deduce methylation status in CSF, which could provide important clinical utilities such as cancer diagnosis, and staging/localization of cancers using deduced methylation signals. The CGN/NCG motif ratio correspond well between plasma and CSF cfDNA in the individual TBR5827, reflecting on similar phenomenon of methylation aware cleavage and similarity in overall methylation levels. Some deviation is observed in TBR5841 between CSF and plasma cfDNA CGN/NCG motif ration. This can potentially be explored further, this difference may be a in tissue composition (e.g., increase in 5hmC modification from neuronal cfDNA which may affect CGN/NCG), or possible effects from the pathological state.

We see that the CGN/NCG motif ratio corresponds well between plasma and CSF. The ratio is similar in both the whole genome and CpG islands, but there is a slight difference in the ratios for whole genome in FIG. 57B. The results show that FRAGMA holds true in CSF. This can indicate the enzymes needed to have similar levels of CGN and NCG are present and that an additional source of DNA besides plasma may be present.

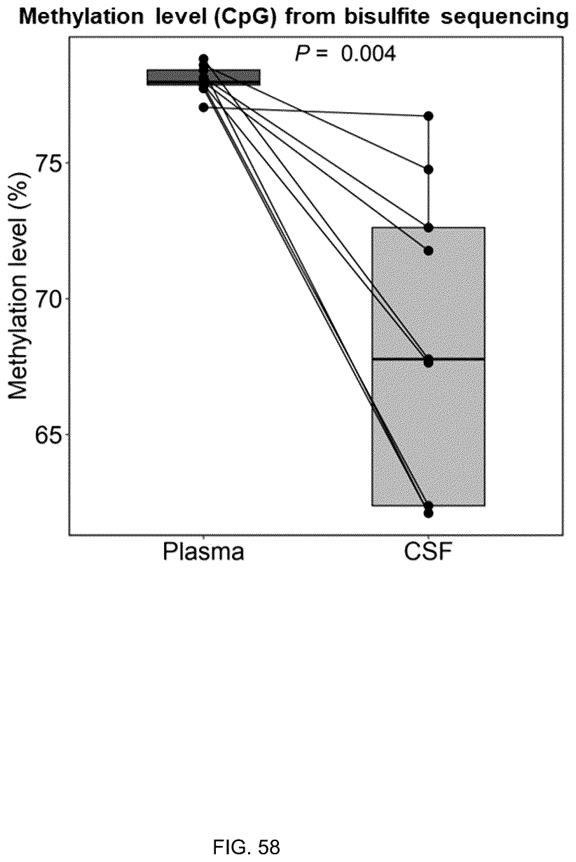

FIG. 58 is a box plot of methylation levels from bisulfite sequencing of paired plasma and CSF cfDNA from patients having benign brain tumors. We observed that plasma cfDNA is approximately 80% methylated at CpG sites whereas CSF cfDNA is approximately 67% methylated at CpG sites. Thus, the mean CpG methylation is significantly lower (p=0.004) in CSF compared to plasma assessed by Wilcoxon matched-pairs signed rank test. These results suggest that CpG sites in CSF are hypomethylated compared to plasma.

To determine whether preferential cutting at methylated CpG sites exists, we next examined the cleavage profile of cfDNA from CSF based on paired bisulfite sequencing in patients having benign brain tumors. We also examined the motif ratio (CGN/NCG) of hypermethylated and hypomethylated CpG sites. Hypermethylated and hypomethylated CpG sites are defined by a methylation index of >70% and <30%, respectively.

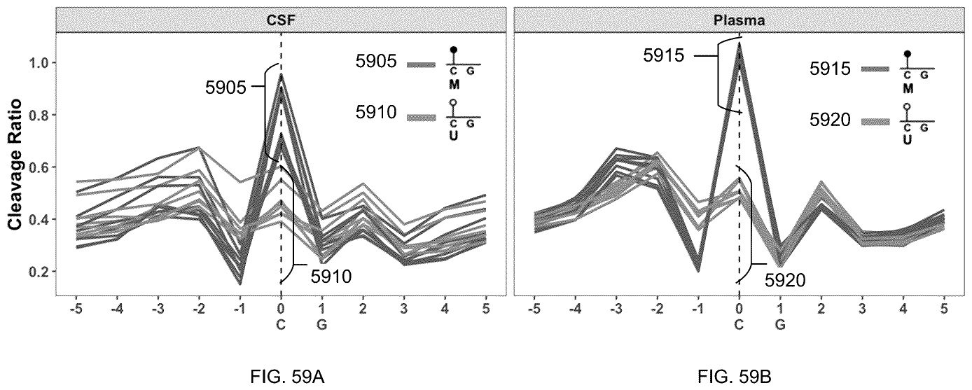

FIG. 59A shows the cleavage profile of methylated and unmethylated CpG sites of CSF cfDNA in a 11-nt cleavage measurement window. Each line in the analysis represents one patients having benign brain tumor. The vertical hashed line represents a CpG site that can be methylated 5905 or unmethylated 5910. Preferential cutting of cfDNA at CpG sites is demonstrated by a higher cleavage ratio at that particular site. The results suggest that CSF shows preferential cleavage at methylated CpG sites 5905, as indicated by a spike in the cleavage ratio at a CpG site. These are in contrast to no distinct increase in cleavage pattern (e.g., spikes in cleavage ratio) for unmethylated CpG sites 5910.

FIG. 59B shows the cleavage profile of methylated and unmethylated CpG sites of plasma cfDNA in a 11-nt cleavage measurement window. Each line in the analysis represents one patients having benign brain tumor. The vertical hashed line represents a CpG site that can be methylated or unmethylated. The results suggest that plasma shows preferential cleavage at methylated CpG sites 5915, as indicated by a spike in the cleavage ratio at a CpG site. These are in contrast to no distinct increase in cleavage pattern (e.g., spikes in cleavage ratio) for unmethylated CpG sites 5910.

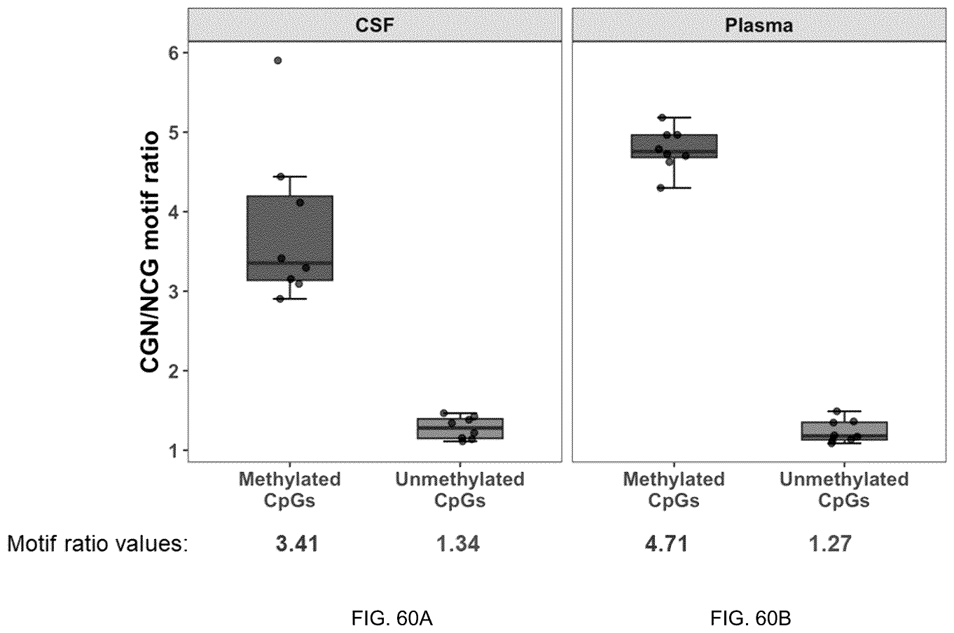

FIG. 60A is a box plot of CGN/NCG motif ratios of methylated and unmethylated CpG sites in CSF cfDNA from patients having benign brain tumors. Motif ratios of 3.41 and 1.43 were observed for methylated and unmethylated CpGs in CSF cfDNA, respective. These data indicate a higher cleavage ratio was observed in hypermethylated sites in CSF cfDNA.

FIG. 60B is a box plot of CGN/NCG motif ratios of methylated and unmethylated CpG sites in plasma cfDNA from patients having benign brain tumors. Motif ratios of 4.71 and 1.27 were observed for methylated and unmethylated CpGs in CSF cfDNA, respectively. These data indicate a higher cleavage ratio was observed in hypermethylated sites in plasma cfDNA.

CGN/NCG ratios in cfDNA from CSF can also inform methylation levels of different genomic regions.

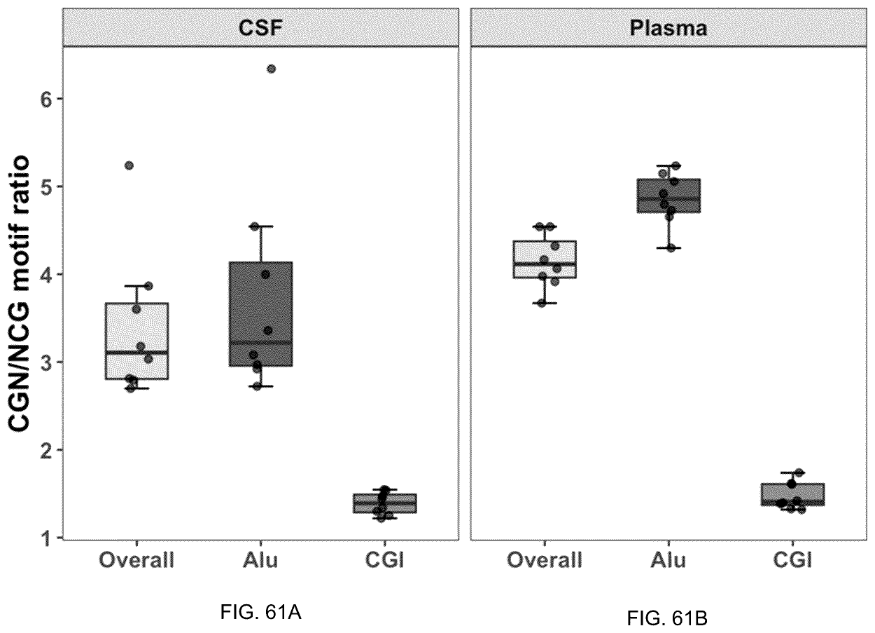

FIG. 61A is a box plot of CGN/NCG motif ratios across the whole genome (overall methylation level), Alu regions, and CpG islands (CGI) in CSF cfDNA from patients with benign brain tumors. The CGN/NCG motif ratios were similar between the whole genome (overall) and Alu elements. The CGN/NCG motif ratio for CGI was reduced compared to overall methylation and methylation at Alu elements.

FIG. 61B is a box plot of CGN/NCG motif ratios across the whole genome (overall methylation level), Alu regions, and CpG islands (CGI) in plasma cfDNA from patients with benign brain tumors. The CGN/NCG motif ratios were similar between the whole genome (overall) and Alu elements. The CGN/NCG motif ratio for CGI was reduced compared to overall methylation and methylation at Alu elements.

These combined results suggest that such CG-related analysis can be used to examine differential methylation sites. Further details can be found in U.S. Patent Publication US2023/0374601.

### C. Differentiating Benign Tumor from Glioma

In some embodiments, CG-related analysis of glioma specific markers can be used to distinguish between patients with benign brain tumors and patients with gliomas. In other embodiments, a methylation density can be used to distinguish between patients with benign brain tumors and patients with gliomas.

We selected 41,198 hyper-methylated CpG sites and 43,642 hypo-methylated CpG sites in glioma and evaluated whether tumor-specific methylation changes can be detected using FRAGMA analysis or of glioma specific markers. To determine such CpG sites, we examined the cleavage profile of cfDNA from CSF based on paired bisulfite sequencing. We have selected hyper-methylated and hypo-methylated CpG sites from CSF cfDNA, which were commonly found in all our cases of glioma.

### 1. CGN and NCG Ratio

We examined the cleavage profile of cfDNA from CSF and plasma based on paired bisulfite sequencing to determining whether tumor-specific methylation changes can be detected using FRAGMA analysis of glioma specific markers.

We first examined 5′ end motif ratios for glioma specific CpG methylation sites in plasma and CSF cfDNA from patients with benign brain tumors and high-grade gliomas.

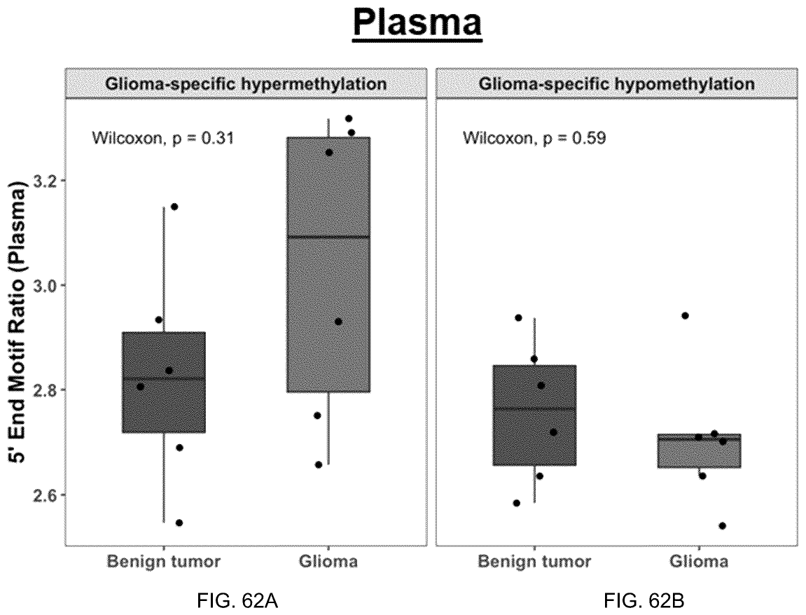

FIG. 62A is a box plot of the 5′ CGN/NCG end motif ratios for glioma specific hypermethylated CpG sites in plasma cfDNA in patients with benign brain tumors or patients with gliomas. It can be appreciated that there is no apparent difference in the 5′ CGN/NCG end motif ratios of glioma specific hypermethylated CpG sites in plasma of patients having benign brain tumors (p=0.31 using Wilcoxon unpaired T-test comparisons).

FIG. 62B is a box plot of the 5′ CGN/NCG end motif ratios for glioma specific hypomethylated CpG sites in plasma cfDNA in patients with benign brain tumors or patients with gliomas. It can be appreciated that there is no apparent difference in the 5′ CGN/NCG end motif ratios of glioma specific hypomethylated CpG sites in plasma of patients having benign brain tumors (p=0.59 using Wilcoxon unpaired T-test comparisons).

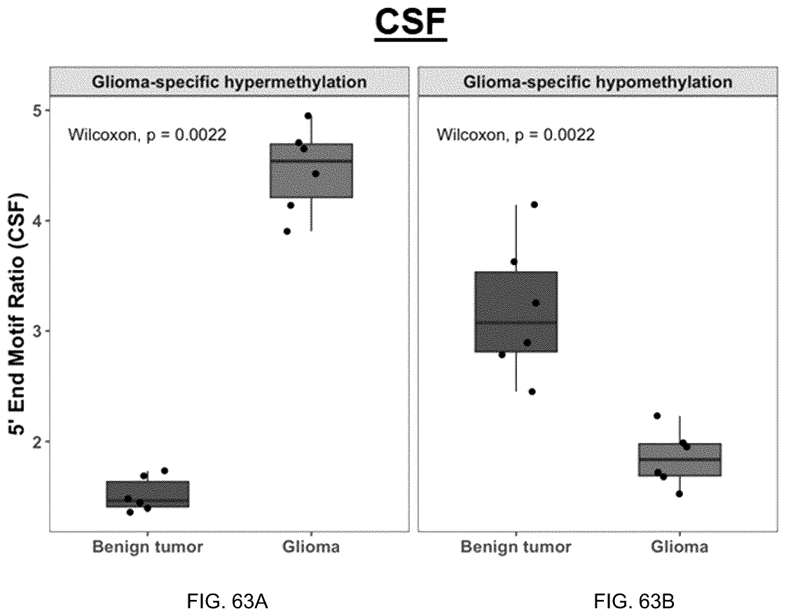

FIG. 63A is a box plot of the 5′ CGN/NCG end motif ratios for glioma specific hypermethylated CpG sites in CSF cfDNA in patients with benign brain tumors or patients with gliomas. It can be appreciated that the 5′ CGN/NCG end motif ratios of glioma specific hypermethylated CpG sites are significantly higher in CSF from patients having gliomas compared to patients having benign brain tumors (p=0.0022 using Wilcoxon unpaired T-test comparisons).

FIG. 63B is a box plot of the 5′ CGN/NCG end motif ratios for glioma specific hypomethylated CpG sites in CSF cfDNA in patients with benign brain tumors or patients with gliomas. It can be appreciated that the 5′ CGN/NCG end motif ratios of glioma specific hypomethylated CpG sites are significantly lower in CSF from patients having gliomas compared to patients having benign brain tumors (p=0.0022 using Wilcoxon unpaired T-test comparisons).

We next examined the frequency of 5′ CGN motifs at glioma specific CpG methylation sites in plasma and CSF cfDNA from patients with benign brain tumors and high-grade gliomas.

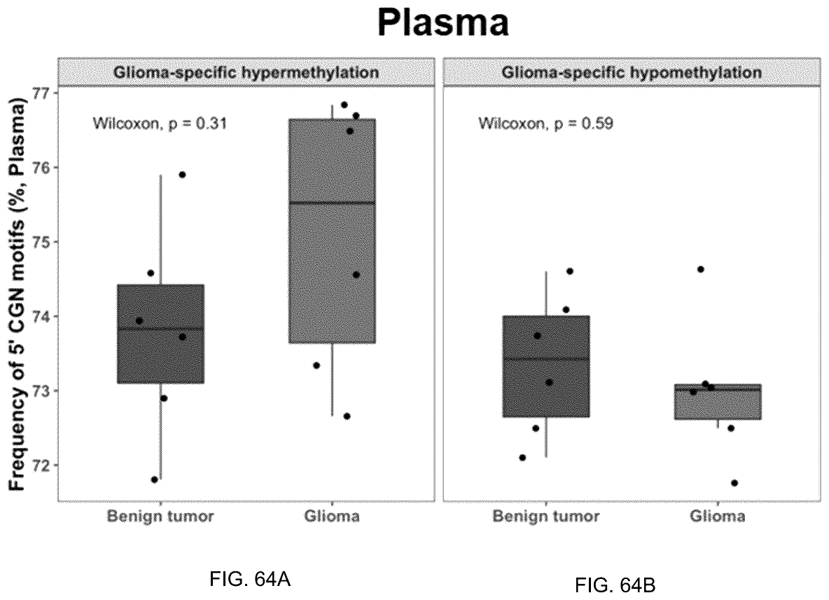

FIG. 64A is a box plot of the frequency of 5′ CGN motifs at glioma specific hypermethylated CGN CpG sites in plasma cfDNA in patients with benign brain tumors or patients with gliomas. It can be appreciated that there is no apparent difference in the frequency of 5′ CGN motif ratios of glioma specific hypermethylated CpG sites in plasma of patients having benign brain tumors (p=0.31 using Wilcoxon unpaired T-test comparisons).

FIG. 64B is a box plot of the frequency of 5′ CGN motifs at glioma specific hypomethylated CGN CpG sites in plasma cfDNA in patients with benign brain tumors or patients with gliomas. It can be appreciated that there is no apparent difference in the frequency of 5′ CGN motif ratios of glioma specific hypomethylated CpG sites in plasma of patients having benign brain tumors (p=0.59 using Wilcoxon unpaired T-test comparisons).

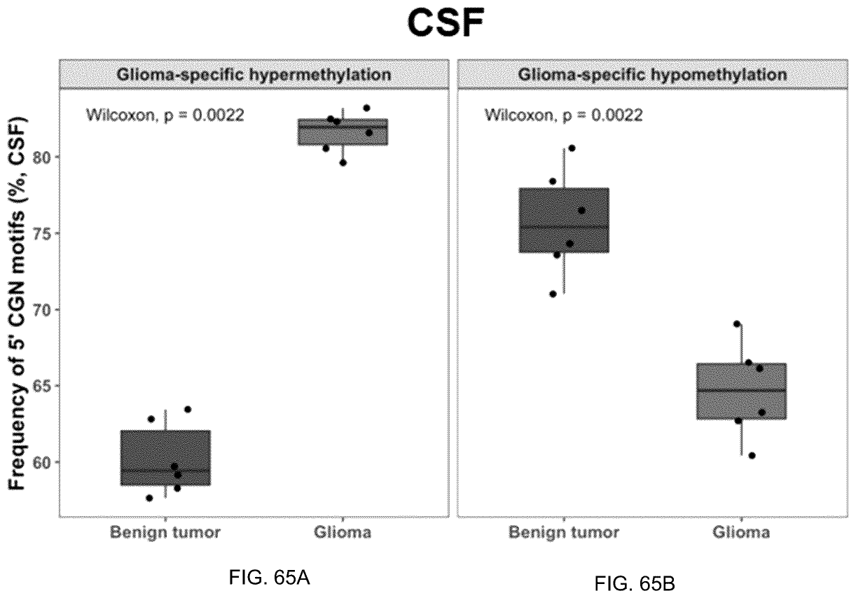

FIG. 65A is a box plot of the frequency of 5′ CGN motifs at glioma specific hypermethylated CGN CpG sites in CSF cfDNA in patients with benign brain tumors or patients with gliomas. It can be appreciated that the frequency of 5′ CGN motif ratios of glioma specific hypermethylated CpG sites are significantly higher in CSF from patients having gliomas compared to patients having benign brain tumors (p=0.0022 using Wilcoxon unpaired T-test comparisons).

FIG. 65B is a box plot of the frequency of 5′ CGN motifs at glioma specific hypomethylated CGN CpG sites in CSF cfDNA in patients with benign brain tumors or patients with gliomas. It can be appreciated that the frequency of 5′ CGN motif ratios of glioma specific hypomethylated CpG site are significantly lower in CSF from patients having gliomas compared to patients having benign brain tumors (p=0.0022 using Wilcoxon unpaired T-test comparisons).

We next examined the frequency of 5′ NCG motifs at glioma specific CpG methylation sites in plasma and CSF cfDNA from patients with benign brain tumors and high-grade gliomas.

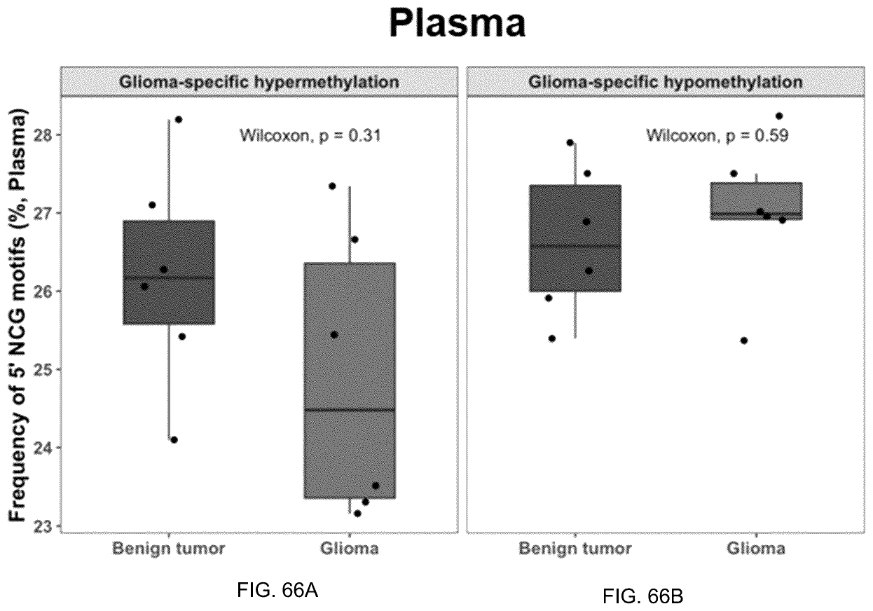

FIG. 66A is a box plot of the frequency of 5′ NCG motifs at glioma specific hypermethylated NCG CpG sites in plasma cfDNA in patients with benign brain tumors or patients with gliomas. It can be appreciated that there is no apparent difference in the frequency of 5′ NCG motif ratios of glioma specific hypermethylated CpG sites in plasma of patients having benign brain tumors (p=0.31 using Wilcoxon unpaired T-test comparisons).

FIG. 66B is a box plot of the frequency of 5′ NCG motifs at glioma specific hypomethylated NCG CpG sites in plasma cfDNA in patients with benign brain tumors or patients with gliomas. It can be appreciated that there is no apparent difference in the frequency of 5′ NCG motif ratios of glioma specific hypomethylated CpG sites in plasma of patients having benign brain tumors (p=0.59 using Wilcoxon unpaired T-test comparisons).

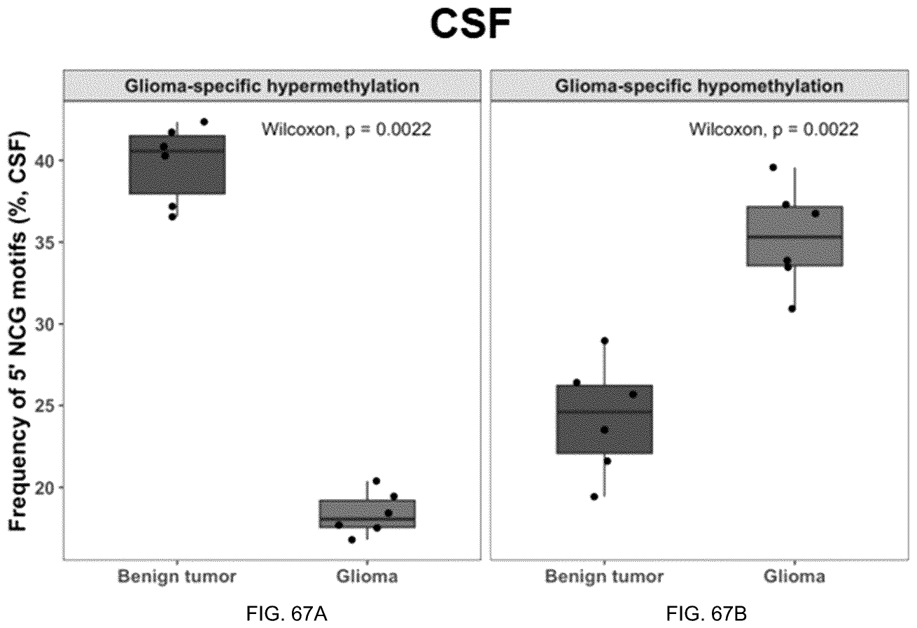

FIG. 67A is a box plot of the frequency of 5′ NCG motifs at glioma specific hypermethylated NCG CpG sites in CSF cfDNA in patients with benign brain tumors or patients with gliomas. It can be appreciated that the frequency of 5′ NCG motif ratios of glioma specific hypermethylated CpG site is significantly lower in CSF from patients having gliomas compared to patients having benign brain tumors (p=0.0022 using Wilcoxon unpaired T-test comparisons).

FIG. 67B is a box plot of the frequency of 5′ NCG motifs at glioma specific hypomethylated NCG CpG sites in CSF cfDNA in patients with benign brain tumors or patients with gliomas. It can be appreciated that the frequency of 5′ NCG motif ratios of glioma specific hypomethylated CpG site is significantly higher in CSF from patients having gliomas compared to patients having benign brain tumors (p=0.0022 using Wilcoxon unpaired T-test comparisons).

The CGN/NCG motif ratio of these sites could differentiate between patients of benign and glioma tumors using cfDNA from CSF, while this effect was not significantly observed in plasma cfDNA. Additionally, the frequency of hypermethylated and hypomethylated 5′ CGN motifs and 5′ NCG motifs differentiated between patients with benign brain tumors and gliomas in CSF. Therefore, FRAGMA based methylation profiling in cfDNA from CSF could profile differential methylation sites in central nervous system related malignancies or pathologies.

### 2. Methylation Density

We next examined whether tumor-specific methylation changes can be detected using methylation density of glioma specific markers. We examined the cleavage profile of cfDNA from CSF based on paired bisulfite sequencing.

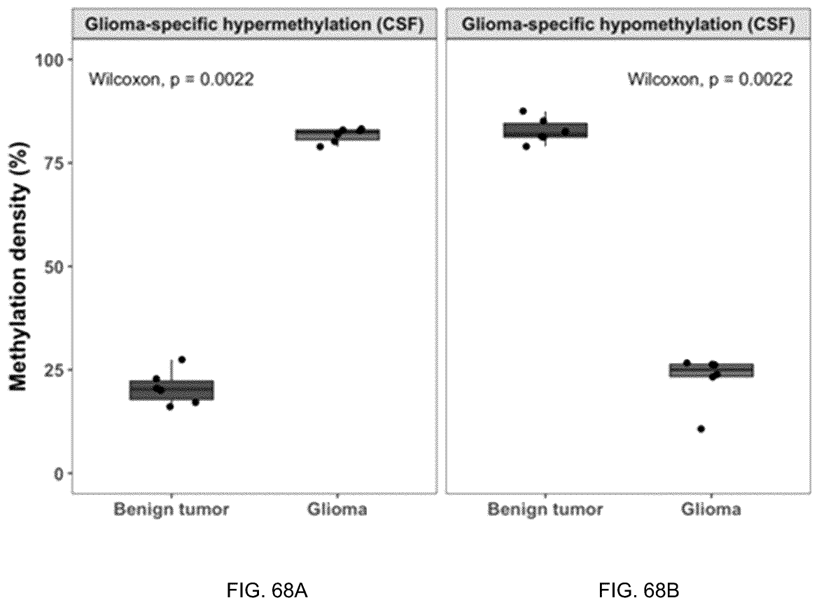

FIG. 68A is a box plot of methylation density for glioma specific hypermethylated CpG sites in CSF cDNA in patients with benign brain tumors or patients with gliomas. Glioma specific hypermethylation is significantly increased in patients with gliomas compared to patients with benign brain tumors (p=0.0022 using Wilcoxon unpaired T-test comparisons).

FIG. 68B is a box plot of methylation density for glioma specific hypomethylated CpG sites in CSF cDNA in patients with benign brain tumors or patients with gliomas. Glioma specific hypomethylation is significantly reduced in patients with gliomas compared to patients with benign brain tumors (p=0.0022 using Wilcoxon unpaired T-test comparisons).

The methylation density of glioma specific hypermethylated and hypomethylated CpG sites could differentiate between patients of benign and glioma tumors using cfDNA from CSF.

### D. Methods

Various methods and corresponding computer readable media and systems can be implemented. For example, one or more end sequence motifs can be used to classify a level of pathology. As another example, one or more methylation levels can be used to classify a level of pathology.

### 1. CG-Motif Related Analysis

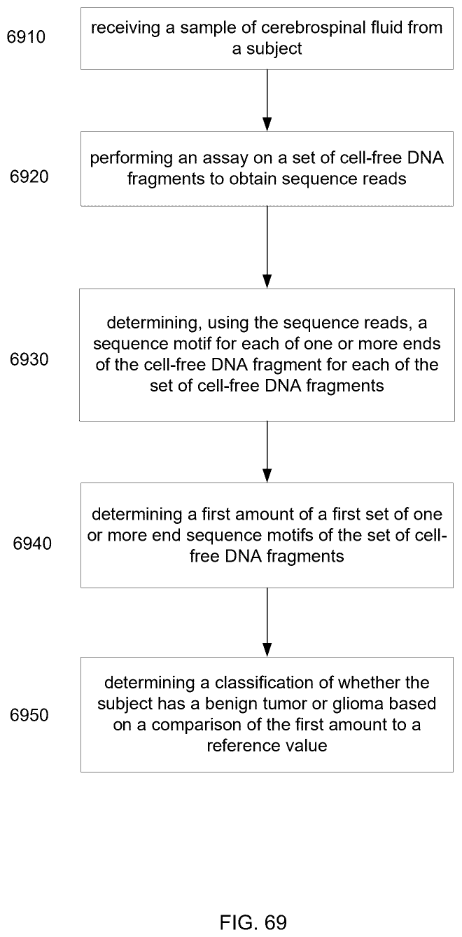

FIG. 69 is a flowchart illustrating a method 6900 for differentiating between brain tumor types in a subject based on methylated CpG sites, according to some embodiments of the present disclosure. Portions or all steps of method 6900 can be performed by a computer system, including one or more processors. The method 6900 can use a trained ML model that was trained by the computer system or another computer system. The computer system can comprise various devices, e.g., one device that performed the training and another that uses the trained model.

At block 6910, method 6900 can include receiving a sample of cerebrospinal fluid from a subject.

At block 6920, method 6900 can include performing an assay on a set of cell-free DNA fragments to obtain sequence reads. An assay can include sequencing or digital PCR.

At block 6930, method 6900 can include determining, using the sequence reads, a sequence motif for each of one or more ends of the cell-free DNA fragment for each of the set of cell-free DNA fragments. An end of a cell-free DNA fragment can have a first position at an outermost position, a second position that is next to the first position, and a third position that is next to the second position. The first set of one or more end sequence motifs can include a plurality of end sequence motifs. The set of cell-free DNA fragments can each be located within one or more regions that can each be hypermethylated or hypomethylated for glioma.

At block 6940, method 6900 can include determining a first amount of a first set of one or more end sequence motifs of the set of cell-free DNA fragments. The first set of one or more end sequence motifs can have C at the first position and G at the second position (e.g., CGN end motifs). Additionally or alternatively, the first set of one or more end sequence motifs can have C at the second position and G at the third position (e.g., NCG end motifs). The first set of one or more end sequence motifs can include all end sequence motifs having C at the first position and G at the second position. The first set of one or more end sequence motifs can include all end sequence motifs having have C at the second position and G at the third position.

A respective amount can be determined for each 3-mer end sequence motif that has a C at the first position and that has G at the second position, thereby determining respective amounts. A respective amount can be determined for each 3-mer end sequence motif that has a C at the second position and that has G at the third position, thereby determining respective amounts. A feature vector can be generated to include the respective amounts. The respective amounts can include the first amount. The feature vector can be inputted into a machine learning model as part of determining the classification. The machine learning model can be trained using the first cohort of reference samples from subjects having the benign tumor and the second cohort of reference samples from subjects having glioma.

A second amount can be determined for a second set of one or more end sequence motifs of the set of cell-free DNA fragments. The second set of one or more end sequence motifs can have C at the first position and G at the second position. The second set of one or more end sequence motifs can have C at the second position and G at the third position.

At block 6950, method 6900 can include determining a classification of whether the subject has a benign tumor or glioma based on a comparison of the first amount to a reference value. The reference value can be trained using a first cohort of reference samples from subjects having a benign tumor and a second cohort of reference samples from subjects having glioma. The classification can use a ratio of the first amount and the second amount, a difference of the first amount and the second amount, or a machine learning model that receives the first amount and the second amount as separate inputs. The classification can be determined using the first amount and the second amount.

Another amount can be determined for another set of one or more end sequence motifs of another set of cell-free DNA fragments. The other set of cell-free DNA fragments can each located within one or more regions that are each hypermethylated for glioma. The classification can also be determined using the other amount of the other set of one or more end sequence motifs. Both amounts can be compared with respective thresholds (e.g., for determining respective classifications, which can be required to match) or for defining a multidimensional point that is compared to a hyperplane (e.g., as part of an SVM model) or threshold surface for differentiating between benign tumors and gliomas.

### 2. Methylation Level Analysis

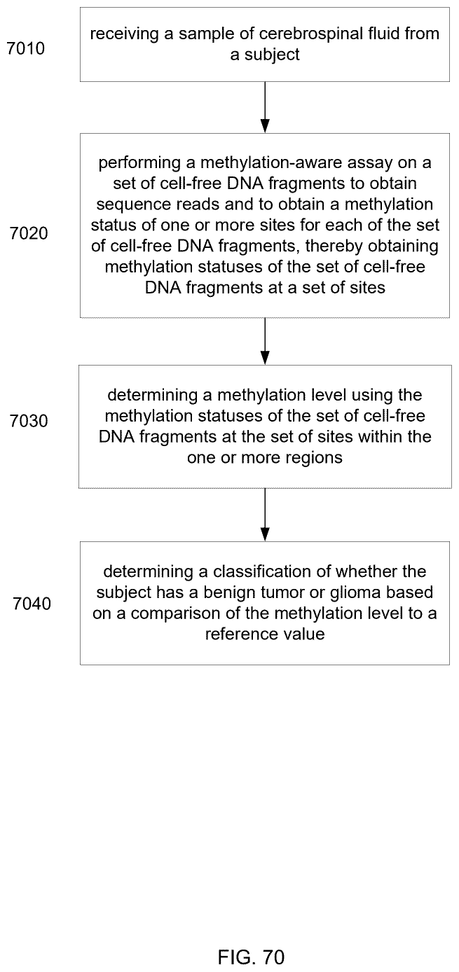

FIG. 70 is a flowchart illustrating a method 7000 for differentiating between brain tumor types in a subject based on methylation level, according to some embodiments of the present disclosure. Portions or all steps of method 7000 can be performed by a computer system, including one or more processors. The method 7000 can use a trained ML model that was trained by the computer system or another computer system. The computer system can comprise various devices, e.g., one device that performed the training and another that uses the trained model.

At block 7010, method 7000 can include receiving a sample of cerebrospinal fluid from a subject.

At block 7020, method 7000 can include performing a methylation-aware assay on a set of cell-free DNA fragments to obtain sequence reads and to obtain a methylation status of one or more sites for each of the set of cell-free DNA fragments, thereby obtaining methylation statuses of the set of cell-free DNA fragments at a set of sites. The set of cell-free DNA fragments can each be located within one or more regions that can each hypermethylated or hypomethylated for glioma. The methylation-aware assay can include methylation-aware sequencing. The methylation-aware sequencing can include bisulfite sequencing, sequencing after treatment using methylation-sensitive restriction enzymes, or single molecule techniques.

At block 7030, method 7000 can include determining a methylation level using the methylation statuses of the set of cell-free DNA fragments at the set of sites within the one or more regions. The one or more regions can each be hypermethylated. The one or more regions can each be hypomethylated. The methylation level can be a methylation density at the set of sites within the one or more regions. The one or more regions can be a plurality of regions.

At block 7040, method 7000 can include determining a classification of whether the subject has a benign tumor or glioma based on a comparison of the methylation level to a reference value. The reference value can be trained using a first cohort of reference samples from subjects having a benign tumor and a second cohort of reference samples from subjects having glioma. The classification can be that the subject has glioma when the methylation level is greater than the reference value. The classification can be that the subject has glioma when the methylation level is less than the reference value.

Another methylation level can be determined using the methylation statuses of another set of cell-free DNA fragments at another set of sites within one or more other regions that are each hypermethylated for glioma. The other methylation level can be used to determine the classification of whether the subject has a benign tumor or glioma. For example, if the first methylation level is determined for hypermethylated sites, then the other methylation level can be determined for hypomethylated sites. The levels for both can be compared with respective thresholds (e.g., for determining respective classifications, which can be required to match) or for defining a multidimensional point that is compared to a hyperplane (e.g., as part of an SVM model) or threshold surface for differentiating between benign tumors and gliomas.

### VI. TISSUE-OF-ORIGIN USING METHYLATION

DNA methylations patterns can also be used to predict an original tissue type or original cancer type from a sample. Briefly, when DNA is treated with bisulfite, unmethylated cytosines are converted into uracils while methylated cytosines remain as cytosines. The bisulfite DNA can then be sequenced. The resulting sequence can be a map of the original DNA methylation pattern. A samples methylation pattern can be compared to a database of known methylation patterns from different tissues and cell types. Based on this comparison, an algorithm can predict the tissue of origin for the sample.

We performed bisulfite sequencing and tissue-of-origin analysis on CSF and plasma cfDNA from patients having a variety of neurological conditions, including high intracranial pressure, benign brain tumors, and high-grade gliomas.

### 1. Multiple Genomic Sites

More genomic sites (e.g., 10 or more) may be used to determine the constitution of the DNA mixture when there are more potential candidate tissues. The accuracy of the estimation of the proportional composition of the DNA mixture is dependent on a number of factors including the number of genomic sites, the specificity of the genomic sites (also called “sites”) to the specific tissues, and the variability of the sites across different candidate tissues and across different individuals used to determine the reference tissue-specific levels. The specificity of a site to a tissue refers to the difference in the methylation density of the genomic sites between the particular tissue and other tissue types.

The larger the difference between their methylation densities, the more specific the site to the particular tissue would be. For example, if a site is completely methylated in the liver (methylation density=100%) and is completely unmethylated in all other tissues (methylation density=0%), this site would be highly specific for the liver. Whereas, the variability of a site across different tissues can be reflected by, for example, but not limited to, the range or standard deviation of methylation densities of the site in different types of tissue. A larger range or higher standard deviation would allow a more precise and accurate determination of the relative contributions of the different organs to the DNA mixture mathematically. The effects of these factors on the accuracy of estimating the proportional contribution of the candidate tissues to the DNA mixture are illustrated in the later sections of this application.

Here, we use mathematical equations to illustrate the deduction of the proportional contribution of different organs to the DNA mixture. The mathematical relationship between the methylation densities of the different sites in the DNA mixture and the methylation densities of the corresponding sites in different tissues can be expressed as:

$${\overline{MD}}_{i} = {\sum }_{k} ( {p}_{k} \times {MD}_{ik} ) ,$$

where M̅D̅ᵢ represents the methylation density of the site i in the DNA mixture; pₖ represents the proportional contribution of tissue k to the DNA mixture; MDᵢₖ represents the methylation density of the site i in the tissue k. When the number of sites is the same or larger than the number of organs, the values of individual pₖ can be determined. The tissue-specific methylation densities can be obtained from other individuals, and the sites can be chosen to have minimal inter-individual variation, as mentioned above.

Additional criteria can be included in the algorithm to improve the accuracy. For example, the aggregated contribution of all tissues can be constrained to be 100%, i.e.

$${\sum }_{k} {p}_{k} = 100 \% .$$

Furthermore, all the organs' contributions can be required to be non-negative:

$${p}_{k} \geq 0 , ∀ k$$

Due to biological variations, the observed overall methylation pattern may not be completely identical to the methylation pattern deduced from the methylation of the tissues. In such a circumstance, mathematical analysis would be required to determine the most likely proportional contribution of the individual tissues. In this regard, the difference between the observed methylation pattern in the DNA and the deduced methylation pattern from the tissues is denoted by W.

$$W = 0 - {\sum }_{k} ( {p}_{k} \times {M}_{k} )$$

where O is the observed methylation pattern for the DNA mixture and Mₖ is the methylation pattern of the individual tissue k. pₖ is the proportional contribution of tissue k to the DNA mixture. The most likely value of each pₖ can be determined by minimizing W, which is the difference between the observed and deduced methylation patterns. This equation can be resolved using mathematical algorithms, for example by using quadratic programming, linear/non-linear regression, expectation-maximization (EM) algorithm, maximum likelihood algorithm, maximum a posteriori estimation, and the least squares method.

### 2. Method of Methylation Deconvolution

As described above, a biological sample including a mixture of cell-free DNA molecules from an organism can be analyzed to determine the composition of the mixture, specifically the contributions from different tissue types. For example, the percentage contribution of the cell-free DNA molecules from the liver can be determined. These measurements of the percentage contributions in the biological sample can be used to make other measurements of the biological sample, e.g., identifications of where a tumor is located, as is described in later sections.

A method for analyzing a DNA mixture of cell-free DNA molecules to determine fractional contributions from various tissue types from methylation levels according to embodiments of the present invention is described herein. The method may be performed using a computer system. A biological sample can include a mixture of cell-free DNA molecules from M tissues types and can be any one of various examples, e.g., as mentioned herein. The number M of tissue types is greater than two. In various embodiments, M can be 3, 7, 10, 20, or more, or any number in between.

In some embodiments, markers are broadly divided into two types (type I and type II). Type I markers have tissue specificity. The methylation level of these markers for a particular group of one or more tissues is different from most of the other tissues. For example, a particular tissue can have a significant methylation level compared with the methylation level of all the other tissues. In another example, two tissues (e.g., tissue A and tissue B) have similar methylation levels, but the methylation levels of tissues A and B are significantly different from those of the remaining tissues.

Type II markers have a high inter-tissue methylation variability. The methylation levels of these markers are highly variable across different tissues. A single marker in this category may not be sufficient to determine the contribution of a particular tissue to the DNA mixture. However, a combination of type II markers, or in combination with one or more type I markers can be used collectively to deduce the contribution of individual tissues. Under the above definition, a particular marker can be a type I marker only, a type II marker only, or be simultaneously both a type I and type II marker.

N genomic sites can be identified for analysis. The N genomic sites can have various attributes, e.g., type I and type II genomic sites. As examples, the N genomic sites can include type I or type II sites only, or a combination of both. The genomic sites can be identified based on analyses of one or more other samples, e.g., based on data obtained from databases about methylation levels measured in various individuals.

In some embodiments, at least 10 of the N genomic sites are type II and each have a coefficient of variation of methylation levels of at least 0.15 across the M tissue types. A more stringent threshold for the coefficient of variation can be used, e.g., 0.25. The at least 10 genomic sites can also each have a difference between a maximum and a minimum methylation level for the M tissue types that exceeds 0.1. A more stringent threshold for the coefficient of variation can be used, e.g., 0.2. The N genomic sites can also include type I sites (e.g., at least 10).

These methylation properties of the genomic loci can be measured for one sample or a set of samples. The set of samples may be for a subpopulation of organisms that includes the instant organism being tested, e.g., a subpopulation having a particular trait that is shared with the instant organism. These other samples can be referred to as reference tissues, and different reference tissues may be used from different samples.

N tissue-specific methylation levels can be obtained at the N genomic sites for each of M tissue types. N is greater than or equal to M, so that the tissue-specific methylation levels can be used in the deconvolution to determine the fractional percentages. The tissue-specific methylation levels can form a matrix A of dimensions N by M. Each column of the matrix A can correspond to a methylation pattern for a particular tissue type, where the pattern is of methylation levels at the N genomic sites.

In various embodiments, the tissue-specific methylation patterns can be retrieved from public database(s) or previous studies. In examples herein, the methylation data for neutrophils and B cells can be downloaded from the Gene Expression Omnibus (Hodges et al. Mol Cell 2011; 44:17-28). Methylation patterns for other tissues (hippocampus, liver, lung, pancreas, atrium, colon (including its various parts, e.g. sigmoid colon, transverse colon, ascending colon, descending colon), adrenal gland, esophagus, small intestines and CD4 T cell) can be downloaded from the RoadMap Epigenomics project (Ziller et al. Nature 2013; 500:477-81). The methylation patterns for the buffy coat, placenta, tumor and plasma data were from published reports (Lun et al. Clin Chem. 2013; 59:1583-94; Chan et al. Proc Natl Acad Sci USA. 2013; 110:18761-8). These tissue-specific methylation patterns can be used to identify the N genomic sites to be used in the deconvolution analysis.

A biological sample including a mixture of cell-free DNA molecules from the M tissues types can be received. The biological sample may be obtained from the patient organism in a variety of ways. The manner of obtaining such samples may be non-invasive or invasive. Examples of non-invasively obtained samples include certain types of fluids (e.g. plasma or serum or urine) or stools. For instance, plasma includes cell-free DNA molecules from many organ tissues, and is thus useful for analyzing many organs via one sample.

Cell-free DNA molecules from the biological sample can then be analyzed to identify their locations in a reference genome corresponding to the organism. For example, the cell-free DNA molecules can be sequenced to obtain sequence reads, and the sequence reads can be mapped (aligned) to the reference genome. If the organism was a human, then the reference genome would be a reference human genome, potentially from a particular subpopulation. As another example, the cell-free DNA molecules can be analyzed with different probes (e.g., following PCR or other amplification), where each probe corresponds to a different genomic site. In some embodiments, the analysis of the cell-free DNA molecules can be performed by receiving sequence reads or other experimental data corresponding to the cell-free DNA molecules, and then analyzing the experimental data.

A statistically significant number of cell-free DNA molecules can be analyzed so as to provide an accurate deconvolution for determining the fractional contributions from the M tissue types. In some embodiments, at least 1,000 cell-free DNA molecules are analyzed. In other embodiments, at least 10,000 or 50,000 or 100,000 or 500,000 or 1,000,000 or 5,000,000 cell-free DNA molecules or more can be analyzed. The total number of molecules to analyze can depend on M and N, and the desired precision (accuracy).

N mixture methylation levels can be measured at the N genomic sites using a first group of cell-free DNA molecules that are each located at any one of N genomic sites of the reference genome. The N mixture methylation levels refer to methylation levels in the mixture of the biological sample. As an example, if a cell-free DNA molecule from the mixture is located at one of the N genomic sites, then a methylation index for that molecule at the site can be included in an overall methylation density for that site. The N mixture methylation levels can form a methylation vector b of length N, where b corresponds to observed values from which the fractional contributions of the tissue types can be determined.

In one embodiment, the methylation levels for the genomic sites in the DNA mixture can be determined using whole genome bisulfite sequencing. In other embodiments, the methylation levels for the genomic sites can be determined using methylation microarray analysis, such as the Illumina HumanMethylation450 system, or by using methylation immunoprecipitation (e.g. using an anti-methylcytosine antibody) or treatment with a methylation-binding protein followed by microarray analysis or DNA sequencing, or by using methylation-sensitive restriction enzyme treatment followed by microarray or DNA sequencing, or by using methylation aware sequencing e.g. using a single molecule sequencing method (e.g. by a nanopore sequencing (Schreiber et al. Proc Natl Acad Sci 2013; 110: 18910-18915) or by the Pacific Biosciences single molecule real time analysis (Flusberg et al. Nat Methods 2010; 7: 461-465)). Tissue-specific methylation levels can be measured in a same way. As other example, targeted bisulfite sequencing, methylation-specific PCR, non-bisulfite based methylation-aware sequencing (e.g. by single molecule sequencing platforms (Powers et al. Efficient and accurate whole genome assembly and methylome profiling of E. coli. BMC Genomics. 2013; 14:675) can be used for the analysis of the methylation level of the plasma DNA for plasma DNA methylation deconvolution analysis. Accordingly, methylation-aware sequencing results can be obtained in a variety of ways.

M values of a composition vector can be determined. Each M value corresponds to a fractional contribution of a particular tissue type of the M tissue types to the DNA mixture. The M values of the composition vector can be solved to provide the N mixture methylation levels (e.g., methylation vector b) given the N×M tissue-specific methylation levels. The M fractional contributions can correspond to a vector x that is determined by solving Ax=b. When N is greater than M, the solution can involve a minimization of errors, e.g., using least-squares.

The composition vector can be used determine an amount of each of the M tissue types in the mixture. The M values of the composition vector may be taken directly as the fractional contributions of the M tissue types. In some implementations, the M values can be converted to percentages. Error terms can be used to shift the M values to higher or lower values. Each of the values of the composition vector can be considered a component, and a first component can correspond to a first tissue type.

### 3. Applications

Fractional contributions can be used in further measurements of the biological sample and other determinations, e.g., whether a particular chromosomal region has a sequence imbalance or whether a particular tissue type is diseased.

A biological sample can be subjected to genome-wide bisulfite sequencing. Plasma DNA tissue mapping uses tissue-specific methylation profiles to determine tissue contribution percentages. Example tissue-specific methylation profiles are shown as liver, blood cells, adipose tissues, lungs, small intestines, and colon. The contribution percentages can be determined as described above and elsewhere, e.g., solving Ax=b. Examples of applications include prenatal testing, cancer detection and monitoring, organ transplant monitoring, and organ damage assessment.

A list of methylation markers (genomic sites) that are useful for determining the contributions of different organs to the plasma DNA can be identified by comparing the methylation profiles of different tissues, including the liver, lungs, esophagus, heart, pancreas, sigmoid colon, small intestines, adipose tissues, adrenal glands, colon, T cells, B cells, neutrophils, brain and placenta. In various examples, whole genome bisulfite sequencing data for the liver, lungs, esophagus, heart, pancreas, colon, small intestines, adipose tissues, adrenal glands, brain and T cells were retrieved from the Human Epigenome Atlas from the Baylor College of Medicine (www.genboree.org/epigenomeatlasfindex.rhtml). The bisulfite sequencing data for B cells and neutrophils were from the publication by Hodges et al. (Hodges et al; Directional DNA methylation changes and complex intermediate states accompany lineage specificity in the adult hematopoietic compartment. Mol Cell 2011; 44: 17-28). The bisulfite sequencing data for the placenta were from Lun et al (Lun et al. Clin Chem 2013; 59:1583-94). In other embodiments, markers can be identified from datasets generated using microarray analyses, e.g. using the Illumina Infinium HumanMethylation450 BeadChip Array

### B. Differences Between CSF and Plasma

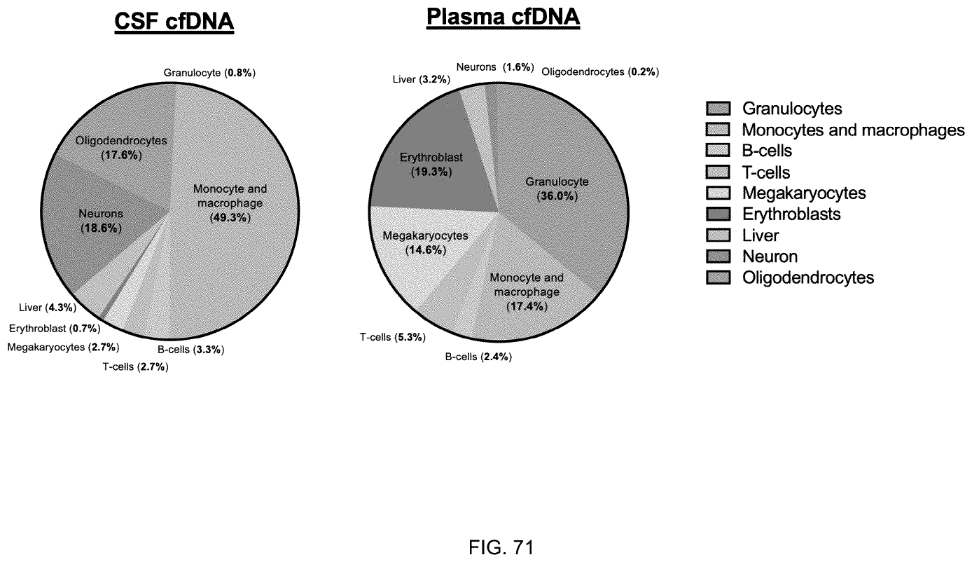

FIG. 71 shows results of tissue-of-origin deconvolution performed using methylation status (bisulfite sequencing of cfDNA samples). The analysis was performed on a representative individual with high intra-cranial pressure. A difference in tissue composition between CSF and plasma cfDNA was observed. In particular, CSF cfDNA showed increased proportions of cell types from the brain (e.g. neurons and oligodendrocytes).

To evaluate the potential tissue origins of cfDNA from the CSF and plasma, we have performed tissue mapping by genome-wide methylation sequencing (bisulfite sequencing) to reference tissue methylomes (Sun et al. 2015). The proportional tissue contributions of cfDNA from CSF and plasma from one individual (TBR5841) is summarized in FIG. 71. CSF cfDNA showed increased tissue contributions from brain-derived sources such as neurons and oligodendrocytes, compared to plasma cfDNA, which supports the application of CSF cfDNA for CNS related pathologies. Increased contributions of monocytes and macrophages was consistent to previous literature that CSF mostly consists of lymphocytes and monocytes, with very small fractions of granulocytes such as neutrophils (Berek et al. 2021). One interesting observation was the contribution of liver in the CSF, which may suggest potential passage of DNA molecules from the blood circulation to CSF.

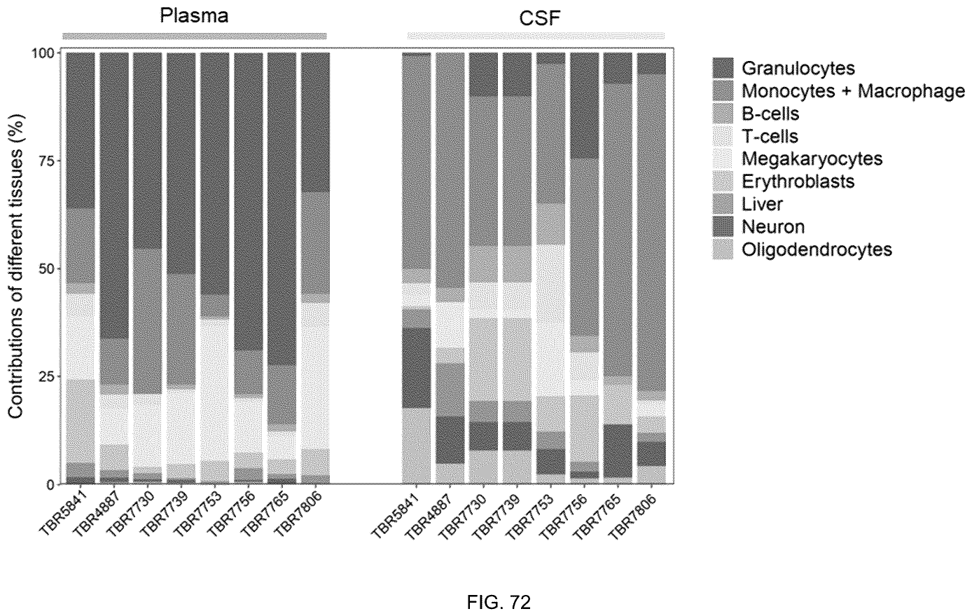

FIG. 72 is a bar chart representing methylation-based cell-type deconvolution analysis of cfDNA from paired plasma and CSF cfDNA samples from cases of benign tumors. Using reference methylation atlases from Loyfer et al. (Loyfer et al. 2023), the contribution of cfDNA in CSF varies largely from plasma, including higher contributions from monocytes, macrophages, neurons and oligodendrocytes, with decreased proportion of granulocytes. Differences in the production, clearance, and circulation of cfDNA in CSF highlights the need for novel fragmentomic markers in tumor staging and sub-typing.

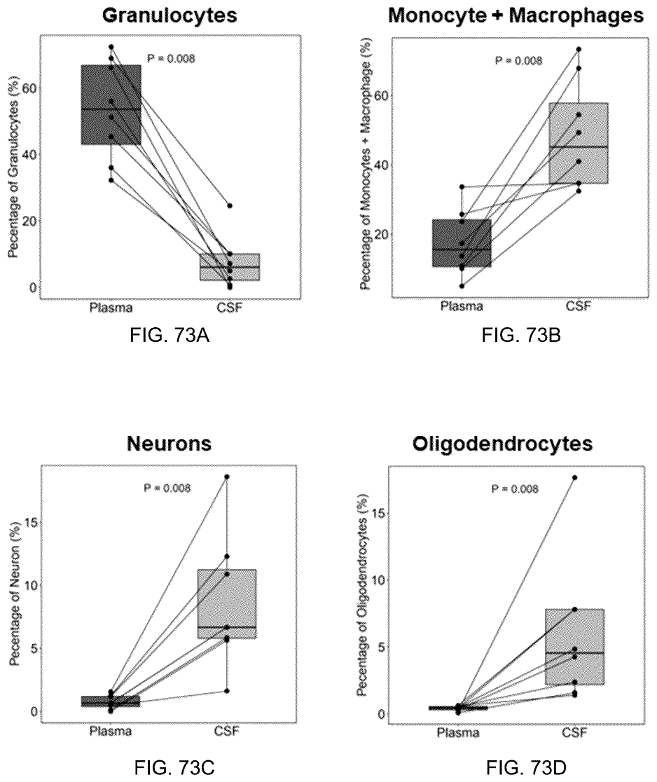

FIG. 73A-73D are box plots showing the percent contribution of specific cell types to the tissue-of-origin analysis from methylation-based cell-type deconvolution analysis of cfDNA from paired plasma and CSF cfDNA. The samples are from eight cases of patients with benign brain tumors. The contribution of granulocytes was significantly reduced (p=0.008) in CSF cfDNA as compared to the contribution in plasma (FIG. 73A). The contribution of monocytes and macrophages (FIG. 73B), neurons (FIG. 73C), and oligodendrocytes (FIG. 73D) were significantly reduced (p=0.008 for each cell type) in CSF cfDNA as compared to the contribution in plasma. Statistical significance was assessed using Wilcoxon matched-pairs signed rank test.

These data indicate that CSF contains larger contributions of neurons, oligodendrocytes, monocytes and macrophages, and decreased contribution of granulocyte, compared to plasma cell-free DNA. Differences in the production, clearance, and circulation of cfDNA in CSF highlights the need for novel fragmentomic markers in tumor staging and sub-typing. Furthermore, support the use of cfDNA in CSF to reflect pathologies related to neurons and other glial cells, including cancer and neurodegeneration.

### C. Differentiation Between Benign and Glioma

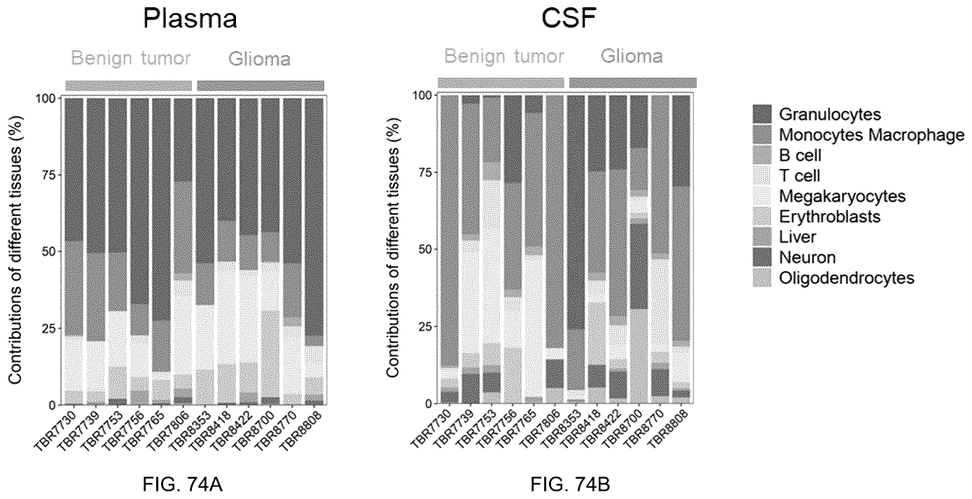

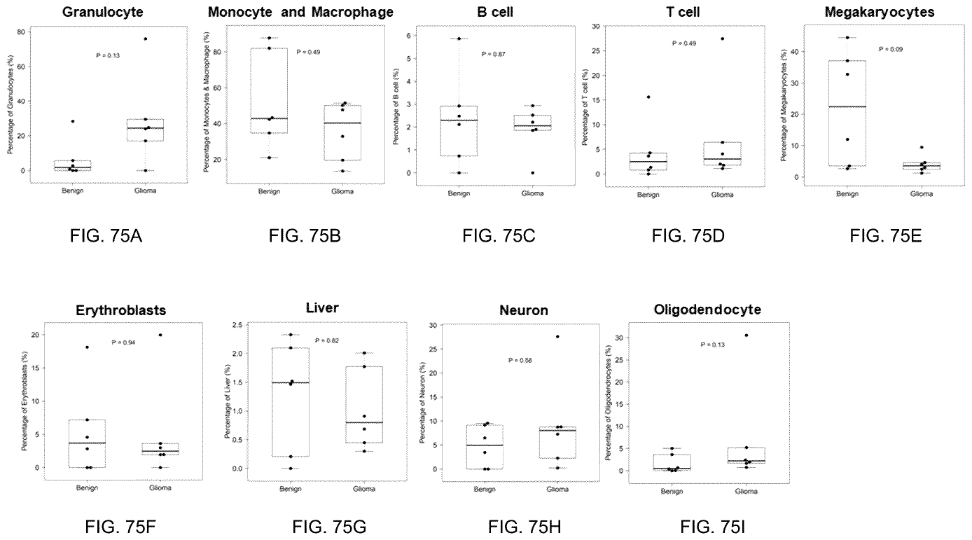

FIG. 74A is a bar chart representing methylation-based cell-type deconvolution analysis of plasma cfDNA samples from patients having benign brain tumors and patients having high-grade gliomas. The data are summarized in FIGS. 75A-75I.

FIG. 74B is a bar chart representing methylation-based cell-type deconvolution analysis of CSF cfDNA samples from patients having benign brain tumors and patients having high-grade gliomas. The data are summarized in FIGS. 75A-75I.

FIG. 75A-75I are box plots showing the percent contribution of specific cell types to the tissue-of-origin analysis from methylation-based cell-type deconvolution analysis of cfDNA from CSF cfDNA. The samples are from six patients with benign brain tumors and six patients with high-grade gliomas. Cell types included granulocytes (FIG. 75A), monocytes and macrophages (FIG. 75B), B cells (FIG. 75C), T cells (FIG. 75D), megakaryocytes (FIG. 75E), erythroblasts (FIG. 75F), liver (FIG. 75G), neurons (FIG. 75H), and oligodendrocytes (FIG. 75I). Interestingly, there was no difference in the contribution of any cell type to the tissue-of-origin analysis between patients with benign brain tumors and patients with high-grade gliomas.

Tissue-of-origin analysis revealed differences between benign and glioma cases (e.g., increased granulocyte contribution in CSF with glioma). Using additional tissue-types in the deconvolution can increase accuracy. For example, no available astrocyte and glial cell markers were used for deconvolution, each of which may show a difference in proportion.

### D. Method

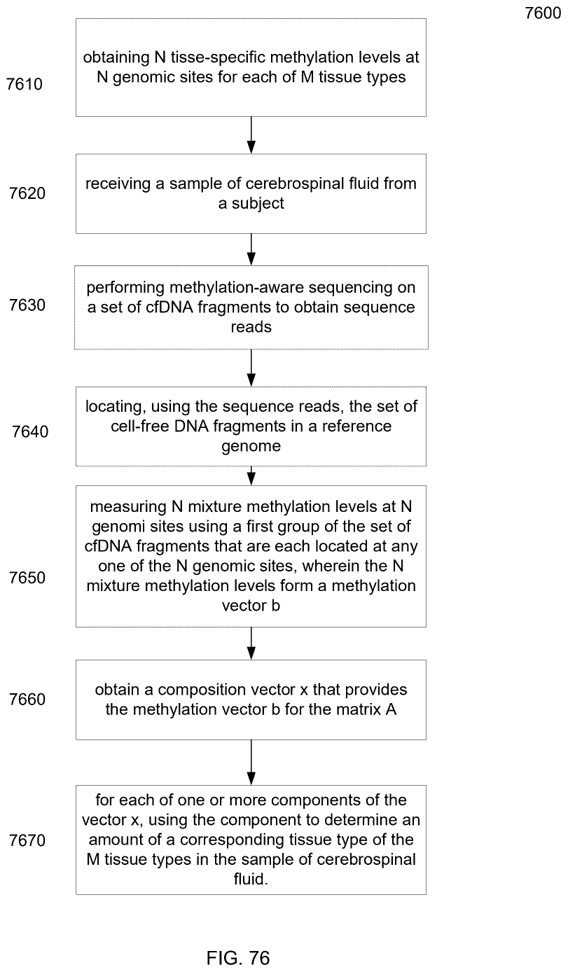

FIG. 76 is a flowchart illustrating a method 7600 for analyzing a sample of cerebrospinal fluid from a subject, according to some embodiments of the present disclosure. The sample can include a mixture of cell-fee DNA from M tissue types, M being greater than two. Portions or all steps of method 7600 can be performed by a computer system, including one or more processors. The method 7600 can use a trained ML model that was trained by the computer system or another computer system. The computer system can comprise various devices, e.g., one device that performed the training and another that uses the trained model.

At block 7610, the method 7600 can include for each of the M tissue types, obtaining N tissue-specific methylation levels at N genomic sites. N can be greater than or equal to M and the tissue-specific methylation levels can form a matrix A of dimensions N by M. In some instances, N may be greater than M. The N tissue-specific methylation levels at the N genomic sites may be obtained from a database.

The method 7600 can further include identifying the N genomic sites. For one or more other samples, a first set of the N genomic sites can each have a coefficient of variation of methylation levels of at least 0.15 across the M tissue types and each have a difference between a maximum and a minimum methylation level for the M tissue types that exceeds 0.1. The first set may include at least 10 genomic sites. The at least 10 of the N genomic sites may each have a coefficient of variation of methylation levels of at least 0.25 across the M tissue types and each have the difference between the maximum and the minimum methylation level for the M tissue types that exceeds 0.2.

A second set of the N genomic sites can each have a methylation level in one tissue type that is different from methylation levels in other tissue types by at least a threshold level. The second set of the N genomic sites may include at least 10 genomic sites. The threshold level may correspond to a difference of the methylation level in the one tissue type from a mean of the methylation levels in the other tissue types by at least a specified number of standard deviations.

At block 7620, the method 7600 can include receiving a sample of cerebrospinal fluid from the subject. The cerebrospinal fluid may be extracted from the subject using lumbar puncture or external ventricular drain as illustrated in FIGS. 3A-3B.

At block 7630, the method 7600 can include performing methylation-aware sequencing on a set of cell-free DNA fragments to obtain sequence reads.

At block 7640, the method 7600 can include locating, using the sequence reads, the set of cell-free DNA fragments in a reference genome. Locating the set of cell-free DNA fragments in the reference genome can include aligning the sequence reads to the reference genome. The N mixture methylation levels may be measured using sequence reads that each aligns to at least one of the N genomic sites of the reference genome.

At block 7650, the method 7600 can include measuring N mixture methylation levels at the N genomic sites using a first group of the set of cell-free DNA fragments that are located at any one of the N genomic sites. The N mixture methylation levels can form a methylation vector b.

At block 7660, the method 7600 can include obtaining a composition vector x that provides the methylation vector b for the matrix A. Obtaining the composition vector x can include solving Ax=b. Solving Ax=b can involve a least squares optimization.

At block 7670, the method 7600 can include for each of one or more components of the composition vector x, using the component to determine an amount of a corresponding tissue type of the M tissue types in the sample of cerebrospinal fluid. A first component of the one or more components may correspond to a brain tissue type. The method 7600 can further include comparing a first amount of the brain tissue type in the mixture to a threshold amount to determine a classification of whether the subject has a brain cancer. The threshold amount can be determined based on amounts of the brain tissue type in mixtures of a first set of organisms that are healthy for the brain tissue type or have high intracranial pressure and of a second set of organisms that have the brain cancer.

### VII. COPY NUMBER ABERRATIONS FOR BRAIN CANCER

Copy number aberrations (CNA) are changes in the number of copies of a particular DNA segment within the genome. CNAs can refer to a large scale genomic change that results in sections of DNA being duplicated (e.g., amplified), or deleted. The changes can range from a few kilobases to entire arms of chromosomes. In cancer, duplications or deletions can drive cancer growth and progression. Analyses of CNAs can provide diagnostic and prognostic information about genetic disorders, including cancers.

### A. Differences Between Plasma and CSF

We first characterized CNA in plasma cfDNA from patients having benign brain tumors.

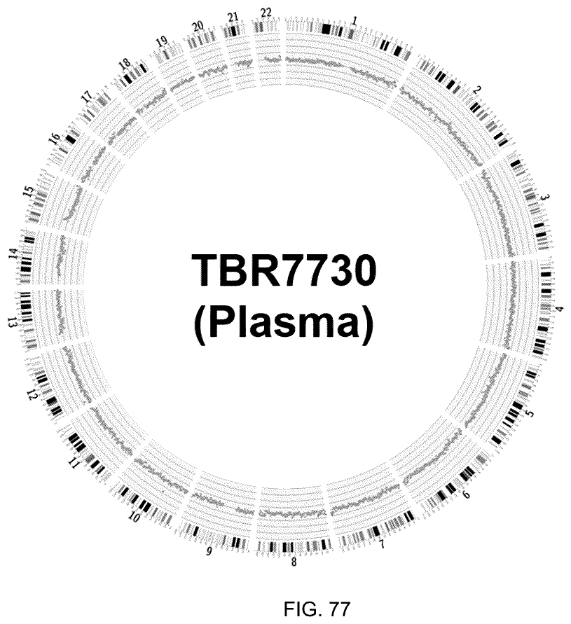

FIG. 77 is a visual representation of copy number alterations in plasma cfDNA from a patient having benign brain tumor. Analyses were performed on six patients with benign brain tumors. A representative visualization of CNA analysis is shown. IchorDNA can be used to detect copy number aberrations and a CNA measurement can be derived from whole genome sequencing. A reference copy number can be used to define deletions or copy gains and amplification events. None of the cases, including the case shown, exhibit significant copy number aberrations in plasma cfDNA.

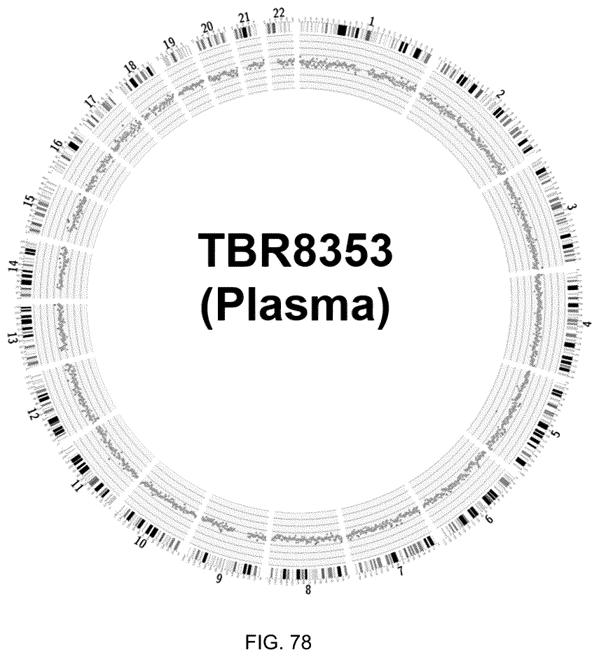

FIG. 78 is a visual representation of copy number alterations in plasma cfDNA from a patient having high-grade glioma. Analyses were performed on six patients with high-grade gliomas. A representative visualization of CNA analysis is shown. Similar to the CNA analysis in plasma cfDNA from patients having benign brain tumors, none of the high-grade glioma cases shown exhibit significant copy number aberrations in plasma cfDNA. Brain tumor fraction using CNA

### 1. Determination of Tumor Fraction

Chromosomal aberrations, for example, amplifications and deletions are frequently observed in cancer genomes. The chromosomal aberrations observed in cancer tissues typically involve subchromosomal regions and these aberrations can be shorter than 1 Mb. And, the cancer-associated chromosomal aberrations are heterogeneous in different patients, and thus different regions may be affected in different patients. It is also not uncommon for tens, hundreds or even thousands of copy number aberrations to be found in a cancer genome. All of these factors make determining tumor DNA concentration difficult.

Embodiments involve the analysis of quantitative changes resulted from tumor-associated chromosomal aberrations. In one embodiment, the DNA samples containing DNA derived from cancer cells and normal cells are sequenced using massively parallel sequencing, for example, by the Illumina HiSeq2000 sequencing platform. The derived DNA may be cell-free DNA in plasma or other suitable biological sample.

Chromosomal regions that are amplified in the tumor tissues would have increased probability of being sequenced and regions that are deleted in the tumor tissues would have reduced probability of being sequenced. As a result, the density of sequence reads aligning to the amplified regions would be increased and that aligning to the deleted regions would be reduced. The degree of variation is proportional to the fractional concentration of the tumor-derived DNA in the DNA mixture. The higher the proportion of DNA from the tumor tissue, the larger the change would be caused by the chromosomal aberrations.

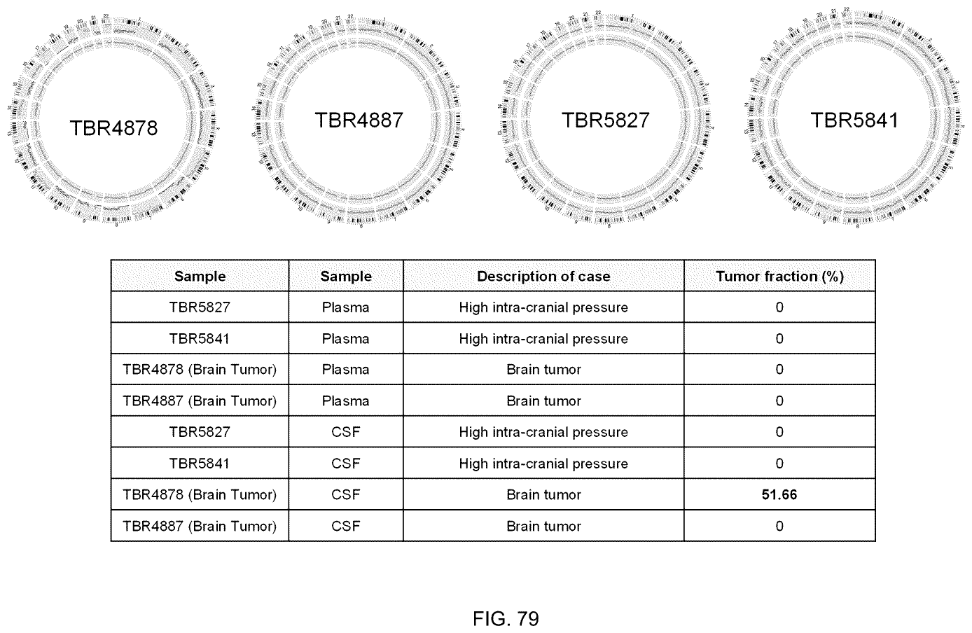

FIG. 79 shows tumor fraction of paired plasma and CSF determined by copy number aberration (CNA) in patients having high intracranial pressure or benign brain tumor. The percent tumor fraction was 0% for all plasma cfDNA samples as well as CSF cfDNA samples from patients having high intracranial pressure. One benign brain tumor sample from CSF cfDNA had a tumor fraction of 51.66%. Interestingly, the other benign brain tumor sample from CSF had a tumor fraction of 0%. We believe this is due to the tumor fraction falling below the detection limit of 5%.

a) Estimation in Sample with High Tumor Concentration

DNA can be extracted from subjects and fragmented using the Covaria DNA sonication system and sequenced using the Illumina HiSeq2000 platform as described (Chan K C et al. Clin Chem 2013; 59:211-24). The sequence reads can be aligned to the human reference genome (hg18). The genome can then be divided into 1 Mb bins (regions) and the sequence read density can be calculated for each bin after adjustment for GC-bias as described (Chen E Z et al. PLoS One. 2011; 6:e21791).

After sequence reads are aligned to a reference genome, a sequence read density can be computed for various regions. In one embodiment, the sequence read density is a proportion determined as the number of reads mapped to a particular bin (e.g., 1 Mb region) divided by the total sequence reads that can be aligned to the reference genome (e.g., to a unique position in the reference genome). Bins that overlap with chromosomal regions amplified in the tumor tissue are expected to have higher sequence read densities than those from bins without such overlaps. On the other hand, bins that overlap with chromosomal regions that are deleted are expected to have lower sequence read densities than those without such overlaps. The magnitude of the difference in sequence read densities between regions with and without chromosomal aberrations is mainly affected by the proportion of tumor-derived DNA in the sample and the degree of amplification/deletion in the tumor cells.

Various statistical models may be used to identify the bins having sequence read densities corresponding to different types of chromosomal aberrations. In one embodiment, a normal mixture model (McLachlan G and Peel D. Multvariate normal mixtures. In Finite mixture models 2004: p 81-116. John Wiley & Sons Press) can be used. Other statistical models, for example the binomial mixture model and Poisson regression model (McLachlan G and Peel D. Mixtures with non-normal components, Finite mixture models 2004: p 135-174. John Wiley & Sons Press), can also be used.

The sequence read density for a bin can be normalized using the sequence read density of the same bin as determined from the sequencing of the buffy coat DNA. The sequence read densities of different bins may be affected by the sequence context of a particular chromosomal region, and thus the normalization can help to more accurately identify regions showing aberration. For example, the mappability (which refers to the probability of aligning a sequence back to its original position) of different chromosomal regions can be different. In addition, the polymorphism of copy number (i.e. copy number variations) would also affect the sequence read densities of the bins. Therefore, normalization with the buffy coat DNA can potentially minimize the variations associated with the difference in the sequence context between different chromosomal regions.

Peaks can be fitted to a distribution curve of sequence read densities to represent the regions with deletion, amplification, and without chromosomal aberrations using the normal mixture model. In one embodiment, the number of peaks can be determined by the Akaike's information criterion (AIC) across different plausible values. A central peak with a log₂R=0 (i.e. R=1) can represent the regions without any chromosomal aberration. The left peak (relative to the central one) represents regions with one copy loss. The right peak (relative to central one) represents regions with one copy amplification.

The fractional concentration of tumor-derived DNA can be reflected by the distance between the peaks representing the amplified and deleted regions. The larger the distance, the higher the fractional concentration of the tumor-derived DNA in the sample would be. The fractional concentration of tumor-derived DNA in the sample can be determined by this genomic representation approach, denoted as FOR, using the following equation: F_{GR}=R_{right}−R_{left}, where R_{right} is the R value of the right peak and R_{left} is the R value of the left peak. The largest difference would be 1, corresponding to 100%.

To verify this result, another method using the genomewide aggregated allele loss (GAAL) analysis can be used to independently determine the fractional concentration of proportion of tumoral DNA (Chan K C et al. Clin Chem 2013; 59:211-24).

b) Estimation in Sample with Low Tumor Concentration

The above analysis has shown that our genomic representation method can be used to measure the fractional concentration of tumor DNA when more than 50% of the sample DNA is tumor-derived, i.e. when the tumor DNA is a majority proportion. In the previous analysis, we have shown that this method can also be applied to samples in which the tumor-derived DNA represents a minor proportion (i.e., below 50%). Samples that may contain a minor proportion of tumor DNA include, but not limited to blood, plasma, serum, urine, pleural fluid, cerebrospinal fluid, tears, saliva, ascitic fluid and feces of cancer patients. In some samples, the fractional concentration of tumor-derived DNA can be 49%, 40%, 30%, 20%, 10%, 5%, 2%, 1%, 0.5%, 0.1% or lower.

For such samples, the peaks of sequence read density representing the regions with amplification and deletion may not be as obvious as in samples containing a relatively high concentration of tumor-derived DNA as illustrated above. In one embodiment, the regions with chromosomal aberrations in the cancer cells can be identified by making comparison to reference samples which are known to not contain cancer DNA. For example, the plasma of subjects without a cancer can be used as references to determine the normative range of sequence read densities for the chromosome regions. The sequence read density of the tested subject can be compared with the value of the reference group. In one embodiment, the mean and standard deviation (SD) of sequence read density can be determined. For each bin, the sequence read density of the tested subject is compared with the mean of the reference group to determine the z-score using the following formula:

$$z - score = \frac{( {GR}_{test} - {\overline{GR}}_{ref} )}{{SD}_{ref}} ,$$

where GRₜₑₛₜ represents the sequence read density of the cancer patient; G̅R̅_{ref} represents the mean sequence read density of the reference subjects and SD_{ref} represents the SD of the sequence read densities for the reference subjects.

Regions with z-score <−3 signifies significant underpresentation of the sequence read density for a particular bin in the cancer patient suggesting the presence of a deletion in the tumor tissue. Regions with z-score >3 signifies significant overpresentation of the sequence read density for a particular bin in the cancer patient suggesting the presence of an amplification in the tumor tissue

Then, the distribution of the z-scores of all the bins can be constructed to identify regions with different numbers of copy gain and loss, for example, deletion of 1 or 2 copies of a chromosome; and amplification, resulting in of 1, 2, 3 and 4 additional copies of a chromosome. In some cases, more than one chromosome or more than one regions of a chromosome may be involved.

A distribution plot of z-scores for all the bins in the plasma of a subject can be created. The peaks (from left to right) representing 1-copy loss, no copy change, 1-copy gain and 2-copy gain are fitted to the z-score distribution. Regions with different types of chromosomal aberrations can then be identified, for example using the normal mixture model as described above.

The fractional concentration of the cancer DNA in the sample (F) can then be inferred from the sequence read densities of the bins that exhibit one-copy gain or one-copy loss. The fractional concentration determined for a particular bin can be calculated as

$$F = \frac{\vert {GR}_{test} - {\overline{GR}}_{ref} \vert \times 2}{{GR}_{ref}} \times 100 \% .$$

This can also be expressed as:

$$F = \frac{\vert z - score \times {SD}_{ref} \vert}{{\overline{GR}}_{ref}} \times 2 ,$$

which can be rewritten as: F=|z-score|×CV×2, where CV is the coefficient of variation for the measurement of the sequence read density of the reference subjects; and

$$CV = \frac{{SD}_{ref}}{{\overline{GR}}_{ref}} .$$

In one embodiment, the results from the bins are combined. For example, the z-scores of bins showing a 1-copy gain can be averaged or the resulting F values averaged. In another implementation, the value of the z-score used for inferring F is determined by a statistical model.

In another embodiment, all bins with z-score <−3 and z-score >3 can be attributed to regions with single copy loss and single copy gain, respectively, because these two types of chromosomal aberrations are the most common. This approximation is most useful when the number of bins with chromosomal aberrations is relatively small and fitting of normal distribution may not be accurate.

### 2. Method

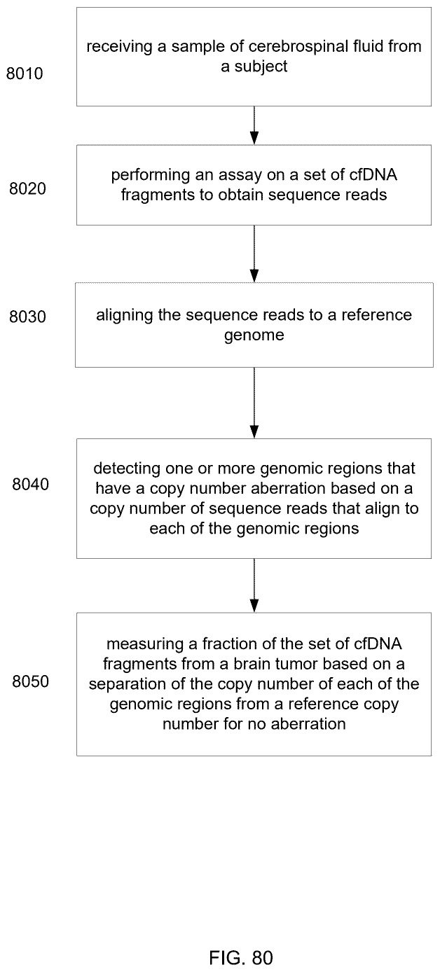

FIG. 80 is a flowchart illustrating a method 8000 for measuring a fraction of cell-free DNA fragments from a brain tumor, according to some embodiments of the present disclosure. The sample can include a mixture of cell-fee DNA from M tissue types, M being greater than two. Portions or all steps of method 8000 can be performed by a computer system, including one or more processors. The method 8000 can use a trained ML model that was trained by the computer system or another computer system. The computer system can comprise various devices, e.g., one device that performed the training and another that uses the trained model.

At block 8010, method 8000 can include receiving a sample of cerebrospinal fluid from a subject. The cerebrospinal fluid may be extracted from the subject using lumbar puncture or external ventricular drain as illustrated in FIGS. 3A-3B.

At block 8020, method 8000 can include performing an assay on a set of cell-free DNA fragments to obtain sequence reads. The assay can include sequencing or digital PCR.

At block 8030, method 8000 can include aligning the sequence reads to a reference genome. Any alignment tool may be used, e.g., as described herein.

At block 8040, method 8000 can include detecting one or more genomic regions that have a copy number aberration based on a copy number of sequence reads that align to each of the one or more genomic regions. The plurality of genomic regions may have a same length or may be non-overlapping or can be overlapping.

Detecting the one or more genomic regions having the copy number aberration can include for each of a plurality of genomic regions, determining a respective amount of DNA fragments within the genomic region from sequence tags having a genomic position within the genomic region, normalizing the respective amount to obtain a respective density, comparing the respective density to a reference density to identify whether the genomic region exhibits a 1-copy loss or a 1-copy gain, and calculating a first density from one or more respective densities identified as exhibiting a 1-copy loss or from one or more respective densities identified as exhibiting a 1-copy gain. All genomic regions determined to exhibit a statistically significant gain in the respective density relative to the reference density can be identified as exhibiting a 1-copy gain.

Normalizing the respective amount to obtain a respective density can include using a same total number of aligned reference tags to determine the respective density and the reference density. Normalizing the respective amount to obtain a respective density can also include dividing the respective amount by a total number of aligned reference tags.

Comparing the respective density to the reference density to identify whether the genomic region exhibits a 1-copy loss or a 1-copy gain can include computing a difference between the respective density and the reference density and comparing the difference to a cutoff value. Comparing the respective density to the reference density to identify whether the genomic region exhibits a 1-copy loss or a 1-copy gain can further include fitting peaks to a distribution curve of a histogram of the respective densities, wherein the first density corresponds to a first peak and the second density corresponds to a second peak.

The first density can be calculated using respective densities identified as exhibiting a 1-copy gain. The other density may be a second density calculated from respective densities identified as exhibiting a 1-copy loss.

At block 8050, the method 8000 can include measuring a fraction of the set of cell-free DNA fragments from a brain tumor based on a separation of the copy number of each of the one or more genomic regions from a reference copy number for no aberration.

Measuring the fraction of the set of cell-free DNA fragments from the brain tumor can include comparing the first density to another density to obtain a differential. The differential may be normalized with the reference density and the other density may be the reference density. Measuring the fraction can further include multiplying the differential by two.

The differential can be normalized with the reference density by computing a first ratio of the first density and the reference density and computing a second ratio of the second density and the reference density, where the differential is between the first ratio and the second ratio.

In some embodiments, the separation of the copy number can be compared to a calibration value, which has a corresponding fraction determined from a calibration sample having a known or measured fraction. The known/measured fraction may be determined using a marker (e.g., allele or methylation) that is specific to the brain tumor of the subject from which the calibration sample was obtained.

### B. Differentiation Between Benign and Glioma

We next characterized CNA in CSF cfDNA from patients having benign brain tumors and patients with glioma.

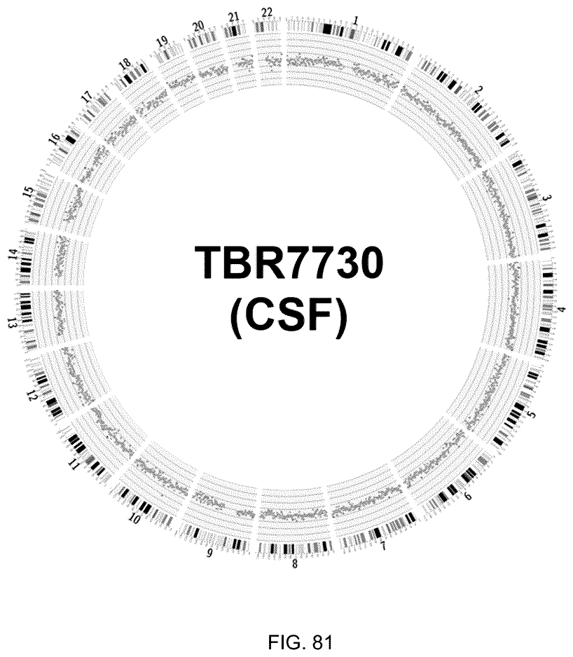

FIG. 81 is a visual representation of copy number alterations in CSF cfDNA from patients having benign brain tumors. Analyses were performed on six patients with benign brain tumors. A representative visualization of CNA analysis is shown for one case. None of the cases, including the case shown, exhibit significant copy number aberrations in plasma cfDNA.

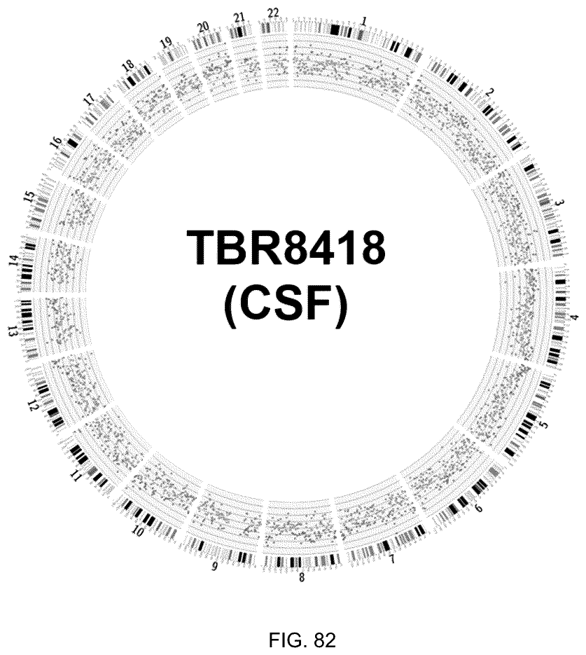

FIG. 82 is a visual representation of copy number alterations in CSF cfDNA from patients having high-grade glioma. Analyses were performed on six patients with high-grade gliomas. A representative visualization of CNA analysis is shown for one case. The six CNA analyses in CSF cfDNA in patients with high-grade gliomas all exhibited genome wide copy loss and gains.

In summary, glioma cases show more significant copy gains and losses compared to plasma. This might suggest that tumor burden and ctDNA release is represented more in CSF compared to plasma.

### 1. Variability Results

As fragmentomic patterns differ between CSF cfDNA of patients with benign brain tumors and high-grade glioma, we explored whether this difference in fragmentation could be linked to variations in genomic coverage. We term this difference in genomic coverage based on fragmentomic difference as ‘fragmentation density’.

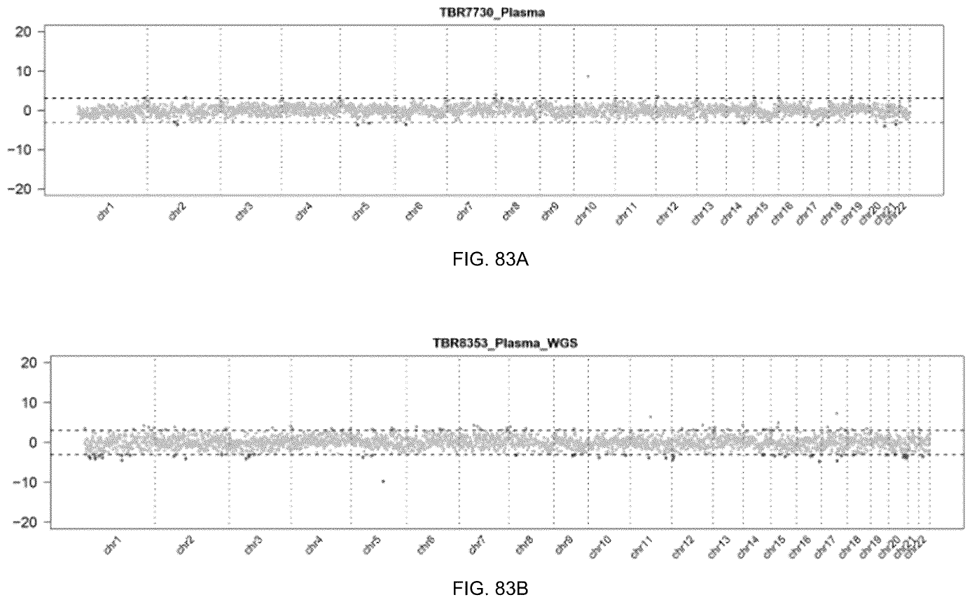

FIGS. 83A and 83B depicts the genomic coverage of cfDNA from plasma for patients with benign or high-grade glioma. Analyses were performed on six patients from each tumor type. A representative visualization of genomic coverage of cfDNA is shown for each tumor type. Each dot represents 1 megabase (Mb) bins of the genome. Bins of higher and lower genomic representations are denoted in green (above the upper horizontal hashed line) and red (below the lower horizontal hashed line), respectively. Genomic coverage is represented as a Z-score. In FIG. 83A, it can be appreciated that very few bins (e.g., ˜14) across the genome exhibited a higher or lower genomic representation in plasma cfDNA of patients with benign brain tumors. It can be appreciated that the relative genomic coverage is very even across the whole genome, with only a few bins exhibiting higher or lower genomic representation in plasma cfDNA of patients with benign brain tumors.

However, FIG. 83B suggests that as compared to benign brain tumors, more bins across the genome exhibited a higher or lower genomic representation in plasma cfDNA of patients with high-grade gliomas.

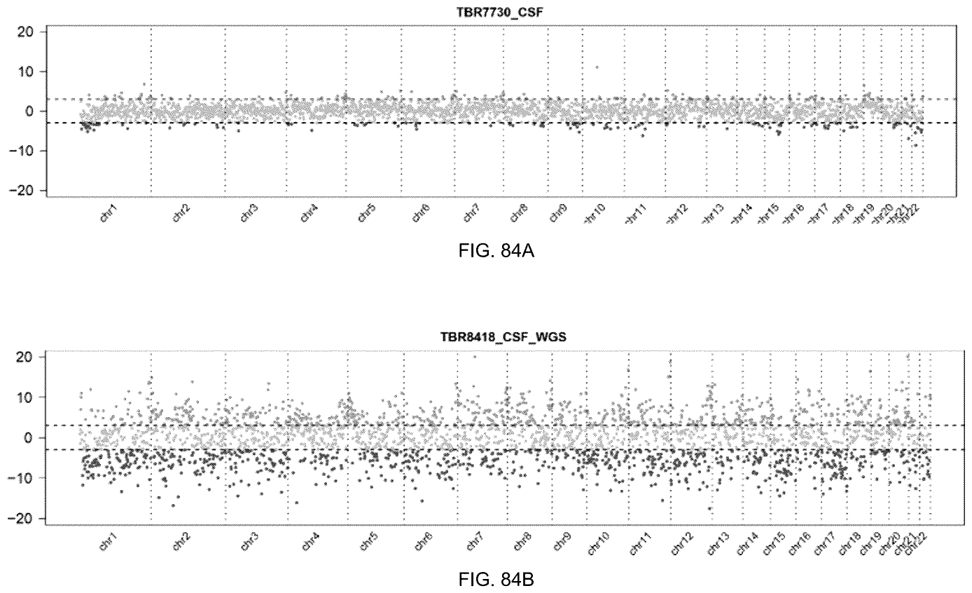

FIGS. 84A and 84B depicts the genomic coverage of cfDNA from CSF for patients with benign or high-grade glioma. Analyses were performed on six patients from each tumor type. A representative visualization of genomic coverage of cfDNA is shown for each tumor type. Each dot represents 1 megabase (Mb) bins of the genome. Bins of higher and lower genomic representations are denoted in green (above the upper horizontal hashed line) and red (below the lower horizontal hashed line), respectively. Genomic coverage is represented as a Z-score. In FIG. 84A it can be appreciated that some bins but still a small portion across the genome exhibited a higher or lower genomic representation in CSF cfDNA of patients with benign brain tumors. However, FIG. 84B many bins across the genome exhibited a higher or lower genomic representation in CSF cfDNA of patients with high-grade gliomas.

Overall, genomic coverage in plasma cfDNA is generally largely even and invariable in plasma cfDNA from patients of benign and high-grade glioma patients. However, cfDNA from CSF show a greater variability in genomic coverage in high-grade glioma patients.

While cfDNA from CSF show a greater variability in high-grade glioma patients. To quantify the variability of genomic coverage, we have analyzed the coefficient of variance (CV) and entropy score (1.0 indicates no variability in genomic coverage, 0 indicates high variability). An entropy score can be calculated as follows:

$$Entropy = - {\sum }_{i = 1}^{n} {P}_{i} * log ( {P}_{i} )$$

where Pᵢ is the proportion of fragments mapped to a particular bin.

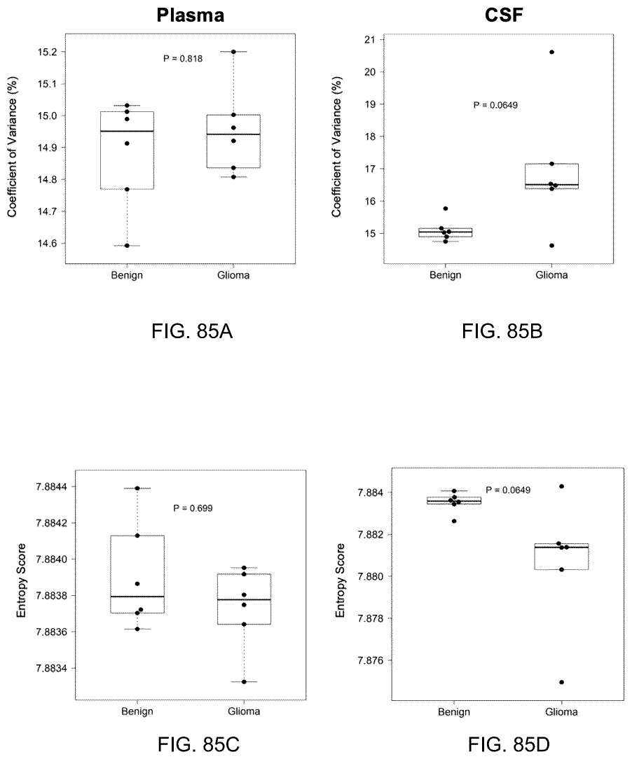

FIGS. 85A and 85B are box plots showing the coefficient of variance (CV) of cfDNA fragmentation density coverage in patients with benign and high-grade glioma, from paired plasma and CSF cfDNA. Analyses were performed on six patients from each tumor type. FIG. 85A shows no significant difference (p=0.818) in the CV of plasma cfDNA fragmentation density coverage between patients with benign brain tumors and patients with gliomas. FIG. 85B shows a significant increase (p=0.0649) in the CV of CSF cfDNA fragmentation density coverage of patients with high-grade gliomas compared to patients with benign brain tumors.

FIGS. 85C and 85D are box plots showing the entropy score of cfDNA fragmentation density coverage in patients with benign and high-grade glioma, from paired plasma and CSF cfDNA. Analyses were performed on six patients from each tumor type. FIG. 85C shows no significant difference (p=0.699) in the entropy score of plasma cfDNA fragmentation density coverage between patients with benign brain tumors and patients with gliomas. FIG. 85B shows a significant decrease (p=0.0649) in the CV of CSF cfDNA fragmentation density coverage of patients with high-grade gliomas compared to patients with benign brain tumors.

CFDNA from CSF overall showed greater variability in genomic coverage in patients with high-grade glioma, which is not reflected in plasma cfDNA. This difference in genomic coverage could be linked to differences in cfDNA fragmentation, resulting in increased or decreased representation of the genome.

### 2. Method

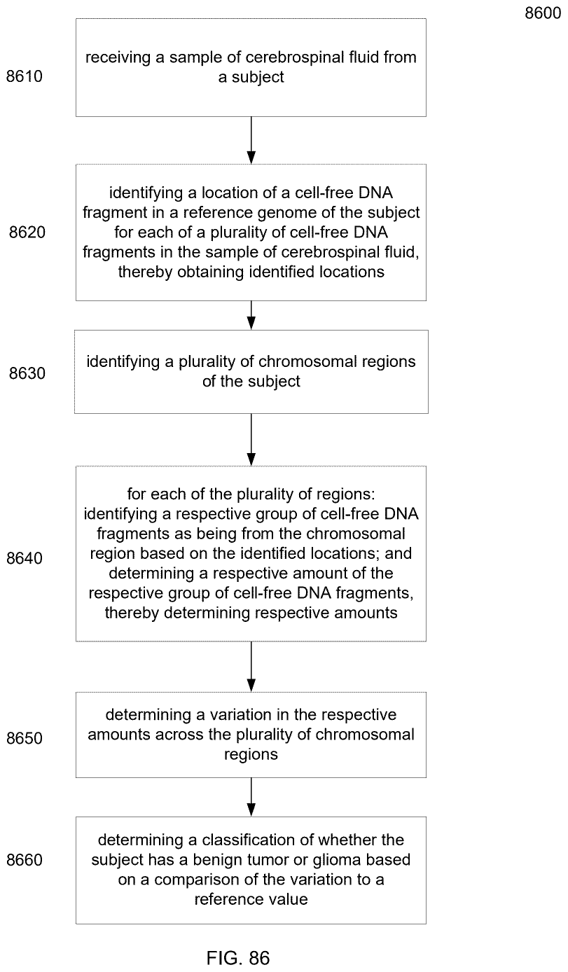

FIG. 86 is a flowchart illustrating a method 8600 for measuring copy number aberrations from a brain tumor, according to some embodiments of the present disclosure. Portions or all steps of method 8600 can be performed by a computer system, including one or more processors. The method 8600 can use a trained ML model that was trained by the computer system or another computer system. The computer system can comprise various devices, e.g., one device that performed the training and another that uses the trained model.

At block 8610, method 8600 can include receiving a sample of cerebrospinal fluid from a subject.

At block 8620, method 8600 can include for each of a plurality of cell-free DNA fragments in the sample of cerebrospinal fluid, identifying a location of the cell-free DNA fragment in a reference genome of the subject, thereby obtaining identified locations. Obtaining the identified locations can include sequencing the plurality of cell-free DNA fragments to obtain sequence reads. The sequence reads can be aligned to the reference genome. As another example, digital PCR can be used, where the particular signal (e.g., a particular color) can indicate a particular location in the genome.

At block 8630, method 8600 can include identifying a plurality of chromosomal regions of the subject. The plurality of chromosomal regions can be non-overlapping or can be overlapping. The plurality of chromosomal regions can cover at least 5%, 10%, 15%, 20%, 25%, 30%, 35%, 40%, 45%, 50%, 55%, 60%, 65%, 70%, 75%, 80%, 85%, 90%, or 95% of a genome of the subject. The plurality of chromosomal regions can be a same size.

At block 8640, method 8600 can include for each of the plurality of regions: identifying a respective group of cell-free DNA fragments as being from the chromosomal region based on the identified locations; and determining a respective amount of the respective group of cell-free DNA fragments, thereby determining respective amounts. The respective amounts can be normalized. The normalization can use a total number of the plurality of cell-free DNA fragments. The normalization can further include a difference from a mean and a scaling by a variation in the normalized amounts across the plurality of chromosomal regions.

At block 8650, method 8600 can include determining a variation in the respective amounts across the plurality of chromosomal regions. The variation can include an entropy term or a root-mean-square deviation (also referred to as a coefficient of variation). The entropy term can include a sum of a proportion of the plurality of cell-free DNA fragments mapped to each of the plurality of chromosomal regions, e.g., as defined above. the sum can be of the proportion multiplied by a log of the proportion.

At block 8660, method 8600 can include determining a classification of whether the subject has a benign tumor or glioma based on a comparison of the variation to a reference value. The reference value can be trained using a first cohort of reference samples from subjects having a benign tumor and a second cohort of reference samples from subjects having glioma.

### VIII. MITOCHONDRIAL DNA

The abundance of mtDNA in plasma cfDNA varies between different cancer types, with a majority showing an increased proportion with cancer and staging (van der Pol et al. 2023). We thus investigated the mtDNA proportion in CSF circulation between patients of benign and high-grade glioma tumors. Various techniques can be used to measure characteristics (e.g., abundance or location in a reference genome) of mtDNA, including sequencing, PCR, or other assays according to some embodiments of the present disclosure.

### A. Difference Between CSF and Plasma

We first assessed the percentage of mtDNA fragments in paired plasma and CSF samples from cases of benign tumors.

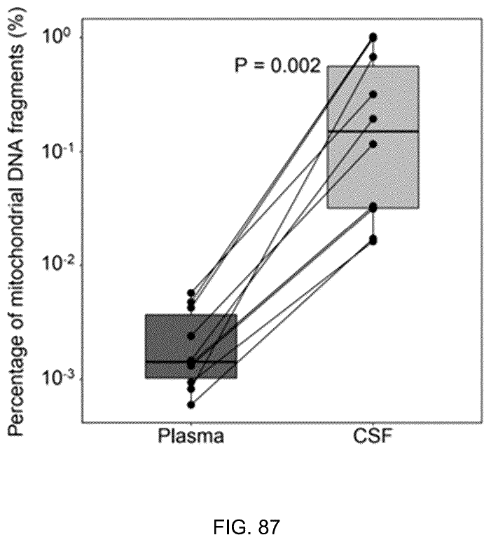

FIG. 87 is a box plot depicting the percentage of mtDNA fragments in paired plasma and CSF samples from cases of benign tumors. Analyses were performed on six patients from each sample type. The percentage of reads mapped to the mitochondrial genome was used for the assessment. The median mitochondrial DNA percentage was 0.0014% in plasma cfDNA and 0.155% in CSF cfDNA. This represents an approximate 109-fold increase in mtDNA in CSF cfDNA compared to plasma cfDNA. These results indicate that CSF cfDNA contains a significantly higher (p=0.002 using paired t-test) proportion of mtDNA (% of reads) compared to paired plasma samples.

We next assessed the mtDNA proportion in dsDNA and ssDNA sequencing libraries in patients with benign brain tumors or high-grade gliomas. We compared the proportion of mtDNA between library types in plasma and CSF cfDNA.

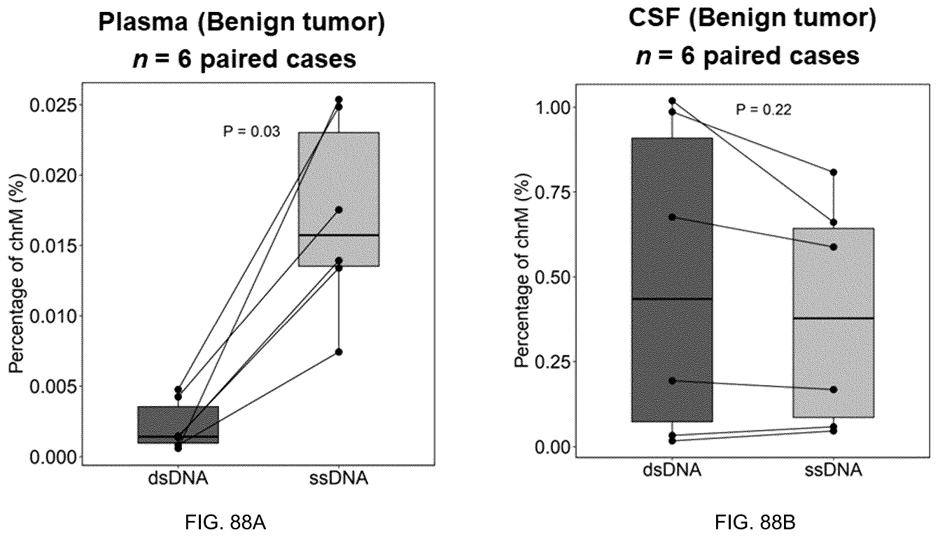

FIGS. 88A and 88B are box plots depicting the mtDNA proportion between dsDNA and ssDNA sequencing libraries from paired plasma or CSF cfDNA from patients with benign brain tumors. Analyses were performed on six patients from each tumor type. The percentage of reads mapped to the mitochondrial genome (also referred to as Chromosome M (chrM)) was used for the assessment. FIG. 88A indicates that ssDNA sequencing libraries have a significant increase (p=0.03 using paired t-test) in the percentage of mtDNA in plasma cfDNA from patients with benign brain tumors. Interestingly, FIG. 88B indicates no difference in the percentage of mtDNA between dsDNA or ssDNA sequencing library preparation methods in CSF cfDNA.

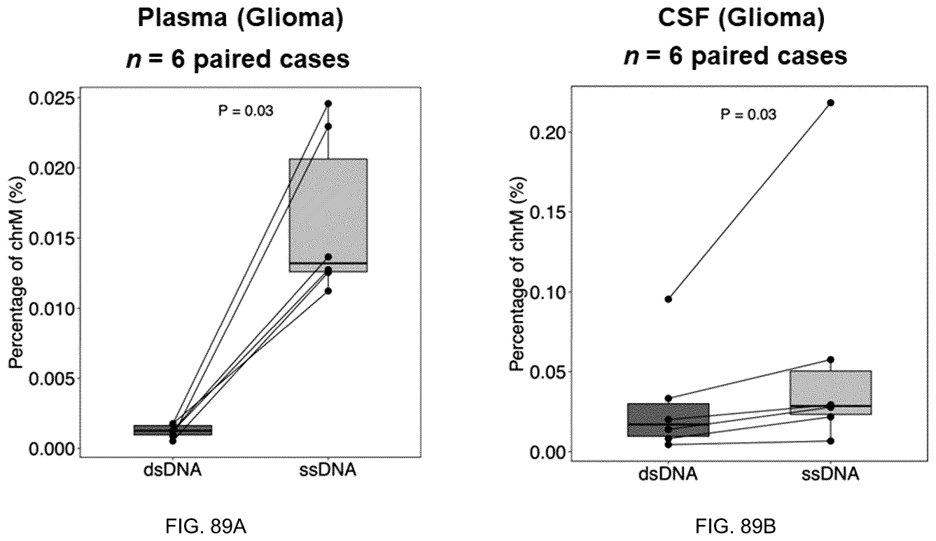

FIGS. 89A and 89B are box plots depicting the mtDNA proportion between dsDNA and ssDNA sequencing libraries from paired plasma or CSF cfDNA from patients with high-grade gliomas. Analyses were performed on six patients from each tumor type. The percentage of reads mapped to the mitochondrial genome (also referred to as Chromosome M (chrM)) was used for the assessment. FIG. 89A indicates that ssDNA sequencing libraries have a significant increase (p=0.03 using paired t-test) in the percentage of mtDNA in plasma cfDNA from patients with benign brain tumors. FIG. 89B also indicates that ssDNA sequencing libraries are enriched for mtDNA compared to dsDNA libraries prepared from CSF cfDNA of patients having high-grade gliomas. This is in contrast to the data from FIG. 88B where no difference was observed in the percentage of mtDNA between dsDNA or ssDNA sequencing library preparation methods in CSF cfDNA.

Combined, these results indicate that plasma cfDNA showed a large enrichment of mtDNA in ssDNA libraries compared to paired dsDNA libraries. This suggests that a large portion of mtDNA in plasma are in single-stranded form. Interestingly, CSF cfDNA shows no significant difference in the mtDNA proportion between dsDNA and ssDNA libraries prepared from benign brain tumors. However, CSF cfDNA is enriched for mtDNA in ssDNA libraries prepared from high-grade gliomas. It is possible that most mtDNA in CSF are in dsDNA form, contrasting to that of plasma.

### B. Differentiation Between Benign and Glioma

To assess whether mtDNA can be used to stage brain tumors, we next examined the mtDNA proportion in dsDNA and ssDNA sequencing libraries in patients with benign brain tumors or high-grade gliomas. We compared the proportion of mtDNA between tumor types in plasma and CSF cfDNA.

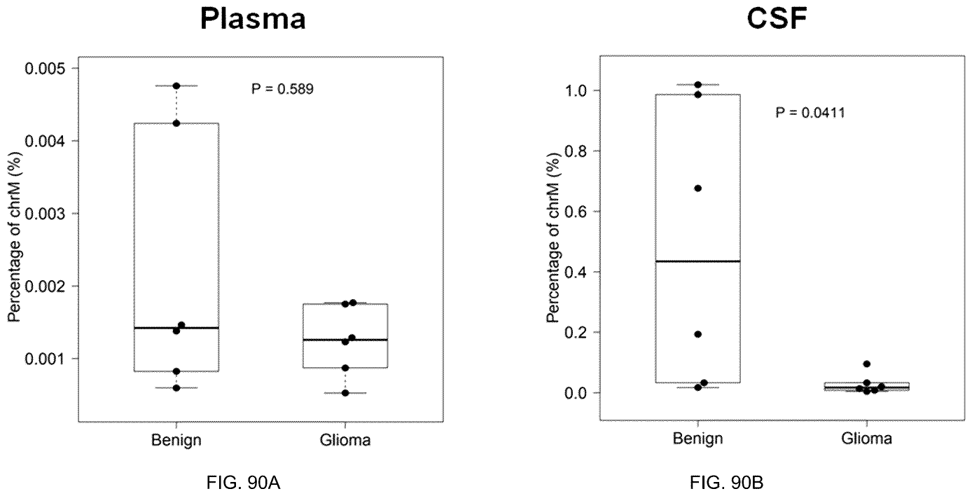

FIGS. 90A and 90B are box plots depicting the percentage of mtDNA fragments in patients with benign and glioma tumors from dsDNA libraries of paired plasma and CSF samples. Analyses were performed on six patients from each tumor type. The percentage of reads mapped to the mitochondrial genome (also referred to as Chromosome M (chrM)) was used for the assessment. FIG. 90A indicates no difference (p=0.0589, unpaired t-test) in the percentage of mtDNA between benign brain tumors and high-grade gliomas prepared using dsDNA libraries from plasma cfDNA. FIG. 90B indicates a significant reduction (p=0.0411, unpaired t-test) in the percentage of mtDNA from high-grade gliomas compared to benign brain tumors for dsDNA libraries from CSF cfDNA.

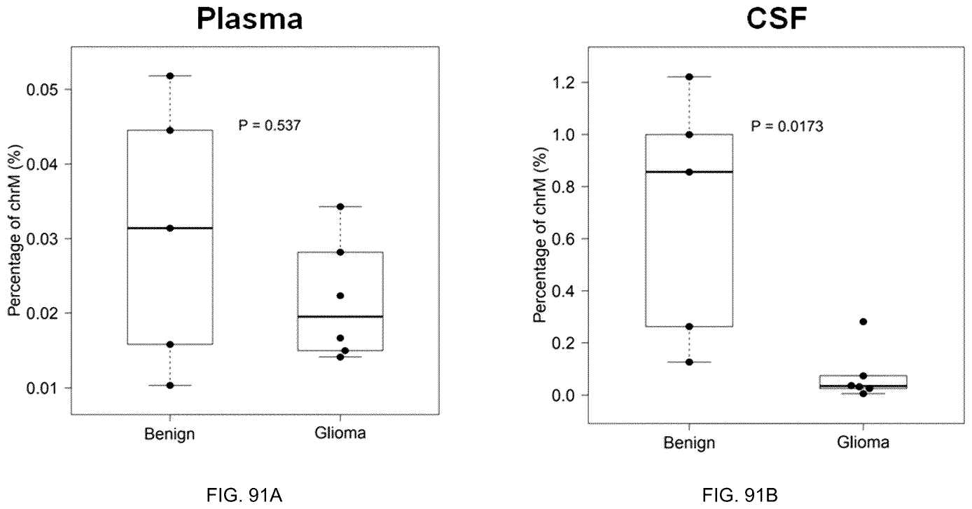

FIGS. 91A and 91B are box plots depicting the percentage of mtDNA fragments in patients with benign and glioma tumors from ssDNA libraries of paired plasma and CSF samples. Analyses were performed on six patients from each tumor type. The percentage of reads mapped to the mitochondrial genome (also referred to as Chromosome M (chrM)) was used for the assessment. FIG. 91A indicates no difference (p=0.537, unpaired t-test) in the percentage of mtDNA between benign brain tumors and high-grade gliomas prepared using ssDNA libraries from plasma cfDNA. FIG. 91B indicates a significant reduction (p=0.0173, unpaired t-test) in the percentage of mtDNA from high-grade gliomas compared to benign brain tumors for ssDNA libraries from CSF cfDNA.

These data suggest that proportion of mtDNA are largely decreased in CSF cfDNA in cases of high-burden GBM tumors, compared to benign tumors. This finding was observed in both dsDNA and ssDNA libraries. This may reflect different modes of DNA release or clearance of mtDNA resulting in differences in mtDNA proportions. These results also indicate that the proportion of mtDNA in CSF could serve as a diagnostic marker in CNS related tumors.

### C. Method

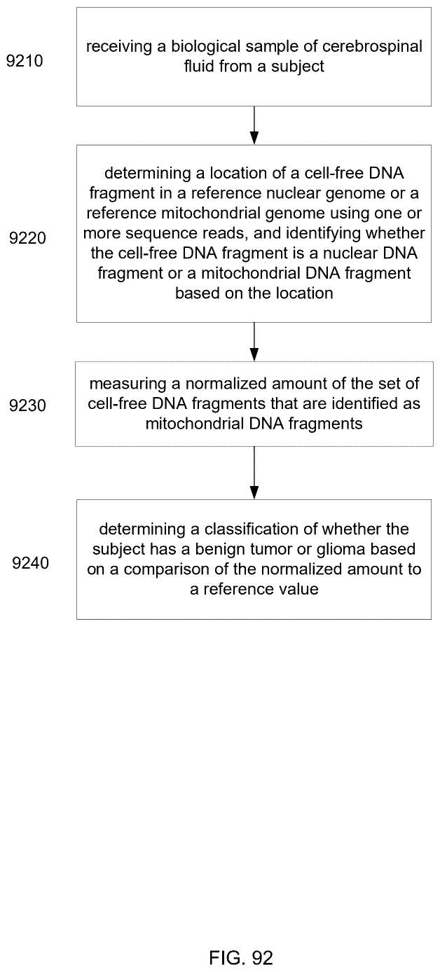

FIG. 92 is a flowchart illustrating a method 9200 for measuring the proportion of mtDNA from a brain tumor to differentiate between brain tumor types, according to some embodiments of the present disclosure. Portions or all steps of method 9200 can be performed by a computer system, including one or more processors. The method 9200 can use a trained ML model that was trained by the computer system or another computer system. The computer system can comprise various devices, e.g., one device that performed the training and another that uses the trained model.

At block 9210, the method 9200 can include receiving a biological sample of cerebrospinal fluid from a subject.

At block 9220, the method 9200 can include for each cell-free DNA fragment of a set of cell-free DNA fragments: determining a location of the cell-free DNA fragment in a reference nuclear genome or a reference mitochondrial genome using one or more sequence reads for the cell-free DNA fragment. In this manner, locations of the set of cell-free DNA fragments can be determined. Given that one can identify a location in a corresponding reference genome, it can be identified whether the cell-free DNA fragment is a nuclear DNA fragment or a mitochondrial DNA fragment based on the location.

Locations of the cell-free DNA fragments can be determined by sequencing a set of cell-free DNA fragments to obtain sequence reads. The sequence reads can be mapped (aligned) to the reference nuclear genome and to the reference mitochondrial genome to determine which sequence reads are located on the reference nuclear genome and which sequence reads are located on the reference mitochondrial genome. The sequencing can be a random sequencing of the set of cell-free DNA fragments from the biological sample. Locations of the cell-free DNA fragments can also be determined using digital PCR.

Determining the locations of the set of cell-free DNA fragments can be performed only for the reference mitochondrial genome, thereby all of the set of cell-free DNA fragments whose location is determined are mitochondrial DNA fragments. For instance, the alignment can only be to the reference mitochondrial genome, and if there is no alignment, then that sequence read (cell-free DNA fragment) can be discarded.

At block 9230, the method 9200 can include measuring a normalized amount of the set of cell-free DNA fragments that are identified as mitochondrial DNA fragments. The normalized amount can be relative to a second amount of the set of cell-free DNA fragments including DNA fragments that can be identified as nuclear DNA fragments. The second amount can also be determined by counting the nuclear DNA fragments.

As one example, the first amount can be measured for the set of cell-free DNA fragments that are identified as mitochondrial DNA fragments. The method can further include measuring a total amount of DNA in the biological sample. The total amount of DNA can be of nuclear DNA fragments and mitochondrial DNA fragments and may be limited to cell-free DNA. The total amount can correspond to the second amount. Measuring the normalized amount can use a ratio of the first amount and the total amount.

The normalized amount of the set of cell-free DNA fragments that are identified as mitochondrial DNA fragments can correspond to a concentration of mitochondrial DNA in the biological sample.

As another example, the normalized amount can be determined as follows. A first amount of the set of cell-free DNA fragments that are mitochondrial DNA fragments can be determined. The second amount of the set of cell-free DNA fragments can be determined by counting the nuclear DNA fragments. A ratio of the first amount and the second amount can be computed. The normalized amount of the set of cell-free DNA fragments that are identified as mitochondrial DNA fragments can be determined using the ratio.

At block 9240, the method 9200 can include determining a classification of whether the subject has a benign tumor or glioma based on a comparison of the normalized amount to a reference value. The reference value can be trained using a first cohort of reference samples from subjects having a benign tumor and a second cohort of reference samples from subjects having glioma. The classification can be that the subject has glioma when the normalized amount is less than the reference value.

### IX. TREATMENTS

Responsive to a classification of a pathology or a fractional concentration of clinically-relevant DNA, various actions might be performed, e.g., physical screening steps or treatment(s).

### A. Further Screening Modalities

Based on any classification, e.g., regarding a pathology or fractional concentration of clinically-relevant DNA, the subject can be referred for additional screening modalities, e.g. using chest X ray, ultrasound, computed tomography, magnetic resonance imaging, or positron emission tomography. Such screening may be performed for cancer.

### B. Treatment Selection

Various embodiments of the present disclosure can accurately predict disease relapse, occurrence, and/or severity thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The treatment of glioma is highly dependent on the stage of diagnosis. Earlier stage gliomas are primarily treated by surgical procedures, termed ‘maximal safe surgical resection’ only. Later stages (stages III to IV, Glioblastoma are usually late stage) require further chemotherapy and/or radiation therapy after surgery.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell therapy, or precision medicine. Based on the determined classification of relapse, a treatment plan can be developed to decrease the risk of harm to the subject and increase overall survival rate. Embodiments may further include treating the subject according to the treatment plan.

### C. Types of Treatments

Various embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment of benign brain tumors and gliomas both rely on craniotomy and surgical resection of the tumor. Most benign CNS tumors can easily be surgically resected if the tumor site is very clearly defined, however gliomas (both low and high-grade) are usually harder to define and integrated within healthy tissues. Both tumor types require craniotomy, but glioma surgery generally requires much more advanced navigation methods such as prior MRI scans and stereotactic methods, or fluorescent guided methods (such as 5-ALA). These approaches are less commonly used for benign tumors. The location of benign tumors also largely governs the type of surgery, some tumors located more within the spinal cord will not require craniotomy, while GBM is always localized in the brain. The treatment of glioma can be highly dependent on the stage of diagnosis. Earlier stage gliomas are primarily treated by surgical procedures, termed ‘maximal safe surgical resection’ only. While later stages (stages III to IV, Glioblastoma are usually late stage) require further chemotherapy and/or radiation therapy after surgery.

After surgical removal of benign CNS tumors, usually no adjuvant therapy is required if tumor is well removed. However, for high-grade gliomas, adjuvant therapies may be required. In some cases, a benign CNS tumors may not be immediately treated if the tumor is small, slow growing, and does not proceed with many symptoms. Gliomas, even at earlier stage, can be treated by surgery to prevent progression.

Treatment may include chemotherapy, which is the use of drugs to destroy cancer cells, usually by keeping the cancer cells from growing and dividing. The drugs may involve, for example but are not limited to, mitomycin-C(available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, temozolomide, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells. Additionally, bevacizumab, an anti-VEGF antibody can be used to treat patients with gliomas.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### X. EXAMPLE SYSTEMS

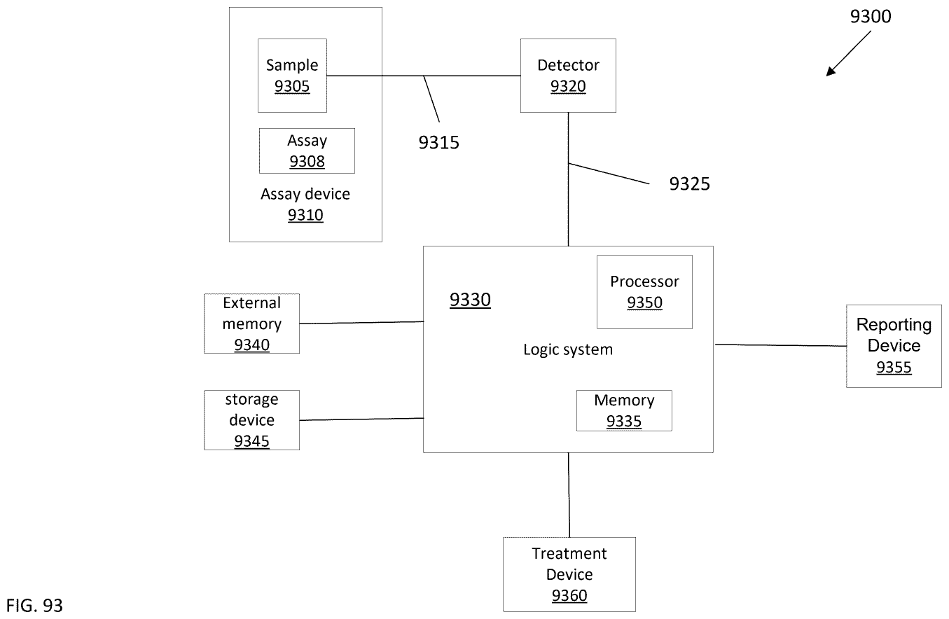

FIG. 93 illustrates a measurement system 9300 according to an embodiment of the present disclosure. The system as shown includes a sample 9305, such as cell-free nucleic acid molecules (e.g., DNA and/or RNA) within an assay device 9310, where an assay 9308 can be performed on sample 9305. For example, sample 9305 can be contacted with reagents of assay 9308 to provide a signal (e.g., an intensity signal) of a physical characteristic 9315 (e.g., sequence information of a cell-free nucleic acid molecule). An example of an assay device can be a flow cell that includes probes and/or primers of an assay or a tube through which a droplet moves (with the droplet including the assay). Physical characteristic 9315 (e.g., a fluorescence intensity, a voltage, or a current), from the sample is detected by detector 9320. Detector 9320 can take a measurement at intervals (e.g., periodic intervals) to obtain data points that make up a data signal. In one embodiment, an analog-to-digital converter converts an analog signal from the detector into digital form at a plurality of times.

Assay device 9310 and detector 9320 can form an assay system, e.g., a PCR system or a sequencing system that performs sequencing according to embodiments described herein. A data signal 9325 is sent from detector 9320 to logic system 9330. As an example, data signal 9325 can be used to determine sequences and/or locations in a reference genome of nucleic acid molecules (e.g., DNA and/or RNA). Data signal 9325 can include various measurements made at a same time, e.g., different colors of fluorescent dyes or different electrical signals for different molecule of sample 9305, and thus data signal 9325 can correspond to multiple signals. Data signal 9325 may be stored in a local memory 9335, an external memory 9340, or a storage device 9345. The assay system can be comprised of multiple assay devices and detectors.

Logic system 9330 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 9330 and the other components may be part of a stand-alone or network connected computer system, or they may be directly attached to or incorporated in a device (e.g., a sequencing device) that includes detector 9320 and/or assay device 9310. Logic system 9330 may also include software that executes in a processor 9350. Logic system 9330 may include a computer readable medium storing instructions for controlling measurement system 9300 to perform any of the methods described herein. For example, logic system 9330 can provide commands to a system that includes assay device 9310 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay. Logic system 9330 can perform any steps of methods described herein that perform computer processing.

Measurement system 9300 may also include a treatment device 9360, which can provide a treatment to the subject. Treatment device 9360 can determine a treatment and/or be used to perform a treatment. Examples of such treatment can include surgery, radiation therapy (e.g., radiosurgery), chemotherapy, immunotherapy, targeted therapy, hormone therapy, and stem cell transplant. Logic system 9330 may be connected to treatment device 9360, e.g., to provide results of a method described herein. The treatment device may receive inputs from other devices, such as an imaging device and user inputs (e.g., to control the treatment, such as controls over a robotic system).

Measurement system 9300 may also include a reporting device 9355, which can present results of any of the methods describe herein, e.g., as determined using the measurement system. Reporting device 9355 can be in communication with a reporting module within logic system 9330 that can aggregate, format, and send a report to reporting device 9355. The reporting module can present information determined using any of the method described herein. The information can be presented by reporting device 9355 in any format that can be recognized and interpreted by a user of the measurement system 9300. For example, the information can be presented by reporting device 9355 in a displayed, printed, or transmitted format, or any combination thereof.

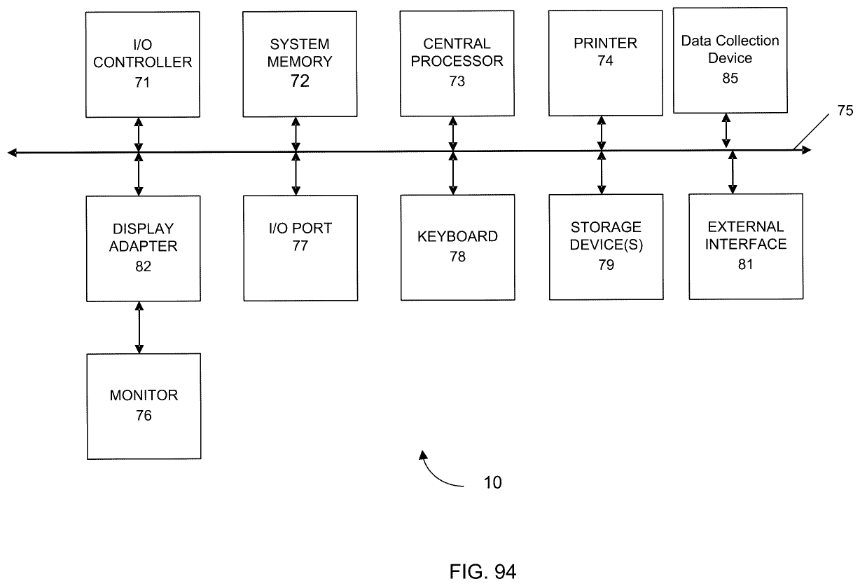

Any of the computer systems mentioned herein may utilize any suitable number of subsystems. Examples of such subsystems are shown in FIG. 94 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 94 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted as prior art. Where a conflict exists between the instant application and a reference provided herein, the instant application shall dominate.

### XI. REFERENCES

Berek K, Bsteh G, Auer M, Di Pauli F, Zinganell A, Berger T, Deisenhammer F, Hegen H. 2021. Cerebrospinal Fluid Findings in 541 Patients With Clinically Isolated Syndrome and Multiple Sclerosis: A Monocentric Study. Front Immunol 12: 675307.
Berzero G, Pieri V, Mortini P, Filippi M, Finocchiaro G. 2023. The coming of age of liquid biopsy in neuro-oncology. Brain 146: 4015-4024.
Bettegowda C, Sausen M, Leary R J, Kinde I, Wang Y, Agrawal N, Bartlett B R, Wang H, Luber B, Alani R M et al. 2014. Detection of circulating tumor DNA in early- and late-stage human malignancies. Sci Transl Med 6: 224ra224.
Choy L Y L, Peng W, Jiang P, Cheng S H, Yu S C Y, Shang H, Olivia Tse O Y, Wong J, Wong V W S, Wong G L H et al. 2022. Single-Molecule Sequencing Enables Long Cell-Free DNA Detection and Direct Methylation Analysis for Cancer Patients. Clin Chem 68: 1151-1163.
Han D S C, Ni M, Chan R W Y, Chan V W H, Lui K O, Chiu R W K, Lo Y M D. 2020. The Biology of Cell-free DNA Fragmentation and the Roles of DNASE1, DNASE1L3, and DFFB. Am J Hum Genet 106: 202-214.
Jiang P, Chan C W, Chan K C, Cheng S H, Wong J, Wong V W, Wong G L, Chan S L, Mok T S, Chan H L et al. 2015. Lengthening and shortening of plasma DNA in hepatocellular carcinoma patients. Proc Natl Acad Sci USA 112: E1317-1325.
Jiang P, Sun K, Peng W, Cheng S H, Ni M, Yeung P C, Heung M M S, Xie T, Shang H, Zhou Z et al. 2020. Plasma DNA End-Motif Profiling as a Fragmentomic Marker in Cancer, Pregnancy, and Transplantation. Cancer Discov 10: 664-673.
Kriaucionis S, Heintz N. 2009. The nuclear DNA base 5-hydroxymethylcytosine is present in Purkinje neurons and the brain. Science 324: 929-930.
Lo Y M, Chan K C, Sun H, Chen E Z, Jiang P, Lun F M, Zheng Y W, Leung T Y, Lau T K, Cantor C R et al. 2010. Maternal plasma DNA sequencing reveals the genome-wide genetic and mutational profile of the fetus. Sci Transl Med 2: 61ra91.
Lo Y M D, Han D S C, Jiang P Y, Chiu R W K. 2021. Epigenetics, fragmentomics, and topology of cell-free DNA in liquid biopsies. Science 372: 144-+.
Mouliere F, Mair R, Chandrananda D, Marass F, Smith C G, Su J, Morris J, Watts C, Brindle K M, Rosenfeld N. 2018. Detection of cell-free DNA fragmentation and copy number alterations in cerebrospinal fluid from glioma patients. EMBO Mol Med 10.
Serpas L, Chan R W Y, Jiang P, Ni M, Sun K, Rashidfarrokhi A, Soni C, Sisirak V, Lee W S, Cheng S H et al. 2019. Dnase113 deletion causes aberrations in length and end-motif frequencies in plasma DNA. Proc Natl Acad Sci USA 116: 641-649.
Sun K, Jiang P, Chan K C, Wong J, Cheng Y K, Liang R H, Chan W K, Ma E S, Chan S L, Cheng S H et al. 2015. Plasma DNA tissue mapping by genome-wide methylation sequencing for noninvasive prenatal, cancer, and transplantation assessments. Proc Natl Acad Sci USA 112: E5503-5512.
Zhao Y, He J Y, Zou Y L, Guo X S, Cui J Z, Guo L, Bu H. 2019. Evaluating the cerebrospinal fluid ctDNA detection by next-generation sequencing in the diagnosis of meningeal Carcinomatosis. BMC Neurol 19: 331.
Zhou Q, Kang G, Jiang P, Qiao R, Lam W K J, Yu S C Y, Ma M L, Ji L, Cheng S H, Gai W et al. 2022. Epigenetic analysis of cell-free DNA by fragmentomic profiling. Proc Natl Acad Sci USA 119: e2209852119.
Zhou Z, Ma M L, Chan R W Y, Lam W K J, Peng W, Gai W, Hu X, Ding S C, Ji L, Zhou Q et al. 2023. Fragmentation landscape of cell-free DNA revealed by deconvolutional analysis of end motifs. Proc Natl Acad Sci USA 120: e2220982120.
Bai Q, He X, Hu T. 2023. Pan-cancer analysis of the deoxyribonuclease gene family. Mol Clin Oncol 18: 19.
Loyfer N, Magenheim J, Peretz A, Cann G, Bredno J, Klochendler A, Fox-Fisher I, Shabi-Porat S, Hecht M, Pelet T et al. 2023. A DNA methylation atlas of normal human cell types. Nature 613: 355-364.
Mattox A K, Douville C, Wang Y, Popoli M, Ptak J, Silliman N, Dobbyn L, Schaefer J, Lu S, Pearlman A H et al. 2023. The Origin of Highly Elevated Cell-Free DNA in Healthy Individuals and Patients with Pancreatic, Colorectal, Lung, or Ovarian Cancer. Cancer Discov 13: 2166-2179.
van der Pol Y, Moldovan N, Ramaker J, Bootsma S, Lenos K J, Vermeulen L, Sandhu S, Bahce I, Pegtel D M, Wong S Q et al. 2023. The landscape of cell-free mitochondrial DNA in liquid biopsy for cancer detection. Genome Biol 24: 229.
van der Pol Y, Mouliere F. 2019. Toward the Early Detection of Cancer by Decoding the Epigenetic and Environmental Fingerprints of Cell-Free DNA. Cancer Cell 36: 350-368.
Zhou Z, Ma M L, Chan R W Y, Lam W K J, Peng W, Gai W, Hu X, Ding S C, Ji L, Zhou Q et al. 2023. Fragmentation landscape of cell-free DNA revealed by deconvolutional analysis of end motifs. Proc Natl Acad Sci USA 120: e2220982120.

1-20. (canceled)

21. A method comprising:
receiving a sample of cerebrospinal fluid from a subject;
sequencing a set of cell-free DNA fragments to obtain sequence reads, wherein the sequence reads include ending sequences corresponding to ends of the set of cell-free DNA fragments;
generating a sample end-motif profile using, for each cell-free DNA fragment of the set of cell-free DNA fragments, an end motif for each of one or more ending sequences of the cell-free DNA fragment, the sample end-motif profile representing one or more end motifs;
comparing the sample end-motif profile to one or more reference end-motif profiles, wherein the one or more reference end-motif profiles include a first reference end-motif profile determined from cell-free DNA fragments in one or more first reference samples of cerebrospinal fluid measured from one or more first reference subjects having a brain tumor; and
detecting a classification of a level of the brain tumor for the subject based on the comparison.

22. The method of claim 21, wherein the sample end-motif profile represents at least four end motifs.

23. The method of claim 21, wherein comparing the sample end-motif profile to the one or more reference end-motif profiles comprises:
comparing a first aggregate for the sample end-motif profile to a second aggregate for the one or more reference end-motif profiles.

24. The method of claim 23, wherein generating the sample end-motif profile comprises generating the first aggregate using the ending sequences matching any one of the one or more end motifs.

25. The method of claim 21, further comprising:
receiving another sample of the subject, wherein the other sample is plasma or serum;
sequencing a plurality of cell-free DNA fragments to obtain other sequence reads; and
generating another end-motif profile using the other sequence reads, the other end-motif profile representing the one or more end motifs in the other sample,
wherein comparing the sample end-motif profile to one or more reference end-motif profiles comprises:
generating a differential end-motif profile between the sample end-motif profile and the other end-motif profile; and
comparing the differential end-motif profile to a reference differential end-motif profile generated using the first reference end-motif profile and another reference end-motif profile.

26. The method of claim 25, wherein the differential end-motif profile comprises a change between the sample of cerebrospinal fluid and the other sample, and wherein the change is compared to a reference change between the first reference end-motif profile and the other reference end-motif profile for the one or more first reference subjects having the brain tumor.

27. The method of claim 21, wherein the sample end-motif profile represents a set of end motifs, and wherein the one or more reference end-motif profiles comprises a set of reference F-profiles, the method further comprising:
storing the set of reference F-profiles, wherein each reference F-profile of the set:
identifies, for each nucleotide of a set of nucleotide, a proportion of cell-free DNA molecules that end in the nucleotide; and
is associated with a type of fragmentation factors,
wherein comparing the sample end-motif profile to the one or more reference end-motif profiles comprises:
determining proportional contributions of the set of reference F-profiles whose proportional aggregation provide the sample end-motif profile, wherein the proportional contributions sum to one, and
wherein detecting the classification of the brain tumor for the subject is based on a proportional contribution associated with a reference F-profile of the set of reference F-profiles.

28. The method of claim 27, wherein the subject is determined to have the brain tumor based on the proportional contribution exceeding a threshold.

29. The method of claim 21, wherein the sample end-motif profile represents a set of end motifs, and wherein comparing the sample end-motif profile to the one or more reference end-motif profiles comprises:
inputting the sample end-motif profile into a machine learning model that is trained using a set of reference size profiles that include the one or more reference end-motif profiles.

30. The method of claim 29, wherein the machine learning model comprises a support vector machine or clustering.

31. The method of claim 27, wherein generating the sample end-motif profile comprises generating, for each end motif of the set of end motifs, an aggregate of the ending sequences having the end motif.

32. The method of claim 21, wherein the one or more reference end-motif profiles further include a second reference end-motif profile determined from cell-free DNA fragments in one or more second reference samples of cerebrospinal fluid measured from one or more second reference subjects having high intra-cranial pressure.

33. The method of claim 21, wherein the one or more end motifs of the sample end-motif profile include pre-end motif(s), EM5 end motif(s), EM3 end motif(s), post-end motif(s), or a combination thereof.

34. The method of claim 21, wherein the classification of the brain tumor is whether the subject has the brain tumor.

35. The method of claim 21, wherein the classification of the brain tumor is whether the brain tumor is benign or glioma, wherein the one or more first reference samples are measured from one or more first reference subjects having a benign tumor or a glioma.

36. The method of claim 35, wherein the one or more first reference samples are measured from a first cohort of reference samples from subjects having a benign tumor and a second cohort of reference samples from subjects having glioma.

37. A method comprising:
receiving a sample of cerebrospinal fluid from a subject;
performing an assay on a set of cell-free DNA fragments to obtain sequence reads, wherein the sequence reads include ending sequences corresponding to ends of the set of cell-free DNA fragments;
for each of the set of cell-free DNA fragments, determining, using the sequence reads, a sequence motif for each of one or more ends of the cell-free DNA fragment, wherein an end of a cell-free DNA fragment has a first position at an outermost position, a second position that is next to the first position, and a third position that is next to the second position;
determining a first amount of a first set of one or more end sequence motifs of the set of cell-free DNA fragments, wherein:
the first set of one or more end sequence motifs have C at the first position and G at the second position, or
the first set of one or more end sequence motifs have C at the second position and G at the third position; and
determining a classification of whether the subject has a benign tumor or glioma based on a comparison of the first amount to a reference value, wherein the reference value is trained using a first cohort of reference samples from subjects having a benign tumor and a second cohort of reference samples from subjects having glioma.

38. The method of claim 37, wherein the first set of one or more end sequence motifs have C at the first position and G at the second position.

39. The method of claim 38, wherein the first set of one or more end sequence motifs includes all end sequence motifs having C at the first position and G at the second position.

40. The method of claim 39, further comprising:
determining a respective amount of each 3-mer end sequence motif that has a C at the first position and that has G at the second position, thereby determining respective amounts;
generating a feature vector including the respective amounts, which include the first amount; and
inputting the feature vector into a machine learning model as part of determining the classification, wherein the machine learning model is trained using the first cohort of reference samples from subjects having the benign tumor and the second cohort of reference samples from subjects having glioma.

41. The method of claim 37, wherein the first set of one or more end sequence motifs have C at the second position and G at the third position.

42. The method of claim 41, wherein the first set of one or more end sequence motifs includes all end sequence motifs having have C at the second position and G at the third position.

43. The method of claim 42, further comprising:
determining a respective amount of each 3-mer end sequence motif that has a C at the second position and G at the third position, thereby determining respective amounts;
generating a feature vector including the respective amounts, which include the first amount; and
inputting the feature vector into a machine learning model as part of determining the classification, wherein the machine learning model is trained using the first cohort of reference samples from subjects having the benign tumor and the second cohort of reference samples from subjects having glioma.

44. The method of claim 37, further comprising:
determining a second amount of a second set of one or more end sequence motifs of the set of cell-free DNA fragments, wherein:
the second set of one or more end sequence motifs have C at the first position and G at the second position, or
the second set of one or more end sequence motifs have C at the second position and G at the third position, wherein the classification is determined using the first amount and the second amount.

45. The method of claim 44, wherein the first set of one or more end sequence motifs have C at the first position and G at the second position, and wherein the second set of one or more end sequence motifs have C at the second position and G at the third position.

46. The method of claim 44, wherein the classification uses a ratio of the first amount and the second amount, uses a difference of the first amount and the second amount, or uses a machine learning model that receives the first amount and the second amount as separate inputs.

47. The method of claim 37, wherein the set of cell-free DNA fragments are each located within one or more regions that are each hypermethylated or hypomethylated for glioma.

48. The method of claim 47, wherein the one or more regions are each hypermethylated.

49. The method of claim 47, wherein the one or more regions are each hypomethylated.

50. The method of claim 49, further comprising:
determining another amount of another set of one or more end sequence motifs of another set of cell-free DNA fragments, wherein the other set of cell-free DNA fragments are each located within one or more regions that are each hypermethylated for glioma,
wherein determining the classification of whether the subject has a benign tumor or glioma further uses the other amount.

51. The method of claim 37, wherein the first set of one or more end sequence motifs includes a plurality of end sequence motifs.

52. The method of claim 37, wherein the assay comprises sequencing or digital PCR.

53. A method comprising:
receiving a sample of cerebrospinal fluid from a subject;
performing a methylation-aware assay on a set of cell-free DNA fragments to obtain sequence reads and to obtain a methylation status of one or more sites for each of the set of cell-free DNA fragments, thereby obtaining methylation statuses of the set of cell-free DNA fragments at a set of sites, wherein the set of cell-free DNA fragments are each located within one or more regions that are each hypermethylated or hypomethylated for glioma;
determining a methylation level using the methylation statuses of the set of cell-free DNA fragments at the set of sites within the one or more regions; and
determining a classification of whether the subject has a benign tumor or glioma based on a comparison of the methylation level to a reference value, wherein the reference value is trained using a first cohort of reference samples from subjects having a benign tumor and a second cohort of reference samples from subjects having glioma.

54. The method of claim 53, wherein the one or more regions are each hypermethylated.

55. The method of claim 54, wherein the classification is that the subject has glioma when the methylation level is greater than the reference value.

56. The method of claim 53, wherein the one or more regions are each hypomethylated.

57. The method of claim 56, wherein the classification is that the subject has glioma when the methylation level is less than the reference value.

58. The method of claim 56, further comprising:
determining another methylation level using the methylation statuses of another set of cell-free DNA fragments at another set of sites within one or more other regions that are each hypermethylated for glioma, wherein determining the classification of whether the subject has a benign tumor or glioma further uses the other methylation level.

59. The method of claim 56, wherein the methylation-aware assay comprises methylation-aware sequencing.

60. The method of claim 59, wherein the methylation-aware sequencing comprises bisulfite sequencing, sequencing after treatment using methylation-sensitive restriction enzymes, or single molecule techniques.

61. The method of claim 53, wherein the methylation level is a methylation density at the set of sites within the one or more regions.

62. The method of claim 53 wherein the one or more regions are a plurality of regions.

63-120. (canceled)
